(19) United States
(12) Patent Application Publication
(10) Pub. No.: US 2026/0201356 A1
(43) Pub. Date: Jul. 16, 2026

(54) CAS12 PROTEIN, CRISPR-CAS SYSTEM AND USES THEREOF

(71) Applicant: GENEDITBIO LIMITED, Hong Kong (CN)

(72) Inventor: Bang Wang, Hong Kong (CN)

(21) Appl. No.: 19/136,412

(22) PCT Filed: Dec. 7, 2023
(86) PCT No.: PCT/IB2023/062353

§ 371 Date: Jun. 6, 2025

(30) Foreign Application Priority Data
Dec. 8, 2022 (WO) PCT/CN2022/137652
Apr. 7, 2023 (WO) PCT/CN2023/087035
May 15, 2023 (WO) PCT/CN2023/094274

(51) Int. Cl. C12N 9/22; C12N 15/11; C12N 15/90

(52) U.S. Cl. CPC C12N 9/226; C12N 15/11; C12N 15/907; C12N 2310/20

(57) ABSTRACT

An engineered, non-naturally occurring Cas12 protein, CRISPR-Cas system and uses thereof are provided. The engineered, non-naturally occurring novel Cas12 proteins comprises an amino acid sequence selected from SEQ ID NOs: 1-13, a homologue thereof having at least 70% sequence identity to the amino acid sequence, or a variant thereof. These Cas12 proteins should enable wider application of CRISPR-Cas systems for gene editing or gene targeting.

### CROSS-REFERENCE TO RELATED APPLICATION

This application claims the priority to PCT application No. PCT/CN2022/137652 filed on Dec. 8, 2022, PCT application No. PCT/CN2023/087035 filed on Apr. 7, 2023, and PCT application No. PCT/CN2023/094274 filed on May 15, 2023. The entire contents of the aforementioned applications are hereby incorporated by reference.

### TECHNICAL FIELD

The present disclosure relates to a Cas12 protein, CRISPR-Cas system and uses thereof. Particularly, the Cas12 protein and CRISPR-Cas system are used for the gene targeting or gene editing.

### BACKGROUND

Various of CRISPR-Cas system had been explored and different CRISPR-Cas systems present different characteristics. Like as CRISPR-Cas12a system, which belongs to the class II of CRISPR-Cas system and is an alternative to the wildly used CRISPR-Cas9. The further studies showed that each subtype of the CRISPR-Cas system itself is also diverse, and some of them are highly controversial in taxonomy. Given the variety and wealth of microbial genomes, it is reasonable countless Cas12 presently have yet to be identified, many of which could exhibit alternate target recognition or enhanced editing efficiency over the commercially available Cas12.

### SUMMARY

There exists a pressing need for alternative Cas12a systems and techniques for gene editing with a wide array of applications. This invention addresses this need and provides related advantages. Mining of new Cas protein will help us to obtain the CRISPR-Cas system with higher gene editing efficiency and/or specificity. Collectively, 13 novel Cas12 proteins are presented and should enable wider application of CRISPR-Cas systems for gene editing and/or gene targeting. The study found that they exhibit some special characteristics. Although phylogenetically more closely related to Cas12a than other subtypes, the tree shows they each have their unique branches, suggesting that they are evolutionarily distinct.

In one aspect, the disclosure provides an engineered, non-naturally occurring Cas12 protein, the Cas12 protein comprises an amino acid sequence selected from SEQ ID NOs: 1-13, a homologue thereof having at least 70% sequence identity to the amino acid sequence, or a variant thereof.

In some embodiments, the Cas12 protein comprises an amino acid sequence having at least 75%, 80%, 85%, 90%, 92%, 95% or 98% sequence identity to any one of SEQ ID NOs: 1-13.

In some embodiments, the Cas12 protein comprises an amino acid sequence having at least 90%, 95% or 98% sequence identity to any one of SEQ ID NOs: 1-13.

In some embodiments, the variant comprises one or more mutations in REC.1 domain, and/or WED.2 domain of any one of SEQ ID Nos: 1-13.

In some embodiments, the variant comprises one or more mutations in REC.1 domain, and/or WED.2 domain of SEQ ID NO: 12.

In some embodiments, the variant comprises one or more mutations in region of 150-200 and/or 513-588 with reference to amino acid position numbering of SEQ ID NO: 12; preferably, the variant comprises one or more mutations in region of 170-190 and/or 520-588 with reference to amino acid position numbering of SEQ ID NO: 12.

In some embodiments, the variant comprises one or more mutations in region of 175-185 and/or 530-588 with reference to amino acid position numbering of SEQ ID NO: 12; preferably, the variant comprises one or more mutations in region of 180-195 and/or 530-588 with reference to amino acid position numbering of SEQ ID NO: 12.

In some embodiments, the variant comprises one or more mutations at the following positions: I182, K532, E535, N536, and/or K586 of SEQ ID NO: 12.

In some embodiments, the variant comprises two or more mutations at the following positions: I182, K532, E535, N536, and/or K586 of SEQ ID NO: 12.

In some embodiments, the variant comprises three or more mutations at the following positions: I182, K532, E535, N536, and/or K586 of SEQ ID NO: 12.

In some embodiments, the variant comprises four or more mutations at the following positions: I182, K532, E535, N536, and/or K586 of SEQ ID NO: 12.

In some embodiments, the variant comprises the mutations at the following positions: I182, K532, E535, N536, and K586 of SEQ ID NO: 12.

In some embodiments, the mutation is a single amino acid substitution.

In some embodiments, the mutation on I182 is I182S or I182T, the mutation on K532 is K532V or K532A, the mutation on E535 is E535N or E535Q, the mutation on N536 is N536R, N536H or N536K, and the mutation on K586 is K586R, K586H or K586K; preferably, the mutation on I182 is I182S, the mutation on K532 is K532V, the mutation on E535 is E535N, the mutation on N536 is N536R, and the mutation on K586 is K586R. In some embodiments, the variant comprises the following mutations: I182S, K532V, E535N, N536R, and K586R of SEQ ID NO: 12.

In some embodiments, PAM region the variant recognized is not a T-rich PAM sequence; optionally, the variant recognizes a PAM sequence which is not a TTTN, where in the “N” represents A, T, G or C; In some embodiments, the variant recognizes a PAM sequence of AATG, ACTG, AGTG, ATTG, CATG, CCTG, CGTG, CTTG, GATG, GCTG, GGTG, GTTG, TATG, TCTG or TGTG. In some embodiments, the variant has a higher preference for recognizing the PAM sequence of AATG, AGTG, ATTG, CATG, CGTG, GATG, GCTG, GGTG, GTTG, TATG or TGTG compared to the wild-type sequence; preferably, the variant has a higher preference for recognizing the PAM sequence of GATG compared to the wild-type sequence. In some embodiments, the variant recognizes a PAM sequence which is not recognized by SEQ ID NO: 12.

In some embodiments, the variant has nuclease activity; In some embodiments, the variant has the double-strand DNA cleavage activity or nickase activity.

In some embodiments, the Cas12 protein further comprises one or more of a nuclear localization signal sequence, a nuclear export signal sequence, a cell penetrating peptide sequence, an affinity tag and/or a fusion base editor protein. In some embodiments, the Cas12 protein comprises an amino acid sequence having at least 70%, 75%, 80%, 85%, 90%, 92%, 95%, 96%, 97%, 98%, 99% or 100% sequence identity to any one of SEQ ID NOs: 34-46, 153-155.

In another aspect, this disclosure provides an engineered, non-naturally occurring Cas12 polynucleotide encoding the Cas12 protein as described herein above.

In some embodiments, the polynucleotide is ribonucleotide sequence or deoxyribonucleotide sequence, or analogs thereof; preferably the polynucleotide is mRNA, and the polynucleotide further comprises 5′cap sequence and poly-A tail sequence. In some embodiments, the polynucleotide has at least 70% sequence identity to any one of the SEQ ID NOs: 14-26. In some embodiments, the polynucleotide has at least 75%, 80%, 85%, 88%, 90%, 92%, 94%, 95%, 96%, 98% or 99% sequence identity to any one of the SEQ ID NOs: 14-26. In some embodiments, the polynucleotide is codon optimized for expression in a cell of interest. In some embodiments, the polynucleotide is codon optimized for expression in a eukaryotic cell. In some embodiments, the polynucleotide has at least 90%, 92%, 95% or 98% sequence identity to any one of SEQ ID NOs: 91-94. In some embodiments, the eukaryotic cell is selected from the group consisting of: a plant cell, a fungal cell, a single cell eukaryotic organism, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and a human cell. In some embodiments, the cell is a mammalian cell, preferably a human cell.

In another aspect, the disclosure provides the engineered, non-naturally occurring Cas12 protein as described herein above, or the Cas12 polynucleotide as described herein above for use as nuclease, preferably, for use as double-strand DNA cleavage nuclease or nickase.

In another aspect, the disclosure provides the engineered, non-naturally occurring Cas12 protein as described herein above, or the Cas12 polynucleotide as described herein above for use in the gene editing.

In another aspect, the disclosure provides the engineered, non-naturally occurring Cas12 protein as described herein above, or the Cas12 polynucleotide as described herein above for use in a therapeutic or treatment or prevention or diagnosis or detection method of disease.

In another aspect, the disclosure provides the engineered, non-naturally occurring Cas12 protein as described herein above, or the Cas12 polynucleotide as described herein above for use as a medicament.

In another aspect, the disclosure provides the engineered, non-naturally occurring Cas12 protein for use in a method of therapeutic treatment of a patient.

In another aspect, the disclosure provides an engineered, non-naturally occurring cell comprising the Cas12 protein of any one of above.

In some embodiments, the cell is a eukaryotic cell or a prokaryotic cell. In some embodiments, the eukaryotic cell is selected from the group consisting of: a plant cell, a fungal cell, a single cell eukaryotic organism, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and a human cell. In some embodiments, the cell is a mammalian cell or a human cell or a plant cell.

In another aspect, the disclosure provides a kit comprising the Cas12 protein of any one of described above.

In another aspect, the disclosure provides an engineered vector comprising the Cas12 polynucleotide of any one of described above.

In some embodiments, the vector is an expression vector. In some embodiments, the vector is an inducible, conditional, or constitutive expression vector.

In another aspect, the disclosure provides a vector system comprising one or more vectors of any one of described above. In some embodiments, the one or more vectors comprise a polynucleotide according to any one of above and one or more polynucleotides which are on a same or on different vectors encoding a guide RNA.

In another aspect, the disclosure provides an engineered cell comprising the Cas12 polynucleotide of any one of above, or comprising the vector of any one of above, or comprising the vector system of any one of above.

In some embodiments, the cell is expressing the Cas12 protein. In some embodiments, the cell transiently expresses or non-transiently expresses the Cas12 protein. In some embodiments, the cell is a eukaryotic cell or a prokaryotic cell. In some embodiments, the cell is a mammalian cell or a human cell or a plant cell.

In another aspect, the disclosure provides a reagent kit comprising the Cas12 protein of any one of above, the Cas12 polynucleotide of any one of above, the vector of any one of above, or the vector system of any one of above.

In another aspect, the disclosure provides a pharmaceutical composition comprising the Cas12 protein of any one of above or the polynucleotide of any one of above or the vector of any one of above or the vector system of any one of above formulated for delivery by AAV (adena-associated viruses), Adenoviruses, retroviruses, HSV (herpes simplex virus), Gammaretrovirus, LV (lentivirus), eCIS (extracellular Contractile Injection System), eVLPs (Engineered virus-like particles), VLP (virus-like particles), liposomes, plasmid, LNPs (lipid nanoparticles), exosomes, microvesicles, nucleic acid nanoassemblies, a gene gun, and/or an implantable device.

In another aspect, the disclosure provides an engineered, non-naturally occurring CRISPR-Cas system comprising: a) the Cas12 protein of any one of above or the polynucleotide encoding the Cas12 protein; b) at least one engineered guide sequence or one or more engineered nucleic acid encoding the at least one engineered guide sequence, and the guide sequence comprises a direct repeat sequence capable of binding the Cas12 protein and a spacer sequence capable of hybridizing to a target sequence.

In some embodiments, the system comprises at least one guide sequences which are capable of hybridizing at least one target sequences or different regions of one target sequence. In some embodiments, the guide sequence hybridizes to one or more target sequences in a prokaryotic cell or in a eukaryotic cell. In some embodiments, the eukaryotic cell is selected from the group consisting of: a plant cell, a fungal cell, a single cell eukaryotic organism, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and a human cell. In some embodiments, the eukaryotic cell comprises a mammalian cell. In some embodiments, the mammalian cell comprises a human cell. In some embodiments, the eukaryotic cell comprises a plant cell.

In some embodiments, the target sequence is DNA or RNA. In some embodiments, the target sequence is selected from: double stranded DNA, double stranded RNA, single stranded DNA, single stranded RNA, genomic DNA, or extrachromosomal DNA.

In some embodiments, the direct repeat sequence comprises a stem-loop structure and the direct repeat sequence comprises a nucleotide sequence having at least 95% identity to any one of SEQ ID NOs: 27-33. In some embodiments, the direct repeat sequence comprises a nucleotide sequence having at least 90% identity to any one of SEQ ID NOs: 27-33. In some embodiments, the direct repeat sequence comprises a nucleotide sequence set forth in any one of SEQ ID NOs: 27-33.

In some embodiments, the spacer sequence is between 18 and 23 nucleotides in length, preferably the spacer sequence is 19 or 23 nucleotides in length. In some embodiments, the spacer sequence comprises a sequence having at least 95%, 99% or 100% identity to any one of SEQ ID NOs: 81-89, 95-136.

In some embodiments, the polynucleotide encoding the Cas12 protein is an mRNA or a DNA. In some embodiments, the polynucleotide encoding the Cas12 protein is operably linked to a promoter. In some embodiments, the promoter is a constitutive promoter, tissue-specific promoter or inducible promoter. In some embodiments, the polynucleotide encoding the Cas12 protein operably linked to a promoter is in a vector.

In some embodiments, the vector is selected from the group consisting of a retroviral vector, a lentiviral vector, a phage vector, an adenoviral vector, an adeno-associated vector, and a herpes simplex vector.

In some embodiments, the system further comprising a donor template nucleic acid, the donor template nucleic acid is a DNA or RNA or DNA-RNA hybrids.

In some embodiments, the targeting of the target sequence by the Cas12 protein and guide sequence results in a modification of the target sequence. In some embodiments, the modification of the target sequence is a cleavage event or a nicking event.

In another aspect, the disclosure provides a delivery system, wherein the system of any one of above is presented in the vehicle selected from the group consisting of AAV (adena-associated viruses), Adenoviruses, retroviruses, HSV (herpes simplex virus), Gammaretrovirus, LV (lentivirus), eCIS (extracellular Contractile Injection System), eVLP (Engineered virus-like particles), VLP (virus-like particles), liposomes, plasmid, LNPs (lipid nanoparticles), exosomes, microvesicles, nucleic acid nanoassemblies, a gene gun, and/or an implantable device.

In another aspect, the disclosure provides an engineered cell comprising the system of any one of above. In some embodiments, the cell is a eukaryotic cell or a prokaryotic cell. In some embodiments, the eukaryotic cell is selected from the group consisting of: a plant cell, a fungal cell, a single cell eukaryotic organism, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and a human cell. In some embodiments, the cell is a mammalian cell or a human cell or a plant cell.

In another aspect, the disclosure provides the engineered, non-naturally occurring CRISPR-Cas system of any one of above, or the delivery system of above for use in a therapeutic or treatment or prevention or diagnosis or detection method of disease.

In another aspect, the disclosure provides the engineered, non-naturally occurring CRISPR-Cas system of any one of above, delivery system of above or cell of any one of above for use as a medicament.

In another aspect, the disclosure provides the engineered, non-naturally occurring CRISPR-Cas system of any one of above, delivery system of above or cell of any one of above for use in a method of therapeutic treatment of a patient.

In another aspect, the disclosure provides a method of modifying or targeting a target DNA locus, the method comprising delivering to said locus a CRISPR-Cas system of any one of above or a delivery system of above. In some embodiments, said modifying or targeting a target locus comprises inducing a DNA strand break. In some embodiments, said modifying or targeting a target locus comprises inducing a DNA double strand break. In some embodiments, said modifying or targeting a target locus comprises altering gene expression of one or more genes. In some embodiments, said modifying or targeting a target locus comprises epigenetic modification of said target DNA locus. In some embodiments, the method is a method of modifying a cell, a cell line, or an organism by manipulation of one or more target sequences at genomic loci of interest. In some embodiments, the cell is a eukaryotic cell or a prokaryotic cell. In some embodiments, the cell is a mammalian cell or a human cell or a plant cell. In some embodiments, the method is in vitro or in vivo.

In another aspect, the disclosure provides a method of targeting and cleaving a double-stranded target DNA, the method comprising contacting the double-stranded target DNA with the system of any one of described above.

In some embodiments, cleaving the target DNA or target sequence results in the formation of an indel or the insertion of a nucleotide sequence. In some embodiments, cleaving the target DNA or target nucleotide comprising cleaving the target DNA or target sequence in two sites, and results in the deletion or inversion of a sequence between the two sites.

In another aspect, the disclosure provides an isolated eukaryotic cell comprising a modified target locus of interest, wherein the target locus of interest has been modified according to a method or via use of a composition or via use of a system of any one of the preceding contents.

In another aspect, the disclosure provides a system for detecting the presence of a nucleic acid target sequence in an in vitro sample, comprising: a) a Cas12 protein of any one of above; b) at least one guide polynucleotide comprising a guide sequence capable of binding the target sequence, and designed to form a complex with the Cas12 protein; and c) a nucleic acid-based masking construct comprising a non-target sequence; and wherein the Cas12 protein exhibits collateral cleavage activity of RNA and/or ssDNA and cleaves the non-target sequence of the nucleic acid-based masking construct activated by the target sequence.

In another aspect, the disclosure provides a method for detecting target nucleic acids in samples comprising: a) contacting one or more samples with a Cas12 protein of any one of above; b) at least one guide polynucleotide comprising a guide sequence designed to have a degree of complementarity with the target sequence, and designed to form a complex with the Cas12 protein; and c) a nucleic acid-based masking construct comprising a non-target sequence, wherein the Cas12 protein exhibits collateral cleavage activity of RNA and/or ssDNA and cleaves the non-target sequence of the nucleic acid-based masking construct activated by the target sequences; and detecting a signal from cleavage of the non-target sequence, thereby detecting the one or more target sequences in the sample.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present disclosure will be obtained by reference to the following detailed description that sets forth illustrative embodiments, in which the principles of the disclosure may be utilized, and the accompanying drawings of which:

FIG. 1 (FIG. 1) shows the phylogenetic tree of the GEB×Cas12 effectors in this disclosure constructed by IQTREE.

FIG. 2 (FIG. 2) shows the percent identity matrix between GEB×Cas12 effectors and the referenced Cas12a (Cpf1).

FIG. 3 (FIG. 3) shows the domains arrangement of the GEB×Cas12 effectors in this disclosure.

FIG. 4 (FIG. 4) shows the secondary structure of the crRNA utilized by GEB×Cas12 effectors in this disclosure.

FIG. 5 (FIG. 5) shows the sequences alignment between TnpB, Cas12f and GEB×0160~0174 in this disclosure; the region of Zinc finger domain and the conserved 4-Cys Zinc finger in Cas12f and TnpB were marked with arrow and star respectively, indicated that GEB×0160~0174 doesn't have the zinc finger structure in their C terminus.

FIG. 6 (FIG. 6) shows the PAM preference of the wild type GEB×0173 in HEK293 cell line.

FIG. 7 (FIG. 7) shows on-target and off-target editing in HEK293T that were targeted 5854 (target1) and Humspacer3(target2).

FIG. 8 (FIG. 8) shows the effect of spacer length on GEB×0173.

FIG. 9 (FIG. 9) shows the schematic of pCasX-gRNA plasmid harbored with the NLS, Cas nucleases CDS and guide RNA, wherein the Cas nucleases CDS is driven under a CMV promoter while the gRNA is driven under a U6 promoter.

FIG. 10 (FIG. 10) shows the indel activity of human HEK293T cells following reverse transfection of pGEB×0173-gRNA plasmid harbored with GEB×0173 CDS and MYOD1 targeted crRNA.

FIG. 11 (FIG. 11) shows the indel activity of GEB×0173 across 23 targets with TTTG-PAM in HEK293T cell line.

FIG. 12 (FIG. 12) shows the comparison of GEB×0173, AsCpf1 and SpCas9 editing efficiency across 5 targets in HEK293T cell line.

FIG. 13 (FIG. 13) shows the editing efficiency of GEB×0173 following transfection of HEK293T cell with lipoplex comprising a fixed amount (30 ng) of crRNA (MYOD1-TTTG-HM7, SEQ ID NO: 147) and different ratios of GEB×0173 mRNA.

FIG. 14 (FIG. 14) shows the editing efficiency of GEB×0173 following transfection of PHH cell with lipoplex comprising a fixed amount (30 ng) of crRNA (MYOD1-TTTG-HM7, SEQ ID NO: 147) and different ratios of GEB×0173 mRNA.

FIG. 15 (FIG. 15) shows the site of 5 mutant residues of GEB×0173-variant, which all located around the putative PAM binding region.

FIG. 16 (FIG. 16) shows the PAM preference of the GEB×0173-variant in HEK293 cell line.

FIG. 17 (FIG. 17) shows the Luciferase reporter assay result of GEB×0173-wt and GEB×0173-v1 variant on NNTG PAM.

FIG. 18 (FIG. 18) shows the indel activity of GEB×0173-v1 across 20 targets with GATG-PAM in HEK293T cell line.

FIG. 19 (FIG. 19) summary of top Guide-seq insertion sites, shows no detectable off-targets at EXM1-TTTG-T1 and TTR-TTTG-T2 sites when using GEB×0173.

### DETAILED DESCRIPTION OF THE PREFERRED EMBODIMENT

The following examples further illustrate the present disclosure, but the present disclosure is not limited thereto.

### General Definitions

Unless defined otherwise, the technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), the Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y 1994), March, Advanced Organic Chemistry Reactions, Mechanisms and Structure 4th ed., John Wiley & Sons (New York, N.Y 1992); and Marten H. Hofker and Jan van Deursen, Transgenic Mouse Methods and Protocols, 2nd edition (2011).

As used herein, the term “a”, “an”, “the”, and “said” and similar terms used in the context of the present disclosure (especially in the context of the claims) are to be construed to cover both the singular and plural unless otherwise indicated herein or clearly contradicted by the context. In addition, it should be noted that the plural form does not necessarily mean that it is plural, and it needs to be understood according to the context in the article.

The term “identity” in the context of two or more nucleic acids or polypeptide sequences refers to two or more sequences or subsequences that are the same or have a specified percentage of amino acid residues or nucleotides that are the same as measured using a BLAST or BLAST 2.0 or FASTA etc. sequence comparison algorithms with default parameters described below.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and those terms such as “consisting essentially of” and “consists essentially of” have the meaning ascribed to them in U.S. Patent law.

As used herein, the term “mutant”, “variant”, “modification”, and similar terms used in the context of the present invention (especially in the context of the claims) are to be construed to the same mean unless otherwise indicated herein or clearly contradicted by the context.

As used herein, the terms “recognized”, “recognizing”, or “recognition” in this context refers to the capability of the Cas12 protein to form a functional complex with a guide RNA at a DNA target site to which the guide RNA hybridizes (i.e. to which the guide sequence of the guide RNA hybridizes) and being flanked by the PAM sequence, and wherein the Cas12 protein is capable of performing its natural function, i.e. DNA cleavage. In this context it is to be noted that such DNA cleavage precludes the Cas12 protein from being a catalytically inactive Cas12 protein. In the case of for instance an inactivated Cas12 protein (e.g., a dead Cas12 protein), a complex between the Cas12 protein, guide RNA and cognate target may nevertheless be formed if the required PAM sequence is present, but such does not result in DNA cleavage.

The term “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about”, “~” as used herein when referring to a measurable value such as a parameter, an amount, a temporal duration, and the like, are meant to encompass variations of and from the specified value, such as variations of +/−10% or less, +/−5% or less, +/−2% or less, +/−1% or less, and +/−0.1% or less of and from the specified value, insofar such variations are appropriate to perform in the disclosed disclosure. It is to be understood that the value to which the modifier “about” or “~” refers is itself also specifically, and preferably, disclosed.

The term “exemplary” is used herein to mean serving as an example, instance, or illustration. Any aspect or design described herein as “exemplary” is not necessarily to be construed as preferred or advantageous over other aspects, embodiments, or designs.

As used herein, a “sample” may contain whole cells and/or live cells and/or cell debris. The sample may contain (or be derived from) a “bodily fluid”. The present disclosure encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Samples include cell cultures, bodily fluids, cell cultures from bodily fluids. Bodily fluids may be obtained from a mammal organism, for example by puncture, or other collecting or sampling procedures.

The terms “subject”, “individual” and “patient” are used interchangeably herein to refer to a vertebrate, preferably a mammal, more preferably a human. Mammals include, but are not limited to murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “in a specific embodiment”, “in some embodiment”, “in certain embodiment” means that a particular feature, structure or characteristic described in connection with the embodiment is included in at least one embodiment of the present disclosure. Thus, appearances of the phrases “a specific embodiment”, “in one embodiment” or “in certain embodiment” in various places throughout this specification are not necessarily all referring to the same embodiment, but may. Furthermore, a particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure. For example, in the appended claims, any one of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

The term “gene” refers to a nucleic acid sequence (used interchangeably with polynucleotide or nucleotide sequence) that encodes a chimeric molecule as described herein. This definition includes various sequence polymorphisms, mutations, and/or sequence variants wherein such alterations do not substantially affect the function of the encoded chimeric molecule. The term “gene” may include not only coding sequences but also regulatory regions such as promoters, enhancers, and termination regions. The term further can include all introns and other DNA sequences spliced from the mRNA transcript, along with variants resulting from alternative splice sites. Gene sequences encoding the molecule can be DNA or RNA that directs the expression of the chimeric molecule. These nucleic acid sequences may be a DNA strand sequence that is transcribed into RNA or an RNA sequence that is translated into protein. The nucleic acid sequences include both the full-length nucleic acid sequences as well as non-full-length sequences derived from the full-length protein. The sequences can also include degenerate codons of the native sequence or sequences that may be introduced to provide codon preference in a specific cell type. Portions of complete gene sequences are referenced throughout the disclosure as is understood by one of ordinary skill in the art.

“Encoding” refers to the property of specific sequences of nucleotides in a gene, such as a cDNA, or an mRNA, to serve as templates for synthesis of other macromolecules such as defined sequences of amino acids. Thus, a gene codes for a protein if transcription and translation of mRNA corresponding to that gene produces the protein in a cell or other biological system. A polynucleotide encoding a protein includes all nucleotide sequences that are degenerate versions of each other and that code for the same amino acid sequence or amino acid sequences of substantially similar form and function.

The terms “polynucleotide”, “nucleotide”, “nucleotide sequence”, “nucleic acid”, “nucleic acid molecule” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

Polynucleotide sequences encoding more than one portion of an expressed chimeric molecule can be operably linked to each other and relevant regulatory sequences. For example, there can be a functional linkage between a regulatory sequence and an exogenous nucleic acid sequence resulting in expression of the latter. For another example, a first nucleic acid sequence can be operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary or helpful, join coding regions, into the same reading frame.

“Homologue” of a protein as used herein is a protein of the same species which perform the same or a similar function as the protein it is a homologue of Homologous proteins may but need not be structurally related, or are only partially structurally related. “Homologue” of a protein as used herein also include sequences having one or more additions, deletions, stop positions, or substitutions, as compared to a sequence disclosed herein. The Homologue protein as used herein perform the same or a similar function as the Cas12 protein disclosed herein.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature. In all aspects and embodiments, whether they include these terms or not, it will be understood that, preferably, may be optional and thus preferably included or not preferably included. Furthermore, the terms “non-naturally occurring” and “engineered” may be used interchangeably and so can therefore be used alone or in combination and one or other may replace mention of both together. In particular, “engineered” is preferred in place of “non-naturally occurring” or “non-naturally occurring and/or engineered” or “engineered, non-naturally occurring”.

The term “cleavage event” as used herein, refers to a DNA break in a target sequence created by a nuclease of a CRISPR system described herein. In some embodiments, the cleavage event is a double-stranded DNA break. In some embodiments, the cleavage event is a single-stranded DNA break.

A “stem-loop structure” refers to a nucleic acid having a secondary structure that includes a region of nucleotides that are known or predicted to form a double strand (stem portion) that is linked on one side by a region of predominantly single-stranded nucleotides (loop portion). The terms “hairpin” and “fold-back” structures are also used herein to refer to stem-loop structures. Such structures are well known in the art and these terms are used consistently with their known meanings in the art. As is known in the art, a stem-loop structure does not require exact base-pairing. Thus, the stem may include one or more base mismatches. Alternatively, the base-pairing may be exact, i.e., not include any mismatches.

The term “donor template nucleic acid” as used herein refers to a nucleic acid molecule that can be used by one or more cellular proteins to alter the structure of a target sequence after a CRISPR enzyme described herein has altered a target nucleic acid. In some embodiments, the donor template nucleic acid is a double-stranded nucleic acid. In some embodiments, the donor template nucleic acid is a single-stranded nucleic acid. In some embodiments, the donor template nucleic acid is linear. In some embodiments, the donor template nucleic acid is circular (e.g., a plasmid). In some embodiments, the donor template nucleic acid is an exogenous nucleic acid molecule. In some embodiments, the donor template nucleic acid is an endogenous nucleic acid molecule (e.g., a chromosome).

As used herein, the term “targeting” refers to the ability of a complex including a CRISPR-associated protein and an RNA guide, to preferentially or specifically bind to, e.g., hybridize to, a specific target sequence compared to other nucleic acids that do not have the same or similar sequence as the target nucleic acid.

As used herein, the term “target sequence” refers to a specific nucleic acid substrate that contains a nucleic acid sequence complement to the entirety or a part of the spacer in an RNA guide. In some embodiments, the target sequence comprises a gene or a sequence within a gene. In certain embodiments, the target sequence comprises a noncoding region (e.g., a promoter). In a specific embodiment, the target sequence is single-stranded. In a specific embodiment, the target sequence is double-stranded.

It will be appreciated that the terms Cas12 enzyme, Cas12 protein, Cas12 effector protein and Cas2 are generally used interchangeably and at all points of reference herein refer by analogy to novel CRISPR effector proteins further described in this application, unless otherwise apparent.

Metagenomic sequencing samples were selected from public databases and then downloaded. And sequencing reads were assembled with assembling tools. To search for potential Cas protein sequences, Cas sequences were downloaded as references and then Cas sequences were analyzed. We mined 13 novel Cas2 proteins via lots of work. The information of the 13 novel Cas2 proteins is showed in table 1.

TABLE 1
The detailed information of the Cas12 proteins

| Engineered | | | | | SEQ |
|---|---|---|---|---|---|
| Cas12 | Type | | | | ID |
| protein | (Predicted) | Source | Sample ID | #Spacers | NO: |
| GEBx0160 | Class II | Paludibacteraceae | GCA_024399835.1 | 9 | 1 |
| | Type V | bacterium | ASM2439983v1 | | |
| GEBx0161 | Class II | Prevotella/Group | GCA_021635885.1 | 5 | 2 |
| | Type V | of uncharacterized | ASM2163588v1 | | |
| | | isolates | | | |
| GEBx0162 | Class II | Prevotella/Group | GCA_020624585.1 | 18 | 3 |
| | Type V | of uncharacterized | ASM2062458v1 | | |
| | | isolates | | | |
| GEBx0163 | Class II | Flavobacteriales | GCA_018401695.1 | 52 | 4 |
| | Type V | bacterium | ASM1840169v1 | | |
| GEBx0165 | Class II | Candidatus | GCA_020060105.1 | 9 | 5 |
| | Type V | Woesebacteria | ASM2006010v1 | | |
| | | bacterium | | | |
| GEBx0166 | Class II | Sphingobacteriales | GCA_018262795.1 | 68 | 6 |
| | Type V | bacterium | ASM1826279v1 | | |
| GEBx0168 | Class II | Bacteria | GCA_020725175.1 | 9 | 7 |
| | Type V | (eubacteria)/NBRP | ASM2072517v1 | | |
| | | sequence | | | |
| GEBx0169 | Class II | Metagenome of | GCA_945639285.1 | 16 | 8 |
| | Type V | pig fecal bacteria | SRR14474048 bin.2 | | |
| GEBx0170 | Class II | Metagenome of | GCA_945638915.1 | 12 | 9 |
| | Type V | pig fecal bacteria | SRR14474044 bin.12 | | |
| GEBx0171 | Class II | candidate division | GCA_943355095.1 | 10 | 10 |
| | Type V | WWE3 bacterium | TH-11nov19-92 | | |
| GEBx0172 | Class II | Marine | GCA_913049555.1 | 8 | 11 |
| | Type V | metagenome | ERR599052_bin.236 | | |
| GEBx0173 | Class II | uncultured | GCA_910579115.1 | 4 | 12 |
| | Type V | Selenomonadaceae | MGBC110822 | | |
| | | bacterium | | | |
| GEBx0174 | Class II | Succinatimonas | GCA_021626595.1 | 9 | 13 |
| | Type V | sp. | ASM2162659v1 | | |

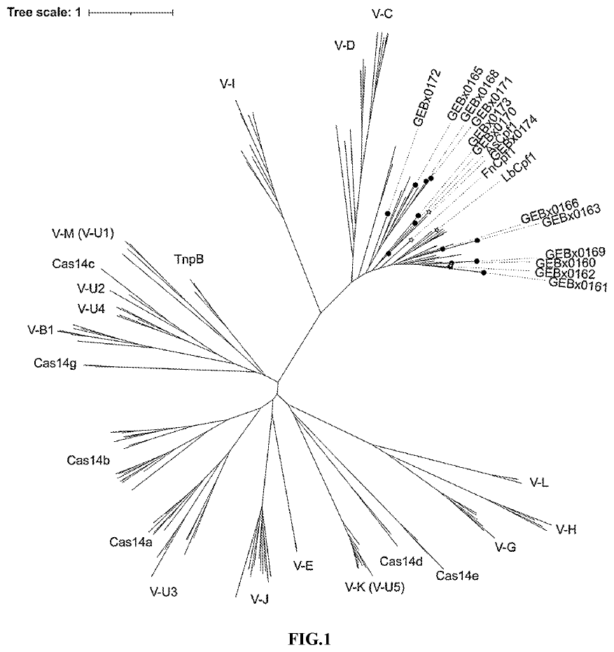

The phylogenetic tree was constructed by IQTREE (FIG. 1) to visualize the relatedness of the orthologs at the primary amino-acid level using 176 Cas12a (V-A), Cas12b (V-B), Cas12c (V-C), Cas12d (V-D), Cas12e (V-E), Cas12f (Cas14, V-U2~4), Cas12g (V-G), Cas12h (V-H), Cas12i (V-I), Cas12j (V-J), Cas12k (V-K or V-U5), Cas12l (V-L), Cas12m (Vm or V-U1) and TnpB sequences from The National Center for Biotechnology Information (NCBI), various publications, and patents. The branches of the tree corresponding to the Cas12 protein disclosed in this invention was marked with a circle while the reference nucleases (AsCpf1, FnCpf1 and LbCpf1; SEQ ID NOs: 60-62) were marked with stars. Although phylogenetically more closely related to Cas12a than other subtypes, they are located on different branches, suggesting that they are evolutionarily distinct.

The tree shows that the engineered Cas12 proteins studied herein are representatives of unique Cas12 clusters. Besides that, the Cas12 proteins share less than 50% identity with the existed Cas protein, some even share less than 40% identity or 30% identity with the existed Cas protein. These features suggest that the Cas12 proteins were independent of the existing Cas12a family.

In one aspect, the disclosure provides an engineered, non-naturally occurring Cas12 protein, wherein the Cas12 protein comprises an amino acid sequence selected from SEQ ID NOs: 1-13, a homologue thereof having at least 70% sequence identity to the amino acid sequence, or a variant thereof.

For example, “at least 70%” can include 70%, 75%, 80%, 85%, 86%, 87%, 88%, 90%, 91%, 92%, 93%, 94%, 95%, 96%, 97%, 98%, 99% or 100%; “at least 80%” can include 85%, 86%, 87%, 88%, 90%, 91%, 92%, 93%, 94%, 95%, 96%, 97%, 98%, 99% or 100%; “at least 85%” can include 85%, 86%, 88%, 89%, 90%, 91%, 92%, 93%, 94%, 95%, 96%, 97%, 98%, 99% or 100%; “at least 90%” can include 90%, 91%, 92%, 93%, 94%, 95%, 96%, 97%, 98%, 99% or 100%; “at least 95%” can include 95%, 96%, 97%, 98%, 99% or 100%; “at least 97%” can include 97%, 98%, 99% or 100%; “at least 98%” can include 98%, 99% or 100%; and so on.

In some embodiments, the Cas12 protein comprises an amino acid sequence having at least 75%, 80%, 85%, 90%, 92%, 95% or 98% sequence identity to any one of SEQ ID NOs: 1-13.

In some embodiments, the Cas12 protein comprises an amino acid sequence having at least 90%, 95% or 98% sequence identity to any one of SEQ ID NOs: 1-13.

In certain embodiments, the amino acid sequence of the Cas12 protein has at least 70% sequence identity to any one of SEQ ID NOs: 1-13. In certain embodiments, the amino acid sequence of the Cas12 protein has at least 75% sequence identity to any one of SEQ ID NOs: 1-13. In certain embodiments, the amino acid sequence of the Cas12 protein has at least 80% sequence identity to any one of SEQ ID NOs: 1-13. In certain embodiments, the amino acid sequence of the Cas12 protein has at least 82% sequence identity to any one of SEQ ID NOs: 1-13. In certain embodiments, the amino acid sequence of the Cas12 protein has at least 85% sequence identity to any one of SEQ ID NOs: 1-13. In certain embodiments, the amino acid sequence of the Cas12 protein has at least 87% sequence identity to any one of SEQ ID NOs: 1-13. In certain embodiments, the amino acid sequence of the Cas112 protein has at least 90% sequence identity to any one of SEQ ID NOs: 1-13. In certain embodiments, the amino acid sequence of the Cas12 protein has at least 92% sequence identity to any one of SEQ ID NOs: 1-13. In certain embodiments, the amino acid sequence of the Cas12 protein has at least 95% sequence identity to any one of SEQ ID NOs: 1-13. In certain embodiments, the amino acid sequence of the Cas112 protein has at least 98% sequence identity to any one of SEQ ID NOs: 1-13. In certain embodiments, the amino acid sequence of the Cas12 protein has at least 99% sequence identity to any one of SEQ TD NOs: 1-13. In certain embodiments, the amino acid sequence of the Cas12 protein has 100% sequence identity to any one of SEQ TD NOs: 1-13. The “100% sequence identity” means the amino acid sequence of the CRISPR-Cas12 protein is selected from any one of SEQ ID NOs: 1-13.

The amino acid sequences of Cas12 proteins and the referenced cpf1 are shown in table 2.

TABLE 2
The amino acid sequences of Cas12 proteins and reference cpf1

| | SEQ ID | |
|---|---|---|
| Name | NO: | N to C |
| GEBx0160 | 1 | KTMTDLTGLYPLSKTLRFELKPIGKTLEHIERKGLIAQDEQRAEDYERVKDLIDKYHKKFISLCLNKCRL |
| | | KVESTGEYDSLEEYVEFAGIAKRTQEEENAFTKVKEMLRKTVADSFKKDPDQFKELFKKEIITEHLPNM |
| | | LLTDGSEEDKKKVESFALFTSYFTGFHQNRENMYSAEEKSTAIAYRLIHQNLPMFLDNIKSFEKIQATIDG |
| | | KDMEELENAFRECLNVEHIADMFQLAYFTEILTQEQIDVYNYIIGGKTEEDGTKIKGINEYVNLYNQQH |
| | | KDSRLPLLKPLYKMILSDRTTLSWLPEQFKSDEEMLTAINEAIKKLNDEVLNTANQDNLQQLLSHLSDY |
| | | DTRHIYITNNHCLTDISQQIFGQYDRYTTAIKEEIKSSLKPTAKEKKDPDLLNDRTRKLFDARKSFSIAELN |
| | | EYAQAEANQTIEVYFAQLGSYDRNGEQQISLLSRLNMAYNEAKEVLDGKYTNLNQSKEATEKIKNLLD |
| | | AYKAIQFFAQPLAGSGDEAEKDNEFDAKLRNLTNTLSEVITPLYDKVRNWLTRKPYSTEKIKLNFENAQ |
| | | LLGGWDVNKEPDCTAILLRKHKQYYLGIMDKKSNHSFDIDELPSEGDCYEKIDYKLLPGANKMLPKVF |
| | | FSKSRIDEFNPSDAIKKAYEKGTHKKGPDFSLADCHRLIDFFKASIAKHEDWKRFPFSFSDTGSYEDINGF |
| | | YQEVEQQGYMIGFRPVSEAYINQLVEEGKLYLFQIWNKDFSEYSKGRPNMHTMYWNMLFDEKNLADV |
| | | IYKLNGQAEVFFRQKSLDIAKTCVHAAQQPINNKNKSNQKQQSTFDYDIIKDRRYTMDKFQFHVPITMN |
| | | FKSKGRENINSQVREIIRNNGIQHVIGIDRGERHLLYLSLIDLKGNIVKQMTLNDIINEYKGNTYATNYKQ |
| | | LLEYREGERTEARRSWQTIENIKELKEGYLSQVVHVIAKMMVEYNAIVVLEDLNGGFMRGRQKIERNV |
| | | YEQFEKKLIDKLNYYVDKQKEATEAGGVLHALQLTSKFESFQKLGKQSGCLFYIPAWNTSKIDPVTGFT |
| | | NQLDTRYQNVEKALQFFGRFDSIRYNKEKDWFEFMLDYDKFTTKAEGTQTRWTLCTYGTRIRSFRNDI |
| | | NKWVPKDVVLTEEFKQAFAAANIDVNGNLKDAICNLNEKAHLEELMHLVGLLVQLRNSKTNTEIDYM |
| | | LSPVADKNGVFYDSRTCPDTLPKDADANGAYNIARKGIWVIRKIQEDQTNDKVDLKITNKEWLAFAQQ |
| | | KPYLKHL |
| GEBx0161 | 2 | EPIGKTADYIKNYQYLETDERLAKESKKVKELADEYHKEFIGDVLSSLELPLSKINELWDIYMSNDTDRE |
| | | IKFKKLQENLRKVIAEAFSKDKRFGNLFKKEIITDILPKFLQDKDDDIKIVNRFKGFTTYFYAFHKNRENM |
| | | YVSEEKSTAIPYRIVNQNLVKYFDNYKTFKEKVMHLLKDKNIVESIERDFKDILNEKSIEDVFGLANFTH |
| | | TLCQADIEKYNTLIGGLVVKNEKKEIKGINQYINEHNQTSKKGNGIPKLKPLFNQILSDRKSLSFTLDDIK |
| | | KTSEAIRTIKDEYENLRDKLATIERLIKSIKEYDLAGIYIKMGEDTSTISQHWFGAYYKIIEAIADAWERRN |
| | | PKKNRESKAYSKYVSSLKSISLQEIDDLKIGEPIENYFATFGTTCSDRTSGVSSLNRIKAAYTEFVNKFPEG |
| | | FEDGDDCNDAYFKANVEVVKNLLDSIKDFQRFVKPLLGNEDERDKDEAFYGEFVPTYTDMDNIITPLY |
| | | NRVRNFATKKPYSTDKIKINFENVVLLKGWDKNKESDYASIILMKDGQYFLGVLRNGSKSTLKTILPNT |
| | | GDCYQKMVYKYFKDIKSNLPRCTTQRKDVKAHFAESSDDYTLLDTKAFVSALTISREVFELYNAPDKE |
| | | KKFKKEYLKNTNDSIGYANAVSVCKRFCLEFLKKYRSTAIYDLSDVETSVDSFDDLSSFYQEIDKRLYSI |
| | | SFENVSVDSVNELVDNGNMLLFRIANKDFSPNSKGRPNLHTIYWRMLFDPANLKDVVYQLNGNAEIFF |
| | | RKASVTRTEPTHPANVAIKNKSEYNKQNKPYSTFKYGLIKDRRYTTDQFEFHVPITMNFKQPESSKLQD |
| | | KLNKQVLDFLKQDGVRHIIGIDRGERNLLYLVMVDMEGKIKKQISLNEIAGNPKNPEFKQDFLALLHER |
| | | EGDRLESRRSWNTIQSIKELKEGYMSLVVHEIANMMLENDAIVVLENLNRSFMQKRGEKSVYQKFEKM |
| | | LIDKLGYIVDKTKDVSDNGGALHAVQLADTFENFNKTQKGAIRQCGFIFYIPAWRTSKIDPVTGFVPML |
| | | RCQYESIVESKKFFGKFDSIYYDATGKYFVFQTDFTKFNTESKGGIQKWDICTYGDRIYAPRTKDRNNNP |
| | | VSERVNLTEEMKSLFVSHNINIQGDIKAGIMQQTDKEFFESLHRLLRLTLQIRNSKKSTGKDYEDYIISPV |
| | | MGKDGRFFDSRNADATQPKDADANGAYNIARKGLMLLRQIQAQEKQDLSNGKWLEFAQR |
| GEBx0162 | 3 | TRKFDDFIHQYSLSKTLRFEARPIGETLHNFIKNGLLERDEHRAESYVKVKKLIDEYHKAFIDRVLNDGC |
| | | LNYEDKGEYDSLTEYYALYSAPRKDEAEREHFKETQQHLRDQVVKKLTGDNAYKHLFGKELIESYKD |
| | | KEDKKKLNQADLVQFINTATPKQRLNFSKEEAIDLVQEFCGFTTYFGDFYQNRENMYSAEEKSTGIAYR |
| | | LINENLPKFIDNMETFKKIAAIPEMEDNLKEIHDNLAEHLNVENIQDMFQLNYYNQLLTQKQIDVYNAII |
| | | GGKTDDEHKEKIKGINEYVNLFNQAHKDAKLPKLKTLFKQILSDKNAISWLPEEFDNDQEVLNAIKDCY |
| | | ARLSENILGKENLKRLLCSLSEYDTKGIFLRNDLQLTSISKKMSGSWTDIPNAIKNDLKDGVPAKKHKES |
| | | EEDYEKRIDNLFKRQDSFSIGYIDDCLNKFDDNNTFTIEGYFKKLGAKDTQSEDIIKQIANAYTDVKPLLN |
| | | STYPKSKNLSQDKENVVKIKRFLDTLMSLVHFVKPLLGKGDESNKDEKFYGELSLLWTELETIVPLYNM |
| | | VRNYMTRKPYSNSKIKLNFDNSQLLGGWDANKESDYASILLRRDGKYYLAIFDKDSKKLLGKSMPSDG |
| | | ECYEKMVYKFFKDVKTMIPKCSTQLKKVVKHFSISDTDFVLSDENFKVPLTITKEVYDLNNTMYGKYK |
| | | KFHKGYLTTTGDIDGYTSAVKTWINFCMDFLGAYGSTMSYDLSSLATKEYSSLESFYQEANRLLYTVNF |
| | | VKVSTSYVSRLVTEGKMYLFQIYNKDFSEYSKGTPNMHTLYWKALFDDRNLKDVVYKLNGQAEMFFR |
| | | KKSIDCNRPTHPANQPIQNKNKDNKKKESVFEYDLIKDRRYTVDKFMFHVPITMNFKSTDKENINLPVM |
| | | EYLQTSNDTHIIGIDRGERHLLYLVVIDLHGNIVEQYTLNDIVNEYNGNTYRTNYNDLLEARGNEQQKA |
| | | SQNWQTIENIKELKEGYLSQVIHKITQLMIKYHAIIVLEDLNMGFKRSRQKVEKQVYQKFEKMLIDKLN |
| | | YLVDKKADIESTGGLLNAYQLTNKFVSFKKLGKQSGFLFYIPAWNTSKIDPVTGFVNLLDTRCQNKNKA |
| | | KAFFAKFDSIRYNKDKDWFEFALDYDKFGSKAEGTRTKWILCTQGKRIKTFRNAEKNFQWYNQVIDLT |
| | | KDLKQLFAQYDIDINGNLKEAISKQTKKTFFDDLMGLLKLTLQMRNSITRTETDYLISPVADEIGNFYDS |
| | | RTCGPSLPENADANGAFNIARKGLMIIEQIKASEDLSKLKFDISNKSWLNFAQQKPYKHE |
| GEBx0163 | 4 | NTSKFIFQNLTHQFQLQKTLRFELIPQGRTLEHIEKNGLLEADELKANRYKQVKEWIDLYHKAFIEKVLR |
| | | EPKLEGLSEYRVFFENKQRNEKDEEAFKKLKSDLRKKISSFFKDEPNKTFYKRLFDKELIKADLPDFLRE |
| | | QGMHEEATMLLEEFGNFTTYFKGFHENRKNLYVAEDKSTAIAFRLIDENLPRYMQNLLLVEKHREVFE |
| | | QFFPQIQERLGKELDGLRVEEFFSEEGFNLCLNGSGIHQYNTILGGVKGEEEEGIPGLNMLINLYRQKHK |
| | | LKSRELPTLFPLHKQILSDRESSSFRPEPYEDGDENTVVEDLLKLGDQWFNPEGFVPQLVAHLTKLKDEE |
| | | QDQLFVNNKANALSAVSQATLGHWAVLGEALSSYWNHVHDVKENALKGKALTAKEKWLKQPYFSFQ |
| | | EIELALRHYMGAEGKENASKWNVWLEKPLVQYFNYGLTKEAEKTKVDTAKAAWGHAENILRELQNH |
| | | ENPKLKTKETKDAIKAYFDACLDLLHFIKPLVVDLEQCEASPTFYGDFLSLYHEMDDLVPLYNKVRNY |
| | | MTKKPYSYEKFKLNFNKGTLLDGWSQSKETDNLCVLFREGNNFFLGIMDRKHNKSFVKPPMAKEEEPV |
| | | MQKVVYNLLPDPSKMLPKCFTAPYFRRIAKEKTKDPEQIVSEYSKEKGLIKPKLDVLDAYAHKKHTKE |
| | | NFQRNGFEQVVEYFKEGAQKHPDWHVFDFKFKPTSAYKDVSDFYQEVTEQGYCLDFVDIPKRYINDLV |
| | | ESGKLYLFQIYNKDFSKYSKGKPNLHTMYWKAIFDPQNLKAPIYKLNGEAEVFFRKKSLEYTQEQLDTG |
| | | HHREELKGKFDYPIVKDRRFAFDKFQFHVPITLNHARKNQAVGPQVLRYIQKHKEELHYIGLDRGERHL |
| | | LYLSVVNAKGEIIEQRSLNKIGNGFVKDGKPYTHDYQGKLLEKERNRDSARKDWDAVESIKELKEGYM |
| | | SIIVHEIAQLVVKYNAVVILEDLNFGFKRGRFKVERQVYQKFERKLIEKLNHLVFKEVAPQEPGGLLKAL |
| | | QLTDQFTSFTKLGKQSGILFYLPAAYTSKIDPLTGFINQVPWKYTNLEAAQKWIGNFKRISYHAELDLFE |
| | | WVLDQRDFLSNPAGCKHQWVLRMDNQERWHWNSKAEGTNGKGAYSPIRLKEQFEDLLARQNISYNG |
| | | NSKLLEDIQAVKDASFFKTLFFFLRTLTQLRYNNGKSGAEEEDFILSAVANPGMEPFDTRKAKQGLPLD |
| | | ADANGAYHIAMKGLMTLGQLKEKPNGKLEIPKLDKSAWLCFMQQRSNQG |
| GEBx0165 | 5 | QTKNFMSFSDFTNLYALSKTLRFELRPIGRTEEFFKKNKVFEKDKTINDSYNQAKLYFDEMHRRLIRDAL |
| | | NTQNTSALDYEKYAKHWKELQKDKGKYIQNWKDAQKEFRECISNLLDQQAEQWKSKYASVGLNKAD |
| | | IKQKGTDFLSSAGMVKILKKEFPKEAENQLVKEGFPSLFIQKAEAPEQKQYIFDSFDRFSTYLAKFNQTR |
| | | DNLYKDDGTPTAIATRVVENFVAFLQNQAIFNNHYISLKSELDFSREEIDCFTANYYVRCLLQEDIDEYN |
| | | KTIGQINARIKELRDRKQKESDFKKSGYPLLKSLNKQILGKIEKKRQFIENKADVFPAFSELIEQANARLT |
| | | ALRQLINNLCEEVFEEAYGGIYIKNTGINTISRRWFASWYDFEIALPQKSKNEEESKVVTFVSLQDIKDTL |
| | | ENKLSGDIFKEEYYKKKIINKEQSNWKQILAILQNEWNELFSEYQKALENTKKLDLNSVFSNSKEQVEII |
| | | KSFADASLRVYQVVKYFALEHKKSDEIPSSYSTDFYEEFDKQYNNFEFIPYYNALRNFLTKKVVGENKI |
| | | KINFEKGNLLNGWAESPEGNAQFCGYILKKENKYFLGITDQPTILDTKDNPSLTNITDKEYYEKMAYRQ |
| | | LKSTTIYGSGYEGLYNSKYANDKKYISESKIIERVKKLLQEKYLTRFPELKQIIEKEYKDANELAKNLGNF |
| | | NLYSLEFIPVNKNYIEKLTHDKNGSIKYLYIFEIANKDLGKITPRKYNIHSLYFKNLFSEDNLEKPVFKLSG |
| | | GAEIFFRPKTVNLPKKKDKAGKDIIDHKRYAQDKLFFHLPIVINFGAGKVGKFNQELNKKLLAGNKNINI |
| | | IGIDRGEKHLAYYSVINQKGEIIEQDSLNEINGVNYYGLLQKREKERLENRKSWLPLQQIKDLKRGYISQ |
| | | VVYKIVRLAIEHNAIIVLEDLNMRFKQIRGGIEKSIYQQLEKALIDKLGYLVFKDRNAKEVGGVLQGYQL |
| | | SAPFESFQKIGKQTGIIFYTQASYTSVTDPLTGFRKNIYVSNSAPIEKIKVAINNFEEIGWDDVVQSYFFNY |
| | | DTKNFSNGNDIQSKKWKIYAKVPRIKREKVNGYWQYNDVDPNDMLEKLFRLYEFKNLRSNMLEQIHA |
| | | KETNGELRGERDFDNKPRNFYQSLVYILNLILQLRNSTSKQIKLNEKEDVYETGEDVDFIASPAYPFFTTL |
| | | SSKSNSNLAGFENRFTGLNHDQEQKKFNGDANGAYNIARKGIIILERITKNSDKPNLYISNEDWDKFVSQ |
| | | SYP |
| GEBx0166 | 6 | ESKTLDRFIGKYQLSKTLRFELIPQGKTKEHIEQKGLLQQDEHRAKAYKMVKKIIDRYHQHFIATALNNL |
| | | ALDGLQSYEELYHKKEKEEADKKNFESLKAGMRKQIGDAFNKSEASKVQEIWKVLFDKELIRKALPEF |
| | | VTSEEDKKLLAEFKDFTTYFTGFHQNRANIYTHEEKATAVAFRLVHENLPKFLDNSRVFDWIQTEYPDL |
| | | DLSAIEKELAPELQGTTLPEIFALSFFNHTLTQNGIDLYNAVLGGKTTEQQNKIRGLNEYINLYNQRRTAE |
| | | AKKEGEKFRRVPQFKELYKQILSDRESISFLPETFENDSEVLSAIENFFATQLVHFEKDNRSISVFAELQKL |
| | | LQELKEDDRSKIFVRNDLALTNISQNIFGDWQVIKNALEHYYTTTVNPLAVGKKLTKAYENGKEKWQK |
| | | QSYYSIYEIEEALLAYSSEADVLKEKNLTAASRPLCNYFATFSKDEEGNNLFQRIETAYTAVKDLLNTDY |
| | | PEEKNLAQDKTNVAAIKTFLDAIKNLLHFIKPLHPARLEGEKDERFYGAFIPLFDQLNLLTPLYNKTRNY |
| | | LTRKPYSTEKVKLNFENSTLLDGWDANKETANTSVLLRKDGLYYLAVMDKKYNKVFEQMPEVAEGE |
| | | KVFEKVVYKLLPGANKMLPKVFFSAKNIQYYNPSTELLENYKNETHKKGATFNLVHCHQLIDFFKASIA |
| | | RHQDWKNFNFQFSDTGSYADLSGFYREVEQQGYKITFHNLPESYINQLVDQGKLYLFQIYNKDFSPFSK |
| | | GRPNLHTMYWKMVFHEGNLTNVVYKLNGQAEIFFRKKSLNYDEKTLREGHHAAALKDKFAYPIISNR |
| | | RFAFDKYQFHVPITINFKSTGRDNVNAEVLSFLQHNPSVNIIGIDRGERHLLYLTLIDQKGNIKKQMSLNQ |
| | | IINSYRNKEGDMVNVKTDYQNKLHLKEEERLKARQGWGLIENIKELKEGYLSHVVHEIAKMMVEHNAI |
| | | VVMEDLNFGFKRGRQKVEKQVYQKFEKMLIDKLNYLVFKDRTATEPGGLLHALQLTGSYTDFMKYKQ |
| | | KQCGFLFYVPAWNTSKIDPLTGFVDFLKPKYESIEKAQNFLAKFKSIRYNPAKQWFEFTFDYKDFTDKA |
| | | EGSRTRWTLCSTNQPRYRWNPKAANGKGAQEAVNVTQALEDLFSAHNIAYGSADDLVTAITAQSSADF |
| | | YKALLRAMVTLLALRHNNGKAGTDEQDYILSPVEPFFDSRLADDSRPKDADANGAYHIAKKGLWVLQ |
| | | QINELQGDDWKNLKLAISNKQWLQFAQQLNADISPKVS |
| GEBx0168 | 7 | SSSIVSVSTEKKQAEVLSNKSVFDQFTRKYALSKTLRFELRPVGKTRENIEKANPNFIRDKEIEDAYQILK |
| | | PVFDKLHEEFITESLESDEAKHLSFEDYFSVYKNLRNEQDREEKKKIEKSLETKEKNLRAAFSDIYKAEG |
| | | EKFKSKVGSDKKGKPILKEDSFKILTEVGILKYVKAKIEEFVVMNLKTHEGKEVKKEDLEKALGTADSK |
| | | GVFEGFFTYLTGFNQNRENYYSTEEKATAVASRIIDENLPKFADNVLEFQKRKDEYLKVYDFLKENGIE |
| | | TIGKNQKGEKVELEKIIADIFEIGYFTNCLSQREIEKYNLNIGNANNLINRYNQQQSEKNKKLRIFKTLYK |
| | | QIGCGEKGDFIPAIKNDDGLRAVLKNVVENGEKYFGQFDGKIEDKVDTVGEFVHYIINHESFEGIFWSDK |
| | | ALNTISGKYFANWFILKKALKEGGVFGGKKKDDEDIKIPQAVELKNLFEILDKTEKWKNKEDGLFKTSL |
| | | FDGEGNKKGIIERAEKPSKALLGMIFADVEKMAKDFSELSPKVLSITTDFKKPEHIENIKMLLDSALYVN |
| | | QIVKYWKVKDKFAVDPILAESLKAILFGDNNPTKFYDIIRNYLTKRPQEGLNKLKLNFDNSVLGGGWDV |
| | | NKEPERWCLILKDGEKKYLAILTKASQKLFGEKAKYKEVVTDENYLKMDYKLLPGPNKMLPKVLLPK |
| | | RNRKEFGATDDILSIYDEGGFKKNQPSFTKEKLWKIIDFYKEGLKVYPSAEKSWQDLFGFNFSPTEKFESI |
| | | DQFYNEVEKQGYKLSWNIISKKLINEAVENGEIYFFEIRNKDNNLKEGKEKTSEKNLHTIYWNEIFGDVA |
| | | NKPKLNGEAEIFYRKTILKKKENETDKEFEIRLKEKLGKKKDKSGKEVLEHKRFANEKFVFHCPITLNFC |
| | | LRSLKFNDEINESMVKLNDDITTIGIDRGEKHLAYYFVVKQTKDGKIETLDQGSFNEINGQNYAEKLEK |
| | | MAGNRDEARKNWKTIGTIKELKDGYISQVVRKIVDLAIYEDVEKKVFRETPAFIVLEDLNIGFKRGRQKI |
| | | EKQVYQKLELALAKKLNFLVDKHAKEGEIGSVTSALQLTPLVNNFGDIEGKKQFGIMFYTKADYTSQT |
| | | DPVTGWRKTIYLKKGSEESIKKQILDNFDDIKFDGKDYYFKYTDRNTNKPWELYSGKDGESLDRYYRE |
| | | LVHENSDKKWVPKEQNLKKILDGLFEGFDKEKSLLEQLRQGKNPNKINDRTSWESLRFVIDLIQQIRNSG |
| | | KTEEDADFILSPVRDKNGNHFDSRKINLNLPTSGDANGAYNIARKGIIMCEHIKRDLSLFISDEEWSAWL |
| | | AGREIWENWIKKNEKNLKRKQ |
| GEBx0169 | 8 | KSIEELIGQYSLTKTLRFELKPIGKTLEQIERKGVLSADQTRADQYKRVKESIDRYHKAFITESLDSLVLK |
| | | VKSTGNTSDSLEEYASLLDNSADDEALDKVKKGLRKQVAERFDKQIRLKKLFKNDLITKILPEFLKVNG |
| | | NEDERKAVESFRNFTTYFSGLNENRKNMYSKEAKSTAIAYRLIDENFPRFHDNMLSFAKIAAVNEVSNH |
| | | FAEIAEAFKSEMVVASVAEMFGLEYFNNTLTQQKIDLYNYVIGGKTEGDKVKIQGINEYVNLYNQNHP |
| | | NDKLPLLKPLYKMILSDRVALSWLPEEFNDDEAMVCAIKEFYNAIQPVLWGEVEDDDNSVCHLLKNIG |
| | | KYDIEHIFISNGAALNDVSRQMFGQYDFLANAIKKDIRQNTKRSSRETDEKFEGKVNTLFKKAKSYSIAH |
| | | LNALIKENRSETIESFYSNLGACDRDGEQKINLLTQIELAHEAAQDVLDGKYADINRDEKATMLIKELLD |
| | | AFKALQYFIKPLLGSGEEASKDNVFCTKLLNAWQALDAVTPLYDKVRNRLTRKPYSTDKIKLYFENKG |
| | | KFLDGWVDSKTDNSDNGTQFGGYILRKKNEIGEYDYFLGISSDTKLLRANPDIQYEHGMYERLDYYQIQ |
| | | SKTFLGSSYKGEYNDDAMRLMSAFVEYGKKSRFDSCVLPTDKEKSLPTYLKRIYTDNNSFYNEAMTDT |
| | | VVAEAYNNIKSHLLLTLAGIKRVEAAVNLSKHNEYNLIQIFDEIGKITSSSKIFSFFPVDTSLVEKLNERDN |
| | | KPLFLFKISNKDLSYAETSSKNIRKSRGKENLHTMYFKALMEQSHNVFDIGTAEVFFRKATPGLDKSTTI |
| | | HKANEALCNKNPHSKKTSSTFGYDIIKDRRYVENKFQLHLSMVINYQGKNPNTDIISPQVLDIIRNNGIK |
| | | HIIGIDRGERNLLYLSLIDLKGNIIKQISLNEIKPLGNDGCVTNYRDILADREKDRKEARRSWKKLENIKD |
| | | LKSGYLSQVVHVIAKMMVDYQAIVVLEDLNSAFMQSRQKIERSVYEQFEHKLINKLNYYVDKSVSDDL |
| | | PGGLLYGLQLASKKTHDVQCGCIFYVSAWNTSKIDPVTGFVNMLDTRFENVSSSRSFFSHEDSIRYNVA |
| | | KDWFEFTFDYNNFHKKLEGTKTRWTLCTYGTRILTQRNDRNQWESREVDITDEFKKAFVAAGIDIHGN |
| | | LKDAICALDKRVHLEQLMRLMKLLLQLRNSIINQDTDYILSPVAENGVFYDSRRHADNLPIDADANGAY |
| | | NIARKGLYLLRKIKADRSAKPELCISNKEWLRFVQDKSNPGD |
| GEBx0170 | 9 | VTIDDFCGQKNGYSRSITLRNKLIPVGKTQENLEKANMLEEDFSRAKAYVEVKKIIDDFHRSFIEDVLSK |
| | | ANLNWKELYEKIDSFQRETDKAKKAKNQKDLQPVQVKMRKAIVKIFKDDERFDKLFKKELLSDLLPEII |
| | | KSAPENEISNKQTALDVFKGFATYFTGFHENRKNMYSEEDKATAISYRIVNENFPKFYANIKLFEKIQKE |
| | | FPVIIEETEKSLASFLAGKKLKDIFTPEAFNQVLTQSGIDFYNTVIGGIAGEAGTEKVQGLNEKINLARQQ |
| | | LPDEEKSKLRGKMVVLYKQILSDRETASFIPVGFENKDEVYKTLQEFKKSTVEKSIEIIRETFKSPENFDLS |
| | | KIYVPAKELTAFSLSVFGRWNILSDGLFAIVEEKSKKALSEKQTEKLANEIAKKDYSLSELQAAYEKYLE |
| | | KNPVEDEGRCRRLSAKNYFELIELRKIDDSAEKEFVNVLSNLDELWSKIDFENPRNLQQEKEAATPIKNY |
| | | LDEVQNLYHFLKLVDYRGEEEKDSEFYSKLTEILDLLSEIVPLYNKVRNFVTKKPGEVKKIKLNFDCSSF |
| | | LKGWGTEFGTKDAFIFIDEKKYYLGVINKKLDSDTVKYLQTPGEHIIKQVFVNFQKPDFKNFPRLFIRSK |
| | | KDRFAPAVEQFNLPVNDILDIYDKGLYKTEYRKENPIIYKESLIKMIDYFKLGISQHESFKGFKLTWKESS |
| | | EYENISDFYADVTASCYQVNFTNVNFDRLLSLANEIEDDNKKIFLFEIYNKDFSTGKNGGNGSTGKKNL |
| | | HTMYFENLFSKENLKDIVLKLSGEAEIFLKSENTELKKIVHKQGTTLVNRTTKDKKLIPENIYQEIYQFKN |
| | | SMIDNISDEAKAWLNSGNVECKIAKFDLIKDKHLIEKSYYLHCSIIQNFKAPEKVFNFNEKVQRYLKDNP |
| | | DVKIIGLDRGERHLIYLSLINQKGEIELQKTLNLVEQVRNDKTVKVNYQEKLVQKEGDRDKARRNWQTI |
| | | GNIKELKEGYLSNVVHEIAKLMVEHNAIVVMEELNFGFKRGRFPVERQVYQKFENMLIGKLNYLVFKD |
| | | KSVTEPGGVLNAYQLTDKSTNVSDVGKQCGWLFYVPAAYTSKIDPKTGFANLLNMKDLTNVAKKRMF |
| | | FSKFKDIHFDSGSNDFAFTFDYKDFDGKGAAEMTQTEWTVWSNGKRIFYNSKHHSTEEINVSEKLKELF |
| | | DASKIDYKTENNIFESIMEVGADLKEGEKPSKEIANFWDSLLWYFKLILQMRNSIPNSETDYLISPVKAED |
| | | GTFFDSRVQKELGKDSKLPIDADANGAYHIALKGLYLLKNDFNLNDKGYIENISNAEWFRFVQERDYLN |
| GEBx0171 | 10 | PNLFDNFTQQYSLTKTLRFELKPEGKTLENMQNAFKFDKDLQTFLVDQDIEDAYQTLKPYIDFIHEEFITE |
| | | SLESDIAKGIDFSEYFENFKIKKDINQKDFQNLEHKLRKEFDQLYIQIANKWKEEKYKQYKWKKGSKVA |
| | | NGSEILSSEDILKLVVDLNQDNNQLKDIVQNTFKGFFTYFSGFNDNRRNYYQVEKEAKSSIATRIVNENL |
| | | PKFCDNFLLFENRKNEYLGVYNTLKLLGKDLVDKDRKTLVTIEEIIFSIKHFNFCLSQKEIENYNKQIGNA |
| | | NLLINLYNQNTSNESDYKRLPLFKTLYKQIGCGKRDSLFFEITDYTKLEAEGKRILGKSAISVEEVLIQVK |
| | | ESGIKYFTEKLDSLLNYILEKETDNYQGLYWSNIAVNSISSKYLANWNNLKELFKGKKEVASYDKGREE |
| | | EIKINEAVDLSILFSILNNTEDWKVKEVFFKENLNEESETKKIIETSKTPSQALLRIVIGDLKKNASNFLNLS |
| | | DNIEQLLESKEKIKESMDYALSVTRMLKYFLVKESKTKGAPLDSKLVEDLNLLLLSDDAPWFMWYDGL |
| | | RNYLTKKPHDDAKENKLKLNFENSTLAGGWDINKEPANYCVILTNSANDQYLAINAKRNNKIFCKELV |
| | | DGKGKDKKLLKNPLYESDEQHWKKMEYKLLPGPNKMLPKCLLPKSNLRKFGASEEVLSIYENGSFKKN |
| | | TPDFSTQDLHKLIDFYKSALLKYEDWKCFHFKFNDTRSYGDISQFYSDLEKQGYLLSFIDINKSVLNQLV |
| | | DSGKIYLFQIRNQDSNISKKINHQDNLHTIYWRAVFEDVFNKPKLNGQAEVFYRKALPQEDLQLLKDKD |
| | | NKEIIANYRFSKEKFLFHVPITLNFCLKNKKINDLANREILSNDNIYFLGIDRGEKHLAYYSLVDQKGNIVI |
| | | DQNGKSIQGTLNIPFLDSNGNQRLVKATKRVMKDGKEDAQVVECKDYNELLEARASDRDYARKNWQ |
| | | TIGAIKDLKDGYISQVVRKIADLAVEYNAYIVLEDLNTGFKRGRQKIEKSVYQKLELALAKKLNFLVDK |
| | | SAKIGEVGSVTNALQLTPPVNNYSDIENRKQFGVVLYTRANYTSQTDPVTGWRKTIYLKTGSEDIIKKQI |
| | | FESFSNIYFDGQDYIFTYTDKNIDRKWELYSGKNGKSLDRYRGRKVNNKGWVIDKINIQEILDKIFINFEI |
| | | CYGEKNNIFEKLKAGVELEKYNEYTAWESLRFAIDIIQQIRNTGIESSDSDFLLSPIRVNNLHFDSRLASEN |
| | | QPNSGDANGAYNIARKGIIMANYIKQGVSPFVSDIDWDNFVTKN |
| GEBx0172 | 11 | NKFLDSFTHQYSLSKTLRFELKPVGKTAEWIQKRDIIGTKKQENGEAQLTGKDAVRAENYEYAKKLLN |
| | | EMHRLFIEDSLAAIKEEQKVELNKLLVKIAESGSLDGFRKPMSNICKELFDSATQDWLLAYSEEMPCFW |
| | | DEDILELTNKAETTENKKNIKFFLKKAEDIRKFKDDPLKAFKKKKHEALNSNTEALKLLEWKIRRGEVLI |
| | | SGKDLGRDESEEILSTKELILILRSFYSFVTYFTGFNENRANIYNLDDKKYISTSIAFRSFEQNLFFHIENIK |
| | | KWHTITNSIEEHTDQLNASDYPIKDKLRELELAFDCKLADLFAVDSFSQFLSQSGIDRYNSIMGGIAQKT |
| | | GKTKVQGLNEIINQARQQTGAKRHKFPPMKLLYKQILSKGEGIFVESYVDDADMLTSIAGFHENHLNES |
| | | LCSNWLHELEELIDSVQESKDTIFIARDKLNRISQEITGSWSAIQDWIVAHQKDQKEVDALTKKKVFSIQE |
| | | LEDYFSKSSDDELFTAKIPEDRSAYLPGEGEGILFQFFKVRAKEILAELQSGWYALDKAQLLQEKQLDKN |
| | | RENEGSKGFEQVGLLKGYLDACNSLSRFIADWNIQKDIKQERCSLWYEFLQKHLDTVPIFDLYNQVRNH |
| | | VSKKAYSTDKLKVNFEKSTLLDGWDRNKESQNFGVLFEKDGAYYLGIMTPDSNGIFEYDVNPDENTKK |
| | | KAIKEELRKQTTPENGEDRFQKINYKLLPGPNKMLPKVFFAKSNEDLFQPSARITEIKENKKYAKAYTDK |
| | | HGKAELHEYIDFCQKSLVRHPEWSVAYGFDQTTFRKATEYISIDEFYREVELLGYKVTFDSIKATYIEEK |
| | | VANGELYLFQIYNKDFSTAKKGGGKDNLHTLYWKSLFSEDNLKDVVFKLNGQAEVFFRPSSIVWSEKV |
| | | MKQGHHYDDDKMLKDKFKYPIIKDFRFTQDKFFFHCPISLNFKAPAKPYGFNYKVHDFLRKNGEINVIG |
| | | IDRGEKHLLYYSITNQQGDIIEQGSLNSIESSYEKGSSSVPHSTNYLEKLQEKEKNRDSARKSWSRIENIKE |
| | | LKAGYLSHVVHKLAKLIIKHNAVVVLEDLNAGFKRGRFKVERQVYQKFEKALIDKLNYLVFKDQEKPS |
| | | APGHPLRAYQLTDPFESFEKLGKQSGILFYTTAAYTSTTDPVTGFLKNVYKPYANVVDSSDFWKSFDEI |
| | | HYDVKKERFEFTYDLRKIKNKQVDRDKDEAKVSRSVWTVCSCVERSRYVKRKANEEQKQGAASKSIG |
| | | KMGEYEVFVVTDKLKALFEKESISYQSGEDIRSQLSNRIDKKDAKLHRELIYYFGTLLNMRVTDKQSES |
| | | GTDENDYILSPIEPFFDSRKGYEKLPVNGDANGAYNIARKGIAILNRIHESDSGDKVDLMIRKKDWQDFV |
| | | QNEEVVQKQMGKLDL |
| GEBx0173 | 12 | ALFSENFVGCYPVSKTLKFELRPVPETKAYLDDDNCPLLAGDLERDKAYPVVKELLDKYYRYFIDICLK |
| | | DKKLSPETIDEAYQAYSSHDEKSQKALKAASQKLRKEVAGFFDKKTRETYCLDKYSNLLVLDKKNDNK |
| | | EKGTPKTIPSVLKKWLENESGFSEKEVEKYSQAVQTFNGFTTYFISYKENRENMFSSEEKASAISYRVVN |
| | | QNMLRFFSNVENFENIKKKYPDLYQTLKEFEESFVPAAYSSLLSQAGIDDYNYRVIGRPAEDEDFAGVN |
| | | RLINEYRQKNAVRNRNLPVMVTLYKQILSDRENGFGRDILASDSEAKEYIRGKYSKAYDTACQLTVFVN |
| | | NYILTEDLDTVYVKTQHLKDISQSIFGEWSVLDDAINNGGIDSKIISLKALAEAFEKYCSALDPDMAQKY |
| | | RDAFSLKEYLKNPPAVEPRLPETAEKVTAYKPAMDELISLIRFYKPFYLYDGKKRLQVPAEGMDFSNEF |
| | | DKYFEDLKEFSHAYDKVRNFATKKPYSVEKIKLNFNQPTLLDGWDVNKEKENASFLFVKEGKYYLGIA |
| | | DKSDKNLFDRSRDAVKEALTLTCGEDYYEKVQYKQLPNIRMNLPRIVFKPKWEKVFAPIITERISAIEKE |
| | | KLYTAGANDRQAVIEWIDFVKSVLALHSEWNNYFQFKFKETSAYKNTKEFYDDLDNQAYILSKIKINAS |
| | | YIHKLVRERKLYLFQIYCKDFSDKKKKTGADNLHTMYWRCLFSDENLAALAQGQAPIIKLNGKAEIFLR |
| | | EPSLKYEATHPKNQPVANKNPLNSKRESLFSYDLAKNRRFTERKLFFHCPLTINFRTAAEGARSFNERVN |
| | | RFVENNPDIRIIGIDRGERHLLYYTVINQSGKILEQGSLNKISSDYRTPEGAEVEKVTDYHELLNRKEGDR |
| | | RAARQTWDTIENIKELKEGYLSQVVHKLTTLMIKHNAVVVLENLNQNFKRSRIKVEKQVYQKFEKALI |
| | | DKLNYLVFKDRPYGAPGSYANGLQLTAPFESFDRLGKQTGALYYVVPSYTSHIDPKTGFVNLLGAKLR |
| | | YESVVKAQETMKKIGSFKFNPERNWFEISFDYGDFGIEMAHPQWTACTHGDAYWGYNPKEKRAVSYD |
| | | VTEEIKKLLTEYGIDYGTGADIKEAAIRQTKPAFWKTLFYMLKLALQLRNTVAGTDNENDYILSPVEYE |
| | | AGKFFDSRKAGEDEPQNADANGAYHIAMKGLQALRRIEDGKVNKFPKNGERKAWLAFMQNREYLK |
| GEBx0173 | 150 | MALFSENFVGCYPVSKTLKFELRPVPETKAYLDDDNCPLLAGDLERDKAYPVVKELLDKYYRYFIDICLKDKKL |
| v1 | | SPETIDEAYQAYSSHDEKSQKALKAASQKLRKEVAGFFDKKTRETYCLDKYSNLLVLDKKNDNKEKGTPKTIPS |
| | | VLKKWLENESGFSEKEVEKYSQAVQTFNGFTTYFSSYKRNRENMFSSEEKASAISYRVVNQNMLRFFSNVENFE |
| | | NIKKKYPDLYQTLKEFEESFVPAAYSSLLSQAGIDDYNYRVIGRPAEDEDFAGVNRLINEYRQKNAVRNRNLPV |
| | | MVTLYKQILSDRENGFGRDILASDSEAKEYIRGKYSKAYDTACQLTVFVNNYILTEDLDTVYVKTQHLKDISQS |
| | | IFGEWSVLDDAINNGGIDSKIISLKALAEAFEKYCSALDPDMAQKYRDAFSLKEYLKNPPAVEPRLPETAEKVT |
| | | AYKPAMDELISLIRFYKPFYLYDGKKRLQVPAEGMDFSNEFDKYFEDLKEFSHAYDKVRNFATKKPYSVEKIKL |
| | | NFNQPTLLRGWDVNVEKNRASFLFVKEGKYYLGIADKSDKNLFDRSRDAVKEALTLTCGEDYYEKVQYRQLPNI |
| | | RMNLPRIVFKPKWEKVFAPIITERISAIEKEKLYTAGANDRQAVIEWIDFVKSVLALHSEWNNYFQFKFKETSA |
| | | YKNTKEFYDDLDNQAYILSKIKINASYIHKLVRERKLYLFQIYCKDFSDKKKKTGADNLHTMYWRCLFSDENLA |
| | | ALAQGQAPIIKLNGKAEIFLREPSLKYEATHPKNQPVANKNPLNSKRESLFSYDLAKNRRFTERKLFFHCPLTI |
| | | NFRTAAEGARSFNERVNRFVENNPDIRIIGIDRGERHLLYYTVINQSGKILEQGSLNKISSDYRTPEGAEVEKV |
| | | TDYHELLNRKEGDRRAARQTWDTIENIKELKEGYLSQVVHKLTTLMIKHNAVVVLENLNQNFKRSRIKVEKQVY |
| | | QKFEKALIDKLNYLVFKDRPYGAPGSYANGLQLTAPFESFDRLGKQTGALYYVVPSYTSHIDPKTGFVNLLGAK |
| | | LRYESVVKAQETMKKIGSFKFNPERNWFEISFDYGDFGIEMAHPQWTACTHGDAYWGYNPKEKRAVSYDVTEEI |
| | | KKLLTEYGIDYGTGADIKEAAIRQTKPAFWKTLFYMLKLALQLRNTVAGTDNENDYILSPVEYEAGKFFDSRKA |
| | | GEDEPQNADANGAYHIAMKGLQALRRIEDGKVNKFPKNGERKAWLAFMQNREYLK |
| GEBx0173 | 151 | MALFSENFVGCYPVSKTLKFELRPVPETKAYLDDDNCPLLAGDLERDKAYPVVKELLDKYYRYFIDICLKDKKL |
| v2 | | SPETIDEAYQAYSSHDEKSQKALKAASQKLRKEVAGFFDKKTRETYCLDKYSNLLVLDKKNDNKEKGTPKTIPS |
| | | VLKKWLENESGFSEKEVEKYSQAVQTFNGFTTYFISYKRNRENMFSSEEKASAISYRVVNQNMLRFFSNVENFE |
| | | NIKKKYPDLYQTLKEFEESFVPAAYSSLLSQAGIDDYNYRVIGRPAEDEDFAGVNRLINEYRQKNAVRNRNLPV |
| | | MVTLYKQILSDRENGFGRDILASDSEAKEYIRGKYSKAYDTACQLTVFVNNYILTEDLDTVYVKTQHLKDISQS |
| | | IFGEWSVLDDAINNGGIDSKIISLKALAEAFEKYCSALDPDMAQKYRDAFSLKEYLKNPPAVEPRLPETAEKVT |
| | | AYKPAMDELISLIRFYKPFYLYDGKKRLQVPAEGMDFSNEFDKYFEDLKEFSHAYDKVRNFATKKPYSVEKIKL |
| | | NFNQPTLLRGWDVNREKENASFLFVKEGKYYLGIADKSDKNLFDRSRDAVKEALTLTCGEDYYEKVQYKQLPNI |
| | | RMNLPRIVFKPKWEKVFAPIITERISAIEKEKLYTAGANDRQAVIEWIDFVKSVLALHSEWNNYFQFKFKETSA |
| | | YKNTKEFYDDLDNQAYILSKIKINASYIHKLVRERKLYLFQIYCKDFSDKKKKTGADNLHTMYWRCLFSDENLA |
| | | ALAQGQAPIIKLNGKAEIFLREPSLKYEATHPKNQPVANKNPLNSKRESLFSYDLAKNRRFTERKLFFHCPLTI |
| | | NFRTAAEGARSFNERVNRFVENNPDIRIIGIDRGERHLLYYTVINQSGKILEQGSLNKISSDYRTPEGAEVEKV |
| | | TDYHELLNRKEGDRRAARQTWDTIENIKELKEGYLSQVVHKLTTLMIKHNAVVVLENLNQNFKRSRIKVEKQVY |
| | | QKFEKALIDKLNYLVFKDRPYGAPGSYANGLQLTAPFESFDRLGKQTGALYYVVPSYTSHIDPKTGFVNLLGAK |
| | | LRYESVVKAQETMKKIGSFKFNPERNWFEISFDYGDFGIEMAHPQWTACTHGDAYWGYNPKEKRAVSYDVTEEI |
| | | KKLLTEYGIDYGTGADIKEAAIRQTKPAFWKTLFYMLKLALQLRNTVAGTDNENDYILSPVEYEAGKFFDSRKA |
| | | GEDEPQNADANGAYHIAMKGLQALRRIEDGKVNKFPKNGERKAWLAFMQNREYLK |
| GEBx0173 | 152 | MALFSENFVGCYPVSKTLKFELRPVPETKAYLDDDNCPLLAGDLERDKAYPVVKELLDKYYRYFIDICLKDKKL |
| v3 | | SPETIDEAYQAYSSHDEKSQKALKAASQKLRKEVAGFFDKKTRETYCLDKYSNLLVLDKKNDNKEKGTPKTIPS |
| | | VLKKWLENESGFSEKEVEKYSQAVQTFNGFTTYFISYKRNRENMFSSEEKASAISYRVVNQNMLRFFSNVENFE |
| | | NIKKKYPDLYQTLKEFEESFVPAAYSSLLSQAGIDDYNYRVIGRPAEDEDFAGVNRLINEYRQKNAVRNRNLPV |
| | | MVTLYKQILSDRENGFGRDILASDSEAKEYIRGKYSKAYDTACQLTVFVNNYILTEDLDTVYVKTQHLKDISQS |
| | | IFGEWSVLDDAINNGGIDSKIISLKALAEAFEKYCSALDPDMAQKYRDAFSLKEYLKNPPAVEPRLPETAEKVT |
| | | AYKPAMDELISLIRFYKPFYLYDGKKRLQVPAEGMDFSNEFDKYFEDLKEFSHAYDKVRNFATKKPYSVEKIKL |
| | | NFNQPTLLRGWDVNREKENASFLFVKEGKYYLGIADKSDKNLFDRSRDAVKEALTLTCGEDYYEKVQYKQLPNI |
| | | RMNLPRIVFKPKWEKVFAPIITERISAIEKEKLYTAGANDRQAVIEWIDFVKSVLALHSEWNNYFQFKFKETSA |
| | | YKNTKEFYDDLDNQAYILSKIKINASYIHKLVRERKLYLFQIYCKDFSDKKKKTGADNLHTMYWRCLFSDENLA |
| | | ALAQGQAPIIKLNGKAEIFLREPSLKYEATHPKNQPVANKNPLNSKRESLFSYDLAKNRRFTERKLFFHCPLTI |
| | | NFRTAAEGARSFNERVNRFVENNPDIRIIGIDRGERHLLYYTVINQSGKILEQGSLNKISSDYRTPEGAEVEKV |
| | | TDYHELLNRKEGDRRAARQTWDTIENIKELKEGYLSQVVHKLTTLMIKHNAVVVLENLNQNFKRSRIKVEKQVY |
| | | QKFEKALIDKLNYLVFKDRPYGAPGSYANGLQLTAPFESFDRLGKQTGALYYVVPSYTSHIDPKTGFVNLLGAK |
| | | LRYESVVKAQETMKKIGSFKFNPERNWFEISFDYGDFGIEMAHPQWTACTHGDAYWGYNPKEKRAVSYDVTEEI |
| | | KKLLTEYGIDYGTGADIKEAAIRQTKPAFWKTLFYMLKLALQLRNTVAGTDNENDYILSPVEYEAGKFFDSRKA |
| | | GEDEPQNADANGAYHIAMKGLQALRRIEDGKVNKFPKNGERKAWLAFMQNREYLK |
| GEBx0174 | 13 | SKLSTFNEHFQKTITLRNELVPVGKTLENIISSNVLINDEKRSEDYKKAKEIIDSYHQEFIEKSLSKVDIEW |
| | | DDLAYCLTKKEPENFDKKKKYQEEINKLQNEKRKKSVEIFKKFSFGVLCDNNGQEVQIKFDDLFKSTLF |
| | | KYVLPNFLKNDEDKTVVRDFSEFTSYFTGFHENRKNLYKSAPMPTAVAYRIVNDNFPKFISNQKIFCAW |
| | | KDNVPHFVEIAKTKLSEVGISDLDIESKFELSNFNSCLNQTGIDYYNDLRGQLNNVISHICQQDKNLGDL |
| | | LRKKRCLKMVPLYKQILSDKDSSFSIDEFDNDESAIQDVISFYKKMVGERCPQRKLSELLRELSSHDREKI |
| | | FIQGKNLNSISRILFGGKNWSLLREAIIEEKSKDKSFKKAIKSNSTDEALEKILSKDEFSISFLSRICHENLSE |
| | | KIDKFIKNQDEQLININSQTWPKTLKNSEEKKIIKEHLDFLLNFYRFAKSFSSNTIDKDMSFYADFDESLSS |
| | | LENVIGLYNKVRNYVTKKPYSLEKIKLNFEIPNLAEGWSKTKELANLTVIFLKDNEYYLGIINKNSKIDFS |
| | | LIKEKTSSENCFRKMEYSLFGKIAQNINRCVFTKAVKNHFSKSPDNFILSDKKKFLKEIEITKEIYDLVKSD |
| | | ESDKKYLKEYKKINEKEFRSALSKWINFCIEFLKSYKNTSQFDFSCIKKAESYDDINDFYDDIEKVTYKIE |
| | | FVDISTSLIDALVDKGQLFLFKIRNKDFAKKASGTPNLHTLYFKSIFDTKNFENGIVKLNGEAEIFYRKKS |
| | | LNVDDTTVHQKGSYIVNKVVVDPSSGKSEQIPDNVYNNIYAFVNGKLKTLSKEDEVYYAKATIKKATH |
| | | EIVKDRRFTEDKFFFHCPITINYKSKEKPSKFNDKVLDFLRKNDDINIIGIDRGERNLIYATVINKNGEIIDC |
| | | KSFNTIKHQSSSVNFDVDYHNKLQEREKNRKEEKRSWNSISKIADLKEGYLSAVIHEIALMMVKYNAIV |
| | | VMENLNQGFKKIRGGIAERSVYQKFEKMLIDKLNYFVIKNENWTNPGGVLNGYQLTNKVSTIKDIGNQ |
| | | CGFLFYVPAAYTSKIDPSTGFVNLINFNKYKNSNDRRKLICSFDKICFVQNENLFKFSIDYEKLCPNSKMP |
| | | VKKWDIFSYGNRIVREDLKHIPMKENPKYDPTEELKTLFSSKEIEYQKGQNLLEIISTKDMTEDKEFWNS |
| | | LFNIFKAILQMRNSLTNSPVDRLLSPVKGKDGSFFDTDKVEGTKFEKLKDADANGAYNIALKGLLVLKK |
| | | NDSVKTDKDMKNVKNVSLEDWLKFVQVTLRV |
| AsCpf1 | 60 | MTQFEGFTNLYQVSKTLRFELIPQGKTLKHIQEQGFIEEDKARNDHYKELKPIIDRIYKTYADQCLQLVQ |
| | | LDWENLSAAIDSYRKEKTEETRNALIEEQATYRNAIHDYFIGRTDNLTDAINKRHAEIYKGLFKAELFNG |
| | | KVLKQLGTVTTTEHENALLRSFDKFTTYFSGFYENRKNVFSAEDISTAIPHRIVQDNFPKFKENCHIFTRL |
| | | ITAVPSLREHFENVKKAIGIFVSTSIEEVFSFPFYNQLLTQTQIDLYNQLLGGISREAGTEKIKGLNEVLNL |
| | | AIQKNDETAHIIASLPHRFIPLFKQILSDRNTLSFILEEFKSDEEVIQSFCKYKTLLRNENVLETAEALFNEL |
| | | NSIDLTHIFISHKKLETISSALCDHWDTLRNALYERRISELTGKITKSAKEKVQRSLKHEDINLQEIISAAG |
| | | KELSEAFKQKTSEILSHAHAALDQPLPTTLKKQEEKEILKSQLDSLLGLYHLLDWFAVDESNEVDPEFSA |
| | | RLTGIKLEMEPSLSFYNKARNYATKKPYSVEKFKLNFQMPTLASGWDVNKEKNNGAILFVKNGLYYLG |
| | | IMPKQKGRYKALSFEPTEKTSEGFDKMYYDYFPDAAKMIPKCSTQLKAVTAHFQTHTTPILLSNNFIEPL |
| | | EITKEIYDLNNPEKEPKKFQTAYAKKTGDQKGYREALCKWIDFTRDFLSKYTKTTSIDLSSLRPSSQYKD |
| | | LGEYYAELNPLLYHISFQRIAEKEIMDAVETGKLYLFQIYNKDFAKGHHGKPNLHTLYWTGLFSPENLA |
| | | KTSIKLNGQAELFYRPKSRMKRMAHRLGEKMLNKKLKDQKTPIPDTLYQELYDYVNHRLSHDLSDEAR |
| | | ALLPNVITKEVSHEIIKDRRFTSDKFFFHVPITLNYQAANSPSKFNQRVNAYLKEHPETPIIGIDRGERNLI |
| | | YITVIDSTGKILEQRSLNTIQQFDYQKKLDNREKERVAARQAWSVVGTIKDLKQGYLSQVIHEIVDLMIH |
| | | YQAVVVLENLNFGFKSKRTGIAEKAVYQQFEKMLIDKLNCLVLKDYPAEKVGGVLNPYQLTDQFTSFA |
| | | KMGTQSGFLFYVPAPYTSKIDPLTGFVDPFVWKTIKNHESRKHFLEGFDFLHYDVKTGDFILHFKMNRN |
| | | LSFQRGLPGFMPAWDIVFEKNETQFDAKGTPFIAGKRIVPVIENHRFTGRYRDLYPANELIALLEEKGIVF |
| | | RDGSNILPKLLENDDSHAIDTMVALIRSVLQMRNSNAATGEDYINSPVRDLNGVCFDSRFQNPEWPMD |
| | | ADANGAYHIALKGQLLLNHLKESKDLKLQNGISNQDWLAYIQELRN |
| FnCpf1 | 61 | MSIYQEFVNKYSLSKTLRFELIPQGKTLENIKARGLILDDEKRAKDYKKAKQIIDKYHQFFIEEILSSVCIS |
| | | EDLLQNYSDVYFKLKKSDDDNLQKDFKSAKDTIKKQISEYIKDSEKFKNLFNQNLIDAKKGQESDLILW |
| | | LKQSKDNGIELFKANSDITDIDEALEIIKSFKGWTTYFKGFHENRKNVYSSNDIPTSIIYRIVDDNLPKFLE |
| | | NKAKYESLKDKAPEAINYEQIKKDLAEELTFDIDYKTSEVNQRVFSLDEVFEIANFNNYLNQSGITKENTI |
| | | IGGKFVNGENTKRKGINEYINLYSQQINDKTLKKYKMSVLFKQILSDTESKSFVIDKLEDDSDVVTTMQS |
| | | FYEQIAAFKTVEEKSIKETLSLLFDDLKAQKLDLSKIYFKNDKSLTDLSQQVFDDYSVIGTAVLEYITQQI |
| | | APKNLDNPSKKEQELIAKKTEKAKYLSLETIKLALEEFNKHRDIDKQCRFEEILANFAAIPMIFDEIAQNK |
| | | DNLAQISIKYQNQGKKDLLQASAEDDVKAIKDLLDQTNNLLHKLKIFHISQSEDKANILDKDEHFYLVFE |
| | | ECYFELANIVPLYNKIRNYITQKPYSDEKFKLNFENSTLANGWDKNKEPDNTAILFIKDDKYYLGVMNK |
| | | KNNKIFDDKAIKENKGEGYKKIVYKLLPGANKMLPKVFFSAKSIKFYNPSEDILRIRNHSTHTKNGSPQK |
| | | GYEKFEFNIEDCRKFIDFYKQSISKHPEWKDFGFRFSDTQRYNSIDEFYREVENQGYKLTFENISESYIDS |
| | | VVNQGKLYLFQIYNKDFSAYSKGRPNLHTLYWKALFDERNLQDVVYKLNGEAELFYRKQSIPKKITHP |
| | | AKEAIANKNKDNPKKESVFEYDLIKDKRFTEDKFFFHCPITINFKSSGANKENDEINLLLKEKANDVHILS |
| | | IDRGERHLAYYTLVDGKGNIIKQDTFNIIGNDRMKTNYHDKLAAIEKDRDSARKDWKKINNIKEMKEG |
| | | YLSQVVHEIAKLVIEYNAIVVFEDLNFGFKRGRFKVEKQVYQKLEKMLIEKLNYLVFKDNEFDKTGGVL |
| | | RAYQLTAPFETFKKMGKQTGIIYYVPAGFTSKICPVTGFVNQLYPKYESVSKSQEFFSKFDKICYNLDKG |
| | | YFEFSFDYKNFGDKAAKGKWTIASFGSRLINFRNSDKNHNWDTREVYPTKELEKLLKDYSIEYGHGECI |
| | | KAAICGESDKKFFAKLTSVLNTILQMRNSKTGTELDYLISPVADVNGNFFDSRQAPKNMPQDADANGA |
| | | YHIGLKGLMLLGRIKNNQEGKKLNLVIKNEEYFEFVQNRNN |
| LbCpf1 | 62 | AASKLEKFTNCYSLSKTLRFKAIPVGKTQENIDNKRLLVEDEKRAEDYKGVKKLLDRYYLSFINDVLHSI |
| | | KLKNLNNYISLFRKKTRTEKENKELENLEINLRKEIAKAFKGAAGYKSLFKKDIIETILPEAADDKDEIAL |
| | | VNSFNGFTTAFTGFFDNRENMFSEEAKSTSIAFRCINENLTRYISNMDIFEKVDAIFDKHEVQEIKEKILNS |
| | | DYDVEDFFEGEFFNFVLTQEGIDVYNAIIGGFVTESGEKIKGLNEYINLYNAKTKQALPKFKPLYKQVLS |
| | | DRESLSFYGEGYTSDEEVLEVFRNTLNKNSEIFSSIKKLEKLFKNFDEYSSAGIFVKNGPAISTISKDIFGE |
| | | WNLIRDKWNAEYDDIHLKKKAVVTEKYEDDRRKSFKKIGSFSLEQLQEYADADLSVVEKLKEIIIQKVD |
| | | EIYKVYGSSEKLFDADFVLEKSLKKNDAVVAIMKDLLDSVKSFENYIKAFFGEGKETNRDESFYGDFVL |
| | | AYDILLKVDHIYDAIRNYVTQKPYSKDKFKLYFQNPQFMGGWDKDKETDYRATILRYGSKYYLAIMDK |
| | | KYAKCLQKIDKDDVNGNYEKINYKLLPGPNKMLPKVFFSKKWMAYYNPSEDIQKIYKNGTFKKGDMF |
| | | NLNDCHKLIDFFKDSISRYPKWSNAYDFNFSETEKYKDIAGFYREVEEQGYKVSFESASKKEVDKLVEE |
| | | GKLYMFQIYNKDFSDKSHGTPNLHTMYFKLLFDENNHGQIRLSGGAELFMRRASLKKEELVVHPANSPI |
| | | ANKNPDNPKKTTTLSYDVYKDKRFSEDQYELHIPIAINKCPKNIFKINTEVRVLLKHDDNPYVIGIDRGE |
| | | RNLLYIVVVDGKGNIVEQYSLNEIINNFNGIRIKTDYHSLLDKKEKERFEARQNWTSIENIKELKAGYISQ |
| | | VVHKICELVEKYDAVIALEDLNSGFKNSRVKVEKQVYQKFEKMLIDKLNYMVDKKSNPCATGGALKG |
| | | YQITNKFESFKSMSTQNGFIFYIPAWLTSKIDPSTGFVNLLKTKYTSIADSKKFISSFDRIMYVPEEDLFEF |
| | | ALDYKNFSRTDADYIKKWKLYSYGNRIRIFAAAKKNNVFAWEEVCLTSAYKELFNKYGINYQQGDIRA |
| | | LLCEQSDKAFYSSFMALMSLMLQMRNSITGRTDVDFLISPVKNSDGIFYDSRNYEAQENAILPKNADAN |
| | | GAYNIARKVLWAIGQFKKAEDEKLDKVKIAISNKEWLEYAQTSVK |

REC is the abbreviation of “recognition”. REC.1 domain is also called Helical I domain and the REC.2 domain is also called Helical II domain. WED is abbreviation of wedge and WED is also called OBD. The WED domain is the oligonucleotide-binding domain. REC lobe, WED lobe and PI (the abbreviation of PAM-interacting domain, also called LHD) can form a cleft. The mutants of the CRISPR-Cas12 protein are explored for obtaining some variants which have an altered PAM, have a modified nuclease activity (e.g., cleavage activity) and/or modify its ability to functionally associate with a target nucleic acid. In some embodiments, the variant can recognize a broader range of PAMs, and PAM preference would be selected. In some embodiments, the variant may comprise one or more mutations that increase the ability of the nuclease to cleave a target nucleic acid. In some embodiments, the variant is a high-fidelity version, and the reduced off-target effects.

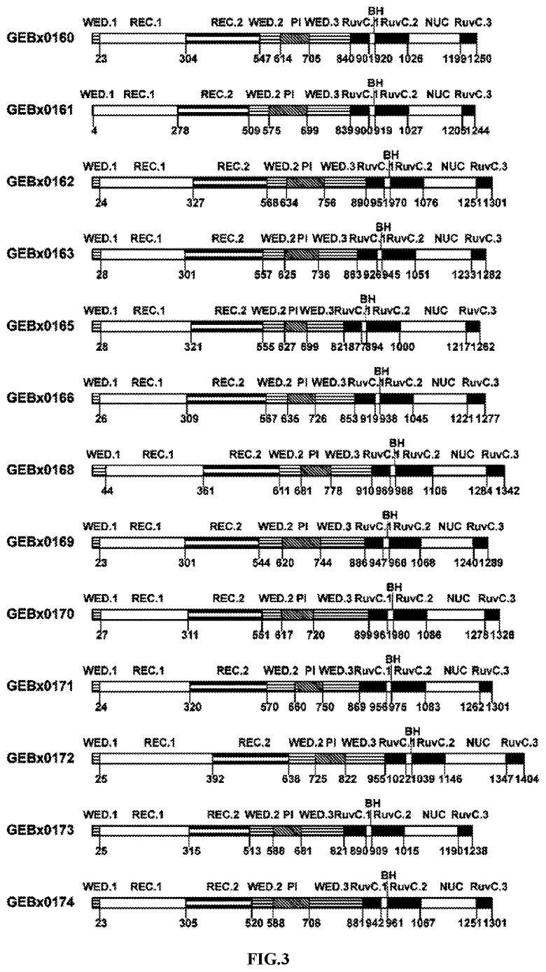

In some embodiments, the variant comprises one or more mutations in REC.1 domain, and/or WED.2 domain of any one of SEQ ID Nos: 1-13. The domains of SEQ ID Nos: 1-13 are shown in FIG. 3.

In some embodiments, the variant comprises one or more mutations in REC.1 domain, and/or WED.2 domain of SEQ ID NO: 12.

In some embodiments, the variant comprises one or more mutations in region of 150-200 and/or 513-588 with reference to amino acid position numbering of SEQ ID NO: 12; preferably, the variant comprises one or more mutations in region of 170-190 and/or 520-588 with reference to amino acid position numbering of SEQ ID NO: 12.

In some embodiments, the variant comprises one or more mutations in region of 175-185 and/or 530-588 with reference to amino acid position numbering of SEQ ID NO: 12; preferably, the variant comprises one or more mutations in region of 180-195 and/or 530-588 with reference to amino acid position numbering of SEQ ID NO: 12.

In some embodiments, the variant comprises one or more mutations at the following positions: I182, K532, E535, N536, and/or K586 of SEQ ID NO: 12.

In some embodiments, the variant comprises one mutation at the following positions: I182, K532, E535, N536, and/or K586 of SEQ ID NO: 12. For example, in an embodiment, the variant comprises one mutation at I182; in an embodiment, the variant comprises one mutation at K532; in an embodiment, the variant comprises one mutation at E535; in an embodiment, the variant comprises one mutation at N536; in an embodiment, the variant comprises one mutation at K586.

In some embodiments, the variant comprises two or more mutations at the following positions: I182, K532, E535, N536, and/or K586 of SEQ ID NO: 12.

In some embodiments, the variant comprises two mutations at the following positions: I182, K532, E535, N536, and/or K586 of SEQ ID NO: 12. For example, in an embodiment, the variant comprises the mutations at I182 and K532; in an embodiment, the variant comprises the mutations at I182 and E535; in an embodiment, the variant comprises the mutations at I182 and N536; in an embodiment, the variant comprises the mutations at I182 and K586; in an embodiment, the variant comprises the mutations at K532 and E535; in an embodiment, the variant comprises the mutations at K532 and N536; in an embodiment, the variant comprises the mutations at K532 and K586; in an embodiment, the variant comprises the mutations at E535 and N536; in an embodiment, the variant comprises the mutations at E535 and K586; in an embodiment, the variant comprises the mutations at N536 and K586.

In some embodiments, the variant comprises three or more mutations at the following positions: I182, K532, E535, N536, and/or K586 of SEQ ID NO: 12.

In some embodiments, the variant comprises three mutations at the following positions: I182, K532, E535, N536, and K586 of SEQ ID NO: 1. For example, in an embodiment, the variant comprises the mutations at I182, K532 and E535; in an embodiment, the variant comprises the mutations at I182, K532 and N536; in an embodiment, the variant comprises the mutations at I182, K532 and K586; in an embodiment, the variant comprises the mutations at K532, E535 and N536; in an embodiment, the variant comprises the mutations at K532, E535 and K586; in an embodiment, the variant comprises the mutations at E535, N536 and K586; in an embodiment, the variant comprises the mutations at I182, E535 and N536; in an embodiment, the variant comprises the mutations at I182, E535 and K586; in an embodiment, the variant comprises the mutations at I182, N536 and K586; in an embodiment, the variant comprises the mutations at K532, N536 and K586.

In some embodiments, the variant comprises four or more mutations at the following positions: I182, K532, E535, N536, and/or K586 of SEQ ID NO: 12.

In some embodiments, the variant comprises four mutations at the following positions: I182, K532, E535, N536, and K586 of SEQ ID NO: 1. For example, in an embodiment, the variant comprises the mutations at I182, K532, E535 and N536; in an embodiment, the variant comprises the mutations at I182, K532, E535, and K586; in an embodiment, the variant comprises the mutations at I182, K532, N536, and K586; in an embodiment, the variant comprises the mutations at I182, E535, N536, and K586; in an embodiment, the variant comprises the mutations at K532, E535, N536, and K586.

In some embodiments, the variant comprises the mutations at the following positions: I182, K532, E535, N536, and K586 of SEQ ID NO: 12.

In some embodiments, the mutation is a single amino acid substitution.

In some embodiments, the mutation on I182 is I182S or I182T, the mutation on K532 is K532V or K532A, the mutation on E535 is E535N or E535Q, the mutation on N536 is N536R, N536H or N536K, and the mutation on K586 is K586R, K586H or K586K. In some embodiments, the mutation on I182 is I182S, the mutation on K532 is K532V, the mutation on E535 is E535N, the mutation on N536 is N536R, and the mutation on K586 is K586R. For example, the variant comprises one or more mutations: I182S, K532V, E535N, N536R, and/or K586R based on amino acid sequence positions of SEQ ID NO: 12. In some embodiments, the variant comprises the following mutations: I182S, K532V, E535N, N536R, and K586R of SEQ ID NO: 12.

In some embodiments, the variant recognizes a PAM sequence which is not recognized by SEQ ID NO: 12.

In some embodiments, the variant recognizes a PAM sequence which is not TTTN, N is A, T, G or C.

In some embodiments, the variant has nuclease activity. In some embodiments, the variant has double-strand DNA cleavage activity or nickase activity.

In some embodiments, the Cas12 protein further comprises one or more of a nuclear localization signal sequence, a nuclear export signal sequence, a cell penetrating peptide sequence, an affinity tag and/or a fusion base editor protein.

The Cas12 protein comprises one or more nuclear localization signal(s) NLS(s). The NLS(s) can locate at the end or other portion of the peptide. The NLS(s) located each end or other portion of the Cas12 amino acid sequence can be same or not. In some embodiments, the NLS of the N-terminal end and the NLS of the C-terminal end are the same. In some embodiments, the NLS of the N-terminal end and the NLS of the C-terminal end are different. In some embodiments, the N-terminal end of the Cas12 amino acid sequence comprising one NLS and the C-terminal end of the Cas12 amino acid sequence comprising one NLS. The amino acid sequence of NLS fused to the N-terminal end or the C-terminal end of the Cas12 amino acid sequence respectively.

NLS is fused to a peptide or non-peptide moiety that allows proteins to enter or localize to a tissue, a cell, or a region of a cell. For instance, NLS maybe an SV40 (simian virus 40) NLS, c-Myc NLS, or other suitable monopartite NLS. The NLS may be fused to an N-terminal and/or a C-terminal of the Cas12 protein.

In some embodiments, the Cas12 protein includes at least one (e.g., 1, 2, 3, 4, 5, 6, 7, 8, 9, or 10) Nuclear Export Signal (NES) attached the N-terminal or C-terminal of the protein. In a preferred embodiment a C-terminal and/or N-terminal NLS or NES is attached for optimal expression and nuclear targeting in eukaryotic cells, e.g., human cells.

Generally, an affinity tag is added for purification of the fusion polypeptide by affinity chromatography.

In another aspect, the disclosure provides the engineered, non-naturally occurring Cas12 protein as described herein above, or the Cas12 polynucleotide as described herein above for use as nuclease, preferably, for use as double-strand DNA cleavage nuclease or nickase.

In another aspect, the disclosure provides the engineered, non-naturally occurring Cas12 protein for use in the gene editing.

In another aspect, the disclosure provides the engineered, non-naturally occurring Cas12 protein for use in a therapeutic or treatment or prevention or diagnosis or detection method of disease.

In another aspect, the disclosure provides the engineered, non-naturally occurring Cas12 protein for use as a medicament.

In another aspect, the disclosure provides the engineered, non-naturally occurring Cas12 protein for use in a method of therapeutic treatment of a patient.

In another aspect, the disclosure provides an engineered, non-naturally occurring cell comprising the Cas12 protein of any one of above.

In some embodiments, the cell is a eukaryotic cell or a prokaryotic cell. In some embodiments, the eukaryotic cell is selected from the group consisting of: a plant cell, a fungal cell, a single cell eukaryotic organism, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and a human cell. In some embodiments, the cell is a mammalian cell or a human cell or a plant cell.

The cell maybe the eukaryotic cell or the prokaryotic cell. In one embodiment, the cell is a eukaryotic cell. In another embodiment, the cell is a vertebrate, mammalian, rodent, goat, pig, bird, chicken, turkey, cow, horse, sheep, fish, primate, or human cell. In one embodiment, the cell is a mammalian cell. In one embodiment, the cell is a human cell. In one embodiment, the cell is a somatic cell, a germ cell, or a prenatal cell. In one embodiment, the cell is a zygotic cell, a blastocyst cell, an embryonic cell, a stem cell, a mitotically competent cell, or a meiotically competent cell. In one embodiment, the cell is not part of a human embryo. In one embodiment, the cell is a somatic cell. In one embodiment, the cell is a T cell, a CD⁸⁺ T cell, a CD⁸⁺ naive T cell, a central memory T cell, an effector memory T cell, a CD⁴⁺ T cell, a stem cell memory T cell, a helper T cell, a regulatory T cell, a cytotoxic T cell, a natural killer T cell, a Hematopoietic Stem Cell, a long term hematopoietic stem cell, a short term hematopoietic stem cell, a multipotent progenitor cell, a lineage restricted progenitor cell, a lymphoid progenitor cell, a myeloid progenitor cell, a common myeloid progenitor cell, an erythroid progenitor cell, a megakaryocyte erythroid progenitor cell, a retinal cell, a photoreceptor cell, a rod cell, a cone cell, a retinal pigmented epithelium cell, a trabecular meshwork cell, a cochlear hair cell, an outer hair cell, an inner hair cell, a pulmonary epithelial cell, a bronchial epithelial cell, an alveolar epithelial cell, a pulmonary epithelial progenitor cell, a striated muscle cell, a cardiac muscle cell, a muscle satellite cell, a neuron, a neuronal stem cell, a mesenchymal stem cell, an induced pluripotent stem (iPS) cell, an embryonic stem cell, a monocyte, a megakaryocyte, a neutrophil, an eosinophil, a basophil, a mast cell, a reticulocyte, a B cell, e.g., a progenitor B cell, a Pre B cell, a Pro B cell, a memory B cell, a plasma B cell, a gastrointestinal epithelial cell, a biliary epithelial cell, a pancreatic ductal epithelial cell, an intestinal stem cell, a hepatocyte, a liver stellate cell, a Kupffer cell, an osteoblast, an osteoclast, an adipocyte, a preadipocyte, a pancreatic islet cell (e.g., a beta cell, an alpha cell, a delta cell), a pancreatic exocrine cell, a Schwann cell, or an oligodendrocyte. In one embodiment, the cell is a T cell, a Hematopoietic Stem Cell, a retinal cell, a cochlear hair cell, a pulmonary epithelial cell, a muscle cell, a neuron, a mesenchymal stem cell, an induced pluripotent stem (iPS) cell, or an embryonic stem cell. In another embodiment, the cell is a plant cell.

In another aspect, the disclosure provides a kit comprising the engineered, non-naturally occurring Cas12 protein of any one of above. In addition, the reagent kit can comprise the other components, for example, a solution or a buffer.

It would be appreciated that the kit may further comprise other suitable excipients such as buffers or reagents for facilitating the application of the kit. Preferably, the kit may be applied in various applications such as medical applications including therapies and diagnosis, researches and the like. Accordingly, the Cas12 protein and the kit of the present invention may be used in the preparation of a medicament for treatment and/or in the preparation of an agent for research study.

In another aspect, the disclosure provides an engineered, non-naturally occurring Cas12 polynucleotide encoding the Cas12 protein of any one of above.

The polynucleotides, may be in the form of RNA or DNA, which includes cDNA, genomic DNA, and synthetic DNA. A polynucleotide may be double stranded or single stranded, and if single stranded, may be the coding strand or non-coding (anti-sense strand). A coding polynucleotide may have a coding sequence identical to a coding sequence known in the art or may have a different coding sequence, which, as the result of the redundancy or degeneracy of the genetic code, or by splicing, can encode the same polypeptide.

The polypeptide may include not only coding sequences but also regulatory regions such as promoters, enhancers, and termination regions. The term further can include all introns and other DNA sequences spliced from the mRNA transcript, along with variants resulting from alternative splice sites. These nucleic acid sequences may be a DNA strand sequence that is transcribed into RNA or an RNA sequence that is translated into protein. The nucleic acid sequences include both the full-length nucleic acid sequences as well as non-full-length sequences derived from the full-length protein. The sequences can also include degenerate codons of the native sequence or sequences that may be introduced to provide codon preference in a specific cell type. The polypeptide sequences are referenced throughout the disclosure as is understood by one of ordinary skill in the art.

In some embodiments, the polynucleotide is ribonucleotide sequence or deoxyribonucleotide sequence or analogs thereof; preferably the polynucleotide is mRNA, and polynucleotide further comprises 5′cap sequence and poly-A tail sequence. In some embodiments, the polynucleotide is codon optimized for expression in a cell of interest. In some embodiments, the polynucleotide is codon optimized for expression in a eukaryotic cell. In some embodiments, the polynucleotide has at least 90%, 92%, 95% or 98% sequence identity to any one of SEQ ID NOs: 91-94. In some embodiments, the polynucleotide has at least 95% sequence identity to any one of SEQ ID NOs: 91-94. In some embodiments, the polynucleotide has the sequence set forth in any one of SEQ ID NOs: 91-94. In some embodiments, the eukaryotic cell is selected from the group consisting of: a plant cell, a fungal cell, a single cell eukaryotic organism, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and a human cell. In some embodiments, the cell is a mammalian cell, preferably a human cell. In some embodiments, the cell is a mammalian cell, preferably a human cell.

In some embodiments, the polynucleotide has at least 70% sequence identity to any one of the SEQ D NOs: 14-26.

In some embodiments, the polynucleotide has at least 75%, 80%, 85%, 88%, 90%, 92%, 94%, 95%, 96%, 98% or 99% sequence identity to any one of the SEQ TD NOs: 14-26.

For example, “at least 70%” can include 70%, 72%, 75%, 80%, 85%, 86%, 87%, 88%, 90%, 91%, 92%, 93%, 94%, 95%, 96%, 97%, 98%, 99%, 100%; “at least 80%” can include 85%, 86%, 87%, 88%, 90%, 91%, 92%, 93%, 94%, 95%, 96%, 97%, 98%, 99% or 100%; “at least 85%” can include 85%, 86%, 88%, 89%, 90%, 91%, 92%, 93%, 94%, 95%, 96%, 97%, 98%, 99% or 100%; “at least 90%” can include 90%, 91%, 92%, 93%, 94%, 95%, 96%, 97%, 98%, 99% or 100%; “at least 95%” can include 95%, 96%, 97%, 98%, 99% or 100%; “at least 97%” can include 97%, 98%, 99% or 100%; “at least 98%” can include 98%, 99% or 100%; and so on.

TABLE 3
The nucleic acid sequences of the example Cas12 proteins

| Cas12 | SEQ ID | |
|---|---|---|
| protein | NO: | 5′ to 3′ |
| GEBx0173 | 25 | GCCCTGTTCAGCGAGAACTTCGTGGGCTGCTACCCCGTGAGCAAGACCCTGAAGTTCGAGCTGAGG |
| | | CCCGTGCCCGAGACCAAGGCCTACCTGGACGACGACAACTGCCCCCTGCTGGCCGGCGACCTGGA |
| | | GAGGGACAAGGCCTACCCCGTGGTGAAGGAGCTGCTGGACAAGTACTACAGGTACTTCATCGACA |
| | | TCTGCCTGAAGGACAAGAAGCTGAGCCCCGAGACCATCGACGAGGCCTACCAGGCCTACAGCAGC |
| | | CACGACGAGAAGAGCCAGAAGGCCCTGAAGGCCGCCAGCCAGAAGCTGAGGAAGGAGGTGGCCG |
| | | GCTTCTTCGACAAGAAGACCAGGGAGACCTACTGCCTGGACAAGTACAGCAACCTGCTGGTGCTGG |
| | | ACAAGAAGAACGACAACAAGGAGAAGGGCACCCCCAAGACCATCCCCAGCGTGCTGAAGAAGTG |
| | | GCTGGAGAACGAGAGCGGCTTCAGCGAGAAGGAGGTGGAGAAGTACAGCCAGGCCGTGCAGACCT |
| | | TCAACGGCTTCACCACCTACTTCATCAGCTACAAGGAGAACAGGGAGAACATGTTCAGCAGCGAG |
| | | GAGAAGGCCAGCGCCATCAGCTACAGGGTGGTGAACCAGAACATGCTGAGGTTCTTCAGCAACGT |
| | | GGAGAACTTCGAGAACATCAAGAAGAAGTACCCCGACCTGTACCAGACCCTGAAGGAGTTCGAGG |
| | | AGAGCTTCGTGCCCGCCGCCTACAGCAGCCTGCTGAGCCAGGCCGGCATCGACGACTACAACTACA |
| | | GGGTGATCGGCAGGCCCGCCGAGGACGAGGACTTCGCCGGCGTGAACAGGCTGATCAACGAGTAC |
| | | AGGCAGAAGAACGCCGTGAGGAACAGGAACCTGCCCGTGATGGTGACCCTGTACAAGCAGATCCT |
| | | GAGCGACAGGGAGAACGGCTTCGGCAGGGACATCCTGGCCAGCGACAGCGAGGCCAAGGAGTAC |
| | | ATCAGGGGCAAGTACAGCAAGGCCTACGACACCGCCTGCCAGCTGACCGTGTTCGTGAACAACTAC |
| | | ATCCTGACCGAGGACCTGGACACCGTGTACGTGAAGACCCAGCACCTGAAGGACATCAGCCAGAG |
| | | CATCTTCGGCGAGTGGAGCGTGCTGGACGACGCCATCAACAACGGCGGCATCGACAGCAAGATCA |
| | | TCAGCCTGAAGGCCCTGGCCGAGGCCTTCGAGAAGTACTGCAGCGCCCTGGACCCCGACATGGCCC |
| | | AGAAGTACAGGGACGCCTTCAGCCTGAAGGAGTACCTGAAGAACCCCCCCGCCGTGGAGCCCAGG |
| | | CTGCCCGAGACCGCCGAGAAGGTGACCGCCTACAAGCCCGCCATGGACGAGCTGATCAGCCTGAT |
| | | CAGGTTCTACAAGCCCTTCTACCTGTACGACGGCAAGAAGAGGCTGCAGGTGCCCGCCGAGGGCAT |
| | | GGACTTCAGCAACGAGTTCGACAAGTACTTCGAGGACCTGAAGGAGTTCAGCCACGCCTACGACA |
| | | AGGTGAGGAACTTCGCCACCAAGAAGCCCTACAGCGTGGAGAAGATCAAGCTGAACTTCAACCAG |
| | | CCCACCCTGCTGGACGGCTGGGACGTGAACAAGGAGAAGGAGAACGCCAGCTTCCTGTTCGTGAA |
| | | GGAGGGCAAGTACTACCTGGGCATCGCCGACAAGAGCGACAAGAACCTGTTCGACAGGAGCAGGG |
| | | ACGCCGTGAAGGAGGCCCTGACCCTGACCTGCGGCGAGGACTACTACGAGAAGGTGCAGTACAAG |
| | | CAGCTGCCCAACATCAGGATGAACCTGCCCAGGATCGTGTTCAAGCCCAAGTGGGAGAAGGTGTTC |
| | | GCCCCCATCATCACCGAGAGGATCAGCGCCATCGAGAAGGAGAAGCTGTACACCGCCGGCGCCAA |
| | | CGACAGGCAGGCCGTGATCGAGTGGATCGACTTCGTGAAGAGCGTGCTGGCCCTGCACAGCGAGT |
| | | GGAACAACTACTTCCAGTTCAAGTTCAAGGAGACCAGCGCCTACAAGAACACCAAGGAGTTCTAC |
| | | GACGACCTGGACAACCAGGCCTACATCCTGAGCAAGATCAAGATCAACGCCAGCTACATCCACAA |
| | | GCTGGTGAGGGAGAGGAAGCTGTACCTGTTCCAGATCTACTGCAAGGACTTCAGCGACAAGAAGA |
| | | AGAAGACCGGCGCCGACAACCTGCACACCATGTACTGGAGGTGCCTGTTCAGCGACGAGAACCTG |
| | | GCCGCCCTGGCCCAGGGCCAGGCCCCCATCATCAAGCTGAACGGCAAGGCCGAGATCTTCCTGAGG |
| | | GAGCCCAGCCTGAAGTACGAGGCCACCCACCCCAAGAACCAGCCCGTGGCCAACAAGAACCCCCT |
| | | GAACAGCAAGAGGGAGAGCCTGTTCAGCTACGACCTGGCCAAGAACAGGAGGTTCACCGAGAGGA |
| | | AGCTGTTCTTCCACTGCCCCCTGACCATCAACTTCAGGACCGCCGCCGAGGGCGCCAGGAGCTTCA |
| | | ACGAGAGGGTGAACAGGTTCGTGGAGAACAACCCCGACATCAGGATCATCGGCATCGACAGGGGC |
| | | GAGAGGCACCTGCTGTACTACACCGTGATCAACCAGAGCGGCAAGATCCTGGAGCAGGGCAGCCT |
| | | GAACAAGATCAGCAGCGACTACAGGACCCCCGAGGGCGCCGAGGTGGAGAAGGTGACCGACTACC |
| | | ACGAGCTGCTGAACAGGAAGGAGGGCGACAGGAGGGCCGCCAGGCAGACCTGGGACACCATCGA |
| | | GAACATCAAGGAGCTGAAGGAGGGCTACCTGAGCCAGGTGGTGCACAAGCTGACCACCCTGATGA |
| | | TCAAGCACAACGCCGTGGTGGTGCTGGAGAACCTGAACCAGAACTTCAAGAGGAGCAGGATCAAG |
| | | GTGGAGAAGCAGGTGTACCAGAAGTTCGAGAAGGCCCTGATCGACAAGCTGAACTACCTGGTGTT |
| | | CAAGGACAGGCCCTACGGCGCCCCCGGCAGCTACGCCAACGGCCTGCAGCTGACCGCCCCCTTCGA |
| | | GAGCTTCGACAGGCTGGGCAAGCAGACCGGCGCCCTGTACTACGTGGTGCCCAGCTACACCAGCCA |
| | | CATCGACCCCAAGACCGGCTTCGTGAACCTGCTGGGCGCCAAGCTGAGGTACGAGAGCGTGGTGA |
| | | AGGCCCAGGAGACCATGAAGAAGATCGGCAGCTTCAAGTTCAACCCCGAGAGGAACTGGTTCGAG |
| | | ATCAGCTTCGACTACGGCGACTTCGGCATCGAGATGGCCCACCCCCAGTGGACCGCCTGCACCCAC |
| | | GGCGACGCCTACTGGGGCTACAACCCCAAGGAGAAGAGGGCCGTGAGCTACGACGTGACCGAGGA |
| | | GATCAAGAAGCTGCTGACCGAGTACGGCATCGACTACGGCACCGGCGCCGACATCAAGGAGGCCG |
| | | CCATCAGGCAGACCAAGCCCGCCTTCTGGAAGACCCTGTTCTACATGCTGAAGCTGGCCCTGCAGC |
| | | TGAGGAACACCGTGGCCGGCACCGACAACGAGAACGACTACATCCTGAGCCCCGTGGAGTACGAG |
| | | GCCGGCAAGTTCTTCGACAGCAGGAAGGCCGGCGAGGACGAGCCCCAGAACGCCGACGCCAACGG |
| | | CGCCTACCACATCGCCATGAAGGGCCTGCAGGCCCTGAGGAGGATCGAGGACGGCAAGGTGAACA |
| | | AGTTCCCCAAGAACGGCGAGAGGAAGGCCTGGCTGGCCTTCATGCAGAACAGGGAGTACCTGAAG |

In table 3, the nucleic acid sequences of the example Cas12 proteins are provided and the nucleic acids are the Non-Human Codon Optimized sequence.

In another aspect, the disclosure provides an engineered vector comprising the Cas12 polynucleotide of any one of above.

In certain aspects, the invention involves vectors. As used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. Common expression vectors of utility in recombinant DNA techniques are often in the form of plasmids. Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell).

In some embodiments, the vector is an expression vector. In some embodiments, the vector is an inducible, conditional, or constitutive expression vector.

In another aspect, the disclosure provides a vector system comprising one or more vectors of any one of above. In some embodiments, one or more vectors comprise a polynucleotide according to any one of above and one or more polynucleotides which are on the same or a different vector encoding a guide RNA.

In another aspect, the disclosure provides an engineered cell comprising the Cas12 polynucleotide of any one of above, or comprising the vector of any one of above, or comprising the vector system of any one of above.

In some embodiments, the cell is expressing the Cas12 protein. In some embodiments, the cell transiently expresses or non-transiently expresses the Cas12 protein. In some embodiments, the cell is a eukaryotic cell or a prokaryotic cell. In some embodiments, the cell is a mammalian cell or a human cell or a plant cell.

In another aspect, the disclosure provides a reagent kit comprising the Cas12 protein of any one of above, or comprising the Cas12 polynucleotide of any one of above, or comprising the vector of any one of above, or comprising the vector system of any one of above.

In another aspect, the disclosure provides a pharmaceutical composition comprising the Cas12 protein of any one of above or the polynucleotide of any one of above or the vector of any one of above or the vector system of any one of above formulated for delivery by AAV (adena-associated viruses), Adenoviruses, retroviruses, HSV (herpes simplex virus), Gammaretrovirus, LV (lentivirus), eCIS (extracellular Contractile Injection System), eVLP (Engineered virus-like particles), VLP (virus-like particles), liposomes, plasmid, lipid nanoparticles (LNPs), exosomes, microvesicles, nucleic acid nanoassemblies, a gene gun, and/or an implantable device.

“Gammaretrovirus” refers to a genus of the retroviridae family. Exemplary gammaretroviruses include mouse stem cell virus, murine leukemia virus, feline leukemia virus, feline sarcoma virus, and avian reticuloendotheliosis viruses.

The CRISPR-Cas12 system of the below or pharmaceutical composition of above described herein, or components thereof, nucleic acid molecules thereof, or nucleic acid molecules encoding or providing components thereof, can be delivered by various delivery systems such as vectors, e.g., plasmids, viral delivery vectors, such as adeno-associated viruses (AAV), lentiviruses, adenoviruses, and other viral vectors, or methods, such as nucleofection or electroporation of ribonucleoprotein complexes consisting of Type V-I effectors and their cognate RNA guide or guides. The proteins and one or more RNA guides can be packaged into one or more vectors, e.g., plasmids or viral vectors. For bacterial applications, the nucleic acids encoding any of the components of the CRISPR systems described herein can be delivered to the bacteria using a phage. Exemplary phages, include, but are not limited to, T4 phage, Mu, λ phage, T5 phage, T7 phage, T3 phage, Φ29, M13, MS2, Qβ, and ΦX174.

In some embodiments, the vectors, e.g., plasmids or viral vectors, are delivered to the tissue of interest by, e.g., intramuscular injection, intravenous administration, transdermal administration, intranasal administration, oral administration, or mucosal administration. Such delivery may be either via a single dose or multiple doses. One skilled in the art understands that the actual dosage to be delivered herein may vary greatly depending upon a variety of factors, such as the vector choices, the target cells, organisms, tissues, the general conditions of the subject to be treated, the degrees of transformation/modification sought, the administration routes, the administration modes, the types of transformation/modification sought, etc.

In certain embodiments, the delivery is via adeno-associated viruses (AAV), e.g., AAV2, AAV8, or AAV9, which can be administered in a single dose containing at least 1×10⁵ particles (also referred to as particle units, pu) of adenoviruses or adeno-associated viruses. In some embodiments, the dose is at least about 1×10⁶ particles, at least about 1×10⁷ particles, at least about 1×10⁸ particles, or at least about 1×10⁹ particles of the adeno-associated viruses. Due to the limited genomic payload of recombinant AAV, the smaller size of the Cas12 proteins described herein enables greater versatility in packaging the effector and RNA guides with the appropriate control sequences (e.g., promoters) required for efficient and cell-type specific expression.

In some embodiments, the delivery is via a recombinant adeno-associated virus (rAAV) vector. For example, in some embodiments, a modified AAV vector may be used for delivery. Modified AAV vectors can be based on one or more of several capsid types, including AAV1, AAV2, AAV5, AAV6, AAV8, AAV8.2. AAV9, AAV rh1O, modified AAV vectors (e.g., modified AAV2, modified AAV3, modified AAV6) and pseudotyped AAV (e.g., AAV2/8, AAV2/5 and AAV2/6). Exemplary AAV vectors and techniques that may be used to produce rAAV particles are known in the art (see, e.g., Aponte-Ubillus et al. (2018) Appl. Microbiol. Biotechnol. 102(3): 1045-54; Zhong et al. (2012) J. Genet. Syndr. Gene Ther. Si: 008; West et al. (1987) Virology 160: 38-47 (1987); Tratschin et al. (1985) Mol. Cell. Biol. 5: 3251-110), each of which is incorporated by reference).

In some embodiments, the delivery is via plasmids. The dosage can be a sufficient number of plasmids to elicit a response. In some cases, suitable quantities of plasmid DNA in plasmid compositions can be from about 0.1 to about 2 mg. Plasmids will generally include (i) a promoter; (ii) a sequence encoding a nucleic acid-targeting CRISPR enzymes, operably linked to the promoter; (iii) a selectable marker; (iv) an origin of replication; and (v) a transcription terminator downstream of and operably linked to (ii). The plasmids can also encode the RNA components of a CRISPR-Cas system, but one or more of these may instead be encoded on different vectors. The frequency of administration is within the ambit of the medical or veterinary practitioner (e.g., physician, veterinarian), or a person skilled in the art.

In another embodiment, lipid nanoparticles (LNPs) are contemplated. LNPs can take different materials to form different forms. For example, the LNP may comprises: a cationic lipid at a molar ratio between 35% and 45%, a polyethylene glycol (PEG) conjugated (PEGylated) lipid at a molar ratio between 0.25% and 2.75%, a cholesterol-based lipid at a molar ratio between 20% and 35%, and a helper lipid at a molar ratio of between 25% and 35%, wherein all the molar ratios are relative to the total lipid content of the LNP. LNP can be made into different sizes, such as an average diameter of 30-200 nm or 80-150 nm.

In another embodiment, the delivery is via liposomes or lipofection formulations and the like, and can be prepared by methods known to those skilled in the art. Such methods are described, for example, in WO 2016205764 and U.S. Pat. Nos. 5,593,972; 5,589,466; and 5,580,859; each of which is incorporated herein by reference in its entirety.

In some embodiments, the delivery is via nanoparticles or exosomes. For example, exosomes have been shown to be particularly useful in the delivery of RNA.

Further means of introducing one or more components of the new CRISPR systems into cells is by using cell penetrating peptides (CPP). In some embodiments, a cell penetrating peptide is linked to the CRISPR enzymes. In some embodiments, the CRISPR enzymes and/or RNA guides are coupled to one or more CPPs to transport them inside cells effectively (e.g., plant protoplasts). In some embodiments, the CRISPR enzymes and/or RNA guide(s) are encoded by one or more circular or non-circular DNA molecules that are coupled to one or more CPPs for cell delivery.

In another aspect, the disclosure provides an engineered, non-naturally occurring CRISPR-Cas system comprising:
a) the Cas12 protein of any one of above or the polynucleotide encoding the Cas12 protein;
b) at least one engineered guide sequence or one or more engineered nucleic acid encoding the at least one engineered guide sequence, and the guide sequence comprises a direct repeat sequence capable of binding the Cas12 protein and a spacer sequence capable of hybridizing to a target sequence.

The engineered Cas12 protein that complexes with the guide sequence to form a CRISPR complex, and wherein in the CRISPR complex the nucleic acid molecule target one or more polynucleotide loci.

In some embodiments, the direct repeat sequence and the spacer sequence are heterologous. “Heterologous”, as used herein, means a nucleotide or polypeptide sequence that is not found in the native nucleic acid or protein, respectively.

In some embodiments, the system comprises at least one guide sequences which are capable of hybridizing at least one target sequences or different regions of one target sequence. In some embodiments, the guide sequence hybridizes to one or more target sequences in a prokaryotic cell or in a eukaryotic cell. In some embodiments, the eukaryotic cell is selected from the group consisting of: a plant cell, a fungal cell, a single cell eukaryotic organism, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and a human cell. In some embodiments, the eukaryotic cell comprises a mammalian cell. In some embodiments, the mammalian cell comprises a human cell. In some embodiments, the eukaryotic cell comprises a plant cell.

In some embodiments, the target sequence is DNA or RNA. In some embodiments, the target sequence is selected from: the target sequence is selected from: double stranded DNA, double stranded RNA, single stranded DNA, single stranded RNA, genomic DNA, or extrachromosomal DNA.

In some embodiments, the direct repeat sequence comprises a stem-loop structure and the direct repeat sequence comprises a nucleotide sequence having at least 95% identity to any one of SEQ ID NOs: 27-33.

In some embodiments, the direct repeat sequence comprises a nucleotide sequence set forth in any one of SEQ ID NOs: 27-33.

In some embodiments, the nucleotide sequence of the direct repeat sequence corresponding to different Cas12 proteins is shown in table 4.

TABLE 4
The Cas12 protein and the direct repeat sequences

| | direct repeat sequence | |
|---|---|---|
| | SEQ ID | |
| Cas12 protein | NO: | 5′-3′ |
| GEBx0160/GEBx0161/ | 27 | AAUUUCUACUAUUGUAGAU |
| GEBx0169 | | |
| GEBx0162 | 28 | AAUUUCUACUAUCGUAGAU |
| GEBx0163/GEBx0166/ | 29 | AAUUUCUACUGUUGUAGAU |
| GEBx0170/GEBx0172/ | | |
| GEBx0173 | | |
| GEBx0165 | 30 | CAUUUCUACUUUUGUAGAU |
| GEBx0168 | 31 | AAUUUCUACUUUUGUAGAU |
| GEBx0171 | 32 | GAUUUCUACUUUUGUAGAU |
| GEBx0174 | 33 | AAUUUCUACUUUCGUAGAU |

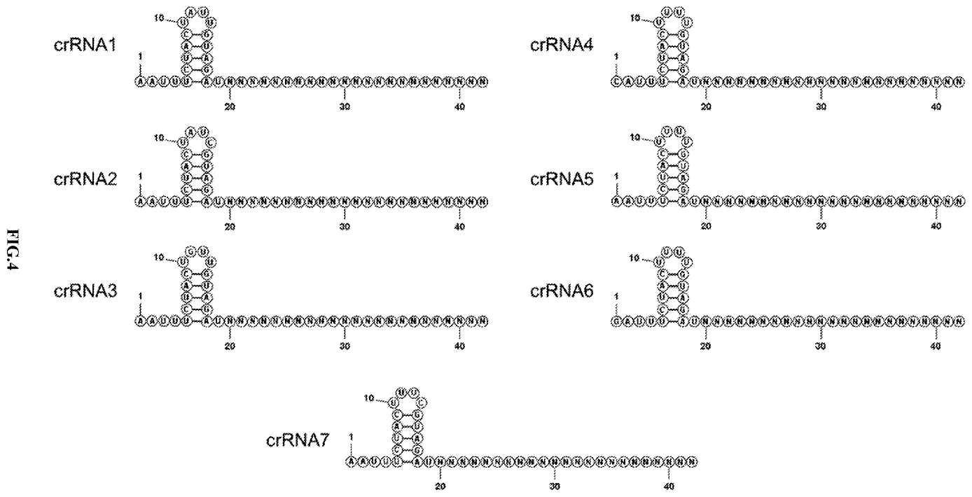

The engineered crRNA or the engineered guide sequence described herein comprises a spacer sequence and a direct repeat sequence. The predicted crRNA secondary structures are shown in FIG. 4. In FIG. 4, N represents the target specific sequence and the number of N is just an example illustration which does not represent its actual nucleotide quantity.

The guide RNA secondary structures of the Cas12 protein suggest that Cas12 protein could process and utilize each other's crRNAs for DNA targeting. A “stem-loop structure” refers to a nucleic acid having a secondary structure that includes a region of nucleotides that are known or predicted to form a double strand (stem portion) that is linked to one side by a region of predominantly single-stranded nucleotides (loop portion). The terms “hairpin” and “fold-back” structures are also used herein to refer to stem-loop structures. Such structures are well known in the art and these terms are used consistently with their known meanings in the art. As is known in the art, a stem-loop structure does not require exact base-pairing. Thus, the stem may include one or more base mismatches. Alternatively, the base-pairing may be exact, i.e., not include any mismatches. The predicted stem loop structure of the direct repeat is illustrated in FIG. 4. In FIG. 4, “N” is just an example illustration and does not represent its actual nucleotide quantity.

In certain embodiments, the Cas12 protein has nuclease activity. In certain embodiments, the Cas12 protein has single-strand RNA cleavage activity, double-strand RNA cleavage activity, single-strand DNA cleavage activity, double-strand DNA cleavage activity, the nucleic acid binding activity, or collateral cleavage activity of RNA and/or DNA. In some embodiments, Cas12 protein has endonuclease activity, nickase activity, and/or exonuclease activity.

In certain embodiments, the Cas12 protein according to the disclosure as described herein, the Cas12 protein may be a deactivated or inactivated Cas12 protein (e.g., “dead” Cas12 protein), wherein catalytic activity is partially or (substantially) completely lost, as described herein elsewhere. Loss of catalytic activity in this context means that the Cas12 protein is not capable of cleaving DNA (e.g., not capable of inducing double strand breaks, or only capable of inducing single strand breaks, such as a nickase). The Cas12 protein may be used to reduce off-target effects, as defined herein elsewhere. The Cas12 protein may also be part of a fusion protein, as defined herein elsewhere. The Cas12 protein may also be described to include a destabilization domain, as defined herein elsewhere. The Cas12 protein may also be a split Cas12 protein, as defined herein elsewhere. The Cas12 protein may also be an inducible Cas12 protein, as defined herein elsewhere. The Cas12 protein may also be part of a self-inactivating system (SIN), as defined herein elsewhere. The Cas12 protein may also be part of a synergistic activator system (SAM) as defined herein elsewhere.

Accordingly, in certain embodiments, the Cas12 protein polypeptide according to the disclosure as described herein is comprised in a fusion protein with a functional domain. In certain embodiments, said functional domain comprises a (transcriptional) activator domain, a (transcriptional) repressor domain, a recombinase, a transposase, a histone remodeler, a DNA methyltransferase, a cryptochrome, a light inducible/controllable domain, or a chemically inducible/controllable domain.

In certain embodiments, the Cas12 polypeptide according to the disclosure as described herein is not capable of inducing a DNA double strand break. In certain embodiments, the Cas12 polypeptide according to the disclosure as described herein is a nickase. In certain embodiments, the Cas12 polypeptide according to the disclosure as described herein is a catalytically inactive Cas12 polypeptide. In certain embodiments, the Cas12 polypeptide according to the disclosure as described herein is not capable of inducing a DNA single strand break. In an exemplary, the Cas12 protein is a dead Cas12 protein having a catalytically inactive. In an exemplary, the Cas12 protein is a nickase having a catalytically inactive.

In some embodiments, a vector encoding Cas12 protein lacks the ability to cleave one or both strands of a target polynucleotide containing a target sequence. In some embodiments, the Cas12 protein lack all DNA cleavage activity when the DNA cleavage activity of the enzyme is about no more than 25%, 10%, 5%, 1%, 0.1%, 0.01%, or less of the DNA cleavage activity. Thus, the Cas12 protein may be used as a generic DNA binding protein with or without fusion to a functional domain. In one aspect of the disclosure, the Cas12 enzyme may be fused to a protein, e.g., a TAG, and/or an inducible/controllable domain such as a chemically inducible/controllable domain. The Cas12 in the disclosure may be a chimeric Cas12 proteins; e.g., a Cas12 having enhanced function by being a chimera. Chimeric Cas12 proteins may be new Cas containing fragments from more than one naturally occurring Cas. In some embodiments, the Cas12 protein has enhanced on target activity without higher off target cutting or for making super cutting nickases, or for combination with a mutation that renders the Cas dead for a super binder.

The Cas12 enzyme provided in this disclosure can recognize a short motif associated in the vicinity of a target DNA called a Protospacer Adjacent Motif (PAM). In some embodiments, the Cas12 enzyme can recognize the canonical PAM comprising or consisting of 5′-TTTN-3′ and the non-canonical sequences, wherein N denotes any nucleotide. For example, the canonical PAM may be TTTA, TTTT, TTTG, or TTTC. In some embodiments, the PAM sequence recognized by the Cas12 enzyme is 5′-TTTG-3′.

In some embodiments, the spacer sequence is between 18 and 23 nucleotides in length, preferably the spacer sequence is 19 or 23 nucleotides in length.

In some embodiments, the polynucleotide encoding the Cas12 protein is a mRNA or a DNA. In some embodiments, the polynucleotide encoding the Cas12 protein is operably linked to a promoter. In some embodiments, the promoter is a constitutive promoter, tissue-specific promoter or inducible promoter. In some embodiments, the polynucleotide encoding the Cas12 protein operably linked to a promoter is in a vector. In some embodiments, the vector is selected from the group consisting of a retroviral vector, a lentiviral vector, a phage vector, an adenoviral vector, an adeno-associated vector, and a herpes simplex vector.

In some embodiments, the system further comprises a donor template nucleic acid, wherein the donor template nucleic acid is a DNA or RNA or DNA-RNA hybrids.

In some embodiments, the targeting of the target sequence by the Cas12 protein and guide sequence results in a modification of the target sequence. In some embodiments, the modification of the target sequence is a cleavage event or a nicking event.

In another aspect, the disclosure provides a delivery system, wherein the system of any one of above is presented in selected from the group consisting of AAV (adena-associated viruses), Adenoviruses, retroviruses, HSV (herpes simplex virus), Gammaretrovirus, LV (lentivirus), eCIS (extracellular Contractile Injection System), eVLP (Engineered virus-like particles), VLP (virus-like particles), liposomes, plasmid, lipid nanoparticles (LNPs), exosomes, microvesicles, nucleic acid nanoassemblies, a gene gun, and/or an implantable device.

In another aspect, the disclosure provides an engineered cell comprising the system of any one of above. In some embodiments, the cell is a eukaryotic cell or a prokaryotic cell. In some embodiments, the eukaryotic cell is selected from the group consisting of: a plant cell, a fungal cell, a single cell eukaryotic organism, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and a human cell. In some embodiments, the cell is a mammalian cell or a human cell or a plant cell.

In another aspect, the disclosure provides the engineered, non-naturally occurring CRISPR-Cas system of any one of above, or the delivery system of above for use in a therapeutic or treatment or prevention or diagnosis or detection method of disease.

In another aspect, the disclosure provides the engineered, non-naturally occurring CRISPR-Cas system of any one of above, delivery system of above or cell of any one of above for use as a medicament.

In another aspect, the disclosure provides the engineered, non-naturally occurring CRISPR-Cas system of any one of above, delivery system of above or cell of any one of above for use in a method of therapeutic treatment of a patient.

In another aspect, the disclosure provides a method of modifying or targeting a target DNA locus, the method comprising delivering to said locus a CRISPR-Cas system of any one of above or a delivery system of above.

In some embodiments, said modifying or targeting a target locus comprises inducing a DNA strand break. In some embodiments, said modifying or targeting a target locus comprises inducing a DNA double strand break or a DNA single strand break. In some embodiments, said modifying or targeting a target locus comprises altering gene expression of one or more genes. In some embodiments, said modifying or targeting a target locus comprises epigenetic modification of said target DNA locus. In some embodiments, the method is a method of modifying a cell, a cell line, or an organism by manipulation of one or more target sequences at genomic loci of interest.

In some embodiments, the cell is a eukaryotic cell or a prokaryotic cell. In some embodiments, the eukaryotic cell is selected from the group consisting of: a plant cell, a fungal cell, a single cell eukaryotic organism, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and a human cell. In some embodiments, the cell is a mammalian cell or a human cell or a plant cell. In some embodiments, the method is in vitro or in vivo.

In another aspect, the disclosure provides a method of targeting and cleaving a double-stranded target DNA, the method comprising: contacting the double-stranded target DNA with a system of any one of above.

In some embodiments, cleaving the target DNA or target sequence results in the formation of an indel or the insertion of a nucleotide sequence. In some embodiments, cleaving the target DNA or target nucleotide comprising cleaving the target DNA or target sequence in two sites, and results in the deletion or inversion of a sequence between the two sites.

In another aspect, the disclosure provides an isolated eukaryotic cell comprising a modified target locus of interest, wherein the target locus of interest has been modified according to a method or via use of a composition or via use of a system of any one of the preceding contents.

The cleavage efficiency of the Cas12 protein on double-stranded DNA (dsDNA) is verified. In some embodiments, the cleavage ratio is 2%-100%. In one embodiment, in vitro cleavage efficiency assay, the range of the cleavage ratio is less than 10%. In one embodiment, in vitro cleavage efficiency assay, the range of the cleavage ratio is less than 5%. In one embodiment, in vitro cleavage efficiency assay, the range of the cleavage ratio is less than 15%. In one embodiment, in vitro cleavage efficiency assay, the range of the cleavage ratio can be less than 20%. In one embodiment, in vitro cleavage efficiency assay, the range of the cleavage ratio is more than 30%. In one embodiment, in vitro cleavage efficiency assay, the range of the cleavage ratio is more than 40%. In one embodiment, in vitro cleavage efficiency assay, the range of the cleavage ratio is more than 50%. In one embodiment, in vitro cleavage efficiency assay, the range of the cleavage ratio is more than 60%. In one embodiment, in vitro cleavage efficiency assay, the range of the cleavage ratio is more than 70%. In one embodiment, in vitro cleavage efficiency assay, the range of the cleavage ratio is more than 80%. In one embodiment, in vitro cleavage efficiency assay, the range of the cleavage ratio is more than 90%. In some embodiments, the cleavage ratio is 50%-100%. In some embodiments, the cleavage ratio is 60%-100%. In some specific embodiments, the cleavage ratio is 70%-90%. In some specific embodiments, the cleavage ratio is 80%-90%. In some specific embodiments, the cleavage ratio is 80%-95%. In some specific embodiments, the cleavage ratio is 85%-95%. In some specific embodiments, the cleavage ratio is 85%-98%. In some specific embodiments, the cleavage ratio is 60%-90%. For another example, in a specific embodiment, the cleavage ratio can be 1%, 2%, 3%, 4%, 5%, 6%, 7%, 8%, 9%, 10%, 12%, 15%, 18%, 20%, 25%, 30%, 35%, 40%, 50%, 55%, 58%, 60%, 65%, 70%, 72%, 73%, 75%, 78%, 80%, 82%, 85%, 87%, 88%, 90%, 92%, 95%, 97%, 98%, 99%, 100% and so on.

In some embodiments, the test of the genome cleavage activity in mammalian cells shows that the gene editing efficiency of the Cas12 protein is 50%-95%. For example, in a specific embodiment, the gene editing efficiency can be 50%, 55%, 58%, 60%, 65%, 67%, 70%, 72%, 73%, 75%, 78%, 80%, 82%, 85%, 87%, 88%, 90%, 92%, 95% and so on.

In some embodiments, the Cas12 protein shows a lower off-targets. In some embodiments, the off-targets are not detected in some Cas12 proteins.

The programmability, specificity, and collateral activity of the Cas12 protein also make it an ideal switchable nuclease for non-specific cleavage of nucleic acids. In one embodiment, a Cas12 protein system is engineered to provide and take advantage of collateral non-specific cleavage of nucleic acids, such as ssDNA. In another embodiment, a Cas12 protein system is engineered to provide and take advantage of collateral non-specific cleavage of ssDNA. Accordingly, engineered Cas12 protein systems provide platforms for nucleic acid detection and transcriptome manipulation, and inducing cell death. Cas12 protein is developed for use as a mammalian transcript knockdown and binding tool. Cas12 protein is capable of robust collateral cleavage of RNA and ssDNA when activated by sequence-specific targeted DNA binding.

In certain embodiments, Cas12 protein is provided or expressed in an in vitro system or in a cell, transiently or stably, and targeted or triggered to non-specifically cleave cellular nucleic acids. In one embodiment, Cas12 protein is engineered to knock down ssDNA, for example viral ssDNA. In another embodiment, Cas12 protein is engineered to knock down RNA. The system can be devised such that the knockdown is dependent on a target DNA present in the cell or in vitro system, or triggered by the addition of a target sequence to the system or cell.

In an embodiment, the Cas12 protein system is engineered to non-specifically cleave RNA in a subset of cells distinguishable by the presence of an aberrant DNA sequence, for instance where cleavage of the aberrant DNA might be incomplete or ineffectual.

Collateral activity was recently leveraged for a highly sensitive and specific nucleic acid detection platform termed SHERLOCK that is useful for many clinical diagnoses (Gootenberg, J. S. et al. Nucleic acid detection with CRISPR-Cas13a/C2c2. Science 356, 438-442 (2017)).

According to the invention, engineered Cas12 protein systems are optimized for DNA or RNA endonuclease activity and can be expressed in mammalian cells and targeted to effectively knock down reporter molecules or transcripts in cells.

The collateral effect of engineered Cas12 protein with isothermal amplification provides a CRISPR-based diagnostic providing rapid DNA or RNA detection with high sensitivity and single-base mismatch specificity. The Cas12 protein-based molecular detection platform is used to detect specific strains of virus, distinguish pathogenic bacteria, genotype human DNA, and identify cell-free tumor DNA mutations. Furthermore, reaction reagents can be lyophilized for cold-chain independence and long-term storage, and readily reconstituted on paper for field applications.

The ability to rapidly detect nucleic acids with high sensitivity and single-base specificity on a portable platform may aid in disease diagnosis and monitoring, epidemiology, and general laboratory tasks. Although methods exist for detecting nucleic acids, they have trade-offs among sensitivity, specificity, simplicity, cost, and speed.

In another aspect, the disclosure provides a system for detecting the presence of a nucleic acid target sequence in an in vitro sample, comprising: a) a Cas12 protein of any one of above; b) at least one guide polynucleotide comprising a guide sequence capable of binding the target sequence, and designed to form a complex with the Cas12 protein; and c) a nucleic acid-based masking construct comprising a non-target sequence; and wherein the Cas12 protein exhibits collateral cleavage activity of RNA and/or ssDNA and cleaves the non-target sequence of the nucleic acid-based masking construct activated by the target sequence.

In some embodiments, the system further comprising nucleic acid amplification reagents to amplify the target sequence. In some embodiments, the amplification reagents are isothermal amplification reagents. In some embodiments, the amplification reagents are nucleic-acid sequenced-based amplification (NASBA), recombinase polymerase amplification (RPA), loop-mediated isothermal amplification (LAMP), strand displacement amplification (SDA), helicase-dependent amplification (HDA), or nicking enzyme amplification reaction (NEAR).

In some embodiments, the target sequence is a target RNA sequence and the system further comprises an DNA polymerase and a primer designed to bind the target RNA sequence and further comprises a DNA polymerase promoter.

In another aspect, the disclosure provides a method for detecting target nucleic acids in samples comprising: a) contacting one or more samples with a Cas12 protein of any one of above; b) at least one guide polynucleotide comprising a guide sequence designed to have a degree of complementarity with the target sequence, and designed to form a complex with the Cas12 protein; and c) a nucleic acid-based masking construct comprising a non-target sequence, wherein the Cas12 protein exhibits collateral cleavage activity of RNA and/or ssDNA and cleaves the non-target sequence of the nucleic acid-based masking construct activated by the target sequences; and detecting a signal from cleavage of the non-target sequence, thereby detecting the one or more target sequences in the sample.

In some embodiments, the method further comprising contacting the one or more samples with reagents for amplifying one or more target sequences. In some embodiments, the amplification reagents are isothermal amplification reagents. In some embodiments, the amplification reagents are nucleic-acid sequenced-based amplification (NASBA), recombinase polymerase amplification (RPA), loop-mediated isothermal amplification (LAMP), strand displacement amplification (SDA), helicase-dependent amplification (HDA), or nicking enzyme amplification reaction (NEAR). In some embodiments, the target sequence is a target RNA sequence and the system further comprises an DNA polymerase and a primer designed to bind the target RNA sequence and further comprises a DNA polymerase promoter. In some embodiments, the masking construct suppresses generation of a detectable positive signal until cleaved or deactivated, or masks a detectable positive signal, or generates a detectable negative signal until the masking construct is deactivated or cleaved. In some embodiments, the masking construct comprises:
a. a silencing RNA that suppresses generation of a gene product encoded by a reporting construct, wherein the gene product generates the detectable positive signal when expressed;
b. a ribozyme that generates the negative detectable signal, and wherein the positive detectable signal is generated when the ribozyme is deactivated; or
c. a ribozyme that converts a substrate to a first color and wherein the substrate converts to a second color when the ribozyme is deactivated;
d. an aptamer and/or comprises a polynucleotide-tethered inhibitor;
e. a polynucleotide to which a detectable ligand and a masking component are attached;
f. a nanoparticle held in aggregate by bridge molecules, wherein at least a portion of the bridge molecules comprises a polynucleotide, and wherein the solution undergoes a color shift when the nanoparticle is disbursed in solution;
g. a quantum dot or fluorophore linked to one or more quencher molecules by a linking molecule, wherein at least a portion of the linking molecule comprises a polynucleotide; q. a polynucleotide in complex with an intercalating agent, wherein the intercalating agent changes absorbance upon cleavage of the polynucleotide; or
h. two fluorophores tethered by a polynucleotide that undergo a shift in fluorescence when released from the polynucleotide.

The following non-limiting examples are provided to further illustrate embodiments of the disclosure disclosed herein. It should be appreciated by those of skill in the art that the techniques disclosed in the examples that follow represent approaches that have been found to function well in the practice of the disclosure, and thus can be considered to constitute examples of modes for its practice. Those of skill in the art should, in light of the present disclosure, appreciate that many changes can be made in the specific embodiments that are disclosed and still obtain a like or similar result without departing from the spirit and scope of the disclosure.

### Example 1: a Method of Metagenomic Analysis for the Proteins

Metagenomic sequence data from public databases were search using Hidden Markov Models generated based on known Cas protein sequences including class II type V Cas effector proteins. CRISPR-Cas protein identified by the search were aligned to known proteins to identify potential active sites. From hundreds of potential sequences, finally, this metagenomic workflow resulted in the delineation of the Cas12 protein as above described and shown in FIG. 1.

The phylogenetic tree was constructed by IQTREE (FIG. 1) to visualize the relatedness of the orthologs at the primary amino-acid level using 176 Cas12a (V-A), Cas12b (V-B), Cas12c (V-C), Cas12d (V-D), Cas12e (V-E), Cas12f (Cas14, V-U2-4), Cas12g (V-G), Cas12h (V-H), Cas12i (V-I), Cas12j (V-J), Cas12k (V-K or V-U5), Cas12l (V-L), Cas12m (Vm or V-U1) and TnpB sequences from The National Center for Biotechnology Information (NCBI), various publications, and patents. The branch of the tree corresponding to the Cas12 proteins provided by this disclosure was marked with a circle while the reference nucleases (AsCpf1, FnCpf1 and LbCpf1) were marked with stars.

Although phylogenetically more closely related to Cas12a than other subtypes, the tree shows that the engineered Cas12 protein studied here are representatives of unique Cas12 clusters. For example, as shown in FIG. 1, GEB×0161, GEB×0162, GEB×0160, and GEB×0169 are more similar and they are representative clusters; GEB×0163 and GEB×0166 are more similar and they are representative clusters; GEB×0170, GEB×0173 and GEB×0174 are more similar and they are representative clusters; GEB×0165, GEB×0168, GEB×0171 and GEB×0172 are more similar and they are representative clusters.

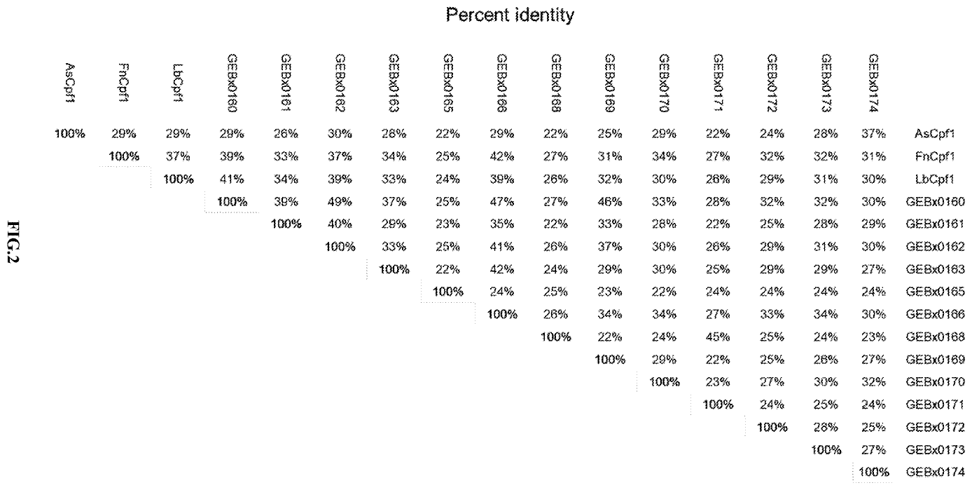

Besides that, as shown FIG. 2, the Cas12 proteins share less than 50% identity with the referenced cpf1 effectors, some even share less than 40% or 30% identity. These features suggest that Cas12 proteins were independent of the existing Cas12a family. Multiple sequence alignments were constructed using MAFFT.

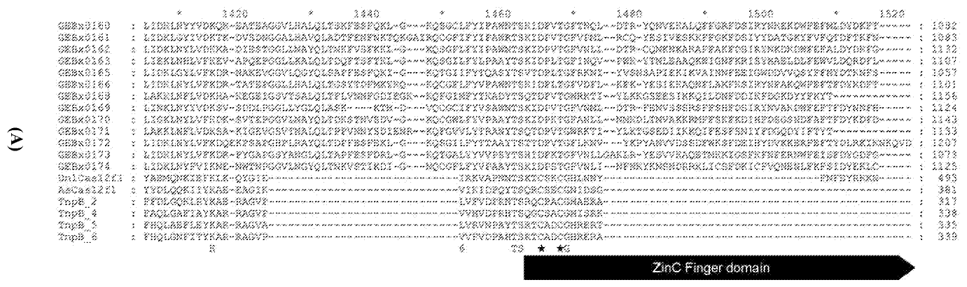

The structure modeling of GEB×Cas12 effectors was achieved by SWISS-MODEL and the model structures were used for domain arrangement analysis (shown in FIG. 3). As shown in FIG. 3, All of the GEB×Cas12 effectors contain three split WED domains, one REC.1 domain, one REC.2 domain, one putative PI domain, three split RuvC domains, one bridge helix (BH) and one NUC domain. The further sequence analysis (FIG. 5) found that there is no Zinc finger domain in any one of the GEB×Cas12 effectors. That is to say, the Cas12 proteins provided by this disclosure are all lack of the Zinc finger domain.

The amino acid sequences of Cas12 proteins and the referenced cpf1 are shown in table 2.

### Example 2: Protocol for Predicted crRNA Folding

Predicted RNA folding of the active single crRNA sequence located at the CRISPR array of Cas12 proteins was computed using the RNAfold webserver developed by Lorenz et al 2011. The folded sgRNAs were shown in FIG. 4, which contains a 5′-handle hairpin and 3′-end spacer sequence.

In FIG. 4, N represents the target specific sequence and the number of N is just an example illustration which does not represent its actual nucleotide quantity.

### Example 3: Protein Expression and Purification

The complete amino acid sequences of Cas12 proteins with nuclear localization signals (NLSs) and FLAG-tagged sequence are shown in SEQ ID NOs: 34-46 (table 5).

TABLE 5
The complete amino acid sequences of Cas12 proteins

| SEQ ID | |
|---|---|
| NO: | N to C |
| 34 | MAPKKKRKVKTMTDLTGLYPLSKTLRFELKPIGKTLEHIERKGLIAQDEQRAEDYERVKDLIDKYHK |
| | KFISLCLNKCRLKVESTGEYDSLEEYVEFAGIAKRTQEEENAFTKVKEMLRKTVADSFKKDPDQFKEL |
| | FKKEIITEHLPNMLLTDGSEEDKKKVESFALFTSYFTGFHQNRENMYSAEEKSTAIAYRLIHQNLPMFL |
| | DNIKSFEKIQATIDGKDMEELENAFRECLNVEHIADMFQLAYFTEILTQEQIDVYNYIIGGKTEEDGTKI |
| | KGINEYVNLYNQQHKDSRLPLLKPLYKMILSDRTTLSWLPEQFKSDEEMLTAINEAIKKLNDEVLNTA |
| | NQDNLQQLLSHLSDYDTRHIYITNNHCLTDISQQIFGQYDRYTTAIKEEIKSSLKPTAKEKKDPDLLND |
| | RTRKLFDARKSFSIAELNEYAQAEANQTIEVYFAQLGSYDRNGEQQISLLSRLNMAYNEAKEVLDGK |
| | YTNLNQSKEATEKIKNLLDAYKAIQFFAQPLAGSGDEAEKDNEFDAKLRNLTNTLSEVITPLYDKVRN |
| | WLTRKPYSTEKIKLNFENAQLLGGWDVNKEPDCTAILLRKHKQYYLGIMDKKSNHSFDIDELPSEGD |
| | CYEKIDYKLLPGANKMLPKVFFSKSRIDEFNPSDAIKKAYEKGTHKKGPDFSLADCHRLIDFFKASIAK |
| | HEDWKRFPFSFSDTGSYEDINGFYQEVEQQGYMIGFRPVSEAYINQLVEEGKLYLFQIWNKDFSEYSK |
| | GRPNMHTMYWNMLFDEKNLADVIYKLNGQAEVFFRQKSLDIAKTCVHAAQQPINNKNKSNQKQQS |
| | TFDYDIIKDRRYTMDKFQFHVPITMNFKSKGRENINSQVREIIRNNGIQHVIGIDRGERHLLYLSLIDLK |
| | GNIVKQMTLNDIINEYKGNTYATNYKQLLEYREGERTEARRSWQTIENIKELKEGYLSQVVHVIAKM |
| | MVEYNAIVVLEDLNGGFMRGRQKIERNVYEQFEKKLIDKLNYYVDKQKEATEAGGVLHALQLTSKF |
| | ESFQKLGKQSGCLFYIPAWNTSKIDPVTGFTNQLDTRYQNVEKALQFFGRFDSIRYNKEKDWFEFML |
| | DYDKFTTKAEGTQTRWTLCTYGTRIRSFRNDINKWVPKDVVLTEEFKQAFAAANIDVNGNLKDAICN |
| | LNEKAHLEELMHLVGLLVQLRNSKTNTEIDYMLSPVADKNGVFYDSRTCPDTLPKDADANGAYNIA |
| | RKGIWVIRKIQEDQTNDKVDLKITNKEWLAFAQQKPYLKHLKRPAATKKAGQAKKKKDYKDDDDK |
| 35 | MAPKKKRKVEPIGKTADYIKNYQYLETDERLAKESKKVKELADEYHKEFIGDVLSSLELPLSKINELW |
| | DIYMSNDTDREIKFKKLQENLRKVIAEAFSKDKRFGNLFKKEIITDILPKFLQDKDDDIKIVNRFKGFTT |
| | YFYAFHKNRENMYVSEEKSTAIPYRIVNQNLVKYFDNYKTFKEKVMHLLKDKNIVESIERDFKDILNE |
| | KSIEDVFGLANFTHTLCQADIEKYNTLIGGLVVKNEKKEIKGINQYINEHNQTSKKGNGIPKLKPLFNQ |
| | ILSDRKSLSFTLDDIKKTSEAIRTIKDEYENLRDKLATIERLIKSIKEYDLAGIYIKMGEDTSTISQHWFG |
| | AYYKIIEAIADAWERRNPKKNRESKAYSKYVSSLKSISLQEIDDLKIGEPIENYFATFGTTCSDRTSGVS |
| | SLNRIKAAYTEFVNKFPEGFEDGDDCNDAYFKANVEVVKNLLDSIKDFQRFVKPLLGNEDERDKDEA |
| | FYGEFVPTYTDMDNIITPLYNRVRNFATKKPYSTDKIKINFENVVLLKGWDKNKESDYASIILMKDGQ |
| | YFLGVLRNGSKSTLKTILPNTGDCYQKMVYKYFKDIKSNLPRCTTQRKDVKAHFAESSDDYTLLDTK |
| | AFVSALTISREVFELYNAPDKEKKFKKEYLKNTNDSIGYANAVSVCKRFCLEFLKKYRSTAIYDLSDV |
| | ETSVDSFDDLSSFYQEIDKRLYSISFENVSVDSVNELVDNGNMLLFRIANKDFSPNSKGRPNLHTIYWR |
| | MLFDPANLKDVVYQLNGNAEIFFRKASVTRTEPTHPANVAIKNKSEYNKQNKPYSTFKYGLIKDRRY |
| | TTDQFEFHVPITMNFKQPESSKLQDKLNKQVLDFLKQDGVRHIIGIDRGERNLLYLVMVDMEGKIKK |
| | QISLNEIAGNPKNPEFKQDFLALLHEREGDRLESRRSWNTIQSIKELKEGYMSLVVHEIANMMLENDA |
| | IVVLENLNRSFMQKRGEKSVYQKFEKMLIDKLGYIVDKTKDVSDNGGALHAVQLADTFENFNKTQK |
| | GAIRQCGFIFYIPAWRTSKIDPVTGFVPMLRCQYESIVESKKFFGKFDSIYYDATGKYFVFQTDFTKEN |
| | TESKGGIQKWDICTYGDRIYAPRTKDRNNNPVSERVNLTEEMKSLFVSHNINIQGDIKAGIMQQTDKE |
| | FFESLHRLLRLTLQIRNSKKSTGKDYEDYIISPVMGKDGRFFDSRNADATQPKDADANGAYNIARKGL |
| | MLLRQIQAQEKQDLSNGKWLEFAQRKRPAATKKAGQAKKKKDYKDDDDK |
| 36 | MAPKKKRKVTRKFDDFIHQYSLSKTLRFEARPIGETLHNFIKNGLLERDEHRAESYVKVKKLIDEYHK |
| | AFIDRVLNDGCLNYEDKGEYDSLTEYYALYSAPRKDEAEREHFKETQQHLRDQVVKKLTGDNAYKH |
| | LFGKELIESYKDKEDKKKLNQADLVQFINTATPKQRLNFSKEEAIDLVQEFCGFTTYFGDFYQNRENM |
| | YSAEEKSTGIAYRLINENLPKFIDNMETFKKIAAIPEMEDNLKEIHDNLAEHLNVENIQDMFQLNYYN |
| | QLLTQKQIDVYNAIIGGKTDDEHKEKIKGINEYVNLFNQAHKDAKLPKLKTLFKQILSDKNAISWLPE |
| | EFDNDQEVLNAIKDCYARLSENILGKENLKRLLCSLSEYDTKGIFLRNDLQLTSISKKMSGSWTDIPNA |
| | IKNDLKDGVPAKKHKESEEDYEKRIDNLFKRQDSFSIGYIDDCLNKFDDNNTFTIEGYFKKLGAKDTQ |
| | SEDIIKQIANAYTDVKPLLNSTYPKSKNLSQDKENVVKIKRFLDTLMSLVHFVKPLLGKGDESNKDEK |
| | FYGELSLLWTELETIVPLYNMVRNYMTRKPYSNSKIKLNFDNSQLLGGWDANKESDYASILLRRDGK |
| | YYLAIFDKDSKKLLGKSMPSDGECYEKMVYKFFKDVKTMIPKCSTQLKKVVKHFSISDTDFVLSDEN |
| | FKVPLTITKEVYDLNNTMYGKYKKFHKGYLTTTGDIDGYTSAVKTWINFCMDFLGAYGSTMSYDLS |
| | SLATKEYSSLESFYQEANRLLYTVNFVKVSTSYVSRLVTEGKMYLFQIYNKDFSEYSKGTPNMHTLY |
| | WKALFDDRNLKDVVYKLNGQAEMFFRKKSIDCNRPTHPANQPIQNKNKDNKKKESVFEYDLIKDRR |
| | YTVDKFMFHVPITMNFKSTDKENINLPVMEYLQTSNDTHIIGIDRGERHLLYLVVIDLHGNIVEQYTLN |
| | DIVNEYNGNTYRTNYNDLLEARGNEQQKASQNWQTIENIKELKEGYLSQVIHKITQLMIKYHAIIVLE |
| | DLNMGFKRSRQKVEKQVYQKFEKMLIDKLNYLVDKKADIESTGGLLNAYQLTNKFVSFKKLGKQSG |
| | FLFYIPAWNTSKIDPVTGFVNLLDTRCQNKNKAKAFFAKFDSIRYNKDKDWFEFALDYDKFGSKAEG |
| | TRTKWILCTQGKRIKTFRNAEKNFQWYNQVIDLTKDLKQLFAQYDIDINGNLKEAISKQTKKTFFDDL |
| | MGLLKLTLQMRNSITRTETDYLISPVADEIGNFYDSRTCGPSLPENADANGAFNIARKGLMIIEQIKAS |
| | EDLSKLKFDISNKSWLNFAQQKPYKHEKRPAATKKAGQAKKKKDYKDDDDK |
| 37 | MAPKKKRKVNTSKFIFQNLTHQFQLQKTLRFELIPQGRTLEHIEKNGLLEADELKANRYKQVKEWIDL |
| | YHKAFIEKVLREPKLEGLSEYRVFFENKQRNEKDEEAFKKLKSDLRKKISSFFKDEPNKTFYKRLFDK |
| | ELIKADLPDFLREQGMHEEATMLLEEFGNFTTYFKGFHENRKNLYVAEDKSTAIAFRLIDENLPRYMQ |
| | NLLLVEKHREVFEQFFPQIQERLGKELDGLRVEEFFSEEGFNLCLNGSGIHQYNTILGGVKGEEEEGIP |
| | GLNMLINLYRQKHKLKSRELPTLFPLHKQILSDRESSSFRPEPYEDGDENTVVEDLLKLGDQWFNPEG |
| | FVPQLVAHLTKLKDEEQDQLFVNNKANALSAVSQATLGHWAVLGEALSSYWNHVHDVKENALKG |
| | KALTAKEKWLKQPYFSFQEIELALRHYMGAEGKENASKWNVWLEKPLVQYFNYGLTKEAEKTKVD |
| | TAKAAWGHAENILRELQNHENPKLKTKETKDAIKAYFDACLDLLHFIKPLVVDLEQCEASPTFYGDF |
| | LSLYHEMDDLVPLYNKVRNYMTKKPYSYEKFKLNFNKGTLLDGWSQSKETDNLCVLFREGNNFFLG |
| | IMDRKHNKSFVKPPMAKEEEPVMQKVVYNLLPDPSKMLPKCFTAPYFRRIAKEKTKDPEQIVSEYSK |
| | EKGLIKPKLDVLDAYAHKKHTKENFQRNGFEQVVEYFKEGAQKHPDWHVFDFKFKPTSAYKDVSDF |
| | YQEVTEQGYCLDFVDIPKRYINDLVESGKLYLFQIYNKDFSKYSKGKPNLHTMYWKAIFDPQNLKAPI |
| | YKLNGEAEVFFRKKSLEYTQEQLDTGHHREELKGKFDYPIVKDRRFAFDKFQFHVPITLNHARKNQA |
| | VGPQVLRYIQKHKEELHYIGLDRGERHLLYLSVVNAKGEIIEQRSLNKIGNGFVKDGKPYTHDYQGK |
| | LLEKERNRDSARKDWDAVESIKELKEGYMSIIVHEIAQLVVKYNAVVILEDLNFGFKRGRFKVERQV |
| | YQKFERKLIEKLNHLVFKEVAPQEPGGLLKALQLTDQFTSFTKLGKQSGILFYLPAAYTSKIDPLTGFI |
| | NQVPWKYTNLEAAQKWIGNFKRISYHAELDLFEWVLDQRDFLSNPAGCKHQWVLRMDNQERWHW |
| | NSKAEGTNGKGAYSPIRLKEQFEDLLARQNISYNGNSKLLEDIQAVKDASFFKTLFFFLRTLTQLRYN |
| | NGKSGAEEEDFILSAVANPGMEPFDTRKAKQGLPLDADANGAYHIAMKGLMTLGQLKEKPNGKLEI |
| | PKLDKSAWLCFMQQRSNQGKRPAATKKAGQAKKKKDYKDDDDK |
| 38 | MAPKKKRKVQTKNFMSFSDFTNLYALSKTLRFELRPIGRTEEFFKKNKVFEKDKTINDSYNQAKLYF |
| | DEMHRRLIRDALNTQNTSALDYEKYAKHWKELQKDKGKYIQNWKDAQKEFRECISNLLDQQAEQW |
| | KSKYASVGLNKADIKQKGTDFLSSAGMVKILKKEFPKEAENQLVKEGFPSLFIQKAEAPEQKQYIFDS |
| | FDRFSTYLAKFNQTRDNLYKDDGTPTAIATRVVENFVAFLQNQAIFNNHYISLKSELDFSREEIDCFTA |
| | NYYVRCLLQEDIDEYNKTIGQINARIKELRDRKQKESDFKKSGYPLLKSLNKQILGKIEKKRQFIENKA |
| | DVFPAFSELIEQANARLTALRQLINNLCEEVFEEAYGGIYIKNTGINTISRRWFASWYDFEIALPQKSKN |
| | EEESKVVTFVSLQDIKDTLENKLSGDIFKEEYYKKKIINKEQSNWKQILAILQNEWNELFSEYQKALEN |
| | TKKLDLNSVFSNSKEQVEIIKSFADASLRVYQVVKYFALEHKKSDEIPSSYSTDFYEEFDKQYNNFEFI |
| | PYYNALRNFLTKKVVGENKIKINFEKGNLLNGWAESPEGNAQFCGYILKKENKYFLGITDQPTILDTK |
| | DNPSLTNITDKEYYEKMAYRQLKSTTIYGSGYEGLYNSKYANDKKYISESKIIERVKKLLQEKYLTRF |
| | PELKQIIEKEYKDANELAKNLGNFNLYSLEFIPVNKNYIEKLTHDKNGSIKYLYIFEIANKDLGKITPRK |
| | YNIHSLYFKNLFSEDNLEKPVFKLSGGAEIFFRPKTVNLPKKKDKAGKDIIDHKRYAQDKLFFHLPIVI |
| | NFGAGKVGKFNQELNKKLLAGNKNINIIGIDRGEKHLAYYSVINQKGEIIEQDSLNEINGVNYYGLLQ |
| | KREKERLENRKSWLPLQQIKDLKRGYISQVVYKIVRLAIEHNAIIVLEDLNMRFKQIRGGIEKSIYQQL |
| | EKALIDKLGYLVFKDRNAKEVGGVLQGYQLSAPFESFQKIGKQTGIIFYTQASYTSVTDPLTGFRKNIY |
| | VSNSAPIEKIKVAINNFEEIGWDDVVQSYFFNYDTKNFSNGNDIQSKKWKIYAKVPRIKREKVNGYW |
| | QYNDVDPNDMLEKLFRLYEFKNLRSNMLEQIHAKETNGELRGERDFDNKPRNFYQSLVYILNLILQL |
| | RNSTSKQIKLNEKEDVYETGEDVDFIASPAYPFFTTLSSKSNSNLAGFENRFTGLNHDQEQKKENGDA |
| | NGAYNIARKGIIILERITKNSDKPNLYISNEDWDKFVSQSYPKRPAATKKAGQAKKKKDYKDDDDK |
| 39 | MAPKKKRKVESKTLDRFIGKYQLSKTLRFELIPQGKTKEHIEQKGLLQQDEHRAKAYKMVKKIIDRY |
| | HQHFIATALNNLALDGLQSYEELYHKKEKEEADKKNFESLKAGMRKQIGDAFNKSEASKVQEIWKV |
| | LFDKELIRKALPEFVTSEEDKKLLAEFKDFTTYFTGFHQNRANIYTHEEKATAVAFRLVHENLPKFLD |
| | NSRVFDWIQTEYPDLDLSAIEKELAPELQGTTLPEIFALSFFNHTLTQNGIDLYNAVLGGKTTEQQNKI |
| | RGLNEYINLYNQRRTAEAKKEGEKFRRVPQFKELYKQILSDRESISFLPETFENDSEVLSAIENFFATQL |
| | VHFEKDNRSISVFAELQKLLQELKEDDRSKIFVRNDLALTNISQNIFGDWQVIKNALEHYYTTTVNPL |
| | AVGKKLTKAYENGKEKWQKQSYYSIYEIEEALLAYSSEADVLKEKNLTAASRPLCNYFATFSKDEEG |
| | NNLFQRIETAYTAVKDLLNTDYPEEKNLAQDKTNVAAIKTFLDAIKNLLHFIKPLHPARLEGEKDERF |
| | YGAFIPLFDQLNLLTPLYNKTRNYLTRKPYSTEKVKLNFENSTLLDGWDANKETANTSVLLRKDGLY |
| | YLAVMDKKYNKVFEQMPEVAEGEKVFEKVVYKLLPGANKMLPKVFFSAKNIQYYNPSTELLENYK |
| | NETHKKGATFNLVHCHQLIDFFKASIARHQDWKNFNFQFSDTGSYADLSGFYREVEQQGYKITFHNL |
| | PESYINQLVDQGKLYLFQIYNKDFSPFSKGRPNLHTMYWKMVFHEGNLTNVVYKLNGQAEIFFRKKS |
| | LNYDEKTLREGHHAAALKDKFAYPIISNRRFAFDKYQFHVPITINFKSTGRDNVNAEVLSFLQHNPSV |
| | NIIGIDRGERHLLYLTLIDQKGNIKKQMSLNQIINSYRNKEGDMVNVKTDYQNKLHLKEEERLKARQG |
| | WGLIENIKELKEGYLSHVVHEIAKMMVEHNAIVVMEDLNFGFKRGRQKVEKQVYQKFEKMLIDKLN |
| | YLVFKDRTATEPGGLLHALQLTGSYTDFMKYKQKQCGFLFYVPAWNTSKIDPLTGFVDFLKPKYESI |
| | EKAQNFLAKFKSIRYNPAKQWFEFTFDYKDFTDKAEGSRTRWTLCSTNQPRYRWNPKAANGKGAQE |
| | AVNVTQALEDLFSAHNIAYGSADDLVTAITAQSSADFYKALLRAMVTLLALRHNNGKAGTDEQDYI |
| | LSPVEPFFDSRLADDSRPKDADANGAYHIAKKGLWVLQQINELQGDDWKNLKLAISNKQWLQFAQQ |
| | LNADISPKVSKRPAATKKAGQAKKKKDYKDDDDK |
| 40 | MAPKKKRKVSSSIVSVSTEKKQAEVLSNKSVFDQFTRKYALSKTLRFELRPVGKTRENIEKANPNFIR |
| | DKEIEDAYQILKPVFDKLHEEFITESLESDEAKHLSFEDYFSVYKNLRNEQDREEKKKIEKSLETKEKN |
| | LRAAFSDIYKAEGEKFKSKVGSDKKGKPILKEDSFKILTEVGILKYVKAKIEEFVVMNLKTHEGKEVK |
| | KEDLEKALGTADSKGVFEGFFTYLTGFNQNRENYYSTEEKATAVASRIIDENLPKFADNVLEFQKRK |
| | DEYLKVYDFLKENGIETIGKNQKGEKVELEKIIADIFEIGYFTNCLSQREIEKYNLNIGNANNLINRYNQ |
| | QQSEKNKKLRIFKTLYKQIGCGEKGDFIPAIKNDDGLRAVLKNVVENGEKYFGQFDGKIEDKVDTVG |
| | EFVHYIINHESFEGIFWSDKALNTISGKYFANWFILKKALKEGGVFGGKKKDDEDIKIPQAVELKNLFE |
| | ILDKTEKWKNKEDGLFKTSLFDGEGNKKGIIERAEKPSKALLGMIFADVEKMAKDFSELSPKVLSITT |
| | DFKKPEHIENIKMLLDSALYVNQIVKYWKVKDKFAVDPILAESLKAILFGDNNPTKFYDIIRNYLTKRP |
| | QEGLNKLKLNFDNSVLGGGWDVNKEPERWCLILKDGEKKYLAILTKASQKLFGEKAKYKEVVTDEN |
| | YLKMDYKLLPGPNKMLPKVLLPKRNRKEFGATDDILSIYDEGGFKKNQPSFTKEKLWKIIDFYKEGL |
| | KVYPSAEKSWQDLFGFNFSPTEKFESIDQFYNEVEKQGYKLSWNIISKKLINEAVENGEIYFFEIRNKD |
| | NNLKEGKEKTSEKNLHTIYWNEIFGDVANKPKLNGEAEIFYRKTILKKKENETDKEFEIRLKEKLGKK |
| | KDKSGKEVLEHKRFANEKFVFHCPITLNFCLRSLKFNDEINESMVKLNDDITTIGIDRGEKHLAYYFVV |
| | KQTKDGKIETLDQGSFNEINGQNYAEKLEKMAGNRDEARKNWKTIGTIKELKDGYISQVVRKIVDLA |
| | IYEDVEKKVFRETPAFIVLEDLNIGFKRGRQKIEKQVYQKLELALAKKLNFLVDKHAKEGEIGSVTSA |
| | LQLTPLVNNFGDIEGKKQFGIMFYTKADYTSQTDPVTGWRKTIYLKKGSEESIKKQILDNFDDIKFDG |
| | KDYYFKYTDRNTNKPWELYSGKDGESLDRYYRELVHENSDKKWVPKEQNLKKILDGLFEGFDKEKS |
| | LLEQLRQGKNPNKINDRTSWESLRFVIDLIQQIRNSGKTEEDADFILSPVRDKNGNHFDSRKINLNLPTS |
| | GDANGAYNIARKGIIMCEHIKRDLSLFISDEEWSAWLAGREIWENWIKKNEKNLKRKQKRPAATKKA |
| | GQAKKKKDYKDDDDK |
| 41 | MAPKKKRKVKSIEELIGQYSLTKTLRFELKPIGKTLEQIERKGVLSADQTRADQYKRVKESIDRYHKA |
| | FITESLDSLVLKVKSTGNTSDSLEEYASLLDNSADDEALDKVKKGLRKQVAERFDKQIRLKKLFKNDL |
| | ITKILPEFLKVNGNEDERKAVESFRNFTTYFSGLNENRKNMYSKEAKSTAIAYRLIDENFPRFHDNMLS |
| | FAKIAAVNEVSNHFAEIAEAFKSEMVVASVAEMFGLEYFNNTLTQQKIDLYNYVIGGKTEGDKVKIQ |
| | GINEYVNLYNQNHPNDKLPLLKPLYKMILSDRVALSWLPEEFNDDEAMVCAIKEFYNAIQPVLWGEV |
| | EDDDNSVCHLLKNIGKYDIEHIFISNGAALNDVSRQMFGQYDFLANAIKKDIRQNTKRSSRETDEKFE |
| | GKVNTLFKKAKSYSIAHLNALIKENRSETIESFYSNLGACDRDGEQKINLLTQIELAHEAAQDVLDGK |
| | YADINRDEKATMLIKELLDAFKALQYFIKPLLGSGEEASKDNVFCTKLLNAWQALDAVTPLYDKVRN |
| | RLTRKPYSTDKIKLYFENKGKFLDGWVDSKTDNSDNGTQFGGYILRKKNEIGEYDYFLGISSDTKLLR |
| | ANPDIQYEHGMYERLDYYQIQSKTFLGSSYKGEYNDDAMRLMSAFVEYGKKSRFDSCVLPTDKEKS |
| | LPTYLKRIYTDNNSFYNEAMTDTVVAEAYNNIKSHLLLTLAGIKRVEAAVNLSKHNEYNLIQIFDEIG |
| | KITSSSKIFSFFPVDTSLVEKLNERDNKPLFLFKISNKDLSYAETSSKNIRKSRGKENLHTMYFKALMEQ |
| | SHNVFDIGTAEVFFRKATPGLDKSTTIHKANEALCNKNPHSKKTSSTFGYDIIKDRRYVENKFQLHLS |
| | MVINYQGKNPNTDIISPQVLDIIRNNGIKHIIGIDRGERNLLYLSLIDLKGNIIKQISLNEIKPLGNDGCVT |
| | NYRDILADREKDRKEARRSWKKLENIKDLKSGYLSQVVHVIAKMMVDYQAIVVLEDLNSAFMQSRQ |
| | KIERSVYEQFEHKLINKLNYYVDKSVSDDLPGGLLYGLQLASKKTHDVQCGCIFYVSAWNTSKIDPV |
| | TGFVNMLDTRFENVSSSRSFFSHFDSIRYNVAKDWFEFTFDYNNFHKKLEGTKTRWTLCTYGTRILTQ |
| | RNDRNQWESREVDITDEFKKAFVAAGIDIHGNLKDAICALDKRVHLEQLMRLMKLLLQLRNSIINQD |
| | TDYILSPVAENGVFYDSRRHADNLPIDADANGAYNIARKGLYLLRKIKADRSAKPELCISNKEWLRFV |
| | QDKSNPGDKRPAATKKAGQAKKKKDYKDDDDK |
| 42 | MAPKKKRKVVTIDDFCGQKNGYSRSITLRNKLIPVGKTQENLEKANMLEEDFSRAKAYVEVKKIIDD |
| | FHRSFIEDVLSKANLNWKELYEKIDSFQRETDKAKKAKNQKDLQPVQVKMRKAIVKIFKDDERFDKL |
| | FKKELLSDLLPEIIKSAPENEISNKQTALDVFKGFATYFTGFHENRKNMYSEEDKATAISYRIVNENFPK |
| | FYANIKLFEKIQKEFPVIIEETEKSLASFLAGKKLKDIFTPEAFNQVLTQSGIDFYNTVIGGIAGEAGTEK |
| | VQGLNEKINLARQQLPDEEKSKLRGKMVVLYKQILSDRETASFIPVGFENKDEVYKTLQEFKKSTVEK |
| | SIEIIRETFKSPENFDLSKIYVPAKELTAFSLSVFGRWNILSDGLFAIVEEKSKKALSEKQTEKLANEIAK |
| | KDYSLSELQAAYEKYLEKNPVEDEGRCRRLSAKNYFELIELRKIDDSAEKEFVNVLSNLDELWSKIDF |
| | ENPRNLQQEKEAATPIKNYLDEVQNLYHFLKLVDYRGEEEKDSEFYSKLTEILDLLSEIVPLYNKVRN |
| | FVTKKPGEVKKIKLNFDCSSFLKGWGTEFGTKDAFIFIDEKKYYLGVINKKLDSDTVKYLQTPGEHIIK |
| | QVFVNFQKPDFKNFPRLFIRSKKDRFAPAVEQFNLPVNDILDIYDKGLYKTEYRKENPIIYKESLIKMID |
| | YFKLGISQHESFKGFKLTWKESSEYENISDFYADVTASCYQVNFTNVNFDRLLSLANEIEDDNKKIFLF |
| | EIYNKDFSTGKNGGNGSTGKKNLHTMYFENLFSKENLKDIVLKLSGEAEIFLKSENTELKKIVHKQGT |
| | TLVNRTTKDKKLIPENIYQEIYQFKNSMIDNISDEAKAWLNSGNVECKIAKFDLIKDKHLIEKSYYLHC |
| | SIIQNFKAPEKVFNFNEKVQRYLKDNPDVKIIGLDRGERHLIYLSLINQKGEIELQKTLNLVEQVRNDK |
| | TVKVNYQEKLVQKEGDRDKARRNWQTIGNIKELKEGYLSNVVHEIAKLMVEHNAIVVMEELNFGFK |
| | RGRFPVERQVYQKFENMLIGKLNYLVFKDKSVTEPGGVLNAYQLTDKSTNVSDVGKQCGWLFYVPA |
| | AYTSKIDPKTGFANLLNMKDLTNVAKKRMFFSKFKDIHFDSGSNDFAFTFDYKDFDGKGAAEMTQT |
| | EWTVWSNGKRIFYNSKHHSTEEINVSEKLKELFDASKIDYKTENNIFESIMEVGADLKEGEKPSKEIAN |
| | FWDSLLWYFKLILQMRNSIPNSETDYLISPVKAEDGTFFDSRVQKELGKDSKLPIDADANGAYHIALK |
| | GLYLLKNDFNLNDKGYIENISNAEWFRFVQERDYLNKRPAATKKAGQAKKKKDYKDDDDK |
| 43 | MAPKKKRKVPNLFDNFTQQYSLTKTLRFELKPEGKTLENMQNAFKFDKDLQTFLVDQDIEDAYQTL |
| | KPYIDFIHEEFITESLESDIAKGIDFSEYFENFKIKKDINQKDFQNLEHKLRKEFDQLYIQIANKWKEEKY |
| | KQYKWKKGSKVANGSEILSSEDILKLVVDLNQDNNQLKDIVQNTFKGFFTYFSGFNDNRRNYYQVE |
| | KEAKSSIATRIVNENLPKFCDNFLLFENRKNEYLGVYNTLKLLGKDLVDKDRKTLVTIEEIIFSIKHFNF |
| | CLSQKEIENYNKQIGNANLLINLYNQNTSNESDYKRLPLFKTLYKQIGCGKRDSLFFEITDYTKLEAEG |
| | KRILGKSAISVEEVLIQVKESGIKYFTEKLDSLLNYILEKETDNYQGLYWSNIAVNSISSKYLANWNNL |
| | KELFKGKKEVASYDKGREEEIKINEAVDLSILFSILNNTEDWKVKEVFFKENLNEESETKKIIETSKTPS |
| | QALLRIVIGDLKKNASNFLNLSDNIEQLLESKEKIKESMDYALSVTRMLKYFLVKESKTKGAPLDSKL |
| | VEDLNLLLLSDDAPWFMWYDGLRNYLTKKPHDDAKENKLKLNFENSTLAGGWDINKEPANYCVILT |
| | NSANDQYLAINAKRNNKIFCKELVDGKGKDKKLLKNPLYESDEQHWKKMEYKLLPGPNKMLPKCL |
| | LPKSNLRKFGASEEVLSIYENGSFKKNTPDFSTQDLHKLIDFYKSALLKYEDWKCFHFKENDTRSYGD |
| | ISQFYSDLEKQGYLLSFIDINKSVLNQLVDSGKIYLFQIRNQDSNISKKINHQDNLHTIYWRAVFEDVFN |
| | KPKLNGQAEVFYRKALPQEDLQLLKDKDNKEIIANYRFSKEKFLFHVPITLNFCLKNKKINDLANREIL |
| | SNDNIYFLGIDRGEKHLAYYSLVDQKGNIVIDQNGKSIQGTLNIPFLDSNGNQRLVKATKRVMKDGK |
| | EDAQVVECKDYNELLEARASDRDYARKNWQTIGAIKDLKDGYISQVVRKIADLAVEYNAYIVLEDL |
| | NTGFKRGRQKIEKSVYQKLELALAKKLNFLVDKSAKIGEVGSVTNALQLTPPVNNYSDIENRKQFGV |
| | VLYTRANYTSQTDPVTGWRKTIYLKTGSEDIIKKQIFESFSNIYFDGQDYIFTYTDKNIDRKWELYSGK |
| | NGKSLDRYRGRKVNNKGWVIDKINIQEILDKIFINFEICYGEKNNIFEKLKAGVELEKYNEYTAWESL |
| | RFAIDIIQQIRNTGIESSDSDFLLSPIRVNNLHFDSRLASENQPNSGDANGAYNIARKGIIMANYIKQGVS |
| | PFVSDIDWDNFVTKNKRPAATKKAGQAKKKKDYKDDDDK |
| 44 | MAPKKKRKVNKFLDSFTHQYSLSKTLRFELKPVGKTAEWIQKRDIIGTKKQENGEAQLTGKDAVRAE |
| | NYEYAKKLLNEMHRLFIEDSLAAIKEEQKVELNKLLVKIAESGSLDGFRKPMSNICKELFDSATQDWL |
| | LAYSEEMPCFWDEDILELTNKAETTENKKNIKFFLKKAEDIRKFKDDPLKAFKKKKHEALNSNTEAL |
| | KLLEWKIRRGEVLISGKDLGRDESEEILSTKELILILRSFYSFVTYFTGFNENRANIYNLDDKKYISTSIA |
| | FRSFEQNLFFHIENIKKWHTITNSIEEHTDQLNASDYPIKDKLRELELAFDCKLADLFAVDSFSQFLSQS |
| | GIDRYNSIMGGIAQKTGKTKVQGLNEIINQARQQTGAKRHKFPPMKLLYKQILSKGEGIFVESYVDDA |
| | DMLTSIAGFHENHLNESLCSNWLHELEELIDSVQESKDTIFIARDKLNRISQEITGSWSAIQDWIVAHQ |
| | KDQKEVDALTKKKVFSIQELEDYFSKSSDDELFTAKIPEDRSAYLPGEGEGILFQFFKVRAKEILAELQ |
| | SGWYALDKAQLLQEKQLDKNRENEGSKGFEQVGLLKGYLDACNSLSRFIADWNIQKDIKQERCSLW |
| | YEFLQKHLDTVPIFDLYNQVRNHVSKKAYSTDKLKVNFEKSTLLDGWDRNKESQNFGVLFEKDGAY |
| | YLGIMTPDSNGIFEYDVNPDENTKKKAIKEELRKQTTPENGEDRFQKINYKLLPGPNKMLPKVFFAKS |
| | NEDLFQPSARITEIKENKKYAKAYTDKHGKAELHEYIDFCQKSLVRHPEWSVAYGFDQTTFRKATEYI |
| | SIDEFYREVELLGYKVTFDSIKATYIEEKVANGELYLFQIYNKDFSTAKKGGGKDNLHTLYWKSLFSE |
| | DNLKDVVFKLNGQAEVFFRPSSIVWSEKVMKQGHHYDDDKMLKDKFKYPIIKDFRFTQDKFFFHCPI |
| | SLNFKAPAKPYGFNYKVHDFLRKNGEINVIGIDRGEKHLLYYSITNQQGDIIEQGSLNSIESSYEKGSSS |
| | VPHSTNYLEKLQEKEKNRDSARKSWSRIENIKELKAGYLSHVVHKLAKLIIKHNAVVVLEDLNAGFK |
| | RGRFKVERQVYQKFEKALIDKLNYLVFKDQEKPSAPGHPLRAYQLTDPFESFEKLGKQSGILFYTTAA |
| | YTSTTDPVTGFLKNVYKPYANVVDSSDFWKSFDEIHYDVKKERFEFTYDLRKIKNKQVDRDKDEAK |
| | VSRSVWTVCSCVERSRYVKRKANEEQKQGAASKSIGKMGEYEVFVVTDKLKALFEKESISYQSGEDI |
| | RSQLSNRIDKKDAKLHRELIYYFGTLLNMRVTDKQSESGTDENDYILSPIEPFFDSRKGYEKLPVNGD |
| | ANGAYNIARKGIAILNRIHESDSGDKVDLMIRKKDWQDFVQNEEVVQKQMGKLDLKRPAATKKAG |
| | QAKKKKDYKDDDDK |
| 45 | MAPKKKRKVALFSENFVGCYPVSKTLKFELRPVPETKAYLDDDNCPLLAGDLERDKAYPVVKELLD |
| | KYYRYFIDICLKDKKLSPETIDEAYQAYSSHDEKSQKALKAASQKLRKEVAGFFDKKTRETYCLDKY |
| | SNLLVLDKKNDNKEKGTPKTIPSVLKKWLENESGFSEKEVEKYSQAVQTFNGFTTYFISYKENRENM |
| | FSSEEKASAISYRVVNQNMLRFFSNVENFENIKKKYPDLYQTLKEFEESFVPAAYSSLLSQAGIDDYNY |
| | RVIGRPAEDEDFAGVNRLINEYRQKNAVRNRNLPVMVTLYKQILSDRENGFGRDILASDSEAKEYIRG |
| | KYSKAYDTACQLTVFVNNYILTEDLDTVYVKTQHLKDISQSIFGEWSVLDDAINNGGIDSKIISLKALA |
| | EAFEKYCSALDPDMAQKYRDAFSLKEYLKNPPAVEPRLPETAEKVTAYKPAMDELISLIRFYKPFYLY |
| | DGKKRLQVPAEGMDFSNEFDKYFEDLKEFSHAYDKVRNFATKKPYSVEKIKLNFNQPTLLDGWDVN |
| | KEKENASFLFVKEGKYYLGIADKSDKNLFDRSRDAVKEALTLTCGEDYYEKVQYKQLPNIRMNLPRI |
| | VFKPKWEKVFAPIITERISAIEKEKLYTAGANDRQAVIEWIDFVKSVLALHSEWNNYFQFKFKETSAY |
| | KNTKEFYDDLDNQAYILSKIKINASYIHKLVRERKLYLFQIYCKDFSDKKKKTGADNLHTMYWRCLF |
| | SDENLAALAQGQAPIIKLNGKAEIFLREPSLKYEATHPKNQPVANKNPLNSKRESLFSYDLAKNRRFT |
| | ERKLFFHCPLTINFRTAAEGARSFNERVNRFVENNPDIRIIGIDRGERHLLYYTVINQSGKILEQGSLNKI |
| | SSDYRTPEGAEVEKVTDYHELLNRKEGDRRAARQTWDTIENIKELKEGYLSQVVHKLTTLMIKHNAV |
| | VVLENLNQNFKRSRIKVEKQVYQKFEKALIDKLNYLVFKDRPYGAPGSYANGLQLTAPFESFDRLGK |
| | QTGALYYVVPSYTSHIDPKTGFVNLLGAKLRYESVVKAQETMKKIGSFKFNPERNWFEISFDYGDFGI |
| | EMAHPQWTACTHGDAYWGYNPKEKRAVSYDVTEEIKKLLTEYGIDYGTGADIKEAAIRQTKPAFWK |
| | TLFYMLKLALQLRNTVAGTDNENDYILSPVEYEAGKFFDSRKAGEDEPQNADANGAYHIAMKGLQA |
| | LRRIEDGKVNKFPKNGERKAWLAFMQNREYLKKRPAATKKAGQAKKKKDYKDDDDK |
| 46 | MAPKKKRKVSKLSTFNEHFQKTITLRNELVPVGKTLENIISSNVLINDEKRSEDYKKAKEIIDSYHQEFI |
| | EKSLSKVDIEWDDLAYCLTKKEPENFDKKKKYQEEINKLQNEKRKKSVEIFKKFSFGVLCDNNGQEV |
| | QIKFDDLFKSTLFKYVLPNFLKNDEDKTVVRDFSEFTSYFTGFHENRKNLYKSAPMPTAVAYRIVNDN |
| | FPKFISNQKIFCAWKDNVPHFVEIAKTKLSEVGISDLDIESKFELSNFNSCLNQTGIDYYNDLRGQLNN |
| | VISHICQQDKNLGDLLRKKRCLKMVPLYKQILSDKDSSFSIDEFDNDESAIQDVISFYKKMVGERCPQR |
| | KLSELLRELSSHDREKIFIQGKNLNSISRILFGGKNWSLLREAIIEEKSKDKSFKKAIKSNSTDEALEKILS |
| | KDEFSISFLSRICHENLSEKIDKFIKNQDEQLININSQTWPKTLKNSEEKKIIKEHLDFLLNFYRFAKSFSS |
| | NTIDKDMSFYADFDESLSSLENVIGLYNKVRNYVTKKPYSLEKIKLNFEIPNLAEGWSKTKELANLTVI |
| | FLKDNEYYLGIINKNSKIDFSLIKEKTSSENCFRKMEYSLFGKIAQNINRCVFTKAVKNHFSKSPDNFIL |
| | SDKKKFLKEIEITKEIYDLVKSDESDKKYLKEYKKINEKEFRSALSKWINFCIEFLKSYKNTSQFDFSCI |
| | KKAESYDDINDFYDDIEKVTYKIEFVDISTSLIDALVDKGQLFLFKIRNKDFAKKASGTPNLHTLYFKSI |
| | FDTKNFENGIVKLNGEAEIFYRKKSLNVDDTTVHQKGSYIVNKVVVDPSSGKSEQIPDNVYNNIYAFV |
| | NGKLKTLSKEDEVYYAKATIKKATHEIVKDRRFTEDKFFFHCPITINYKSKEKPSKFNDKVLDFLRKN |
| | DDINIIGIDRGERNLIYATVINKNGEIIDCKSFNTIKHQSSSVNFDVDYHNKLQEREKNRKEEKRSWNSI |
| | SKIADLKEGYLSAVIHEIALMMVKYNAIVVMENLNQGFKKIRGGIAERSVYQKFEKMLIDKLNYFVIK |
| | NENWTNPGGVLNGYQLTNKVSTIKDIGNQCGFLFYVPAAYTSKIDPSTGFVNLINFNKYKNSNDRRK |
| | LICSFDKICFVQNENLFKFSIDYEKLCPNSKMPVKKWDIFSYGNRIVREDLKHIPMKENPKYDPTEELK |
| | TLFSSKEIEYQKGQNLLEIISTKDMTEDKEFWNSLFNIFKAILQMRNSLTNSPVDRLLSPVKGKDGSFF |
| | DTDKVEGTKFEKLKDADANGAYNIALKGLLVLKKNDSVKTDKDMKNVKNVSLEDWLKFVQVTLR |
| | VKRPAATKKAGQAKKKKDYKDDDDK |
| 153 | MAPKKKRKVALFSENFVGCYPVSKTLKFELRPVPETKAYLDDDNCPLLAGDLERDKAYPVVKELLD |
| GEBx0173 | KYYRYFIDICLKDKKLSPETIDEAYQAYSSHDEKSQKALKAASQKLRKEVAGFFDKKTRETYCLDKY |
| v1 | SNLLVLDKKNDNKEKGTPKTIPSVLKKWLENESGESEKEVEKYSQAVQTFNGFTTYFSSYKRNRENM |
| with NLS | FSSEEKASAISYRVVNQNMLRFFSNVENFENIKKKYPDLYQTLKEFEESFVPAAYSSLLSQAGIDDYNY |
| and | RVIGRPAEDEDFAGVNRLINEYRQKNAVRNRNLPVMVTLYKQILSDRENGFGRDILASDSEAKEYIRG |
| FLAG | KYSKAYDTACQLTVFVNNYILTEDLDTVYVKTQHLKDISQSIFGEWSVLDDAINNGGIDSKIISLKALA |
| | EAFEKYCSALDPDMAQKYRDAFSLKEYLKNPPAVEPRLPETAEKVTAYKPAMDELISLIRFYKPFYLY |
| | DGKKRLQVPAEGMDFSNEFDKYFEDLKEFSHAYDKVRNFATKKPYSVEKIKLNFNQPTLLRGWDVN |
| | VEKNRASFLFVKEGKYYLGIADKSDKNLFDRSRDAVKEALTLTCGEDYYEKVQYRQLPNIRMNLPRI |
| | VFKPKWEKVFAPIITERISAIEKEKLYTAGANDRQAVIEWIDFVKSVLALHSEWNNYFQFKFKETSAY |
| | KNTKEFYDDLDNQAYILSKIKINASYIHKLVRERKLYLFQIYCKDFSDKKKKTGADNLHTMYWRCLF |
| | SDENLAALAQGQAPIIKLNGKAEIFLREPSLKYEATHPKNQPVANKNPLNSKRESLFSYDLAKNRRFT |
| | ERKLFFHCPLTINFRTAAEGARSFNERVNRFVENNPDIRIIGIDRGERHLLYYTVINQSGKILEQGSLNKI |
| | SSDYRTPEGAEVEKVTDYHELLNRKEGDRRAARQTWDTIENIKELKEGYLSQVVHKLTTLMIKHNAV |
| | VVLENLNQNFKRSRIKVEKQVYQKFEKALIDKLNYLVFKDRPYGAPGSYANGLQLTAPFESFDRLGK |
| | QTGALYYVVPSYTSHIDPKTGFVNLLGAKLRYESVVKAQETMKKIGSFKFNPERNWFEISFDYGDFGI |
| | EMAHPQWTACTHGDAYWGYNPKEKRAVSYDVTEEIKKLLTEYGIDYGTGADIKEAAIRQTKPAFWK |
| | TLFYMLKLALQLRNTVAGTDNENDYILSPVEYEAGKFFDSRKAGEDEPQNADANGAYHIAMKGLQA |
| | LRRIEDGKVNKFPKNGERKAWLAFMQNREYLKKRPAATKKAGQAKKKKDYKDDDDK* |
| 154 | MAPKKKRKVALFSENFVGCYPVSKTLKFELRPVPETKAYLDDDNCPLLAGDLERDKAYPVVKELLD |
| GEBx0173 | KYYRYFIDICLKDKKLSPETIDEAYQAYSSHDEKSQKALKAASQKLRKEVAGFFDKKTRETYCLDKY |
| v2 | SNLLVLDKKNDNKEKGTPKTIPSVLKKWLENESGFSEKEVEKYSQAVQTFNGFTTYFISYKRNRENM |
| with NLS | FSSEEKASAISYRVVNQNMLRFFSNVENFENIKKKYPDLYQTLKEFEESFVPAAYSSLLSQAGIDDYNY |
| and | RVIGRPAEDEDFAGVNRLINEYRQKNAVRNRNLPVMVTLYKQILSDRENGFGRDILASDSEAKEYIRG |
| FLAG | KYSKAYDTACQLTVFVNNYILTEDLDTVYVKTQHLKDISQSIFGEWSVLDDAINNGGIDSKIISLKALA |
| | EAFEKYCSALDPDMAQKYRDAFSLKEYLKNPPAVEPRLPETAEKVTAYKPAMDELISLIRFYKPFYLY |
| | DGKKRLQVPAEGMDFSNEFDKYFEDLKEFSHAYDKVRNFATKKPYSVEKIKLNFNQPTLLRGWDVN |
| | REKENASFLFVKEGKYYLGIADKSDKNLFDRSRDAVKEALTLTCGEDYYEKVQYKQLPNIRMNLPRI |
| | VFKPKWEKVFAPIITERISAIEKEKLYTAGANDRQAVIEWIDFVKSVLALHSEWNNYFQFKFKETSAY |
| | KNTKEFYDDLDNQAYILSKIKINASYIHKLVRERKLYLFQIYCKDFSDKKKKTGADNLHTMYWRCLF |
| | SDENLAALAQGQAPIIKLNGKAEIFLREPSLKYEATHPKNQPVANKNPLNSKRESLFSYDLAKNRRFT |
| | ERKLFFHCPLTINFRTAAEGARSFNERVNRFVENNPDIRIIGIDRGERHLLYYTVINQSGKILEQGSLNKI |
| | SSDYRTPEGAEVEKVTDYHELLNRKEGDRRAARQTWDTIENIKELKEGYLSQVVHKLTTLMIKHNAV |
| | VVLENLNQNFKRSRIKVEKQVYQKFEKALIDKLNYLVFKDRPYGAPGSYANGLQLTAPFESFDRLGK |
| | QTGALYYVVPSYTSHIDPKTGFVNLLGAKLRYESVVKAQETMKKIGSFKFNPERNWFEISFDYGDFGI |
| | EMAHPQWTACTHGDAYWGYNPKEKRAVSYDVTEEIKKLLTEYGIDYGTGADIKEAAIRQTKPAFWK |
| | TLFYMLKLALQLRNTVAGTDNENDYILSPVEYEAGKFFDSRKAGEDEPQNADANGAYHIAMKGLQA |
| | LRRIEDGKVNKFPKNGERKAWLAFMQNREYLKKRPAATKKAGQAKKKKDYKDDDDK* |
| 155 | MAPKKKRKVALFSENFVGCYPVSKTLKFELRPVPETKAYLDDDNCPLLAGDLERDKAYPVVKELLD |
| GEBx0173 | KYYRYFIDICLKDKKLSPETIDEAYQAYSSHDEKSQKALKAASQKLRKEVAGFFDKKTRETYCLDKY |
| v3 | SNLLVLDKKNDNKEKGTPKTIPSVLKKWLENESGFSEKEVEKYSQAVQTFNGFTTYFISYKRNRENM |
| with NLS | FSSEEKASAISYRVVNQNMLRFFSNVENFENIKKKYPDLYQTLKEFEESFVPAAYSSLLSQAGIDDYNY |
| and | RVIGRPAEDEDFAGVNRLINEYRQKNAVRNRNLPVMVTLYKQILSDRENGFGRDILASDSEAKEYIRG |
| FLAG | KYSKAYDTACQLTVFVNNYILTEDLDTVYVKTQHLKDISQSIFGEWSVLDDAINNGGIDSKIISLKALA |
| | EAFEKYCSALDPDMAQKYRDAFSLKEYLKNPPAVEPRLPETAEKVTAYKPAMDELISLIRFYKPFYLY |
| | DGKKRLQVPAEGMDFSNEFDKYFEDLKEFSHAYDKVRNFATKKPYSVEKIKLNFNQPTLLRGWDVN |
| | REKENASFLFVKEGKYYLGIADKSDKNLFDRSRDAVKEALTLTCGEDYYEKVQYKQLPNIRMNLPRI |
| | VFKPKWEKVFAPIITERISAIEKEKLYTAGANDRQAVIEWIDFVKSVLALHSEWNNYFQFKFKETSAY |
| | KNTKEFYDDLDNQAYILSKIKINASYIHKLVRERKLYLFQIYCKDFSDKKKKTGADNLHTMYWRCLF |
| | SDENLAALAQGQAPIIKLNGKAEIFLREPSLKYEATHPKNQPVANKNPLNSKRESLFSYDLAKNRRFT |
| | ERKLFFHCPLTINFRTAAEGARSFNERVNRFVENNPDIRIIGIDRGERHLLYYTVINQSGKILEQGSLNKI |
| | SSDYRTPEGAEVEKVTDYHELLNRKEGDRRAARQTWDTIENIKELKEGYLSQVVHKLTTLMIKHNAV |
| | VVLENLNQNFKRSRIKVEKQVYQKFEKALIDKLNYLVFKDRPYGAPGSYANGLQLTAPFESFDRLGK |
| | QTGALYYVVPSYTSHIDPKTGFVNLLGAKLRYESVVKAQETMKKIGSFKFNPERNWFEISFDYGDFGI |
| | EMAHPQWTACTHGDAYWGYNPKEKRAVSYDVTEEIKKLLTEYGIDYGTGADIKEAAIRQTKPAFWK |
| | TLFYMLKLALQLRNTVAGTDNENDYILSPVEYEAGKFFDSRKAGEDEPQNADANGAYHIAMKGLQA |
| | LRRIEDGKVNKFPKNGERKAWLAFMQNREYLKKRPAATKKAGQAKKKKDYKDDDDK* |

The DNA fragments (SEQ ID NOs: 47-59, Table 6) encoding the Cas12 proteins, together with 3′ and 5′ nuclear localization signals (NLSs) and FLAG-tagged sequences, were synthesized by GenScript and assembled by Gibson assembly into pEASY-Blunt E2 expression plasmid.

TABLE 6
The nucleotide sequences encoding the Cas12 proteins with NLSs and
FLAG-tag

| SEQ ID | |
|---|---|
| NO: | 5′ to 3′ |
| 47 | atggcccccaagaaaaagcggaaggtgAAGACCATGACCGACCTGACCGGCCTGTACCCCCTGAGCAAGACCCTGAGGTTCGAGC |
| | TGAAGCCCATCGGCAAGACCCTGGAGCACATCGAGAGGAAGGGCCTGATCGCCCAGGACGAGCAGAGGGCCGAG |
| | GACTACGAGAGGGTGAAGGACCTGATCGACAAGTACCACAAGAAGTTCATCAGCCTGTGCCTGAACAAGTGCAGG |
| | CTGAAGGTGGAGAGCACCGGCGAGTACGACAGCCTGGAGGAGTACGTGGAGTTCGCCGGCATCGCCAAGAGGACC |
| | CAGGAGGAGGAGAACGCCTTCACCAAGGTGAAGGAGATGCTGAGGAAGACCGTGGCCGACAGCTTCAAGAAGGA |
| | CCCCGACCAGTTCAAGGAGCTGTTCAAGAAGGAGATCATCACCGAGCACCTGCCCAACATGCTGCTGACCGACGGC |
| | AGCGAGGAGGACAAGAAGAAGGTGGAGAGCTTCGCCCTGTTCACCAGCTACTTCACCGGCTTCCACCAGAACAGG |
| | GAGAACATGTACAGCGCCGAGGAGAAGAGCACCGCCATCGCCTACAGGCTGATCCACCAGAACCTGCCCATGTTCC |
| | TGGACAACATCAAGAGCTTCGAGAAGATCCAGGCCACCATCGACGGCAAGGACATGGAGGAGCTGGAGAACGCCT |
| | TCAGGGAGTGCCTGAACGTGGAGCACATCGCCGACATGTTCCAGCTGGCCTACTTCACCGAGATCCTGACCCAGGA |
| | GCAGATCGACGTGTACAACTACATCATCGGCGGCAAGACCGAGGAGGACGGCACCAAGATCAAGGGCATCAACGA |
| | GTACGTGAACCTGTACAACCAGCAGCACAAGGACAGCAGGCTGCCCCTGCTGAAGCCCCTGTACAAGATGATCCTG |
| | AGCGACAGGACCACCCTGAGCTGGCTGCCCGAGCAGTTCAAGAGCGACGAGGAGATGCTGACCGCCATCAACGAG |
| | GCCATCAAGAAGCTGAACGACGAGGTGCTGAACACCGCCAACCAGGACAACCTGCAGCAGCTGCTGAGCCACCTG |
| | AGCGACTACGACACCAGGCACATCTACATCACCAACAACCACTGCCTGACCGACATCAGCCAGCAGATCTTCGGCC |
| | AGTACGACAGGTACACCACCGCCATCAAGGAGGAGATCAAGAGCAGCCTGAAGCCCACCGCCAAGGAGAAGAAG |
| | GACCCCGACCTGCTGAACGACAGGACCAGGAAGCTGTTCGACGCCAGGAAGAGCTTCAGCATCGCCGAGCTGAAC |
| | GAGTACGCCCAGGCCGAGGCCAACCAGACCATCGAGGTGTACTTCGCCCAGCTGGGCAGCTACGACAGGAACGGC |
| | GAGCAGCAGATCAGCCTGCTGAGCAGGCTGAACATGGCCTACAACGAGGCCAAGGAGGTGCTGGACGGCAAGTAC |
| | ACCAACCTGAACCAGAGCAAGGAGGCCACCGAGAAGATCAAGAACCTGCTGGACGCCTACAAGGCCATCCAGTTC |
| | TTCGCCCAGCCCCTGGCCGGCAGCGGCGACGAGGCCGAGAAGGACAACGAGTTCGACGCCAAGCTGAGGAACCT |
| | GACCAACACCCTGAGCGAGGTGATCACCCCCCTGTACGACAAGGTGAGGAACTGGCTGACCAGGAAGCCCTACAG |
| | CACCGAGAAGATCAAGCTGAACTTCGAGAACGCCCAGCTGCTGGGCGGCTGGGACGTGAACAAGGAGCCCGACTG |
| | CACCGCCATCCTGCTGAGGAAGCACAAGCAGTACTACCTGGGCATCATGGACAAGAAGAGCAACCACAGCTTCGAC |
| | ATCGACGAGCTGCCCAGCGAGGGCGACTGCTACGAGAAGATCGACTACAAGCTGCTGCCCGGCGCCAACAAGATG |
| | CTGCCCAAGGTGTTCTTCAGCAAGAGCAGGATCGACGAGTTCAACCCCAGCGACGCCATCAAGAAGGCCTACGAG |
| | AAGGGCACCCACAAGAAGGGCCCCGACTTCAGCCTGGCCGACTGCCACAGGCTGATCGACTTCTTCAAGGCCAGC |
| | ATCGCCAAGCACGAGGACTGGAAGAGGTTCCCCTTCAGCTTCAGCGACACCGGCAGCTACGAGGACATCAACGGC |
| | TTCTACCAGGAGGTGGAGCAGCAGGGCTACATGATCGGCTTCAGGCCCGTGAGCGAGGCCTACATCAACCAGCTGG |
| | TGGAGGAGGGCAAGCTGTACCTGTTCCAGATCTGGAACAAGGACTTCAGCGAGTACAGCAAGGGCAGGCCCAACA |
| | TGCACACCATGTACTGGAACATGCTGTTCGACGAGAAGAACCTGGCCGACGTGATCTACAAGCTGAACGGCCAGGC |
| | CGAGGTGTTCTTCAGGCAGAAGAGCCTGGACATCGCCAAGACCTGCGTGCACGCCGCCCAGCAGCCCATCAACAA |
| | CAAGAACAAGAGCAACCAGAAGCAGCAGAGCACCTTCGACTACGACATCATCAAGGACAGGAGGTACACCATGGA |
| | CAAGTTCCAGTTCCACGTGCCCATCACCATGAACTTCAAGAGCAAGGGCAGGGAGAACATCAACAGCCAGGTGAG |
| | GGAGATCATCAGGAACAACGGCATCCAGCACGTGATCGGCATCGACAGGGGCGAGAGGCACCTGCTGTACCTGAG |
| | CCTGATCGACCTGAAGGGCAACATCGTGAAGCAGATGACCCTGAACGACATCATCAACGAGTACAAGGGCAACACC |
| | TACGCCACCAACTACAAGCAGCTGCTGGAGTACAGGGAGGGCGAGAGGACCGAGGCCAGGAGGAGCTGGCAGAC |
| | CATCGAGAACATCAAGGAGCTGAAGGAGGGCTACCTGAGCCAGGTGGTGCACGTGATCGCCAAGATGATGGTGGA |
| | GTACAACGCCATCGTGGTGCTGGAGGACCTGAACGGCGGCTTCATGAGGGGCAGGCAGAAGATCGAGAGGAACGT |
| | GTACGAGCAGTTCGAGAAGAAGCTGATCGACAAGCTGAACTACTACGTGGACAAGCAGAAGGAGGCCACCGAGGC |
| | CGGCGGCGTGCTGCACGCCCTGCAGCTGACCAGCAAGTTCGAGAGCTTCCAGAAGCTGGGCAAGCAGAGCGGCTG |
| | CCTGTTCTACATCCCCGCCTGGAACACCAGCAAGATCGACCCCGTGACCGGCTTCACCAACCAGCTGGACACCAGG |
| | TACCAGAACGTGGAGAAGGCCCTGCAGTTCTTCGGCAGGTTCGACAGCATCAGGTACAACAAGGAGAAGGACTGG |
| | TTCGAGTTCATGCTGGACTACGACAAGTTCACCACCAAGGCCGAGGGCACCCAGACCAGGTGGACCCTGTGCACCT |
| | ACGGCACCAGGATCAGGAGCTTCAGGAACGACATCAACAAGTGGGTGCCCAAGGACGTGGTGCTGACCGAGGAGT |
| | TCAAGCAGGCCTTCGCCGCCGCCAACATCGACGTGAACGGCAACCTGAAGGACGCCATCTGCAACCTGAACGAGA |
| | AGGCCCACCTGGAGGAGCTGATGCACCTGGTGGGCCTGCTGGTGCAGCTGAGGAACAGCAAGACCAACACCGAGA |
| | TCGACTACATGCTGAGCCCCGTGGCCGACAAGAACGGCGTGTTCTACGACAGCAGGACCTGCCCCGACACCCTGCC |
| | CAAGGACGCCGACGCCAACGGCGCCTACAACATCGCCAGGAAGGGCATCTGGGTGATCAGGAAGATCCAGGAGGA |
| | CCAGACCAACGACAAGGTGGACCTGAAGATCACCAACAAGGAGTGGCTGGCCTTCGCCCAGCAGAAGCCCTACCT |
| | GAAGCACCTGaaaagacctgccgccacaaagaaagccggacaggccaagaagaaaaaggattacaaagacgatgacgataagtga |
| 48 | atggcccccaagaaaaagcggaaggtgGAGCCCATCGGCAAGACCGCCGACTACATCAAGAACTACCAGTACCTGGAGACCGACG |
| | AGAGGCTGGCCAAGGAGAGCAAGAAGGTGAAGGAGCTGGCCGACGAGTACCACAAGGAGTTCATCGGCGACGTG |
| | CTGAGCAGCCTGGAGCTGCCCCTGAGCAAGATCAACGAGCTGTGGGACATCTACATGAGCAACGACACCGACAGG |
| | GAGATCAAGTTCAAGAAGCTGCAGGAGAACCTGAGGAAGGTGATCGCCGAGGCCTTCAGCAAGGACAAGAGGTTC |
| | GGCAACCTGTTCAAGAAGGAGATCATCACCGACATCCTGCCCAAGTTCCTGCAGGACAAGGACGACGACATCAAG |
| | ATCGTGAACAGGTTCAAGGGCTTCACCACCTACTICTACGCCTTCCACAAGAACAGGGAGAACATGTACGTGAGCG |
| | AGGAGAAGAGCACCGCCATCCCCTACAGGATCGTGAACCAGAACCTGGTGAAGTACTTCGACAACTACAAGACCTT |
| | CAAGGAGAAGGTGATGCACCTGCTGAAGGACAAGAACATCGTGGAGAGCATCGAGAGGGACTTCAAGGACATCCT |
| | GAACGAGAAGAGCATCGAGGACGTGTTCGGCCTGGCCAACTTCACCCACACCCTGTGCCAGGCCGACATCGAGAA |
| | GTACAACACCCTGATCGGCGGCCTGGTGGTGAAGAACGAGAAGAAGGAGATCAAGGGCATCAACCAGTACATCAA |
| | CGAGCACAACCAGACCAGCAAGAAGGGCAACGGCATCCCCAAGCTGAAGCCCCTGTTCAACCAGATCCTGAGCGA |
| | CAGGAAGAGCCTGAGCTTCACCCTGGACGACATCAAGAAGACCAGCGAGGCCATCAGGACCATCAAGGACGAGTA |
| | CGAGAACCTGAGGGACAAGCTGGCCACCATCGAGAGGCTGATCAAGAGCATCAAGGAGTACGACCTGGCCGGCAT |
| | CTACATCAAGATGGGCGAGGACACCAGCACCATCAGCCAGCACTGGTTCGGCGCCTACTACAAGATCATCGAGGCC |
| | ATCGCCGACGCCTGGGAGAGGAGGAACCCCAAGAAGAACAGGGAGAGCAAGGCCTACAGCAAGTACGTGAGCAG |
| | CCTGAAGAGCATCAGCCTGCAGGAGATCGACGACCTGAAGATCGGCGAGCCCATCGAGAACTACTTCGCCACCTTC |
| | GGCACCACCTGCAGCGACAGGACCAGCGGCGTGAGCAGCCTGAACAGGATCAAGGCCGCCTACACCGAGTTCGTG |
| | AACAAGTTCCCCGAGGGCTTCGAGGACGGCGACGACTGCAACGACGCCTACTTCAAGGCCAACGTGGAGGTGGTG |
| | AAGAACCTGCTGGACAGCATCAAGGACTTCCAGAGGTTCGTGAAGCCCCTGCTGGGCAACGAGGACGAGAGGGAC |
| | AAGGACGAGGCCTTCTACGGCGAGTTCGTGCCCACCTACACCGACATGGACAACATCATCACCCCCCTGTACAACA |
| | GGGTGAGGAACTTCGCCACCAAGAAGCCCTACAGCACCGACAAGATCAAGATCAACTTCGAGAACGTGGTGCTGC |
| | TGAAGGGCTGGGACAAGAACAAGGAGAGCGACTACGCCAGCATCATCCTGATGAAGGACGGCCAGTACTTCCTGG |
| | GCGTGCTGAGGAACGGCAGCAAGAGCACCCTGAAGACCATCCTGCCCAACACCGGCGACTGCTACCAGAAGATGG |
| | TGTACAAGTACTTCAAGGACATCAAGAGCAACCTGCCCAGGTGCACCACCCAGAGGAAGGACGTGAAGGCCCACT |
| | TCGCCGAGAGCAGCGACGACTACACCCTGCTGGACACCAAGGCCTTCGTGAGCGCCCTGACCATCAGCAGGGAGG |
| | TGTTCGAGCTGTACAACGCCCCCGACAAGGAGAAGAAGTTCAAGAAGGAGTACCTGAAGAACACCAACGACAGCA |
| | TCGGCTACGCCAACGCCGTGAGCGTGTGCAAGAGGTTCTGCCTGGAGTTCCTGAAGAAGTACAGGAGCACCGCCAT |
| | CTACGACCTGAGCGACGTGGAGACCAGCGTGGACAGCTTCGACGACCTGAGCAGCTTCTACCAGGAGATCGACAA |
| | GAGGCTGTACAGCATCAGCTTCGAGAACGTGAGCGTGGACAGCGTGAACGAGCTGGTGGACAACGGCAACATGCT |
| | GCTGTTCAGGATCGCCAACAAGGACTTCAGCCCCAACAGCAAGGGCAGGCCCAACCTGCACACCATCTACTGGAG |
| | GATGCTGTTCGACCCCGCCAACCTGAAGGACGTGGTGTACCAGCTGAACGGCAACGCCGAGATCTTCTTCAGGAAG |
| | GCCAGCGTGACCAGGACCGAGCCCACCCACCCCGCCAACGTGGCCATCAAGAACAAGAGCGAGTACAACAAGCA |
| | GAACAAGCCCTACAGCACCTTCAAGTACGGCCTGATCAAGGACAGGAGGTACACCACCGACCAGTTCGAGTTCCAC |
| | GTGCCCATCACCATGAACTTCAAGCAGCCCGAGAGCAGCAAGCTGCAGGACAAGCTGAACAAGCAGGTGCTGGAC |
| | TTCCTGAAGCAGGACGGCGTGAGGCACATCATCGGCATCGACAGGGGCGAGAGGAACCTGCTGTACCTGGTGATGG |
| | TGGACATGGAGGGCAAGATCAAGAAGCAGATCAGCCTGAACGAGATCGCCGGCAACCCCAAGAACCCCGAGTTCA |
| | AGCAGGACTTCCTGGCCCTGCTGCACGAGAGGGAGGGCGACAGGCTGGAGAGCAGGAGGAGCTGGAACACCATC |
| | CAGAGCATCAAGGAGCTGAAGGAGGGCTACATGAGCCTGGTGGTGCACGAGATCGCCAACATGATGCTGGAGAAC |
| | GACGCCATCGTGGTGCTGGAGAACCTGAACAGGAGCTTCATGCAGAAGAGGGGCGAGAAGAGCGTGTACCAGAAG |
| | TTCGAGAAGATGCTGATCGACAAGCTGGGCTACATCGTGGACAAGACCAAGGACGTGAGCGACAACGGCGGCGCC |
| | CTGCACGCCGTGCAGCTGGCCGACACCTTCGAGAACTTCAACAAGACCCAGAAGGGCGCCATCAGGCAGTGCGGC |
| | TTCATCTTCTACATCCCCGCCTGGAGGACCAGCAAGATCGACCCCGTGACCGGCTTCGTGCCCATGCTGAGGTGCCA |
| | GTACGAGAGCATCGTGGAGAGCAAGAAGTTCTTCGGCAAGTTCGACAGCATCTACTACGACGCCACCGGCAAGTAC |
| | TTCGTGTTCCAGACCGACTTCACCAAGTTCAACACCGAGAGCAAGGGCGGCATCCAGAAGTGGGACATCTGCACCT |
| | ACGGCGACAGGATCTACGCCCCCAGGACCAAGGACAGGAACAACAACCCCGTGAGCGAGAGGGTGAACCTGACC |
| | GAGGAGATGAAGAGCCTGTTCGTGAGCCACAACATCAACATCCAGGGCGACATCAAGGCCGGCATCATGCAGCAG |
| | ACCGACAAGGAGTTCTTCGAGAGCCTGCACAGGCTGCTGAGGCTGACCCTGCAGATCAGGAACAGCAAGAAGAGC |
| | ACCGGCAAGGACTACGAGGACTACATCATCAGCCCCGTGATGGGCAAGGACGGCAGGTTCTTCGACAGCAGGAAC |
| | GCCGACGCCACCCAGCCCAAGGACGCCGACGCCAACGGCGCCTACAACATCGCCAGGAAGGGCCTGATGCTGCTG |
| | AGGCAGATCCAGGCCCAGGAGAAGCAGGACCTGAGCAACGGCAAGTGGCTGGAGTTCGCCCAGAGGaaaagacctgccg |
| | ccacaaagaaagccggacaggccaagaagaaaaaggattacaaagacgatgacgataagtga |
| 49 | atggcccccaagaaaaagcggaaggtgACCAGGAAGTTCGACGACTTCATCCACCAGTACAGCCTGAGCAAGACCCTGAGGTTCG |
| | AGGCCAGGCCCATCGGCGAGACCCTGCACAACTTCATCAAGAACGGCCTGCTGGAGAGGGACGAGCACAGGGCCG |
| | AGAGCTACGTGAAGGTGAAGAAGCTGATCGACGAGTACCACAAGGCCTTCATCGACAGGGTGCTGAACGACGGCT |
| | GCCTGAACTACGAGGACAAGGGCGAGTACGACAGCCTGACCGAGTACTACGCCCTGTACAGCGCCCCCAGGAAGG |
| | ACGAGGCCGAGAGGGAGCACTTCAAGGAGACCCAGCAGCACCTGAGGGACCAGGTGGTGAAGAAGCTGACCGGC |
| | GACAACGCCTACAAGCACCTGTTCGGCAAGGAGCTGATCGAGAGCTACAAGGACAAGGAGGACAAGAAGAAGCT |
| | GAACCAGGCCGACCTGGTGCAGTTCATCAACACCGCCACCCCCAAGCAGAGGCTGAACTTCAGCAAGGAGGAGGC |
| | CATCGACCTGGTGCAGGAGTTCTGCGGCTTCACCACCTACTTCGGCGACTTCTACCAGAACAGGGAGAACATGTAC |
| | AGCGCCGAGGAGAAGAGCACCGGCATCGCCTACAGGCTGATCAACGAGAACCTGCCCAAGTTCATCGACAACATG |
| | GAGACCTTCAAGAAGATCGCCGCCATCCCCGAGATGGAGGACAACCTGAAGGAGATCCACGACAACCTGGCCGAG |
| | CACCTGAACGTGGAGAACATCCAGGACATGTTCCAGCTGAACTACTACAACCAGCTGCTGACCCAGAAGCAGATCG |
| | ACGTGTACAACGCCATCATCGGCGGCAAGACCGACGACGAGCACAAGGAGAAGATCAAGGGCATCAACGAGTACG |
| | TGAACCTGTTCAACCAGGCCCACAAGGACGCCAAGCTGCCCAAGCTGAAGACCCTGTTCAAGCAGATCCTGAGCG |
| | ACAAGAACGCCATCAGCTGGCTGCCCGAGGAGTTCGACAACGACCAGGAGGTGCTGAACGCCATCAAGGACTGCT |
| | ACGCCAGGCTGAGCGAGAACATCCTGGGCAAGGAGAACCTGAAGAGGCTGCTGTGCAGCCTGAGCGAGTACGACA |
| | CCAAGGGCATCTTCCTGAGGAACGACCTGCAGCTGACCAGCATCAGCAAGAAGATGAGCGGCAGCTGGACCGACA |
| | TCCCCAACGCCATCAAGAACGACCTGAAGGACGGCGTGCCCGCCAAGAAGCACAAGGAGAGCGAGGAGGACTAC |
| | GAGAAGAGGATCGACAACCTGTTCAAGAGGCAGGACAGCTTCAGCATCGGCTACATCGACGACTGCCTGAACAAG |
| | TTCGACGACAACAACACCTTCACCATCGAGGGCTACTTCAAGAAGCTGGGCGCCAAGGACACCCAGAGCGAGGAC |
| | ATCATCAAGCAGATCGCCAACGCCTACACCGACGTGAAGCCCCTGCTGAACAGCACCTACCCCAAGAGCAAGAACC |
| | TGAGCCAGGACAAGGAGAACGTGGTGAAGATCAAGAGGTTCCTGGACACCCTGATGAGCCTGGTGCACTTCGTGA |
| | AGCCCCTGCTGGGCAAGGGCGACGAGAGCAACAAGGACGAGAAGTTCTACGGCGAGCTGAGCCTGCTGTGGACC |
| | GAGCTGGAGACCATCGTGCCCCTGTACAACATGGTGAGGAACTACATGACCAGGAAGCCCTACAGCAACAGCAAG |
| | ATCAAGCTGAACTTCGACAACAGCCAGCTGCTGGGCGGCTGGGACGCCAACAAGGAGAGCGACTACGCCAGCATC |
| | CTGCTGAGGAGGGACGGCAAGTACTACCTGGCCATCTTCGACAAGGACAGCAAGAAGCTGCTGGGCAAGAGCATG |
| | CCCAGCGACGGCGAGTGCTACGAGAAGATGGTGTACAAGTTCTTCAAGGACGTGAAGACCATGATCCCCAAGTGCA |
| | GCACCCAGCTGAAGAAGGTGGTGAAGCACTTCAGCATCAGCGACACCGACTTCGTGCTGAGCGACGAGAACTTCA |
| | AGGTGCCCCTGACCATCACCAAGGAGGTGTACGACCTGAACAACACCATGTACGGCAAGTACAAGAAGTTCCACA |
| | AGGGCTACCTGACCACCACCGGCGACATCGACGGCTACACCAGCGCCGTGAAGACCTGGATCAACTTCTGCATGGA |
| | CTTCCTGGGCGCCTACGGCAGCACCATGAGCTACGACCTGAGCAGCCTGGCCACCAAGGAGTACAGCAGCCTGGA |
| | GAGCTTCTACCAGGAGGCCAACAGGCTGCTGTACACCGTGAACTTCGTGAAGGTGAGCACCAGCTACGTGAGCAG |
| | GCTGGTGACCGAGGGCAAGATGTACCTGTTCCAGATCTACAACAAGGACTTCAGCGAGTACAGCAAGGGCACCCCC |
| | AACATGCACACCCTGTACTGGAAGGCCCTGTTCGACGACAGGAACCTGAAGGACGTGGTGTACAAGCTGAACGGC |
| | CAGGCCGAGATGTTCTTCAGGAAGAAGAGCATCGACTGCAACAGGCCCACCCACCCCGCCAACCAGCCCATCCAG |
| | AACAAGAACAAGGACAACAAGAAGAAGGAGAGCGTGTTCGAGTACGACCTGATCAAGGACAGGAGGTACACCGT |
| | GGACAAGTTCATGTTCCACGTGCCCATCACCATGAACTTCAAGAGCACCGACAAGGAGAACATCAACCTGCCCGTG |
| | ATGGAGTACCTGCAGACCAGCAACGACACCCACATCATCGGCATCGACAGGGGCGAGAGGCACCTGCTGTACCTGG |
| | TGGTGATCGACCTGCACGGCAACATCGTGGAGCAGTACACCCTGAACGACATCGTGAACGAGTACAACGGCAACAC |
| | CTACAGGACCAACTACAACGACCTGCTGGAGGCCAGGGGCAACGAGCAGCAGAAGGCCAGCCAGAACTGGCAGA |
| | CCATCGAGAACATCAAGGAGCTGAAGGAGGGCTACCTGAGCCAGGTGATCCACAAGATCACCCAGCTGATGATCAA |
| | GTACCACGCCATCATCGTGCTGGAGGACCTGAACATGGGCTTCAAGAGGAGCAGGCAGAAGGTGGAGAAGCAGGT |
| | GTACCAGAAGTTCGAGAAGATGCTGATCGACAAGCTGAACTACCTGGTGGACAAGAAGGCCGACATCGAGAGCAC |
| | CGGCGGCCTGCTGAACGCCTACCAGCTGACCAACAAGTTCGTGAGCTTCAAGAAGCTGGGCAAGCAGAGCGGCTT |
| | CCTGTTCTACATCCCCGCCTGGAACACCAGCAAGATCGACCCCGTGACCGGCTTCGTGAACCTGCTGGACACCAGG |
| | TGCCAGAACAAGAACAAGGCCAAGGCCTTCTTCGCCAAGTTCGACAGCATCAGGTACAACAAGGACAAGGACTGG |
| | TTCGAGTTCGCCCTGGACTACGACAAGTTCGGCAGCAAGGCCGAGGGCACCAGGACCAAGTGGATCCTGTGCACC |
| | CAGGGCAAGAGGATCAAGACCTTCAGGAACGCCGAGAAGAACTTCCAGTGGTACAACCAGGTGATCGACCTGACC |
| | AAGGACCTGAAGCAGCTGTTCGCCCAGTACGACATCGACATCAACGGCAACCTGAAGGAGGCCATCAGCAAGCAG |
| | ACCAAGAAGACCTTCTTCGACGACCTGATGGGCCTGCTGAAGCTGACCCTGCAGATGAGGAACAGCATCACCAGG |
| | ACCGAGACCGACTACCTGATCAGCCCCGTGGCCGACGAGATCGGCAACTTCTACGACAGCAGGACCTGCGGCCCCA |
| | GCCTGCCCGAGAACGCCGACGCCAACGGCGCCTTCAACATCGCCAGGAAGGGCCTGATGATCATCGAGCAGATCAA |
| | GGCCAGCGAGGACCTGAGCAAGCTGAAGTTCGACATCAGCAACAAGAGCTGGCTGAACTTCGCCCAGCAGAAGCC |
| | CTACAAGCACGAGaaaagacctgccgccacaaagaaagccggacaggccaagaagaaaaaggattacaaagacgatgacgataa |
| | gtga |
| 50 | atggcccccaagaaaaagcggaaggtgAACACCAGCAAGTTCATCTTCCAGAACCTGACCCACCAGTTCCAGCTGCAGAAGACCC |
| | TGAGGTTCGAGCTGATCCCCCAGGGCAGGACCCTGGAGCACATCGAGAAGAACGGCCTGCTGGAGGCCGACGAGC |
| | TGAAGGCCAACAGGTACAAGCAGGTGAAGGAGTGGATCGACCTGTACCACAAGGCCTTCATCGAGAAGGTGCTGA |
| | GGGAGCCCAAGCTGGAGGGCCTGAGCGAGTACAGGGTGTTCTTCGAGAACAAGCAGAGGAACGAGAAGGACGAG |
| | GAGGCCTTCAAGAAGCTGAAGAGCGACCTGAGGAAGAAGATCAGCAGCTTCTTCAAGGACGAGCCCAACAAGAC |
| | CTTCTACAAGAGGCTGTTCGACAAGGAGCTGATCAAGGCCGACCTGCCCGACTTCCTGAGGGAGCAGGGCATGCAC |
| | GAGGAGGCCACCATGCTGCTGGAGGAGTTCGGCAACTTCACCACCTACTTCAAGGGCTTCCACGAGAACAGGAAG |
| | AACCTGTACGTGGCCGAGGACAAGAGCACCGCCATCGCCTTCAGGCTGATCGACGAGAACCTGCCCAGGTACATGC |
| | AGAACCTGCTGCTGGTGGAGAAGCACAGGGAGGTGTTCGAGCAGTTCTTCCCCCAGATCCAGGAGAGGCTGGGCA |
| | AGGAGCTGGACGGCCTGAGGGTGGAGGAGTTCTTCAGCGAGGAGGGCTTCAACCTGTGCCTGAACGGCAGCGGCA |
| | TCCACCAGTACAACACCATCCTGGGCGGCGTGAAGGGCGAGGAGGAGGAGGGCATCCCCGGCCTGAACATGCTGA |
| | TCAACCTGTACAGGCAGAAGCACAAGCTGAAGAGCAGGGAGCTGCCCACCCTGTTCCCCCTGCACAAGCAGATCC |
| | TGAGCGACAGGGAGAGCAGCAGCTTCAGGCCCGAGCCCTACGAGGACGGCGACGAGAACACCGTGGTGGAGGAC |
| | CTGCTGAAGCTGGGCGACCAGTGGTTCAACCCCGAGGGCTTCGTGCCCCAGCTGGTGGCCCACCTGACCAAGCTG |
| | AAGGACGAGGAGCAGGACCAGCTGTTCGTGAACAACAAGGCCAACGCCCTGAGCGCCGTGAGCCAGGCCACCCT |
| | GGGCCACTGGGCCGTGCTGGGCGAGGCCCTGAGCAGCTACTGGAACCACGTGCACGACGTGAAGGAGAACGCCCT |
| | GAAGGGCAAGGCCCTGACCGCCAAGGAGAAGTGGCTGAAGCAGCCCTACTTCAGCTTCCAGGAGATCGAGCTGGC |
| | CCTGAGGCACTACATGGGCGCCGAGGGCAAGGAGAACGCCAGCAAGTGGAACGTGTGGCTGGAGAAGCCCCTGGT |
| | GCAGTACTTCAACTACGGCCTGACCAAGGAGGCCGAGAAGACCAAGGTGGACACCGCCAAGGCCGCCTGGGGCCA |
| | CGCCGAGAACATCCTGAGGGAGCTGCAGAACCACGAGAACCCCAAGCTGAAGACCAAGGAGACCAAGGACGCCA |
| | TCAAGGCCTACTTCGACGCCTGCCTGGACCTGCTGCACTTCATCAAGCCCCTGGTGGTGGACCTGGAGCAGTGCGA |
| | GGCCAGCCCCACCTTCTACGGCGACTTCCTGAGCCTGTACCACGAGATGGACGACCTGGTGCCCCTGTACAACAAG |
| | GTGAGGAACTACATGACCAAGAAGCCCTACAGCTACGAGAAGTTCAAGCTGAACTTCAACAAGGGCACCCTGCTG |
| | GACGGCTGGAGCCAGAGCAAGGAGACCGACAACCTGTGCGTGCTGTTCAGGGAGGGCAACAACTTCTTCCTGGGC |
| | ATCATGGACAGGAAGCACAACAAGAGCTTCGTGAAGCCCCCCATGGCCAAGGAGGAGGAGCCCGTGATGCAGAAG |
| | GTGGTGTACAACCTGCTGCCCGACCCCAGCAAGATGCTGCCCAAGTGCTTCACCGCCCCCTACTTCAGGAGGATCG |
| | CCAAGGAGAAGACCAAGGACCCCGAGCAGATCGTGAGCGAGTACAGCAAGGAGAAGGGCCTGATCAAGCCCAAG |
| | CTGGACGTGCTGGACGCCTACGCCCACAAGAAGCACACCAAGGAGAACTTCCAGAGGAACGGCTTCGAGCAGGTG |
| | GTGGAGTACTTCAAGGAGGGCGCCCAGAAGCACCCCGACTGGCACGTGTTCGACTTCAAGTTCAAGCCCACCAGC |
| | GCCTACAAGGACGTGAGCGACTTCTACCAGGAGGTGACCGAGCAGGGCTACTGCCTGGACTTCGTGGACATCCCCA |
| | AGAGGTACATCAACGACCTGGTGGAGAGCGGCAAGCTGTACCTGTTCCAGATCTACAACAAGGACTTCAGCAAGTA |
| | CAGCAAGGGCAAGCCCAACCTGCACACCATGTACTGGAAGGCCATCTTCGACCCCCAGAACCTGAAGGCCCCCATC |
| | TACAAGCTGAACGGCGAGGCCGAGGTGTTCTTCAGGAAGAAGAGCCTGGAGTACACCCAGGAGCAGCTGGACACC |
| | GGCCACCACAGGGAGGAGCTGAAGGGCAAGTTCGACTACCCCATCGTGAAGGACAGGAGGTTCGCCTTCGACAAG |
| | TTCCAGTTCCACGTGCCCATCACCCTGAACCACGCCAGGAAGAACCAGGCCGTGGGCCCCCAGGTGCTGAGGTACA |
| | TCCAGAAGCACAAGGAGGAGCTGCACTACATCGGCCTGGACAGGGGCGAGAGGCACCTGCTGTACCTGAGCGTGG |
| | TGAACGCCAAGGGCGAGATCATCGAGCAGAGGAGCCTGAACAAGATCGGCAACGGCTTCGTGAAGGACGGCAAGC |
| | CCTACACCCACGACTACCAGGGCAAGCTGCTGGAGAAGGAGAGGAACAGGGACAGCGCCAGGAAGGACTGGGAC |
| | GCCGTGGAGAGCATCAAGGAGCTGAAGGAGGGCTACATGAGCATCATCGTGCACGAGATCGCCCAGCTGGTGGTG |
| | AAGTACAACGCCGTGGTGATCCTGGAGGACCTGAACTTCGGCTTCAAGAGGGGCAGGTTCAAGGTGGAGAGGCAG |
| | GTGTACCAGAAGTTCGAGAGGAAGCTGATCGAGAAGCTGAACCACCTGGTGTTCAAGGAGGTGGCCCCCCAGGAG |
| | CCCGGCGGCCTGCTGAAGGCCCTGCAGCTGACCGACCAGTTCACCAGCTTCACCAAGCTGGGCAAGCAGAGCGGC |
| | ATCCTGTTCTACCTGCCCGCCGCCTACACCAGCAAGATCGACCCCCTGACCGGCTTCATCAACCAGGTGCCCTGGAA |
| | GTACACCAACCTGGAGGCCGCCCAGAAGTGGATCGGCAACTTCAAGAGGATCAGCTACCACGCCGAGCTGGACCT |
| | GTTCGAGTGGGTGCTGGACCAGAGGGACTTCCTGAGCAACCCCGCCGGCTGCAAGCACCAGTGGGTGCTGAGGAT |
| | GGACAACCAGGAGAGGTGGCACTGGAACAGCAAGGCCGAGGGCACCAACGGCAAGGGCGCCTACAGCCCCATCA |
| | GGCTGAAGGAGCAGTTCGAGGACCTGCTGGCCAGGCAGAACATCAGCTACAACGGCAACAGCAAGCTGCTGGAGG |
| | ACATCCAGGCCGTGAAGGACGCCAGCTTCTTCAAGACCCTGTTCTTCTTCCTGAGGACCCTGACCCAGCTGAGGTA |
| | CAACAACGGCAAGAGCGGCGCCGAGGAGGAGGACTTCATCCTGAGCGCCGTGGCCAACCCCGGCATGGAGCCCTT |
| | CGACACCAGGAAGGCCAAGCAGGGCCTGCCCCTGGACGCCGACGCCAACGGCGCCTACCACATCGCCATGAAGGG |
| | CCTGATGACCCTGGGCCAGCTGAAGGAGAAGCCCAACGGCAAGCTGGAGATCCCCAAGCTGGACAAGAGCGCCTG |
| | GCTGTGCTTCATGCAGCAGAGGAGCAACCAGGGCaaaagacctgccgccacaaagaaagccggacaggccaagaagaaaaagga |
| | ttacaaagacgatgacgataagtga |
| 51 | atggcccccaagaaaaagcggaaggtgCAGACCAAGAACTTCATGAGCTTCAGCGACTTCACCAACCTGTACGCCCTGAGCAAGA |
| | CCCTGAGGTTCGAGCTGAGGCCCATCGGCAGGACCGAGGAGTTCTTCAAGAAGAACAAGGTGTTCGAGAAGGACA |
| | AGACCATCAACGACAGCTACAACCAGGCCAAGCTGTACTTCGACGAGATGCACAGGAGGCTGATCAGGGACGCCC |
| | TGAACACCCAGAACACCAGCGCCCTGGACTACGAGAAGTACGCCAAGCACTGGAAGGAGCTGCAGAAGGACAAG |
| | GGCAAGTACATCCAGAACTGGAAGGACGCCCAGAAGGAGTTCAGGGAGTGCATCAGCAACCTGCTGGACCAGCAG |
| | GCCGAGCAGTGGAAGAGCAAGTACGCCAGCGTGGGCCTGAACAAGGCCGACATCAAGCAGAAGGGCACCGACTT |
| | CCTGAGCAGCGCCGGCATGGTGAAGATCCTGAAGAAGGAGTTCCCCAAGGAGGCCGAGAACCAGCTGGTGAAGGA |
| | GGGCTTCCCCAGCCTGTTCATCCAGAAGGCCGAGGCCCCCGAGCAGAAGCAGTACATCTTCGACAGCTTCGACAGG |
| | TTCAGCACCTACCTGGCCAAGTTCAACCAGACCAGGGACAACCTGTACAAGGACGACGGCACCCCCACCGCCATCG |
| | CCACCAGGGTGGTGGAGAACTTCGTGGCCTTCCTGCAGAACCAGGCCATCTTCAACAACCACTACATCAGCCTGAA |
| | GAGCGAGCTGGACTTCAGCAGGGAGGAGATCGACTGCTTCACCGCCAACTACTACGTGAGGTGCCTGCTGCAGGA |
| | GGACATCGACGAGTACAACAAGACCATCGGCCAGATCAACGCCAGGATCAAGGAGCTGAGGGACAGGAAGCAGA |
| | AGGAGAGCGACTTCAAGAAGAGCGGCTACCCCCTGCTGAAGAGCCTGAACAAGCAGATCCTGGGCAAGATCGAGA |
| | AGAAGAGGCAGTTCATCGAGAACAAGGCCGACGTGTTCCCCGCCTTCAGCGAGCTGATCGAGCAGGCCAACGCCA |
| | GGCTGACCGCCCTGAGGCAGCTGATCAACAACCTGTGCGAGGAGGTGTTCGAGGAGGCCTACGGCGGCATCTACAT |
| | CAAGAACACCGGCATCAACACCATCAGCAGGAGGTGGTTCGCCAGCTGGTACGACTTCGAGATCGCCCTGCCCCAG |
| | AAGAGCAAGAACGAGGAGGAGAGCAAGGTGGTGACCTTCGTGAGCCTGCAGGACATCAAGGACACCCTGGAGAA |
| | CAAGCTGAGCGGCGACATCTTCAAGGAGGAGTACTACAAGAAGAAGATCATCAACAAGGAGCAGAGCAACTGGAA |
| | GCAGATCCTGGCCATCCTGCAGAACGAGTGGAACGAGCTGTTCAGCGAGTACCAGAAGGCCCTGGAGAACACCAA |
| | GAAGCTGGACCTGAACAGCGTGTTCAGCAACAGCAAGGAGCAGGTGGAGATCATCAAGAGCTTCGCCGACGCCAG |
| | CCTGAGGGTGTACCAGGTGGTGAAGTACTTCGCCCTGGAGCACAAGAAGAGCGACGAGATCCCCAGCAGCTACAG |
| | CACCGACTTCTACGAGGAGTTCGACAAGCAGTACAACAACTTCGAGTTCATCCCCTACTACAACGCCCTGAGGAAC |
| | TTCCTGACCAAGAAGGTGGTGGGCGAGAACAAGATCAAGATCAACTTCGAGAAGGGCAACCTGCTGAACGGCTGG |
| | GCCGAGAGCCCCGAGGGCAACGCCCAGTTCTGCGGCTACATCCTGAAGAAGGAGAACAAGTACTTCCTGGGCATC |
| | ACCGACCAGCCCACCATCCTGGACACCAAGGACAACCCCAGCCTGACCAACATCACCGACAAGGAGTACTACGAG |
| | AAGATGGCCTACAGGCAGCTGAAGAGCACCACCATCTACGGCAGCGGCTACGAGGGCCTGTACAACAGCAAGTAC |
| | GCCAACGACAAGAAGTACATCAGCGAGAGCAAGATCATCGAGAGGGTGAAGAAGCTGCTGCAGGAGAAGTACCTG |
| | ACCAGGTTCCCCGAGCTGAAGCAGATCATCGAGAAGGAGTACAAGGACGCCAACGAGCTGGCCAAGAACCTGGGC |
| | AACTTCAACCTGTACAGCCTGGAGTTCATCCCCGTGAACAAGAACTACATCGAGAAGCTGACCCACGACAAGAACG |
| | GCAGCATCAAGTACCTGTACATCTTCGAGATCGCCAACAAGGACCTGGGCAAGATCACCCCCAGGAAGTACAACAT |
| | CCACAGCCTGTACTTCAAGAACCTGTTCAGCGAGGACAACCTGGAGAAGCCCGTGTTCAAGCTGAGCGGCGGCGC |
| | CGAGATCTTCTTCAGGCCCAAGACCGTGAACCTGCCCAAGAAGAAGGACAAGGCCGGCAAGGACATCATCGACCA |
| | CAAGAGGTACGCCCAGGACAAGCTGTTCTTCCACCTGCCCATCGTGATCAACTTCGGCGCCGGCAAGGTGGGCAAG |
| | TTCAACCAGGAGCTGAACAAGAAGCTGCTGGCCGGCAACAAGAACATCAACATCATCGGCATCGACAGGGGCGAG |
| | AAGCACCTGGCCTACTACAGCGTGATCAACCAGAAGGGCGAGATCATCGAGCAGGACAGCCTGAACGAGATCAAC |
| | GGCGTGAACTACTACGGCCTGCTGCAGAAGAGGGAGAAGGAGAGGCTGGAGAACAGGAAGAGCTGGCTGCCCCT |
| | GCAGCAGATCAAGGACCTGAAGAGGGGCTACATCAGCCAGGTGGTGTACAAGATCGTGAGGCTGGCCATCGAGCA |
| | CAACGCCATCATCGTGCTGGAGGACCTGAACATGAGGTTCAAGCAGATCAGGGGCGGCATCGAGAAGAGCATCTAC |
| | CAGCAGCTGGAGAAGGCCCTGATCGACAAGCTGGGCTACCTGGTGTTCAAGGACAGGAACGCCAAGGAGGTGGGC |
| | GGCGTGCTGCAGGGCTACCAGCTGAGCGCCCCCTTCGAGAGCTTCCAGAAGATCGGCAAGCAGACCGGCATCATCT |
| | TCTACACCCAGGCCAGCTACACCAGCGTGACCGACCCCCTGACCGGCTTCAGGAAGAACATCTACGTGAGCAACAG |
| | CGCCCCCATCGAGAAGATCAAGGTGGCCATCAACAACTTCGAGGAGATCGGCTGGGACGACGTGGTGCAGAGCTA |
| | CTTCTTCAACTACGACACCAAGAACTTCAGCAACGGCAACGACATCCAGAGCAAGAAGTGGAAGATCTACGCCAA |
| | GGTGCCCAGGATCAAGAGGGAGAAGGTGAACGGCTACTGGCAGTACAACGACGTGGACCCCAACGACATGCTGGA |
| | GAAGCTGTTCAGGCTGTACGAGTTCAAGAACCTGAGGAGCAACATGCTGGAGCAGATCCACGCCAAGGAGACCAA |
| | CGGCGAGCTGAGGGGCGAGAGGGACTTCGACAACAAGCCCAGGAACTTCTACCAGAGCCTGGTGTACATCCTGAA |
| | CCTGATCCTGCAGCTGAGGAACAGCACCAGCAAGCAGATCAAGCTGAACGAGAAGGAGGACGTGTACGAGACCGG |
| | CGAGGACGTGGACTTCATCGCCAGCCCCGCCTACCCCTTCTTCACCACCCTGAGCAGCAAGAGCAACAGCAACCTG |
| | GCCGGCTTCGAGAACAGGTTCACCGGCCTGAACCACGACCAGGAGCAGAAGAAGTTCAACGGCGACGCCAACGG |
| | CGCCTACAACATCGCCAGGAAGGGCATCATCATCCTGGAGAGGATCACCAAGAACAGCGACAAGCCCAACCTGTAC |
| | ATCAGCAACGAGGACTGGGACAAGTTCGTGAGCCAGAGCTACCCCaaaagacctgccgccacaaagaaagccggacaggccaag |
| | aagaaaaaggattacaaagacgatgacgataagtga |
| 52 | atggcccccaagaaaaagcggaaggtgGAGAGCAAGACCCTGGACAGGTTCATCGGCAAGTACCAGCTGAGCAAGACCCTGAGG |
| | TTCGAGCTGATCCCCCAGGGCAAGACCAAGGAGCACATCGAGCAGAAGGGCCTGCTGCAGCAGGACGAGCACAGG |
| | GCCAAGGCCTACAAGATGGTGAAGAAGATCATCGACAGGTACCACCAGCACTTCATCGCCACCGCCCTGAACAACC |
| | TGGCCCTGGACGGCCTGCAGAGCTACGAGGAGCTGTACCACAAGAAGGAGAAGGAGGAGGCCGACAAGAAGAAC |
| | TTCGAGAGCCTGAAGGCCGGCATGAGGAAGCAGATCGGCGACGCCTTCAACAAGAGCGAGGCCAGCAAGGTGCA |
| | GGAGATCTGGAAGGTGCTGTTCGACAAGGAGCTGATCAGGAAGGCCCTGCCCGAGTTCGTGACCAGCGAGGAGGA |
| | CAAGAAGCTGCTGGCCGAGTTCAAGGACTTCACCACCTACTTCACCGGCTTCCACCAGAACAGGGCCAACATCTAC |
| | ACCCACGAGGAGAAGGCCACCGCCGTGGCCTTCAGGCTGGTGCACGAGAACCTGCCCAAGTTCCTGGACAACAGC |
| | AGGGTGTTCGACTGGATCCAGACCGAGTACCCCGACCTGGACCTGAGCGCCATCGAGAAGGAGCTGGCCCCCGAG |
| | CTGCAGGGCACCACCCTGCCCGAGATCTTCGCCCTGAGCTTCTTCAACCACACCCTGACCCAGAACGGCATCGACC |
| | TGTACAACGCCGTGCTGGGCGGCAAGACCACCGAGCAGCAGAACAAGATCAGGGGCCTGAACGAGTACATCAACC |
| | TGTACAACCAGAGGAGGACCGCCGAGGCCAAGAAGGAGGGCGAGAAGTTCAGGAGGGTGCCCCAGTTCAAGGAG |
| | CTGTACAAGCAGATCCTGAGCGACAGGGAGAGCATCAGCTTCCTGCCCGAGACCTTCGAGAACGACAGCGAGGTG |
| | CTGAGCGCCATCGAGAACTTCTTCGCCACCCAGCTGGTGCACTTCGAGAAGGACAACAGGAGCATCAGCGTGTTCG |
| | CCGAGCTGCAGAAGCTGCTGCAGGAGCTGAAGGAGGACGACAGGAGCAAGATCTTCGTGAGGAACGACCTGGCC |
| | CTGACCAACATCAGCCAGAACATCTTCGGCGACTGGCAGGTGATCAAGAACGCCCTGGAGCACTACTACACCACCA |
| | CCGTGAACCCCCTGGCCGTGGGCAAGAAGCTGACCAAGGCCTACGAGAACGGCAAGGAGAAGTGGCAGAAGCAG |
| | AGCTACTACAGCATCTACGAGATCGAGGAGGCCCTGCTGGCCTACAGCAGCGAGGCCGACGTGCTGAAGGAGAAG |
| | AACCTGACCGCCGCCAGCAGGCCCCTGTGCAACTACTTCGCCACCTTCAGCAAGGACGAGGAGGGCAACAACCTG |
| | TTCCAGAGGATCGAGACCGCCTACACCGCCGTGAAGGACCTGCTGAACACCGACTACCCCGAGGAGAAGAACCTG |
| | GCCCAGGACAAGACCAACGTGGCCGCCATCAAGACCTTCCTGGACGCCATCAAGAACCTGCTGCACTTCATCAAGC |
| | CCCTGCACCCCGCCAGGCTGGAGGGCGAGAAGGACGAGAGGTTCTACGGCGCCTTCATCCCCCTGTTCGACCAGCT |
| | GAACCTGCTGACCCCCCTGTACAACAAGACCAGGAACTACCTGACCAGGAAGCCCTACAGCACCGAGAAGGTGAA |
| | GCTGAACTTCGAGAACAGCACCCTGCTGGACGGCTGGGACGCCAACAAGGAGACCGCCAACACCAGCGTGCTGCT |
| | GAGGAAGGACGGCCTGTACTACCTGGCCGTGATGGACAAGAAGTACAACAAGGTGTTCGAGCAGATGCCCGAGGT |
| | GGCCGAGGGCGAGAAGGTGTTCGAGAAGGTGGTGTACAAGCTGCTGCCCGGCGCCAACAAGATGCTGCCCAAGGT |
| | GTTCTTCAGCGCCAAGAACATCCAGTACTACAACCCCAGCACCGAGCTGCTGGAGAACTACAAGAACGAGACCCA |
| | CAAGAAGGGCGCCACCTTCAACCTGGTGCACTGCCACCAGCTGATCGACTTCTTCAAGGCCAGCATCGCCAGGCAC |
| | CAGGACTGGAAGAACTTCAACTTCCAGTTCAGCGACACCGGCAGCTACGCCGACCTGAGCGGCTTCTACAGGGAG |
| | GTGGAGCAGCAGGGCTACAAGATCACCTTCCACAACCTGCCCGAGAGCTACATCAACCAGCTGGTGGACCAGGGC |
| | AAGCTGTACCTGTTCCAGATCTACAACAAGGACTTCAGCCCCTTCAGCAAGGGCAGGCCCAACCTGCACACCATGT |
| | ACTGGAAGATGGTGTTCCACGAGGGCAACCTGACCAACGTGGTGTACAAGCTGAACGGCCAGGCCGAGATCTTCTT |
| | CAGGAAGAAGAGCCTGAACTACGACGAGAAGACCCTGAGGGAGGGCCACCACGCCGCCGCCCTGAAGGACAAGT |
| | TCGCCTACCCCATCATCAGCAACAGGAGGTTCGCCTTCGACAAGTACCAGTTCCACGTGCCCATCACCATCAACTTC |
| | AAGAGCACCGGCAGGGACAACGTGAACGCCGAGGTGCTGAGCTTCCTGCAGCACAACCCCAGCGTGAACATCATC |
| | GGCATCGACAGGGGCGAGAGGCACCTGCTGTACCTGACCCTGATCGACCAGAAGGGCAACATCAAGAAGCAGATG |
| | AGCCTGAACCAGATCATCAACAGCTACAGGAACAAGGAGGGCGACATGGTGAACGTGAAGACCGACTACCAGAAC |
| | AAGCTGCACCTGAAGGAGGAGGAGAGGCTGAAGGCCAGGCAGGGCTGGGGCCTGATCGAGAACATCAAGGAGCT |
| | GAAGGAGGGCTACCTGAGCCACGTGGTGCACGAGATCGCCAAGATGATGGTGGAGCACAACGCCATCGTGGTGAT |
| | GGAGGACCTGAACTTCGGCTTCAAGAGGGGCAGGCAGAAGGTGGAGAAGCAGGTGTACCAGAAGTTCGAGAAGA |
| | TGCTGATCGACAAGCTGAACTACCTGGTGTTCAAGGACAGGACCGCCACCGAGCCCGGCGGCCTGCTGCACGCCCT |
| | GCAGCTGACCGGCAGCTACACCGACTTCATGAAGTACAAGCAGAAGCAGTGCGGCTTCCTGTTCTACGTGCCCGCC |
| | TGGAACACCAGCAAGATCGACCCCCTGACCGGCTTCGTGGACTTCCTGAAGCCCAAGTACGAGAGCATCGAGAAG |
| | GCCCAGAACTTCCTGGCCAAGTTCAAGAGCATCAGGTACAACCCCGCCAAGCAGTGGTTCGAGTTCACCTTCGACT |
| | ACAAGGACTTCACCGACAAGGCCGAGGGCAGCAGGACCAGGTGGACCCTGTGCAGCACCAACCAGCCCAGGTAC |
| | AGGTGGAACCCCAAGGCCGCCAACGGCAAGGGCGCCCAGGAGGCCGTGAACGTGACCCAGGCCCTGGAGGACCT |
| | GTTCAGCGCCCACAACATCGCCTACGGCAGCGCCGACGACCTGGTGACCGCCATCACCGCCCAGAGCAGCGCCGA |
| | CTTCTACAAGGCCCTGCTGAGGGCCATGGTGACCCTGCTGGCCCTGAGGCACAACAACGGCAAGGCCGGCACCGA |
| | CGAGCAGGACTACATCCTGAGCCCCGTGGAGCCCTTCTTCGACAGCAGGCTGGCCGACGACAGCAGGCCCAAGGA |
| | CGCCGACGCCAACGGCGCCTACCACATCGCCAAGAAGGGCCTGTGGGTGCTGCAGCAGATCAACGAGCTGCAGGG |
| | CGACGACTGGAAGAACCTGAAGCTGGCCATCAGCAACAAGCAGTGGCTGCAGTTCGCCCAGCAGCTGAACGCCGA |
| | CATCAGCCCCAAGGTGAGCaaaagacctgccgccacaaagaaagccggacaggccaagaagaaaaaggattacaaagacgatga |
| | cgataagtga |
| 53 | atggcccccaagaaaaagcggaaggtgAGCAGCAGCATCGTGAGCGTGAGCACCGAGAAGAAGCAGGCCGAGGTGCTGAGCAAC |
| | AAGAGCGTGTTCGACCAGTTCACCAGGAAGTACGCCCTGAGCAAGACCCTGAGGTTCGAGCTGAGGCCCGTGGGC |
| | AAGACCAGGGAGAACATCGAGAAGGCCAACCCCAACTTCATCAGGGACAAGGAGATCGAGGACGCCTACCAGATC |
| | CTGAAGCCCGTGTTCGACAAGCTGCACGAGGAGTTCATCACCGAGAGCCTGGAGAGCGACGAGGCCAAGCACCTG |
| | AGCTTCGAGGACTACTTCAGCGTGTACAAGAACCTGAGGAACGAGCAGGACAGGGAGGAGAAGAAGAAGATCGA |
| | GAAGAGCCTGGAGACCAAGGAGAAGAACCTGAGGGCCGCCTTCAGCGACATCTACAAGGCCGAGGGCGAGAAGT |
| | TCAAGAGCAAGGTGGGCAGCGACAAGAAGGGCAAGCCCATCCTGAAGGAGGACAGCTTCAAGATCCTGACCGAG |
| | GTGGGCATCCTGAAGTACGTGAAGGCCAAGATCGAGGAGTTCGTGGTGATGAACCTGAAGACCCACGAGGGCAAG |
| | GAGGTGAAGAAGGAGGACCTGGAGAAGGCCCTGGGCACCGCCGACAGCAAGGGCGTGTTCGAGGGCTTCTTCAC |
| | CTACCTGACCGGCTTCAACCAGAACAGGGAGAACTACTACAGCACCGAGGAGAAGGCCACCGCCGTGGCCAGCAG |
| | GATCATCGACGAGAACCTGCCCAAGTTCGCCGACAACGTGCTGGAGTTCCAGAAGAGGAAGGACGAGTACCTGAA |
| | GGTGTACGACTTCCTGAAGGAGAACGGCATCGAGACCATCGGCAAGAACCAGAAGGGCGAGAAGGTGGAGCTGG |
| | AGAAGATCATCGCCGACATCTTCGAGATCGGCTACTTCACCAACTGCCTGAGCCAGAGGGAGATCGAGAAGTACAA |
| | CCTGAACATCGGCAACGCCAACAACCTGATCAACAGGTACAACCAGCAGCAGAGCGAGAAGAACAAGAAGCTGA |
| | GGATCTTCAAGACCCTGTACAAGCAGATCGGCTGCGGCGAGAAGGGCGACTTCATCCCCGCCATCAAGAACGACGA |
| | CGGCCTGAGGGCCGTGCTGAAGAACGTGGTGGAGAACGGCGAGAAGTACTTCGGCCAGTTCGACGGCAAGATCGA |
| | GGACAAGGTGGACACCGTGGGCGAGTTCGTGCACTACATCATCAACCACGAGAGCTTCGAGGGCATCTTCTGGAGC |
| | GACAAGGCCCTGAACACCATCAGCGGCAAGTACTTCGCCAACTGGTTCATCCTGAAGAAGGCCCTGAAGGAGGGC |
| | GGCGTGTTCGGCGGCAAGAAGAAGGACGACGAGGACATCAAGATCCCCCAGGCCGTGGAGCTGAAGAACCTGTTC |
| | GAGATCCTGGACAAGACCGAGAAGTGGAAGAACAAGGAGGACGGCCTGTTCAAGACCAGCCTGTTCGACGGCGA |
| | GGGCAACAAGAAGGGCATCATCGAGAGGGCCGAGAAGCCCAGCAAGGCCCTGCTGGGCATGATCTTCGCCGACGT |
| | GGAGAAGATGGCCAAGGACTTCAGCGAGCTGAGCCCCAAGGTGCTGAGCATCACCACCGACTTCAAGAAGCCCGA |
| | GCACATCGAGAACATCAAGATGCTGCTGGACAGCGCCCTGTACGTGAACCAGATCGTGAAGTACTGGAAGGTGAAG |
| | GACAAGTTCGCCGTGGACCCCATCCTGGCCGAGAGCCTGAAGGCCATCCTGTTCGGCGACAACAACCCCACCAAGT |
| | TCTACGACATCATCAGGAACTACCTGACCAAGAGGCCCCAGGAGGGCCTGAACAAGCTGAAGCTGAACTTCGACA |
| | ACAGCGTGCTGGGCGGCGGCTGGGACGTGAACAAGGAGCCCGAGAGGTGGTGCCTGATCCTGAAGGACGGCGAG |
| | AAGAAGTACCTGGCCATCCTGACCAAGGCCAGCCAGAAGCTGTTCGGCGAGAAGGCCAAGTACAAGGAGGTGGTG |
| | ACCGACGAGAACTACCTGAAGATGGACTACAAGCTGCTGCCCGGCCCCAACAAGATGCTGCCCAAGGTGCTGCTGC |
| | CCAAGAGGAACAGGAAGGAGTTCGGCGCCACCGACGACATCCTGAGCATCTACGACGAGGGCGGCTTCAAGAAGA |
| | ACCAGCCCAGCTTCACCAAGGAGAAGCTGTGGAAGATCATCGACTTCTACAAGGAGGGCCTGAAGGTGTACCCCA |
| | GCGCCGAGAAGAGCTGGCAGGACCTGTTCGGCTTCAACTTCAGCCCCACCGAGAAGTTCGAGAGCATCGACCAGT |
| | TCTACAACGAGGTGGAGAAGCAGGGCTACAAGCTGAGCTGGAACATCATCAGCAAGAAGCTGATCAACGAGGCCG |
| | TGGAGAACGGCGAGATCTACTTCTTCGAGATCAGGAACAAGGACAACAACCTGAAGGAGGGCAAGGAGAAGACC |
| | AGCGAGAAGAACCTGCACACCATCTACTGGAACGAGATCTTCGGCGACGTGGCCAACAAGCCCAAGCTGAACGGC |
| | GAGGCCGAGATCTTCTACAGGAAGACCATCCTGAAGAAGAAGGAGAACGAGACCGACAAGGAGTTCGAGATCAGG |
| | CTGAAGGAGAAGCTGGGCAAGAAGAAGGACAAGAGCGGCAAGGAGGTGCTGGAGCACAAGAGGTTCGCCAACG |
| | AGAAGTTCGTGTTCCACTGCCCCATCACCCTGAACTTCTGCCTGAGGAGCCTGAAGTTCAACGACGAGATCAACGA |
| | GAGCATGGTGAAGCTGAACGACGACATCACCACCATCGGCATCGACAGGGGCGAGAAGCACCTGGCCTACTACTTC |
| | GTGGTGAAGCAGACCAAGGACGGCAAGATCGAGACCCTGGACCAGGGCAGCTTCAACGAGATCAACGGCCAGAA |
| | CTACGCCGAGAAGCTGGAGAAGATGGCCGGCAACAGGGACGAGGCCAGGAAGAACTGGAAGACCATCGGCACCA |
| | TCAAGGAGCTGAAGGACGGCTACATCAGCCAGGTGGTGAGGAAGATCGTGGACCTGGCCATCTACGAGGACGTGG |
| | AGAAGAAGGTGTTCAGGGAGACCCCCGCCTTCATCGTGCTGGAGGACCTGAACATCGGCTTCAAGAGGGGCAGGC |
| | AGAAGATCGAGAAGCAGGTGTACCAGAAGCTGGAGCTGGCCCTGGCCAAGAAGCTGAACTTCCTGGTGGACAAGC |
| | ACGCCAAGGAGGGCGAGATCGGCAGCGTGACCAGCGCCCTGCAGCTGACCCCCCTGGTGAACAACTTCGGCGACA |
| | TCGAGGGCAAGAAGCAGTTCGGCATCATGTTCTACACCAAGGCCGACTACACCAGCCAGACCGACCCCGTGACCGG |
| | CTGGAGGAAGACCATCTACCTGAAGAAGGGCAGCGAGGAGAGCATCAAGAAGCAGATCCTGGACAACTTCGACGA |
| | CATCAAGTTCGACGGCAAGGACTACTACTTCAAGTACACCGACAGGAACACCAACAAGCCCTGGGAGCTGTACAGC |
| | GGCAAGGACGGCGAGAGCCTGGACAGGTACTACAGGGAGCTGGTGCACGAGAACAGCGACAAGAAGTGGGTGCC |
| | CAAGGAGCAGAACCTGAAGAAGATCCTGGACGGCCTGTTCGAGGGCTTCGACAAGGAGAAGAGCCTGCTGGAGC |
| | AGCTGAGGCAGGGCAAGAACCCCAACAAGATCAACGACAGGACCAGCTGGGAGAGCCTGAGGTTCGTGATCGACC |
| | TGATCCAGCAGATCAGGAACAGCGGCAAGACCGAGGAGGACGCCGACTTCATCCTGAGCCCCGTGAGGGACAAGA |
| | ACGGCAACCACTTCGACAGCAGGAAGATCAACCTGAACCTGCCCACCAGCGGCGACGCCAACGGCGCCTACAACA |
| | TCGCCAGGAAGGGCATCATCATGTGCGAGCACATCAAGAGGGACCTGAGCCTGTTCATCAGCGACGAGGAGTGGA |
| | GCGCCTGGCTGGCCGGCAGGGAGATCTGGGAGAACTGGATCAAGAAGAACGAGAAGAACCTGAAGAGGAAGCAG |
| | aaaagacctgccgccacaaagaaagccggacaggccaagaagaaaaaggattacaaagacgatgacgataagtga |
| 54 | atggcccccaagaaaaagcggaaggtgAAGAGCATCGAGGAGCTGATCGGCCAGTACAGCCTGACCAAGACCCTGAGGTTCGAG |
| | CTGAAGCCCATCGGCAAGACCCTGGAGCAGATCGAGAGGAAGGGCGTGCTGAGCGCCGACCAGACCAGGGCCGA |
| | CCAGTACAAGAGGGTGAAGGAGAGCATCGACAGGTACCACAAGGCCTTCATCACCGAGAGCCTGGACAGCCTGGT |
| | GCTGAAGGTGAAGAGCACCGGCAACACCAGCGACAGCCTGGAGGAGTACGCCAGCCTGCTGGACAACAGCGCCG |
| | ACGACGAGGCCCTGGACAAGGTGAAGAAGGGCCTGAGGAAGCAGGTGGCCGAGAGGTTCGACAAGCAGATCAGG |
| | CTGAAGAAGCTGTTCAAGAACGACCTGATCACCAAGATCCTGCCCGAGTTCCTGAAGGTGAACGGCAACGAGGAC |
| | GAGAGGAAGGCCGTGGAGAGCTTCAGGAACTTCACCACCTACTTCAGCGGCCTGAACGAGAACAGGAAGAACATG |
| | TACAGCAAGGAGGCCAAGAGCACCGCCATCGCCTACAGGCTGATCGACGAGAACTTCCCCAGGTTCCACGACAAC |
| | ATGCTGAGCTTCGCCAAGATCGCCGCCGTGAACGAGGTGAGCAACCACTTCGCCGAGATCGCCGAGGCCTTCAAGA |
| | GCGAGATGGTGGTGGCCAGCGTGGCCGAGATGTTCGGCCTGGAGTACTTCAACAACACCCTGACCCAGCAGAAGAT |
| | CGACCTGTACAACTACGTGATCGGCGGCAAGACCGAGGGCGACAAGGTGAAGATCCAGGGCATCAACGAGTACGT |
| | GAACCTGTACAACCAGAACCACCCCAACGACAAGCTGCCCCTGCTGAAGCCCCTGTACAAGATGATCCTGAGCGAC |
| | AGGGTGGCCCTGAGCTGGCTGCCCGAGGAGTTCAACGACGACGAGGCCATGGTGTGCGCCATCAAGGAGTTCTAC |
| | AACGCCATCCAGCCCGTGCTGTGGGGCGAGGTGGAGGACGACGACAACAGCGTGTGCCACCTGCTGAAGAACATC |
| | GGCAAGTACGACATCGAGCACATCTTCATCAGCAACGGCGCCGCCCTGAACGACGTGAGCAGGCAGATGTTCGGCC |
| | AGTACGACTTCCTGGCCAACGCCATCAAGAAGGACATCAGGCAGAACACCAAGAGGAGCAGCAGGGAGACCGAC |
| | GAGAAGTTCGAGGGCAAGGTGAACACCCTGTTCAAGAAGGCCAAGAGCTACAGCATCGCCCACCTGAACGCCCTG |
| | ATCAAGGAGAACAGGAGCGAGACCATCGAGAGCTTCTACAGCAACCTGGGCGCCTGCGACAGGGACGGCGAGCA |
| | GAAGATCAACCTGCTGACCCAGATCGAGCTGGCCCACGAGGCCGCCCAGGACGTGCTGGACGGCAAGTACGCCGA |
| | CATCAACAGGGACGAGAAGGCCACCATGCTGATCAAGGAGCTGCTGGACGCCTTCAAGGCCCTGCAGTACTTCATC |
| | AAGCCCCTGCTGGGCAGCGGCGAGGAGGCCAGCAAGGACAACGTGTTCTGCACCAAGCTGCTGAACGCCTGGCAG |
| | GCCCTGGACGCCGTGACCCCCCTGTACGACAAGGTGAGGAACAGGCTGACCAGGAAGCCCTACAGCACCGACAAG |
| | ATCAAGCTGTACTTCGAGAACAAGGGCAAGTTCCTGGACGGCTGGGTGGACAGCAAGACCGACAACAGCGACAAC |
| | GGCACCCAGTTCGGCGGCTACATCCTGAGGAAGAAGAACGAGATCGGCGAGTACGACTACTTCCTGGGCATCAGCA |
| | GCGACACCAAGCTGCTGAGGGCCAACCCCGACATCCAGTACGAGCACGGCATGTACGAGAGGCTGGACTACTACC |
| | AGATCCAGAGCAAGACCTTCCTGGGCAGCAGCTACAAGGGCGAGTACAACGACGACGCCATGAGGCTGATGAGCG |
| | CCTTCGTGGAGTACGGCAAGAAGAGCAGGTTCGACAGCTGCGTGCTGCCCACCGACAAGGAGAAGAGCCTGCCCA |
| | CCTACCTGAAGAGGATCTACACCGACAACAACAGCTTCTACAACGAGGCCATGACCGACACCGTGGTGGCCGAGGC |
| | CTACAACAACATCAAGAGCCACCTGCTGCTGACCCTGGCCGGCATCAAGAGGGTGGAGGCCGCCGTGAACCTGAG |
| | CAAGCACAACGAGTACAACCTGATCCAGATCTTCGACGAGATCGGCAAGATCACCAGCAGCAGCAAGATCTTCAGC |
| | TTCTTCCCCGTGGACACCAGCCTGGTGGAGAAGCTGAACGAGAGGGACAACAAGCCCCTGTTCCTGTTCAAGATCA |
| | GCAACAAGGACCTGAGCTACGCCGAGACCAGCAGCAAGAACATCAGGAAGAGCAGGGGCAAGGAGAACCTGCAC |
| | ACCATGTACTTCAAGGCCCTGATGGAGCAGAGCCACAACGTGTTCGACATCGGCACCGCCGAGGTGTTCTTCAGGA |
| | AGGCCACCCCCGGCCTGGACAAGAGCACCACCATCCACAAGGCCAACGAGGCCCTGTGCAACAAGAACCCCCACA |
| | GCAAGAAGACCAGCAGCACCTTCGGCTACGACATCATCAAGGACAGGAGGTACGTGGAGAACAAGTTCCAGCTGC |
| | ACCTGAGCATGGTGATCAACTACCAGGGCAAGAACCCCAACACCGACATCATCAGCCCCCAGGTGCTGGACATCAT |
| | CAGGAACAACGGCATCAAGCACATCATCGGCATCGACAGGGGCGAGAGGAACCTGCTGTACCTGAGCCTGATCGAC |
| | CTGAAGGGCAACATCATCAAGCAGATCAGCCTGAACGAGATCAAGCCCCTGGGCAACGACGGCTGCGTGACCAAC |
| | TACAGGGACATCCTGGCCGACAGGGAGAAGGACAGGAAGGAGGCCAGGAGGAGCTGGAAGAAGCTGGAGAACAT |
| | CAAGGACCTGAAGAGCGGCTACCTGAGCCAGGTGGTGCACGTGATCGCCAAGATGATGGTGGACTACCAGGCCATC |
| | GTGGTGCTGGAGGACCTGAACAGCGCCTTCATGCAGAGCAGGCAGAAGATCGAGAGGAGCGTGTACGAGCAGTTC |
| | GAGCACAAGCTGATCAACAAGCTGAACTACTACGTGGACAAGAGCGTGAGCGACGACCTGCCCGGCGGCCTGCTG |
| | TACGGCCTGCAGCTGGCCAGCAAGAAGACCCACGACGTGCAGTGCGGCTGCATCTTCTACGTGAGCGCCTGGAACA |
| | CCAGCAAGATCGACCCCGTGACCGGCTTCGTGAACATGCTGGACACCAGGTTCGAGAACGTGAGCAGCAGCAGGA |
| | GCTTCTTCAGCCACTTCGACAGCATCAGGTACAACGTGGCCAAGGACTGGTTCGAGTTCACCTTCGACTACAACAA |
| | CTTCCACAAGAAGCTGGAGGGCACCAAGACCAGGTGGACCCTGTGCACCTACGGCACCAGGATCCTGACCCAGAG |
| | GAACGACAGGAACCAGTGGGAGAGCAGGGAGGTGGACATCACCGACGAGTTCAAGAAGGCCTTCGTGGCCGCCG |
| | GCATCGACATCCACGGCAACCTGAAGGACGCCATCTGCGCCCTGGACAAGAGGGTGCACCTGGAGCAGCTGATGA |
| | GGCTGATGAAGCTGCTGCTGCAGCTGAGGAACAGCATCATCAACCAGGACACCGACTACATCCTGAGCCCCGTGGC |
| | CGAGAACGGCGTGTTCTACGACAGCAGGAGGCACGCCGACAACCTGCCCATCGACGCCGACGCCAACGGCGCCTA |
| | CAACATCGCCAGGAAGGGCCTGTACCTGCTGAGGAAGATCAAGGCCGACAGGAGCGCCAAGCCCGAGCTGTGCAT |
| | CAGCAACAAGGAGTGGCTGAGGTTCGTGCAGGACAAGAGCAACCCCGGCGACaaaagacctgccgccacaaagaaagccggaca |
| | ggccaagaagaaaaaggattacaaagacgatgacgataagtga |
| 55 | atggcccccaagaaaaagcggaaggtgGTGACCATCGACGACTTCTGCGGCCAGAAGAACGGCTACAGCAGGAGCATCACCCTGA |
| | GGAACAAGCTGATCCCCGTGGGCAAGACCCAGGAGAACCTGGAGAAGGCCAACATGCTGGAGGAGGACTTCAGC |
| | AGGGCCAAGGCCTACGTGGAGGTGAAGAAGATCATCGACGACTTCCACAGGAGCTTCATCGAGGACGTGCTGAGC |
| | AAGGCCAACCTGAACTGGAAGGAGCTGTACGAGAAGATCGACAGCTTCCAGAGGGAGACCGACAAGGCCAAGAA |
| | GGCCAAGAACCAGAAGGACCTGCAGCCCGTGCAGGTGAAGATGAGGAAGGCCATCGTGAAGATCTTCAAGGACGA |
| | CGAGAGGTTCGACAAGCTGTTCAAGAAGGAGCTGCTGAGCGACCTGCTGCCCGAGATCATCAAGAGCGCCCCCGA |
| | GAACGAGATCAGCAACAAGCAGACCGCCCTGGACGTGTTCAAGGGCTTCGCCACCTACTTCACCGGCTTCCACGAG |
| | AACAGGAAGAACATGTACAGCGAGGAGGACAAGGCCACCGCCATCAGCTACAGGATCGTGAACGAGAACTTCCCC |
| | AAGTTCTACGCCAACATCAAGCTGTTCGAGAAGATCCAGAAGGAGTTCCCCGTGATCATCGAGGAGACCGAGAAGA |
| | GCCTGGCCAGCTTCCTGGCCGGCAAGAAGCTGAAGGACATCTTCACCCCCGAGGCCTTCAACCAGGTGCTGACCCA |
| | GAGCGGCATCGACTTCTACAACACCGTGATCGGCGGCATCGCCGGCGAGGCCGGCACCGAGAAGGTGCAGGGCCT |
| | GAACGAGAAGATCAACCTGGCCAGGCAGCAGCTGCCCGACGAGGAGAAGAGCAAGCTGAGGGGCAAGATGGTGG |
| | TGCTGTACAAGCAGATCCTGAGCGACAGGGAGACCGCCAGCTTCATCCCCGTGGGCTTCGAGAACAAGGACGAGG |
| | TGTACAAGACCCTGCAGGAGTTCAAGAAGAGCACCGTGGAGAAGAGCATCGAGATCATCAGGGAGACCTTCAAGA |
| | GCCCCGAGAACTTCGACCTGAGCAAGATCTACGTGCCCGCCAAGGAGCTGACCGCCTTCAGCCTGAGCGTGTTCGG |
| | CAGGTGGAACATCCTGAGCGACGGCCTGTTCGCCATCGTGGAGGAGAAGAGCAAGAAGGCCCTGAGCGAGAAGCA |
| | GACCGAGAAGCTGGCCAACGAGATCGCCAAGAAGGACTACAGCCTGAGCGAGCTGCAGGCCGCCTACGAGAAGTA |
| | CCTGGAGAAGAACCCCGTGGAGGACGAGGGCAGGTGCAGGAGGCTGAGCGCCAAGAACTACTTCGAGCTGATCG |
| | AGCTGAGGAAGATCGACGACAGCGCCGAGAAGGAGTTCGTGAACGTGCTGAGCAACCTGGACGAGCTGTGGAGC |
| | AAGATCGACTTCGAGAACCCCAGGAACCTGCAGCAGGAGAAGGAGGCCGCCACCCCCATCAAGAACTACCTGGAC |
| | GAGGTGCAGAACCTGTACCACTTCCTGAAGCTGGTGGACTACAGGGGCGAGGAGGAGAAGGACAGCGAGTTCTAC |
| | AGCAAGCTGACCGAGATCCTGGACCTGCTGAGCGAGATCGTGCCCCTGTACAACAAGGTGAGGAACTTCGTGACC |
| | AAGAAGCCCGGCGAGGTGAAGAAGATCAAGCTGAACTTCGACTGCAGCAGCTTCCTGAAGGGCTGGGGCACCGAG |
| | TTCGGCACCAAGGACGCCTTCATCTTCATCGACGAGAAGAAGTACTACCTGGGCGTGATCAACAAGAAGCTGGACA |
| | GCGACACCGTGAAGTACCTGCAGACCCCCGGCGAGCACATCATCAAGCAGGTGTTCGTGAACTTCCAGAAGCCCG |
| | ACTTCAAGAACTTCCCCAGGCTGTTCATCAGGAGCAAGAAGGACAGGTTCGCCCCCGCCGTGGAGCAGTTCAACCT |
| | GCCCGTGAACGACATCCTGGACATCTACGACAAGGGCCTGTACAAGACCGAGTACAGGAAGGAGAACCCCATCATC |
| | TACAAGGAGAGCCTGATCAAGATGATCGACTACTTCAAGCTGGGCATCAGCCAGCACGAGAGCTTCAAGGGCTTCA |
| | AGCTGACCTGGAAGGAGAGCAGCGAGTACGAGAACATCAGCGACTTCTACGCCGACGTGACCGCCAGCTGCTACC |
| | AGGTGAACTTCACCAACGTGAACTTCGACAGGCTGCTGAGCCTGGCCAACGAGATCGAGGACGACAACAAGAAGA |
| | TCTTCCTGTTCGAGATCTACAACAAGGACTTCAGCACCGGCAAGAACGGCGGCAACGGCAGCACCGGCAAGAAGA |
| | ACCTGCACACCATGTACTTCGAGAACCTGTTCAGCAAGGAGAACCTGAAGGACATCGTGCTGAAGCTGAGCGGCG |
| | AGGCCGAGATCTTCCTGAAGAGCGAGAACACCGAGCTGAAGAAGATCGTGCACAAGCAGGGCACCACCCTGGTGA |
| | ACAGGACCACCAAGGACAAGAAGCTGATCCCCGAGAACATCTACCAGGAGATCTACCAGTTCAAGAACAGCATGAT |
| | CGACAACATCAGCGACGAGGCCAAGGCCTGGCTGAACAGCGGCAACGTGGAGTGCAAGATCGCCAAGTTCGACCT |
| | GATCAAGGACAAGCACCTGATCGAGAAGAGCTACTACCTGCACTGCAGCATCATCCAGAACTTCAAGGCCCCCGAG |
| | AAGGTGTTCAACTTCAACGAGAAGGTGCAGAGGTACCTGAAGGACAACCCCGACGTGAAGATCATCGGCCTGGAC |
| | AGGGGCGAGAGGCACCTGATCTACCTGAGCCTGATCAACCAGAAGGGCGAGATCGAGCTGCAGAAGACCCTGAAC |
| | CTGGTGGAGCAGGTGAGGAACGACAAGACCGTGAAGGTGAACTACCAGGAGAAGCTGGTGCAGAAGGAGGGCGA |
| | CAGGGACAAGGCCAGGAGGAACTGGCAGACCATCGGCAACATCAAGGAGCTGAAGGAGGGCTACCTGAGCAACG |
| | TGGTGCACGAGATCGCCAAGCTGATGGTGGAGCACAACGCCATCGTGGTGATGGAGGAGCTGAACTTCGGCTTCAA |
| | GAGGGGCAGGTTCCCCGTGGAGAGGCAGGTGTACCAGAAGTTCGAGAACATGCTGATCGGCAAGCTGAACTACCT |
| | GGTGTTCAAGGACAAGAGCGTGACCGAGCCCGGCGGCGTGCTGAACGCCTACCAGCTGACCGACAAGAGCACCAA |
| | CGTGAGCGACGTGGGCAAGCAGTGCGGCTGGCTGTTCTACGTGCCCGCCGCCTACACCAGCAAGATCGACCCCAAG |
| | ACCGGCTTCGCCAACCTGCTGAACATGAAGGACCTGACCAACGTGGCCAAGAAGAGGATGTTCTTCAGCAAGTTCA |
| | AGGACATCCACTTCGACAGCGGCAGCAACGACTTCGCCTTCACCTTCGACTACAAGGACTTCGACGGCAAGGGCGC |
| | CGCCGAGATGACCCAGACCGAGTGGACCGTGTGGAGCAACGGCAAGAGGATCTTCTACAACAGCAAGCACCACAG |
| | CACCGAGGAGATCAACGTGAGCGAGAAGCTGAAGGAGCTGTTCGACGCCAGCAAGATCGACTACAAGACCGAGA |
| | ACAACATCTTCGAGAGCATCATGGAGGTGGGCGCCGACCTGAAGGAGGGCGAGAAGCCCAGCAAGGAGATCGCCA |
| | ACTTCTGGGACAGCCTGCTGTGGTACTTCAAGCTGATCCTGCAGATGAGGAACAGCATCCCCAACAGCGAGACCGA |
| | CTACCTGATCAGCCCCGTGAAGGCCGAGGACGGCACCTTCTTCGACAGCAGGGTGCAGAAGGAGCTGGGCAAGGA |
| | CAGCAAGCTGCCCATCGACGCCGACGCCAACGGCGCCTACCACATCGCCCTGAAGGGCCTGTACCTGCTGAAGAAC |
| | GACTTCAACCTGAACGACAAGGGCTACATCGAGAACATCAGCAACGCCGAGTGGTTCAGGTTCGTGCAGGAGAGG |
| | GACTACCTGAACaaaagacctgccgccacaaagaaagccggacaggccaagaagaaaaaggattacaaagacgatgacgataag |
| | tga |
| 56 | atggcccccaagaaaaagcggaaggtgCCCAACCTGTTCGACAACTTCACCCAGCAGTACAGCCTGACCAAGACCCTGAGGTTCG |
| | AGCTGAAGCCCGAGGGCAAGACCCTGGAGAACATGCAGAACGCCTTCAAGTTCGACAAGGACCTGCAGACCTTCC |
| | TGGTGGACCAGGACATCGAGGACGCCTACCAGACCCTGAAGCCCTACATCGACTTCATCCACGAGGAGTTCATCAC |
| | CGAGAGCCTGGAGAGCGACATCGCCAAGGGCATCGACTTCAGCGAGTACTTCGAGAACTTCAAGATCAAGAAGGA |
| | CATCAACCAGAAGGACTTCCAGAACCTGGAGCACAAGCTGAGGAAGGAGTTCGACCAGCTGTACATCCAGATCGC |
| | CAACAAGTGGAAGGAGGAGAAGTACAAGCAGTACAAGTGGAAGAAGGGCAGCAAGGTGGCCAACGGCAGCGAG |
| | ATCCTGAGCAGCGAGGACATCCTGAAGCTGGTGGTGGACCTGAACCAGGACAACAACCAGCTGAAGGACATCGTG |
| | CAGAACACCTTCAAGGGCTTCTTCACCTACTTCAGCGGCTTCAACGACAACAGGAGGAACTACTACCAGGTGGAGA |
| | AGGAGGCCAAGAGCAGCATCGCCACCAGGATCGTGAACGAGAACCTGCCCAAGTTCTGCGACAACTTCCTGCTGTT |
| | CGAGAACAGGAAGAACGAGTACCTGGGCGTGTACAACACCCTGAAGCTGCTGGGCAAGGACCTGGTGGACAAGG |
| | ACAGGAAGACCCTGGTGACCATCGAGGAGATCATCTTCAGCATCAAGCACTTCAACTTCTGCCTGAGCCAGAAGGA |
| | GATCGAGAACTACAACAAGCAGATCGGCAACGCCAACCTGCTGATCAACCTGTACAACCAGAACACCAGCAACGA |
| | GAGCGACTACAAGAGGCTGCCCCTGTTCAAGACCCTGTACAAGCAGATCGGCTGCGGCAAGAGGGACAGCCTGTT |
| | CTTCGAGATCACCGACTACACCAAGCTGGAGGCCGAGGGCAAGAGGATCCTGGGCAAGAGCGCCATCAGCGTGGA |
| | GGAGGTGCTGATCCAGGTGAAGGAGAGCGGCATCAAGTACTTCACCGAGAAGCTGGACAGCCTGCTGAACTACATC |
| | CTGGAGAAGGAGACCGACAACTACCAGGGCCTGTACTGGAGCAACATCGCCGTGAACAGCATCAGCAGCAAGTAC |
| | CTGGCCAACTGGAACAACCTGAAGGAGCTGTTCAAGGGCAAGAAGGAGGTGGCCAGCTACGACAAGGGCAGGGA |
| | GGAGGAGATCAAGATCAACGAGGCCGTGGACCTGAGCATCCTGTTCAGCATCCTGAACAACACCGAGGACTGGAA |
| | GGTGAAGGAGGTGTTCTTCAAGGAGAACCTGAACGAGGAGAGCGAGACCAAGAAGATCATCGAGACCAGCAAGA |
| | CCCCCAGCCAGGCCCTGCTGAGGATCGTGATCGGCGACCTGAAGAAGAACGCCAGCAACTTCCTGAACCTGAGCG |
| | ACAACATCGAGCAGCTGCTGGAGAGCAAGGAGAAGATCAAGGAGAGCATGGACTACGCCCTGAGCGTGACCAGGA |
| | TGCTGAAGTACTTCCTGGTGAAGGAGAGCAAGACCAAGGGCGCCCCCCTGGACAGCAAGCTGGTGGAGGACCTGA |
| | ACCTGCTGCTGCTGAGCGACGACGCCCCCTGGTTCATGTGGTACGACGGCCTGAGGAACTACCTGACCAAGAAGCC |
| | CCACGACGACGCCAAGGAGAACAAGCTGAAGCTGAACTTCGAGAACAGCACCCTGGCCGGCGGCTGGGACATCA |
| | ACAAGGAGCCCGCCAACTACTGCGTGATCCTGACCAACAGCGCCAACGACCAGTACCTGGCCATCAACGCCAAGA |
| | GGAACAACAAGATCTTCTGCAAGGAGCTGGTGGACGGCAAGGGCAAGGACAAGAAGCTGCTGAAGAACCCCCTG |
| | TACGAGAGCGACGAGCAGCACTGGAAGAAGATGGAGTACAAGCTGCTGCCCGGCCCCAACAAGATGCTGCCCAAG |
| | TGCCTGCTGCCCAAGAGCAACCTGAGGAAGTTCGGCGCCAGCGAGGAGGTGCTGAGCATCTACGAGAACGGCAGC |
| | TTCAAGAAGAACACCCCCGACTTCAGCACCCAGGACCTGCACAAGCTGATCGACTTCTACAAGAGCGCCCTGCTGA |
| | AGTACGAGGACTGGAAGTGCTTCCACTTCAAGTTCAACGACACCAGGAGCTACGGCGACATCAGCCAGTTCTACAG |
| | CGACCTGGAGAAGCAGGGCTACCTGCTGAGCTTCATCGACATCAACAAGAGCGTGCTGAACCAGCTGGTGGACAG |
| | CGGCAAGATCTACCTGTTCCAGATCAGGAACCAGGACAGCAACATCAGCAAGAAGATCAACCACCAGGACAACCT |
| | GCACACCATCTACTGGAGGGCCGTGTTCGAGGACGTGTTCAACAAGCCCAAGCTGAACGGCCAGGCCGAGGTGTT |
| | CTACAGGAAGGCCCTGCCCCAGGAGGACCTGCAGCTGCTGAAGGACAAGGACAACAAGGAGATCATCGCCAACTA |
| | CAGGTTCAGCAAGGAGAAGTTCCTGTTCCACGTGCCCATCACCCTGAACTTCTGCCTGAAGAACAAGAAGATCAAC |
| | GACCTGGCCAACAGGGAGATCCTGAGCAACGACAACATCTACTTCCTGGGCATCGACAGGGGCGAGAAGCACCTG |
| | GCCTACTACAGCCTGGTGGACCAGAAGGGCAACATCGTGATCGACCAGAACGGCAAGAGCATCCAGGGCACCCTG |
| | AACATCCCCTTCCTGGACAGCAACGGCAACCAGAGGCTGGTGAAGGCCACCAAGAGGGTGATGAAGGACGGCAAG |
| | GAGGACGCCCAGGTGGTGGAGTGCAAGGACTACAACGAGCTGCTGGAGGCCAGGGCCAGCGACAGGGACTACGC |
| | CAGGAAGAACTGGCAGACCATCGGCGCCATCAAGGACCTGAAGGACGGCTACATCAGCCAGGTGGTGAGGAAGAT |
| | CGCCGACCTGGCCGTGGAGTACAACGCCTACATCGTGCTGGAGGACCTGAACACCGGCTTCAAGAGGGGCAGGCA |
| | GAAGATCGAGAAGAGCGTGTACCAGAAGCTGGAGCTGGCCCTGGCCAAGAAGCTGAACTTCCTGGTGGACAAGAG |
| | CGCCAAGATCGGCGAGGTGGGCAGCGTGACCAACGCCCTGCAGCTGACCCCCCCCGTGAACAACTACAGCGACAT |
| | CGAGAACAGGAAGCAGTTCGGCGTGGTGCTGTACACCAGGGCCAACTACACCAGCCAGACCGACCCCGTGACCGG |
| | CTGGAGGAAGACCATCTACCTGAAGACCGGCAGCGAGGACATCATCAAGAAGCAGATCTTCGAGAGCTTCAGCAA |
| | CATCTACTTCGACGGCCAGGACTACATCTTCACCTACACCGACAAGAACATCGACAGGAAGTGGGAGCTGTACAGC |
| | GGCAAGAACGGCAAGAGCCTGGACAGGTACAGGGGCAGGAAGGTGAACAACAAGGGCTGGGTGATCGACAAGAT |
| | CAACATCCAGGAGATCCTGGACAAGATCTTCATCAACTTCGAGATCTGCTACGGCGAGAAGAACAACATCTTCGAG |
| | AAGCTGAAGGCCGGCGTGGAGCTGGAGAAGTACAACGAGTACACCGCCTGGGAGAGCCTGAGGTTCGCCATCGAC |
| | ATCATCCAGCAGATCAGGAACACCGGCATCGAGAGCAGCGACAGCGACTTCCTGCTGAGCCCCATCAGGGTGAACA |
| | ACCTGCACTTCGACAGCAGGCTGGCCAGCGAGAACCAGCCCAACAGCGGCGACGCCAACGGCGCCTACAACATCG |
| | CCAGGAAGGGCATCATCATGGCCAACTACATCAAGCAGGGCGTGAGCCCCTTCGTGAGCGACATCGACTGGGACAA |
| | CTTCGTGACCAAGAACaaaagacctgccgccacaaagaaagccggacaggccaagaagaaaaaggattacaaagacgatgacga |
| | taagtga |
| 57 | atggcccccaagaaaaagcggaaggtgAACAAGTTCCTGGACAGCTTCACCCACCAGTACAGCCTGAGCAAGACCCTGAGGTTCG |
| | AGCTGAAGCCCGTGGGCAAGACCGCCGAGTGGATCCAGAAGAGGGACATCATCGGCACCAAGAAGCAGGAGAAC |
| | GGCGAGGCCCAGCTGACCGGCAAGGACGCCGTGAGGGCCGAGAACTACGAGTACGCCAAGAAGCTGCTGAACGA |
| | GATGCACAGGCTGTTCATCGAGGACAGCCTGGCCGCCATCAAGGAGGAGCAGAAGGTGGAGCTGAACAAGCTGCT |
| | GGTGAAGATCGCCGAGAGCGGCAGCCTGGACGGCTTCAGGAAGCCCATGAGCAACATCTGCAAGGAGCTGTTCGA |
| | CAGCGCCACCCAGGACTGGCTGCTGGCCTACAGCGAGGAGATGCCCTGCTTCTGGGACGAGGACATCCTGGAGCTG |
| | ACCAACAAGGCCGAGACCACCGAGAACAAGAAGAACATCAAGTTCTTCCTGAAGAAGGCCGAGGACATCAGGAA |
| | GTTCAAGGACGACCCCCTGAAGGCCTTCAAGAAGAAGAAGCACGAGGCCCTGAACAGCAACACCGAGGCCCTGA |
| | AGCTGCTGGAGTGGAAGATCAGGAGGGGCGAGGTGCTGATCAGCGGCAAGGACCTGGGCAGGGACGAGAGCGAG |
| | GAGATCCTGAGCACCAAGGAGCTGATCCTGATCCTGAGGAGCTTCTACAGCTTCGTGACCTACTTCACCGGCTTCAA |
| | CGAGAACAGGGCCAACATCTACAACCTGGACGACAAGAAGTACATCAGCACCAGCATCGCCTTCAGGAGCTTCGA |
| | GCAGAACCTGTTCTTCCACATCGAGAACATCAAGAAGTGGCACACCATCACCAACAGCATCGAGGAGCACACCGAC |
| | CAGCTGAACGCCAGCGACTACCCCATCAAGGACAAGCTGAGGGAGCTGGAGCTGGCCTTCGACTGCAAGCTGGCC |
| | GACCTGTTCGCCGTGGACAGCTTCAGCCAGTTCCTGAGCCAGAGCGGCATCGACAGGTACAACAGCATCATGGGCG |
| | GCATCGCCCAGAAGACCGGCAAGACCAAGGTGCAGGGCCTGAACGAGATCATCAACCAGGCCAGGCAGCAGACCG |
| | GCGCCAAGAGGCACAAGTTCCCCCCCATGAAGCTGCTGTACAAGCAGATCCTGAGCAAGGGCGAGGGCATCTTCGT |
| | GGAGAGCTACGTGGACGACGCCGACATGCTGACCAGCATCGCCGGCTTCCACGAGAACCACCTGAACGAGAGCCT |
| | GTGCAGCAACTGGCTGCACGAGCTGGAGGAGCTGATCGACAGCGTGCAGGAGAGCAAGGACACCATCTTCATCGC |
| | CAGGGACAAGCTGAACAGGATCAGCCAGGAGATCACCGGCAGCTGGAGCGCCATCCAGGACTGGATCGTGGCCCA |
| | CCAGAAGGACCAGAAGGAGGTGGACGCCCTGACCAAGAAGAAGGTGTTCAGCATCCAGGAGCTGGAGGACTACTT |
| | CAGCAAGAGCAGCGACGACGAGCTGTTCACCGCCAAGATCCCCGAGGACAGGAGCGCCTACCTGCCCGGCGAGGG |
| | CGAGGGCATCCTGTTCCAGTTCTTCAAGGTGAGGGCCAAGGAGATCCTGGCCGAGCTGCAGAGCGGCTGGTACGCC |
| | CTGGACAAGGCCCAGCTGCTGCAGGAGAAGCAGCTGGACAAGAACAGGGAGAACGAGGGCAGCAAGGGCTTCGA |
| | GCAGGTGGGCCTGCTGAAGGGCTACCTGGACGCCTGCAACAGCCTGAGCAGGTTCATCGCCGACTGGAACATCCA |
| | GAAGGACATCAAGCAGGAGAGGTGCAGCCTGTGGTACGAGTTCCTGCAGAAGCACCTGGACACCGTGCCCATCTT |
| | CGACCTGTACAACCAGGTGAGGAACCACGTGAGCAAGAAGGCCTACAGCACCGACAAGCTGAAGGTGAACTTCGA |
| | GAAGAGCACCCTGCTGGACGGCTGGGACAGGAACAAGGAGAGCCAGAACTTCGGCGTGCTGTTCGAGAAGGACG |
| | GCGCCTACTACCTGGGCATCATGACCCCCGACAGCAACGGCATCTTCGAGTACGACGTGAACCCCGACGAGAACAC |
| | CAAGAAGAAGGCCATCAAGGAGGAGCTGAGGAAGCAGACCACCCCCGAGAACGGCGAGGACAGGTTCCAGAAGA |
| | TCAACTACAAGCTGCTGCCCGGCCCCAACAAGATGCTGCCCAAGGTGTTCTTCGCCAAGAGCAACGAGGACCTGTT |
| | CCAGCCCAGCGCCAGGATCACCGAGATCAAGGAGAACAAGAAGTACGCCAAGGCCTACACCGACAAGCACGGCAA |
| | GGCCGAGCTGCACGAGTACATCGACTTCTGCCAGAAGAGCCTGGTGAGGCACCCCGAGTGGAGCGTGGCCTACGG |
| | CTTCGACCAGACCACCTTCAGGAAGGCCACCGAGTACATCAGCATCGACGAGTTCTACAGGGAGGTGGAGCTGCTG |
| | GGCTACAAGGTGACCTTCGACAGCATCAAGGCCACCTACATCGAGGAGAAGGTGGCCAACGGCGAGCTGTACCTGT |
| | TCCAGATCTACAACAAGGACTTCAGCACCGCCAAGAAGGGCGGCGGCAAGGACAACCTGCACACCCTGTACTGGA |
| | AGAGCCTGTTCAGCGAGGACAACCTGAAGGACGTGGTGTTCAAGCTGAACGGCCAGGCCGAGGTGTTCTTCAGGC |
| | CCAGCAGCATCGTGTGGAGCGAGAAGGTGATGAAGCAGGGCCACCACTACGACGACGACAAGATGCTGAAGGACA |
| | AGTTCAAGTACCCCATCATCAAGGACTTCAGGTTCACCCAGGACAAGTTCTTCTTCCACTGCCCCATCAGCCTGAAC |
| | TTCAAGGCCCCCGCCAAGCCCTACGGCTTCAACTACAAGGTGCACGACTTCCTGAGGAAGAACGGCGAGATCAAC |
| | GTGATCGGCATCGACAGGGGCGAGAAGCACCTGCTGTACTACAGCATCACCAACCAGCAGGGCGACATCATCGAGC |
| | AGGGCAGCCTGAACAGCATCGAGAGCAGCTACGAGAAGGGCAGCAGCAGCGTGCCCCACAGCACCAACTACCTGG |
| | AGAAGCTGCAGGAGAAGGAGAAGAACAGGGACAGCGCCAGGAAGAGCTGGAGCAGGATCGAGAACATCAAGGA |
| | GCTGAAGGCCGGCTACCTGAGCCACGTGGTGCACAAGCTGGCCAAGCTGATCATCAAGCACAACGCCGTGGTGGT |
| | GCTGGAGGACCTGAACGCCGGCTTCAAGAGGGGCAGGTTCAAGGTGGAGAGGCAGGTGTACCAGAAGTTCGAGA |
| | AGGCCCTGATCGACAAGCTGAACTACCTGGTGTTCAAGGACCAGGAGAAGCCCAGCGCCCCCGGCCACCCCCTGA |
| | GGGCCTACCAGCTGACCGACCCCTTCGAGAGCTTCGAGAAGCTGGGCAAGCAGAGCGGCATCCTGTTCTACACCAC |
| | CGCCGCCTACACCAGCACCACCGACCCCGTGACCGGCTTCCTGAAGAACGTGTACAAGCCCTACGCCAACGTGGTG |
| | GACAGCAGCGACTTCTGGAAGAGCTTCGACGAGATCCACTACGACGTGAAGAAGGAGAGGTTCGAGTTCACCTAC |
| | GACCTGAGGAAGATCAAGAACAAGCAGGTGGACAGGGACAAGGACGAGGCCAAGGTGAGCAGGAGCGTGTGGAC |
| | CGTGTGCAGCTGCGTGGAGAGGAGCAGGTACGTGAAGAGGAAGGCCAACGAGGAGCAGAAGCAGGGCGCCGCCA |
| | GCAAGAGCATCGGCAAGATGGGCGAGTACGAGGTGTTCGTGGTGACCGACAAGCTGAAGGCCCTGTTCGAGAAGG |
| | AGAGCATCAGCTACCAGAGCGGCGAGGACATCAGGAGCCAGCTGAGCAACAGGATCGACAAGAAGGACGCCAAG |
| | CTGCACAGGGAGCTGATCTACTACTTCGGCACCCTGCTGAACATGAGGGTGACCGACAAGCAGAGCGAGAGCGGC |
| | ACCGACGAGAACGACTACATCCTGAGCCCCATCGAGCCCTTCTTCGACAGCAGGAAGGGCTACGAGAAGCTGCCCG |
| | TGAACGGCGACGCCAACGGCGCCTACAACATCGCCAGGAAGGGCATCGCCATCCTGAACAGGATCCACGAGAGCG |
| | ACAGCGGCGACAAGGTGGACCTGATGATCAGGAAGAAGGACTGGCAGGACTTCGTGCAGAACGAGGAGGTGGTG |
| | CAGAAGCAGATGGGCAAGCTGGACCTGaaaagacctgccgccacaaagaaagccggacaggccaagaagaaaaaggattacaaa |
| | gacgatgacgataagtga |
| 58 | atggcccccaagaaaaagcggaaggtgGCCCTGTTCAGCGAGAACTTCGTGGGCTGCTACCCCGTGAGCAAGACCCTGAAGTTCG |
| | AGCTGAGGCCCGTGCCCGAGACCAAGGCCTACCTGGACGACGACAACTGCCCCCTGCTGGCCGGCGACCTGGAGA |
| | GGGACAAGGCCTACCCCGTGGTGAAGGAGCTGCTGGACAAGTACTACAGGTACTTCATCGACATCTGCCTGAAGGA |
| | CAAGAAGCTGAGCCCCGAGACCATCGACGAGGCCTACCAGGCCTACAGCAGCCACGACGAGAAGAGCCAGAAGG |
| | CCCTGAAGGCCGCCAGCCAGAAGCTGAGGAAGGAGGTGGCCGGCTTCTTCGACAAGAAGACCAGGGAGACCTACT |
| | GCCTGGACAAGTACAGCAACCTGCTGGTGCTGGACAAGAAGAACGACAACAAGGAGAAGGGCACCCCCAAGACC |
| | ATCCCCAGCGTGCTGAAGAAGTGGCTGGAGAACGAGAGCGGCTTCAGCGAGAAGGAGGTGGAGAAGTACAGCCA |
| | GGCCGTGCAGACCTTCAACGGCTTCACCACCTACTTCATCAGCTACAAGGAGAACAGGGAGAACATGTTCAGCAGC |
| | GAGGAGAAGGCCAGCGCCATCAGCTACAGGGTGGTGAACCAGAACATGCTGAGGTTCTTCAGCAACGTGGAGAAC |
| | TTCGAGAACATCAAGAAGAAGTACCCCGACCTGTACCAGACCCTGAAGGAGTTCGAGGAGAGCTTCGTGCCCGCC |
| | GCCTACAGCAGCCTGCTGAGCCAGGCCGGCATCGACGACTACAACTACAGGGTGATCGGCAGGCCCGCCGAGGAC |
| | GAGGACTTCGCCGGCGTGAACAGGCTGATCAACGAGTACAGGCAGAAGAACGCCGTGAGGAACAGGAACCTGCC |
| | CGTGATGGTGACCCTGTACAAGCAGATCCTGAGCGACAGGGAGAACGGCTTCGGCAGGGACATCCTGGCCAGCGA |
| | CAGCGAGGCCAAGGAGTACATCAGGGGCAAGTACAGCAAGGCCTACGACACCGCCTGCCAGCTGACCGTGTTCGT |
| | GAACAACTACATCCTGACCGAGGACCTGGACACCGTGTACGTGAAGACCCAGCACCTGAAGGACATCAGCCAGAG |
| | CATCTTCGGCGAGTGGAGCGTGCTGGACGACGCCATCAACAACGGCGGCATCGACAGCAAGATCATCAGCCTGAAG |
| | GCCCTGGCCGAGGCCTTCGAGAAGTACTGCAGCGCCCTGGACCCCGACATGGCCCAGAAGTACAGGGACGCCTTC |
| | AGCCTGAAGGAGTACCTGAAGAACCCCCCCGCCGTGGAGCCCAGGCTGCCCGAGACCGCCGAGAAGGTGACCGCC |
| | TACAAGCCCGCCATGGACGAGCTGATCAGCCTGATCAGGTTCTACAAGCCCTTCTACCTGTACGACGGCAAGAAGA |
| | GGCTGCAGGTGCCCGCCGAGGGCATGGACTTCAGCAACGAGTTCGACAAGTACTTCGAGGACCTGAAGGAGTTCA |
| | GCCACGCCTACGACAAGGTGAGGAACTTCGCCACCAAGAAGCCCTACAGCGTGGAGAAGATCAAGCTGAACTTCA |
| | ACCAGCCCACCCTGCTGGACGGCTGGGACGTGAACAAGGAGAAGGAGAACGCCAGCTTCCTGTTCGTGAAGGAGG |
| | GCAAGTACTACCTGGGCATCGCCGACAAGAGCGACAAGAACCTGTTCGACAGGAGCAGGGACGCCGTGAAGGAGG |
| | CCCTGACCCTGACCTGCGGCGAGGACTACTACGAGAAGGTGCAGTACAAGCAGCTGCCCAACATCAGGATGAACCT |
| | GCCCAGGATCGTGTTCAAGCCCAAGTGGGAGAAGGTGTTCGCCCCCATCATCACCGAGAGGATCAGCGCCATCGAG |
| | AAGGAGAAGCTGTACACCGCCGGCGCCAACGACAGGCAGGCCGTGATCGAGTGGATCGACTTCGTGAAGAGCGTG |
| | CTGGCCCTGCACAGCGAGTGGAACAACTACTTCCAGTTCAAGTTCAAGGAGACCAGCGCCTACAAGAACACCAAG |
| | GAGTTCTACGACGACCTGGACAACCAGGCCTACATCCTGAGCAAGATCAAGATCAACGCCAGCTACATCCACAAGC |
| | TGGTGAGGGAGAGGAAGCTGTACCTGTTCCAGATCTACTGCAAGGACTTCAGCGACAAGAAGAAGAAGACCGGCG |
| | CCGACAACCTGCACACCATGTACTGGAGGTGCCTGTTCAGCGACGAGAACCTGGCCGCCCTGGCCCAGGGCCAGG |
| | CCCCCATCATCAAGCTGAACGGCAAGGCCGAGATCTTCCTGAGGGAGCCCAGCCTGAAGTACGAGGCCACCCACCC |
| | CAAGAACCAGCCCGTGGCCAACAAGAACCCCCTGAACAGCAAGAGGGAGAGCCTGTTCAGCTACGACCTGGCCAA |
| | GAACAGGAGGTTCACCGAGAGGAAGCTGTTCTTCCACTGCCCCCTGACCATCAACTTCAGGACCGCCGCCGAGGG |
| | CGCCAGGAGCTTCAACGAGAGGGTGAACAGGTTCGTGGAGAACAACCCCGACATCAGGATCATCGGCATCGACAG |
| | GGGCGAGAGGCACCTGCTGTACTACACCGTGATCAACCAGAGCGGCAAGATCCTGGAGCAGGGCAGCCTGAACAA |
| | GATCAGCAGCGACTACAGGACCCCCGAGGGCGCCGAGGTGGAGAAGGTGACCGACTACCACGAGCTGCTGAACAG |
| | GAAGGAGGGCGACAGGAGGGCCGCCAGGCAGACCTGGGACACCATCGAGAACATCAAGGAGCTGAAGGAGGGCT |
| | ACCTGAGCCAGGTGGTGCACAAGCTGACCACCCTGATGATCAAGCACAACGCCGTGGTGGTGCTGGAGAACCTGA |
| | ACCAGAACTTCAAGAGGAGCAGGATCAAGGTGGAGAAGCAGGTGTACCAGAAGTTCGAGAAGGCCCTGATCGACA |
| | AGCTGAACTACCTGGTGTTCAAGGACAGGCCCTACGGCGCCCCCGGCAGCTACGCCAACGGCCTGCAGCTGACCGC |
| | CCCCTTCGAGAGCTTCGACAGGCTGGGCAAGCAGACCGGCGCCCTGTACTACGTGGTGCCCAGCTACACCAGCCAC |
| | ATCGACCCCAAGACCGGCTTCGTGAACCTGCTGGGCGCCAAGCTGAGGTACGAGAGCGTGGTGAAGGCCCAGGAG |
| | ACCATGAAGAAGATCGGCAGCTTCAAGTTCAACCCCGAGAGGAACTGGTTCGAGATCAGCTTCGACTACGGCGACT |
| | TCGGCATCGAGATGGCCCACCCCCAGTGGACCGCCTGCACCCACGGCGACGCCTACTGGGGCTACAACCCCAAGGA |
| | GAAGAGGGCCGTGAGCTACGACGTGACCGAGGAGATCAAGAAGCTGCTGACCGAGTACGGCATCGACTACGGCAC |
| | CGGCGCCGACATCAAGGAGGCCGCCATCAGGCAGACCAAGCCCGCCTTCTGGAAGACCCTGTTCTACATGCTGAAG |
| | CTGGCCCTGCAGCTGAGGAACACCGTGGCCGGCACCGACAACGAGAACGACTACATCCTGAGCCCCGTGGAGTAC |
| | GAGGCCGGCAAGTTCTTCGACAGCAGGAAGGCCGGCGAGGACGAGCCCCAGAACGCCGACGCCAACGGCGCCTA |
| | CCACATCGCCATGAAGGGCCTGCAGGCCCTGAGGAGGATCGAGGACGGCAAGGTGAACAAGTTCCCCAAGAACGG |
| | CGAGAGGAAGGCCTGGCTGGCCTTCATGCAGAACAGGGAGTACCTGAAGaaaagacctgccgccacaaagaaagccggacaggc |
| | caagaagaaaaaggattacaaagacgatgacgataagtga |
| 59 | atggcccccaagaaaaagcggaaggtgAGCAAGCTGAGCACCTTCAACGAGCACTTCCAGAAGACCATCACCCTGAGGAACGAG |
| | CTGGTGCCCGTGGGCAAGACCCTGGAGAACATCATCAGCAGCAACGTGCTGATCAACGACGAGAAGAGGAGCGAG |
| | GACTACAAGAAGGCCAAGGAGATCATCGACAGCTACCACCAGGAGTTCATCGAGAAGAGCCTGAGCAAGGTGGAC |
| | ATCGAGTGGGACGACCTGGCCTACTGCCTGACCAAGAAGGAGCCCGAGAACTTCGACAAGAAGAAGAAGTACCAG |
| | GAGGAGATCAACAAGCTGCAGAACGAGAAGAGGAAGAAGAGCGTGGAGATCTTCAAGAAGTTCAGCTTCGGCGT |
| | GCTGTGCGACAACAACGGCCAGGAGGTGCAGATCAAGTTCGACGACCTGTTCAAGAGCACCCTGTTCAAGTACGT |
| | GCTGCCCAACTTCCTGAAGAACGACGAGGACAAGACCGTGGTGAGGGACTTCAGCGAGTTCACCAGCTACTTCAC |
| | CGGCTTCCACGAGAACAGGAAGAACCTGTACAAGAGCGCCCCCATGCCCACCGCCGTGGCCTACAGGATCGTGAA |
| | CGACAACTTCCCCAAGTTCATCAGCAACCAGAAGATCTTCTGCGCCTGGAAGGACAACGTGCCCCACTTCGTGGAG |
| | ATCGCCAAGACCAAGCTGAGCGAGGTGGGCATCAGCGACCTGGACATCGAGAGCAAGTTCGAGCTGAGCAACTTC |
| | AACAGCTGCCTGAACCAGACCGGCATCGACTACTACAACGACCTGAGGGGCCAGCTGAACAACGTGATCAGCCAC |
| | ATCTGCCAGCAGGACAAGAACCTGGGCGACCTGCTGAGGAAGAAGAGGTGCCTGAAGATGGTGCCCCTGTACAAG |
| | CAGATCCTGAGCGACAAGGACAGCAGCTTCAGCATCGACGAGTTCGACAACGACGAGAGCGCCATCCAGGACGTG |
| | ATCAGCTTCTACAAGAAGATGGTGGGCGAGAGGTGCCCCCAGAGGAAGCTGAGCGAGCTGCTGAGGGAGCTGAGC |
| | AGCCACGACAGGGAGAAGATCTTCATCCAGGGCAAGAACCTGAACAGCATCAGCAGGATCCTGTTCGGCGGCAAG |
| | AACTGGAGCCTGCTGAGGGAGGCCATCATCGAGGAGAAGAGCAAGGACAAGAGCTTCAAGAAGGCCATCAAGAG |
| | CAACAGCACCGACGAGGCCCTGGAGAAGATCCTGAGCAAGGACGAGTTCAGCATCAGCTTCCTGAGCAGGATCTG |
| | CCACGAGAACCTGAGCGAGAAGATCGACAAGTTCATCAAGAACCAGGACGAGCAGCTGATCAACATCAACAGCCA |
| | GACCTGGCCCAAGACCCTGAAGAACAGCGAGGAGAAGAAGATCATCAAGGAGCACCTGGACTTCCTGCTGAACTT |
| | CTACAGGTTCGCCAAGAGCTTCAGCAGCAACACCATCGACAAGGACATGAGCTTCTACGCCGACTTCGACGAGAGC |
| | CTGAGCAGCCTGGAGAACGTGATCGGCCTGTACAACAAGGTGAGGAACTACGTGACCAAGAAGCCCTACAGCCTG |
| | GAGAAGATCAAGCTGAACTTCGAGATCCCCAACCTGGCCGAGGGCTGGAGCAAGACCAAGGAGCTGGCCAACCTG |
| | ACCGTGATCTTCCTGAAGGACAACGAGTACTACCTGGGCATCATCAACAAGAACAGCAAGATCGACTTCAGCCTGA |
| | TCAAGGAGAAGACCAGCAGCGAGAACTGCTTCAGGAAGATGGAGTACAGCCTGTTCGGCAAGATCGCCCAGAACA |
| | TCAACAGGTGCGTGTTCACCAAGGCCGTGAAGAACCACTTCAGCAAGAGCCCCGACAACTTCATCCTGAGCGACA |
| | AGAAGAAGTTCCTGAAGGAGATCGAGATCACCAAGGAGATCTACGACCTGGTGAAGAGCGACGAGAGCGACAAG |
| | AAGTACCTGAAGGAGTACAAGAAGATCAACGAGAAGGAGTTCAGGAGCGCCCTGAGCAAGTGGATCAACTTCTGC |
| | ATCGAGTTCCTGAAGAGCTACAAGAACACCAGCCAGTTCGACTTCAGCTGCATCAAGAAGGCCGAGAGCTACGAC |
| | GACATCAACGACTTCTACGACGACATCGAGAAGGTGACCTACAAGATCGAGTTCGTGGACATCAGCACCAGCCTGA |
| | TCGACGCCCTGGTGGACAAGGGCCAGCTGTTCCTGTTCAAGATCAGGAACAAGGACTTCGCCAAGAAGGCCAGCG |
| | GCACCCCCAACCTGCACACCCTGTACTTCAAGAGCATCTTCGACACCAAGAACTTCGAGAACGGCATCGTGAAGCT |
| | GAACGGCGAGGCCGAGATCTTCTACAGGAAGAAGAGCCTGAACGTGGACGACACCACCGTGCACCAGAAGGGCA |
| | GCTACATCGTGAACAAGGTGGTGGTGGACCCCAGCAGCGGCAAGAGCGAGCAGATCCCCGACAACGTGTACAACA |
| | ACATCTACGCCTTCGTGAACGGCAAGCTGAAGACCCTGAGCAAGGAGGACGAGGTGTACTACGCCAAGGCCACCA |
| | TCAAGAAGGCCACCCACGAGATCGTGAAGGACAGGAGGTTCACCGAGGACAAGTTCTTCTTCCACTGCCCCATCAC |
| | CATCAACTACAAGAGCAAGGAGAAGCCCAGCAAGTTCAACGACAAGGTGCTGGACTTCCTGAGGAAGAACGACG |
| | ACATCAACATCATCGGCATCGACAGGGGCGAGAGGAACCTGATCTACGCCACCGTGATCAACAAGAACGGCGAGAT |
| | CATCGACTGCAAGAGCTTCAACACCATCAAGCACCAGAGCAGCAGCGTGAACTTCGACGTGGACTACCACAACAA |
| | GCTGCAGGAGAGGGAGAAGAACAGGAAGGAGGAGAAGAGGAGCTGGAACAGCATCAGCAAGATCGCCGACCTGA |
| | AGGAGGGCTACCTGAGCGCCGTGATCCACGAGATCGCCCTGATGATGGTGAAGTACAACGCCATCGTGGTGATGGA |
| | GAACCTGAACCAGGGCTTCAAGAAGATCAGGGGCGGCATCGCCGAGAGGAGCGTGTACCAGAAGTTCGAGAAGAT |
| | GCTGATCGACAAGCTGAACTACTTCGTGATCAAGAACGAGAACTGGACCAACCCCGGCGGCGTGCTGAACGGCTAC |
| | CAGCTGACCAACAAGGTGAGCACCATCAAGGACATCGGCAACCAGTGCGGCTTCCTGTTCTACGTGCCCGCCGCCT |
| | ACACCAGCAAGATCGACCCCAGCACCGGCTTCGTGAACCTGATCAACTTCAACAAGTACAAGAACAGCAACGACA |
| | GGAGGAAGCTGATCTGCAGCTTCGACAAGATCTGCTTCGTGCAGAACGAGAACCTGTTCAAGTTCAGCATCGACTA |
| | CGAGAAGCTGTGCCCCAACAGCAAGATGCCCGTGAAGAAGTGGGACATCTTCAGCTACGGCAACAGGATCGTGAG |
| | GGAGGACCTGAAGCACATCCCCATGAAGGAGAACCCCAAGTACGACCCCACCGAGGAGCTGAAGACCCTGTTCAG |
| | CAGCAAGGAGATCGAGTACCAGAAGGGCCAGAACCTGCTGGAGATCATCAGCACCAAGGACATGACCGAGGACAA |
| | GGAGTTCTGGAACAGCCTGTTCAACATCTTCAAGGCCATCCTGCAGATGAGGAACAGCCTGACCAACAGCCCCGTG |
| | GACAGGCTGCTGAGCCCCGTGAAGGGCAAGGACGGCAGCTTCTTCGACACCGACAAGGTGGAGGGCACCAAGTTC |
| | GAGAAGCTGAAGGACGCCGACGCCAACGGCGCCTACAACATCGCCCTGAAGGGCCTGCTGGTGCTGAAGAAGAAC |
| | GACAGCGTGAAGACCGACAAGGACATGAAGAACGTGAAGAACGTGAGCCTGGAGGACTGGCTGAAGTTCGTGCA |
| | GGTGACCCTGAGGGTGaaaagacctgccgccacaaagaaagccggacaggccaagaagaaaaaggattacaaagacgatgacga |
| | taagtga |

The nucleotide sequences of the Cas12 protein were synthesized commercially (like by Ruibiotech).

Cas12 proteins were expressed as FLAG-tagged fusion proteins from an inducible T7 promoter (pEASY-Blunt E2 expression plasmid) in a protease deficient E. coli B strain. Cells expressing the FLAG-tagged proteins were lysed by sonication. The supernatant was loaded on the Ni²⁺-charged HisTrap HP column (GE Healthcare) and eluted with a linear gradient of increasing imidazole concentration (from 0 to 500 mM) in 20 mM Tris-HCl, pH 7.5 at 25° C., 0.5 M NaCl Buffer on an AKTA Pure25 FPLC (Inscinstech). The eluate was resolved by SDS-PAGE on BeyoGel Plus PAGE (Beyotime) and stained with Feto SDS-PAGE staining buffer (H&Z lifescience). Purity was determined using densitometry of the protein band with ImageLab software (Bio-Rad). Purified endonucleases were dialyzed into a storage buffer composed of 20 mM CH₃COONa, 500 mM NaCl, 0.1 mM EDTA, 0.1 mM TCEP, 50% glycerol; pH 6.0 and stored at −80° C.

### Example 4: In Vitro Cleavage Efficiency

Target DNAs containing protospacer sequences (5′-gagaagTcaTTcaaTaaggccac-3′, SEQ ID NO:63) and PAM sequences were constructed by DNA synthesis. A single representative PAM was chosen for testing when the PAM has degenerate bases. The target DNAs were comprised of 515 bp of linear DNA derived from a plasmid via PCR amplification with a PAM and protospacer located 700 bp from one end. Successful cleavage results in fragments of ~200 and ~300 bp. The target DNA, in vitro transcribed single RNA, and purified recombinant protein were combined in a cleavage buffer (NEBuffer 2.1) with an excess of protein and RNA and were incubated for 5 minutes to 3 hours, usually 1 hour. The reaction was stopped via addition of RNase A and incubation at 60 minutes. The reaction was then resolved on a 2% TAE agarose gel and the fraction of cleaved target DNA was quantified in ImageLab software.

The cleavage efficiency is represented by cutting ratio. The cutting ratio is calculated by the Gray value analysis and the formula like this: The cutting ratio (%)=100×(1−sqrt(1−(b+c)/(a+b+c)), “a” represents the uncut band gray value, “b” and “c” respectively represent the gray value of the two short sequences that be cut, “sqrt” is abbreviation for Square Root Calculations. In this application, cutting ratio can be also called cleavage ratio.

### Example 5: PAM Determination in Mammalian Cell Line

In a set of experiments, the HEK293T cells were cultured in DMEM media supplemented with 10% fetal bovine serum (Gibco™). For reverse transfection, the HEK293T cells were cultured in DMEM media supplemented with 10% fetal bovine serum (Gibco™). A volume of 450 μL of cells with a density of 100,000 cells/well was mixed with 50 μL mixture containing Lipofectamine™ 3000 (ThermoFisher Scientific, Cat. L3000008), Opti-Mem (Volume refill to 50 μL), 1 μL dsODN (10 pM), 100 ng (~1 μL) pgRNA (SEQ ID NO: 64+SEQ ID NO: 65) harbored Humanspacer3 spacer (SEQ ID NO: 65) and 400 ng (~1 μL) pCasX plasmid harbored GEB×0173 CDS (with NLS and FLAG, SEQ ID NO: 92) per the manufacturer's protocol. Then seeded the cell mixture onto a 24-well plate and cultured at 37° C. and 5% CO₂. 10 pM dsODN was annealed using dsODN-Top and dsODN-BoT oligonucleotides pre-transfection. The corresponding nucleotide sequence of gRNA in this example is AATTTCTACTGTTGTAGATGGCCAGGCACAGTGGCTCAC (SEQ ID NO: 149)

72 hours post-transfection, the supernatant was removed and the cell layer was washed by PBS. Then the genomic DNA was extracted from each well of a 24-well plate using DNA Extraction solution (Denogen (Beijing) Bio Sci & Tech Co. Ltd, Cat. DNS033-48) per manufacturer's protocol. All DNA samples (500 ng, 260/280 value: 1.8~2.0) were subjected to Guide-Seq NGS analyses.

The basic method of Guide-Seq library preparation is described by Nikolay et. al (Nat. Protoc. 2021). The extracted DNA sample were first sheared using KAPA Frag Kit (Cat #KK8602, Roche). Fragmented DNA was purified and then phosphorated using T4 Polynucleotide Kinase (Cat #M0201S, NEB). An SS5-adapter (generated by annealing 10 μM SS5TOP oligo with 10 μM SS5BTM oligo) was ligated to the fragmented DNA using Quick Ligation™ Kit (Cat #M2200S, NEB), followed by two steps off-target PCR to add chemistry for sequencing.

For off-target PCR1 was performed using Platinum™ Taq DNA Polymerase (Cat #15966005, Invitrogen) with GSP1 (a mixture of GSP1-Top and GSP1-BoT) and Y_XX oligos. For off-target PCR2 was performed using Platinum™ Taq DNA Polymerase with GSP2 (a mixture of GSP2-TopA/B/C and GSP1-BoTA/B/C), Y_XX (Same to PCR1) and i753_XX oligos. The DNA product in each step described above need purification using SPRI Select (Cat #B23318, Beckman Coulter). The final library was quantified with qPCR and sequenced on Illumina NextSeq 1000. The reads were aligned to a reference genome after eliminating those having low quality scores. Q30 rate is more than 0.9. The reads length is between 130 bp-140 bp. The resulting files containing the reads were mapped to the reference genome (BAM files), where reads that overlapped the target region of interest were selected. The relevant nucleotide sequences are shown in table 7.

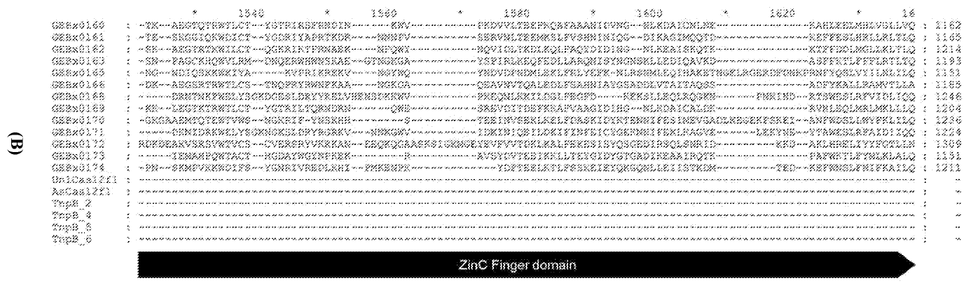

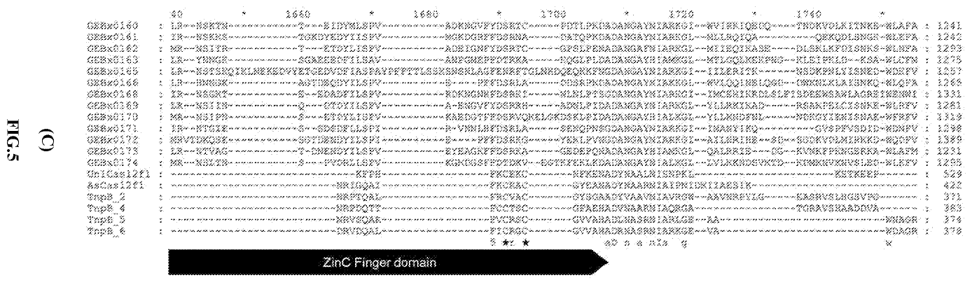

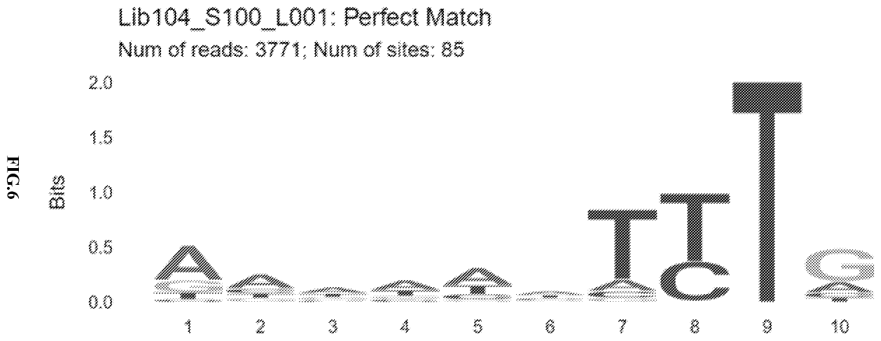

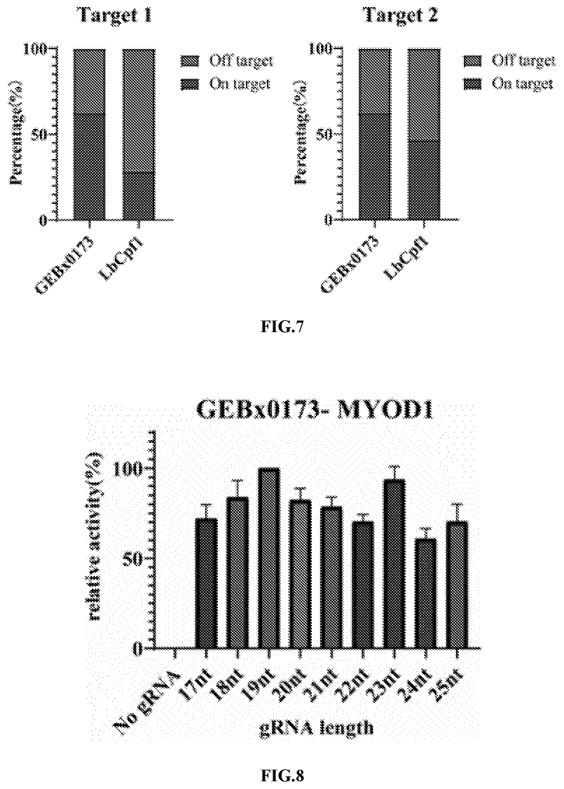

The PAM preference of the wild type GEB×0173 in HEK293 cell line is shown in FIG. 6. As shown in FIG. 6, GEB×0173 recognizes a PAM having a sequence TYTG (Y is T or C). The percentage of the off-target site of GEB×0173 for 5854 and Humspacer3 site is shown in FIG. 7, demonstrated a lower off-target activity compared with LbCpf1 on both targets.

TABLE 7
The nucleotide sequences referred above

| | SEQ ID | |
|---|---|---|
| Name | NO: | 5′-3′ |
| Cas protein | 64 | AATTTCTACTGTTGTAGAT |
| DR | | |
| Humanspacer | 65 | GGCCAGGCACAGTGGCTCAC |
| 3 | | |
| 5854 | 66 | GGCCAGGTGCGGTGGCTCAC |
| dsODN-Top | 67 | pACTTCGGTACGCGTCGACGTACGGTCGACGAGTCGCGCGTACGACGATCGA*G*A*C |
| dsODN-BOT | 68 | pGTCTCGATCGTCGTACGCGCGACTCGTCGACCGTACGTCGACGCGTACCGA*A*G*T |
| SS5TOP | 69 | A*A*C*TACACGACGCTCTTCCGATCTNNNWNNNWNNAGTGACAC |
| SS5BTM | 70 | NNNNNNGTGTCACTNNWNNNWNNNAGATCGGAAGAGCGTCGTGTAGTTp |
| GSP1-Top | 71 | GTCGACGAGTCGCGCGTA |
| GSP1-BOT | 72 | TCGTCGACCGTACGTCGA |
| GSP2-TopA | 73 | GTGACTGGAGTTCAGACGTGTGCTCTTCCGATCTCGCGCGTACGACGAT*C*G*A |
| GSP2-TopB | 74 | GTGACTGGAGTTCAGACGTGTGCTCTTCCGATCTATCGCGCGTACGACGAT*C*G*A |
| GSP2-TopC | 75 | GTGACTGGAGTTCAGACGTGTGCTCTTCCGATCTTAACGCGCGTACGACGAT*C*G*A |
| GSP2-BoTA | 76 | GTGACTGGAGTTCAGACGTGTGCTCTTCCGATCTTACGTCGACGCGTAC*C*G*A |
| GSP2-BoTB | 77 | GTGACTGGAGTTCAGACGTGTGCTCTTCCGATCTGCTACGTCGACGCGTAC*C*G*A |
| GSP2-BoTC | 78 | GTGACTGGAGTTCAGACGTGTGCTCTTCCGATCTATGTACGTCGACGCGTAC*C*G*A |
| Y_XX | 79 | AATGATACGGCGACCACCGAGATCTACACNNNNNNNNACACTCTTTCCCTACACGACGCTCTTC |
| | | CGATCT |
| i753_XX | 80 | CAAGCAGAAGACGGCATACGAGATNNNNNNNNGTGACTGGAGTTCAGACGTGTGCTCTTCCGA |
| | | TCT |

Note:
p: phosphorylation modification; *: phosphorothioate (PS) bond; “N” may be any natural or non-natural nucleotide.

### Example 6: In Vitro Gene Editing Effect of the CRISPR-Cas12 in Mammalian Cell Line

In a set of experiments, the HEK293T cells were cultured in DMEM media supplemented with 10% fetal bovine serum (Gibco™). the HEK293T cells were cultured in DMEM media supplemented with 10% fetal bovine serum (Gibco™). A volume of 450 μL of cells with a density of 100,000 cells/well was mixed with 50 μL mixture of Lipofectamine™ 3000 (ThermoFisher Scientific, Cat. L3000008), Opti-Mem (Volume refill to 50 μL), and pgRNA/pCasX plasmid (200 ng and 800 ng, respectively) or pCasX-gRNA plasmid (2 g) per the manufacturer's protocol. Then seeded the cell mixture onto a 24-well plate and cultured at 37° C. and 5% CO₂.

72 hours post-transfection, the supernatant was removed and the cell layer was washed by PBS. Then the genomic DNA was extracted from each well of a 24-well plate using DNA Extraction solution (Denogen (Beijing) Bio Sci & Tech Co. Ltd, Cat. DNS033-48) per manufacturer's protocol. All DNA samples (500 ng, 260/280 value: 1.8~2.0) were subjected to amplicons NGS analyses.

To quantitatively determine the efficiency of editing at the target location in the genome, NGS was utilized to identify the presence of insertions and deletions introduced by gene editing. Primers used for NGS which around the target area within the MYOD1 genes were designed. Additional PCR was performed per the manufacturer's protocols (Illumina) to add chemistry for sequencing. The amplicons were sequenced on Illumina iSeq 100. The reads were aligned to a reference genome after eliminating those having low quality scores. Q30 rate is more than 0.9. The reads length is between 130 bp-140 bp. The resulting files containing the reads were mapped to the reference genome (BAM files), where reads that overlapped the target region of interest were selected and the number of wild types reads versus the number of reads which contain an insertion, substitution, or deletion was calculated. The number of the reads mapped the reference genome is more than 1000.

In this in vitro experiment, GEB×0173 were tested on TTTG-MYOD1 target in HEK293T cell line. pCasX plasmid harbored GEB×0173 CDS (with NLS and FLAG, SEQ ID NO: 92) were co-transfected with the pgRNA plasmid harbored different length of TTTG-MYOD1 spacer (17 nt~25 nt, table 8). The nucleotide sequences of the pgRNA used in this example are composed of the Cas protein DR (SEQ ID NO: 64) and the corresponding spacers (SEQ ID NO: 81-89) arranged from 5′-3′ direction. And the structure of the example gRNA with 20 nt is shown in table 10 (SEQ ID NO:144). The result is shown in FIG. 8, wherein the relative activity was normalized to each spacer editing efficiency aligned to the highest editing efficiency. As shown in FIG. 8, the highest editing efficiency is appeared in 19 nt TTTG-MYOD1 spacer, and the next is 23 nt.

TABLE 8
Different length of TTTG-MYOD1 spacer

| | SEQ ID | |
|---|---|---|
| length | NO: | 5′-3′ |
| 17 bp | 81 | CCACAACGGACGACTTC |
| 18 bp | 82 | CCACAACGGACGACTTCT |
| 19 bp | 83 | CCACAACGGACGACTTCTA |
| 20 bp | 84 | CCACAACGGACGACTTCTAT |
| 21 bp | 85 | CCACAACGGACGACTTCTATG |
| 22 bp | 86 | CCACAACGGACGACTTCTATGA |
| 23 bp | 87 | CCACAACGGACGACTTCTATGAC |
| 24 bp | 88 | CCACAACGGACGACTTCTATGACG |
| 25 bp | 89 | CCACAACGGACGACTTCTATGACGA |

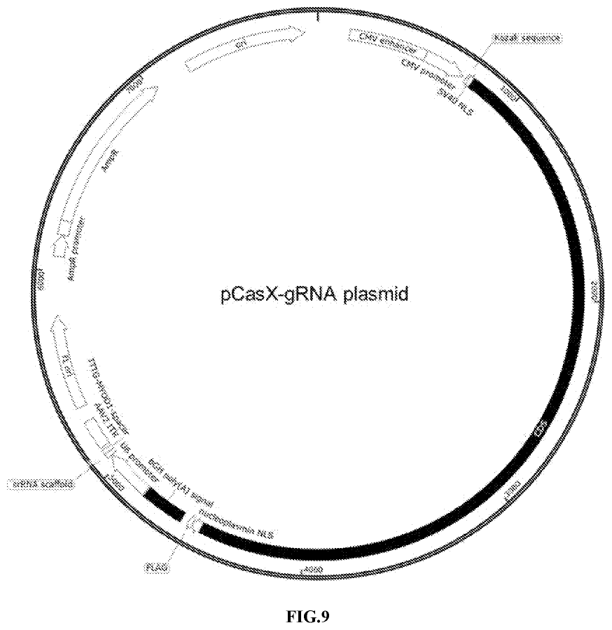

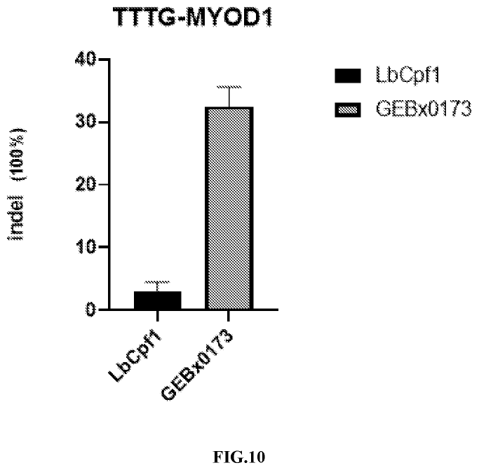

In another in vitro experiment, pCasX-gRNA plasmid (FIG. 9) harbored GEB×0173 CDS (with NLS and FLAG, SEQ ID NO: 92) and the 20 nt TTTG-MYOD1 guide (table 8) were transfected in HEK293T cell line. The result is shown in FIG. 10, demonstrated a 32.5% editing efficiency of GEB×0173 on TTTG-MYOD1 target, which is ten folds higher than LbCpf1 positive control.

In this example, the direct repeat sequence (DR) which existed in gRNA is same to example 5.

### Example 7 In Vitro Gene Editing Activity Screening of GEB×0173

In set of experiments, the HEK293T was cultured in DMEM media supplemented with 10% fetal bovine serum (Gibco™). For lipoplex transfection. A volume of 200 μL of cells with a density of 50,000 cells/well were seeded 24 hours pre-transfection. Cells were transfected with a lipoplex containing Lipofectamine™ 3000 (0.4 μL/well), P3000 (2 μL/well), pgRNA/pCasX plasmid (125 ng/well and 375 ng/well, respectively) and Opti-Mem up to 50 μL/well per the manufacturer's protocol. Plated cells were allowed to settle and adhere for 72 hours in a tissue culture incubator at 37° C. and 5% CO₂ atmosphere. The nucleotide sequences of the pgRNA used in this example are composed of the Cas protein DR (SEQ ID NO: 64) and the corresponding spacers (SEQ ID NO:84, 95-116) arranged from 5′-3′ direction. And the structure of the corresponding gRNAs are shown in table 10 (SEQ ID NO:144)

72 hours post-transfection, the supernatant was removed and the cell layer was washed by PBS. Then the genomic DNA was extracted from each well of a 48-well plate using DNA Extraction solution (Denogen (Beijing) Bio Sci & Tech Co. Ltd, Cat. DNS033-48) per manufacturer's protocol. All DNA samples (500 ng, 260/280 value: 1.8-2.0) were subjected to amplicons NGS analyses to quantitatively determine the efficiency of editing at the target location in the genome.

For NGS, 50 ng of total genomic DNA was input for two-step PCR using KAPA Hifi HotStart ReadyMix Kit (Roche). First-step PCR (PCR 1) resulted in a ~200 bp product, followed by indexing PCR (PCR 2) yielding final fragments flanking the Illumina sequencing barcodes for subsequent Next-Seq or iSeq (Illumina, San Diego, CA, USA). PCR 1 reactions were carried out as follows: 98° C. for 5 min, then 20 cycles of [98° C. for 20 sec; 60° C. for 20 sec; 72° C. for 20 sec], followed by a final extension at 72° C. for 3 min. The indexing PCR 2 reactions were carried out as follows: 98° C. for 5 min, then 15 cycles of [98° C. for 20 sec; 62° C. for 20 sec; 72° C. for 20 sec], followed by a final extension at 72° C. for 3 min. PCR 2 products were purified by SPRI beads and quantified by VAHTS Library Quantification Kit for Illumina (Vazyme, Cat.NQ101) on a StepOnePlus Real-time PCR system (Thermo Fisher Scientific). The amplicons were sequenced on an Illumina iSeq 100 or NextSeq instrument. The reads were aligned to a reference genome after eliminating those having low quality scores. Q30 rate is more than 0.9. The reads length is between 130 bp-140 bp. The resulting files containing the reads were mapped to the reference genome (BAM files), where reads that overlapped the target region of interest were selected and the number of wild types reads versus the number of reads which contain an insertion, substitution, or deletion was calculated. The number of the reads mapped the reference genome is more than 1000.

For Indel frequency determination, qualified reads were mapped to the referenced amplicons sequence using CRISPResso2 with default parameters, then subjected to filtering those reads not spanning the corresponding spacer regions. The resulting reads were then estimated the desired and undesired insertion and deletion occurring on the whole spacer region. Total editing frequency was calculated as: [count of total reads] divided by [count of reads with any insertions or deletions]. Out-of-frame frequency was calculated as: [count of edited reads] divided by [count of reads with those insertions or deletions indivisible by 3].

FIG. 11 shows in vitro human cell genome editing efficiency of GEB×0173 on MYOD1-TTTG and additional 22 targets. 6 targets indicate over 20% indel while 10 targets indicate 10%~20% indel which provides valuable insights into the potential application of GEB×0173.

TABLE 9
the spacers of different targets

| SEQ ID | name | 5′-3′ |
|---|---|---|
| 84 | MYOD1-TTTG | CCACAACGGACGACTTCTAT |
| 95 | CD34-TTTG-T1 | ACTGTCGTTTCTGGAAGAGA |
| 96 | CD34-TTTG-T2 | TGTATCTATGAGCACATGCA |
| 97 | CD34-TTTG-T3 | GGGAGTTAACAGTTACTTCG |
| 98 | CFTR-TTTG-T1 | GCATTAGGAGCTTGAGCCCA |
| 99 | CFTR-TTTG-T2 | GAGACAACGCTGGCCTTTTC |
| 100 | DNMT1-TTTG-T1 | AGGAGTGTTCAGTCTCCGTG |
| 101 | DNMT1-TTTG-T2 | GCTCAGCAGGCACCTGCCTC |
| 102 | EMX1-TTTG-T1 | TGGTTGCCCACCCTAGTCAT |
| 103 | EMX1-TTTG-T2 | GGGAGGCCTGGAGTCATGGC |
| 104 | HBB-TTTG-T1 | CTTCTGACACAACTGTGTTC |
| 105 | HBB-TTTG-T2 | AGGTTGCTAGTGAACACAGT |
| 106 | LPA-TTTG-T1 | TTCTTTACCATAAGCGAAGG |
| 107 | LPA-TTTG-T2 | GGTAGTTTTCTGGGGTCCGA |
| 108 | LPA-TTTG-T3 | TTGCCCAGGCTGGAGTGCAG |
| 109 | POLQ-TTTG-T1 | CTTGGACAAGTCCTGGAAGG |
| 110 | POLQ-TTTG-T2 | AATGGCAGGCAGAGTGCCTT |
| 111 | POLQ-TTTG-T3 | GATACCTGAATAAACTAAAT |
| 112 | RNF2-TTTG-T1 | GGTCTGGCCTTAGTGATCTT |
| 113 | TTR-TTTG-T1 | ACCATCAGAGGACACTTGGA |
| 114 | TTR-TTTG-T2 | CCTCTGGGTAAGTTGCCAAA |
| 115 | TTR-TTTG-T3 | GCAACTTACCCAGAGGCAAA |
| 116 | VEGFA-TTTG-T1 | CTTGCCATTCCCCACTTGAA |
| 117 | CD34-GATG-T1 | TTGCAAGCCACCAGAGCTAT |
| 118 | CD34-GATG-T2 | TCACTTAGGATAGGAGAAGA |
| 119 | CD34-GATG-T3 | TTGCAAGGCTAGTGCTAGTG |
| 120 | CFTR-GATG-T1 | TCCAATCTTGATTCCACTGA |
| 121 | CFTR-GATG-T2 | GATGAAGTACAGGTAGCAAC |
| 122 | DNMT1-GATG-T1 | gaattggtagggcgtcacgt |
| 123 | DNMT1-GATG-T2 | aaaacctatacaacacggaa |
| 124 | EMX1-GATG-T1 | TTTCTCGGCAACCTTGGCCC |
| 125 | EMX1-GATG-T2 | CCTGTACAAATCAAGGCCAC |
| 126 | HBB-GATG-T1 | GCCTGGCTCACCTGGACAAC |
| 127 | HBB-GATG-T2 | CTGTTATGGGCAACCCTAAG |
| 128 | LPA-GATG-T1 | CAGAAGCCTTTAGTGCTACA |
| 129 | LPA-GATG-T2 | GCAGACAAGCTGGCATTACT |
| 130 | LPA-GATG-T3 | CCAATGTGGTGTCATAGACG |
| 131 | POLQ-GATG-T1 | AAGCTAACTAGGAAGTTGTT |
| 132 | POLQ-GATG-T2 | AAGGTCAAGATCCATTGGTG |
| 133 | RNF2-GATG-T1 | AGTGCATCAAAGTTTGGGTC |
| 134 | TTR-GATG-T1 | tcacagaaacactcaccgta |
| 135 | VEGFA-GATG-T1 | TCTATCAGCGCAGCTACTGC |
| 136 | VEGFA-GATG-T2 | AGATCGAGTACATCTTCAAG |
| 137 | TTR-NGG-T2 | ttctttggcaacttacccag |
| 138 | TTR-NGG-T3 | gcaacttacccagaggcaaa |
| 139 | EMX1-NGG-T1 | TGGTTGCCCACCCTAGTCAT |
| 140 | CFTR-NGG-T1 | ATTAGGAGCTTGAGCCCAGA |
| 141 | POLQ-NGG-T1 | TTGCTTGGACAAGTCCTGGA |

### Example 8 Comparison of GEB×0173, AsCpf1 and SpCas9

In set of experiments, the HEK293T was cultured in DMEM media supplemented with 10% fetal bovine serum (Gibco™). For lipoplex transfection. A volume of 200 μL of cells with a density of 50,000 cells/well were seeded 24 hours pre-transfection. Cells were transfected with a lipoplex containing Lipofectamine™ 3000 (0.4 μL/well), P3000 (2 μL/well), pCasX-gRNA plasmid (500 ng/well) and Opti-Mem up to 50 μL/well per the manufacturer's protocol. Plated cells were allowed to settle and adhere for 72 hours in a tissue culture incubator at 37° C. and 5% CO₂ atmosphere. Method of genomic DNA extraction and NGS is identical to that described in Example 7. The nucleotide sequences of the pgRNA of GEB×0173 are composed of the Cas protein DR (SEQ ID NO: 64) and the corresponding spacers (SEQ ID NO: 98, 102, 109-111) arranged from 5′-3′ direction. The nucleotide sequences of the pgRNA plasmids of AsCpf1 are composed of the Cas protein DR (AATTTCTACTCTTGTAGAT, SEQ ID NO: 142) and the corresponding spacers (SEQ ID NO: 98, 102, 109-111) arranged from 5′-3′ direction. The nucleotide sequences of the pgRNA of SpCas9 are composed of the corresponding spacers (SEQ ID NO: 137-141) and Cas protein DR (GTTTTAGAGCTAGAAATAGCAAGTTAAAATAAGGCTAGTCCGTTATCAACT TGAAAAAGTGGCACCGAGTCGGTGC, SEQ ID NO: 143) the arranged from 5′-3′ direction. The structure of the corresponding gRNAs with are shown in table 10 (SEQ ID NO:144-146)

FIG. 12 shows the comparison of GEB×0173, AsCpf1 and SpCas9 editing efficiency across 5 targets which had the same spacer sequences with a 5′-TTTG PAM for GEB×0173 and AsCpf1 and an NGG-3′ PAM for SpCas9 in HEK293T cell line. Higher indel frequencies for GEB×0173 is observed than those for SpCas9 on EMX1-T1 target sites, indicating that GEB×0173 exhibits genome-editing activities comparable or even higher with those of SpCas9 and AsCpf1.

TABLE 10
The gRNA of the Cas proteins.

| SEQ | | |
|---|---|---|
| ID | Name | Sequence |
| 144 | GEBx0173 | AAUUUCUACUGUUGUAGAUNNNNNNNNNNNNNNNN |
| | crRNA | NNNN |
| 145 | AsCpf1 | AAUUUCUACUCUUGUAGAUNNNNNNNNNNNNNNNN |
| | crRNA | NNNN |
| 146 | SpCas9 | NNNNNNNNNNNNNNNNNNNNGUUUUAGAGCUAGAA |
| | sgRNA | AUAGCAAGUUAAAAUAAGGCUAGUCCGUUAUCAAC |
| | | UUGAAAAAGUGGCACCGAGUCGGUGC |

The continuous “N” represents the target sequence of the crRNA corresponding to the sequences of the spacers in table 9.

### Example 9. In Vitro Editing Using GEB×0173 mRNA

In set of experiments, the HEK293T was cultured in DMEM media supplemented with 10% fetal bovine serum (Gibco™). A volume of 100 μL of cells with a density of 20,000 cells/well were seeded in 96-well plates 24 hours pre-transfection. Cells were transfected with a lipoplex containing Lipofectamine™ RNAiMAX (Invitrogen™) and RNA (30 ng crRNA, crRNA:mRNA=1:1~1:16 w/w) and Opti-Mem up to 25 μL/well per the manufacturer's protocol. Plated cells were allowed to settle and adhere for 72 hours in a tissue culture incubator at 37° C. and 5% CO₂ atmosphere. The crRNA and mRNA sequences are shown in Table 11. The mRNA used in this example is N1-methyl-pseudouridine modified of the uracil.

Primary human liver hepatocytes (PHH) cells were thawed and resuspended in hepatocyte thawing medium with supplements (Lonza, Cat. MCHT50) followed by centrifugation at 100 g for 10 minutes. The supernatant was discarded and the pelleted cells resuspended in hepatocyte plating medium (Lonza, Cat. MP100) plus 10% fetal bovine serum. Cells were counted and plated on Ultra Low Adsorption Cell Culture 96-well plates (Liver Biotech, Cat. LV-ULA002-96W) at a density of 40,000 cells/well. Plated cells were allowed to settle and adhere for 24 hours in a tissue culture incubator at 37° C. and 500 CO₂ atmosphere. After incubation cells were checked for monolayer formation and media was replaced with hepatocyte culture medium (Lonza, Cat. CC-3198) plus 10% fetal bovine serum. Cells were transfected with a lipoplex containing Lipofectamine™ RNAiMAX (Invitrogen™) and RNA (30 ng crRNA, crRNA:GEB×0173 mRNA=1:1-1:16 w/w) and Opti-Mem up to 25 μL/well per the manufacturer's protocol. Plated cells were allowed to settle and adhere for 72 hours in a tissue culture incubator at 37° C. and 500 CO₂ atmosphere.

Method of genomic DNA extraction and NGS is identical to that described in Example 7.

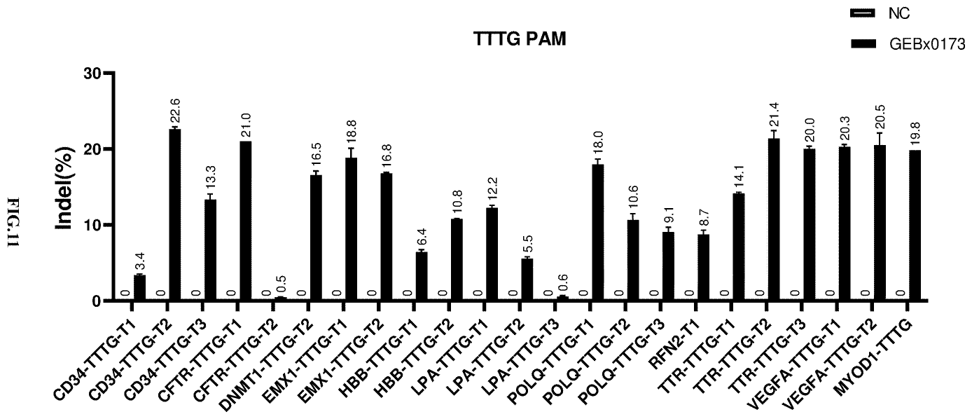

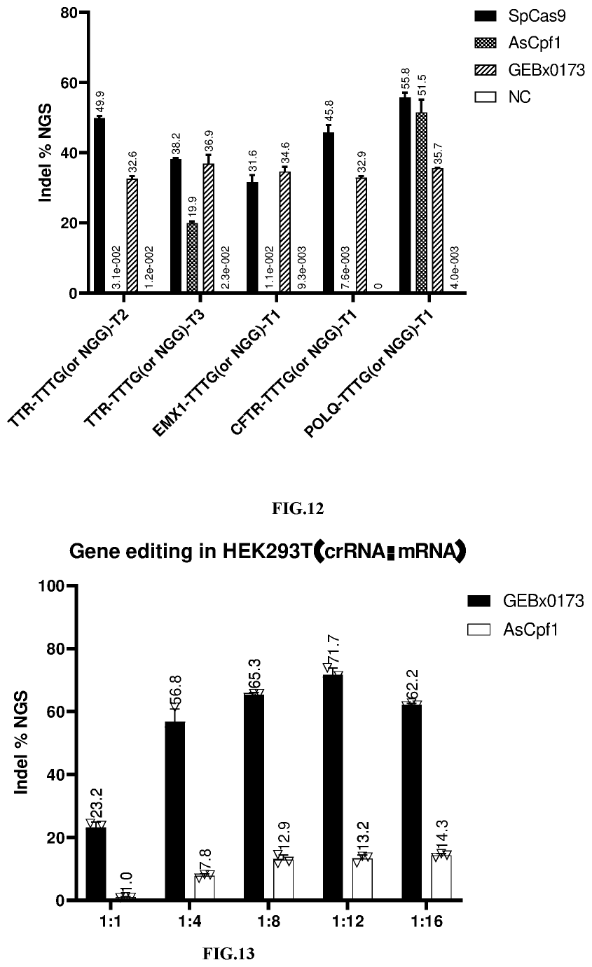

FIG. 13 and FIG. 14 show the editing efficiency following transfection of HEK293T or PITH with modified GEB×0173 mRNA and crRNA harbored MYOD1-TTTG spacers.

TABLE 11
the crRNA and mRNA sequences used in example 9

| SEQ ID | | |
|---|---|---|
| NO. | Name | 5′-3′ |
| 147 | crRNA: | AAUUUCUACUGUUGUAGAUCCACAACGGACGACUf |
| | MYOD1- | UfCfUfAfU |
| | TTTG-HM7 | |
| 148 | mRNA | AGGAGUCUAGAGUUAAGAAGCUGGGCCGCCACCauggccccaa |
| | GEBx0173 | agaagaagcggaaggUcGCCUUGUUUUCCGAGAACUUUGUUGGGU |
| | | GUUAUCCAGUUUCUAAGACUUUGAAGUUUGAACUGCGCC |
| | | CAGUGCCCGAAACGAAAGCAUACCUGGAUGAUGAUAACU |
| | | GUCCACUGUUGGCUGGUGAUCUGGAGAGAGAUAAAGCCU |
| | | ACCCGGUGGUGAAGGAAUUGUUGGACAAGUACUAUAGGU |
| | | AUUUCAUUGACAUUUGUCUUAAAGAUAAAAAGUUGUCCC |
| | | CCGAAACCAUUGACGAGGCCUACCAGGCCUACAGUAGCCA |
| | | CGACGAGAAAUCUCAGAAGGCACUGAAGGCCGCCAGUCAG |
| | | AAGCUGAGGAAGGAGGUCGCCGGAUUUUUCGAUAAAAAA |
| | | ACCCGCGAAACCUACUGUCUCGACAAGUACAGUAAUUUGU |
| | | UGGUGCUCGACAAGAAAAACGAUAAUAAAGAGAAGGGAA |
| | | CGCCCAAGACGAUCCCGUCAGUUCUCAAGAAAUGGCUUGA |
| | | GAAUGAGUCAGGAUUCUCCGAGAAAGAGGUGGAAAAAUA |
| | | CUCCCAGGCUGUGCAGACGUUUAACGGUUUUACCACAUAC |
| | | UUCAUUUCCUACAAAGAAAACAGGGAGAACAUGUUCAGC |
| | | AGUGAGGAGAAAGCCUCUGCCAUCUCUUACAGAGUUGUU |
| | | AAUCAGAACAUGCUGAGAUUUUUCUCCAAUGUGGAAAAU |
| | | UUCGAAAACAUAAAAAAGAAGUACCCUGACCUUUAUCAG |
| | | ACACUGAAAGAAUUUGAGGAAUCCUUUGUGCCUGCCGCCU |
| | | ACUCAAGCCUCCUUUCUCAGGCCGGUAUAGAUGACUAUAA |
| | | UUACAGAGUUAUCGGACGACCUGCAGAAGACGAGGAUUU |
| | | CGCCGGAGUCAAUCGCCUGAUAAAUGAGUAUAGGCAGAA |
| | | AAAUGCCGUGCGAAACCGGAACCUGCCAGUGAUGGUUACU |
| | | UUGUACAAACAGAUUCUCAGCGAUCGGGAAAAUGGUUUC |
| | | GGGCGGGACAUCUUGGCUUCCGACUCAGAAGCAAAAGAG |
| | | UAUAUUCGGGGGAAGUACUCCAAGGCUUAUGACACCGCCU |
| | | GCCAACUGACUGUAUUUGUCAAUAAUUACAUACUGACAG |
| | | AAGAUCUGGACACUGUGUACGUUAAGACCCAGCACCUGAA |
| | | GGAUAUCUCUCAAAGCAUUUUCGGAGAAUGGUCCGUCCU |
| | | UGAUGACGCUAUCAACAACGGAGGGAUCGACAGCAAGAU |
| | | UAUCUCAUUGAAGGCUCUGGCUGAGGCAUUCGAGAAAUA |
| | | UUGUUCAGCCCUUGAUCCCGAUAUGGCCCAAAAAUACCGG |
| | | GACGCCUUCAGUUUGAAGGAGUAUCUCAAGAAUCCGCCUG |
| | | CUGUGGAACCCAGACUGCCUGAAACGGCCGAGAAGGUUAC |
| | | CGCUUAUAAGCCCGCUAUGGAUGAGUUGAUCUCUCUGAU |
| | | AAGGUUUUACAAGCCUUUCUAUUUGUACGACGGGAAGAA |
| | | GAGACUGCAGGUGCCUGCCGAGGGAAUGGAUUUCUCUAA |
| | | UGAGUUCGACAAAUAUUUCGAGGACUUGAAGGAAUUUAG |
| | | UCACGCAUACGAUAAAGUUCGAAAUUUUGCAACCAAGAA |
| | | ACCCUACAGCGUAGAGAAGAUAAAACUCAAUUUCAACCAG |
| | | CCAACACUCCUGGACGGGUGGGACGUGAACAAGGAGAAA |
| | | GAGAAUGCAUCAUUUCUCUUUGUCAAAGAGGGCAAGUAC |
| | | UACCUGGGAAUUGCCGACAAGUCCGAUAAAAAUCUGUUC |
| | | GAUCGGAGUCGGGAUGCCGUCAAAGAGGCACUCACUCUUA |
| | | CUUGCGGCGAAGAUUACUAUGAGAAGGUGCAGUAUAAGC |
| | | AGCUUCCGAACAUACGGAUGAACCUGCCUCGCAUAGUUUU |
| | | UAAGCCUAAAUGGGAGAAGGUCUUUGCUCCUAUAAUCAC |
| | | UGAGAGAAUCAGCGCCAUUGAGAAAGAAAAGCUUUACAC |
| | | CGCAGGUGCCAACGAUAGGCAGGCGGUGAUCGAGUGGAU |
| | | AGACUUCGUCAAGUCUGUCCUUGCGCUGCACUCCGAGUGG |
| | | AACAACUAUUUCCAGUUUAAGUUUAAGGAGACGUCCGCA |
| | | UAUAAAAAUACAAAAGAGUUCUACGACGAUCUUGAUAAC |
| | | CAGGCUUACAUCUUGAGUAAGAUCAAAAUCAACGCCUCAU |
| | | AUAUCCACAAACUGGUUCGGGAGCGCAAAUUGUAUCUGU |
| | | UUCAGAUCUAUUGUAAAGACUUUUCUGAUAAGAAAAAAA |
| | | AGACCGGCGCCGACAAUCUCCAUACCAUGUAUUGGAGAUG |
| | | CCUGUUCUCCGAUGAAAACUUGGCAGCUCUCGCACAGGGA |
| | | CAGGCCCCAAUCAUAAAGUUGAACGGGAAAGCUGAAAUC |
| | | UUCCUCCGCGAGCCUUCUCUCAAAUAUGAGGCGACGCAUC |
| | | CGAAGAACCAGCCAGUGGCCAACAAGAAUCCCCUGAACUC |
| | | AAAACGGGAGAGCCUGUUCAGCUACGAUCUGGCAAAAAA |
| | | UCGCCGCUUUACCGAGAGGAAGUUGUUCUUCCACUGCCCC |
| | | CUGACCAUCAACUUUAGGACAGCUGCGGAAGGUGCGCGGA |
| | | GCUUCAAUGAGAGAGUUAAUAGGUUCGUCGAGAACAACC |
| | | CCGAUAUCAGAAUAAUAGGCAUUGAUAGGGGCGAGAGAC |
| | | ACCUGCUGUACUACACUGUGAUCAACCAGAGCGGCAAGAU |
| | | UCUGGAGCAGGGAAGUCUGAACAAAAUUAGCUCAGAUUA |
| | | CCGAACUCCUGAGGGCGCCGAGGUGGAAAAAGUCACUGAC |
| | | UAUCACGAGCUCUUGAACCGGAAAGAAGGAGAUAGACGA |
| | | GCUGCCCGACAGACAUGGGACACCAUAGAAAAUAUCAAGG |
| | | AGCUUAAAGAGGGAUACCUCAGCCAGGUGGUGCAUAAGC |
| | | UGACUACACUGAUGAUUAAACACAAUGCUGUGGUCGUUU |
| | | UGGAGAACCUUAAUCAAAACUUCAAACGCUCCCGCAUUAA |
| | | GGUCGAGAAGCAGGUCUAUCAGAAGUUCGAGAAGGCUCU |
| | | GAUCGAUAAACUGAAUUAUUUGGUUUUCAAGGAUAGACC |
| | | UUACGGAGCUCCCGGUUCCUAUGCCAAUGGGCUGCAGCUU |
| | | ACGGCUCCUUUCGAAUCCUUUGAUAGACUCGGCAAGCAGA |
| | | CAGGGGCUCUCUAUUACGUGGUGCCCAGCUACACAUCCCA |
| | | CAUUGAUCCUAAGACUGGGUUUGUGAACCUCCUGGGCGCA |
| | | AAGCUCCGGUACGAAAGCGUGGUGAAAGCUCAGGAAACA |
| | | AUGAAGAAGAUCGGGAGUUUCAAGUUCAACCCCGAGAGG |
| | | AAUUGGUUUGAAAUCAGCUUCGACUACGGCGACUUCGGG |
| | | AUCGAGAUGGCACAUCCCCAGUGGACCGCAUGUACUCACG |
| | | GCGAUGCUUAUUGGGGAUACAAUCCCAAGGAAAAAAGGG |
| | | CCGUUAGCUACGACGUGACGGAAGAGAUUAAAAAGCUGC |
| | | UGACAGAAUACGGAAUUGAUUAUGGGACUGGAGCAGAUA |
| | | UCAAGGAGGCCGCUAUUCGACAGACCAAGCCCGCUUUUUG |
| | | GAAGACCCUGUUUUACAUGCUCAAGCUGGCAUUGCAGUU |
| | | GAGGAAUACCGUGGCUGGCACUGAUAACGAGAACGACUA |
| | | CAUUCUGUCACCAGUCGAGUAUGAGGCCGGGAAGUUCUU |
| | | UGAUUCUAGAAAGGCUGGAGAGGACGAACCCCAGAAUGC |
| | | AGACGCCAAUGGAGCGUACCACAUCGCGAUGAAAGGAUU |
| | | GCAGGCAUUGCGGCGCAUCGAGGACGGCAAAGUGAACAA |
| | | AUUCCCGAAGAACGGUGAGCGAAAAGCUUGGCUGGCCUU |
| | | UAUGCAAAAUAGGGAAUAUUUGAAAaaacgacccgccgccacaaaaa |
| | | aggccgggcaggcaaagaaaaaaaaggaUUacaaagacgaUgacgacaagUaaUAGC |
| | | GGCCGCGGCCCGAUCCAGUGGGCAGGCCAAGGCCUGCUGG |
| | | GCCCCCGCGGACCCAGGUGCUCUGGGUCACGGUCCCUGUC |
| | | CCCGCGCCCCCGCUUCUGUCUGCCCCAUUGUGGCUCCUCA |
| | | GGCUCUCUCCCCUGCUCUCCCACCUCUACCUCCACCCCCAC |
| | | GGAUCCUCAGGAAAAAAAAAAAAAAAAAAAAAAAAAAAA |
| | | AAAAAAAAAAAAAAAAAAAAAAAAAAAAAAAAAUGCAUA |
| | | AAAAAAAAAAAAAAAAAAAAAAAAAAAAAAAAAAAAAAA |
| | | AAAAAAAAAAAAAAAAAAAA |

### Example 10: Structure-Guide Engineering of the CRISPR-Cas12 for PAM Expansion

In the context of genome editing, the requirement to recognize PAM reduces CRISPR targeting resolution and leaves some genome sites inaccessible to editing.

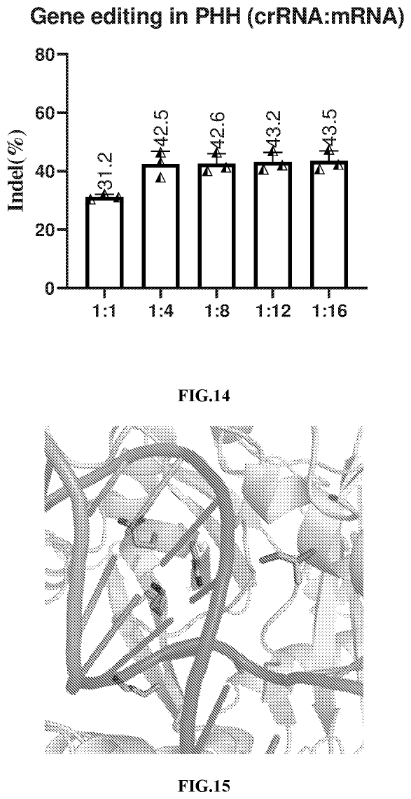

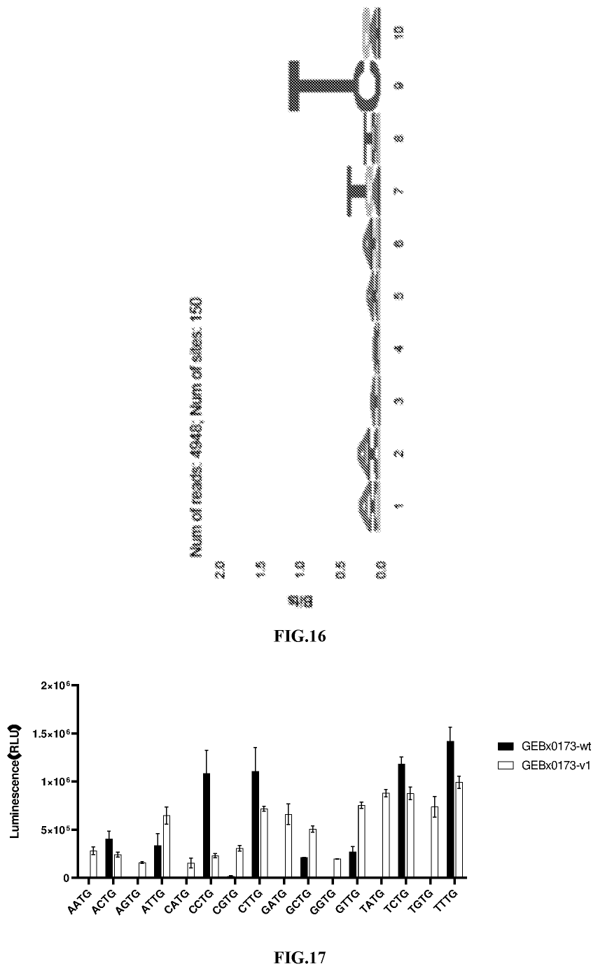

To expand the number of PAMs accessible to CRISPR enzymes, structure-guided engineering was performed to generate additional GEB×0173 variants. As shown in FIG. 15, 5 residues in REC1 and WED domain which located around the putative PAM binding site of GEB×0173 were mutated to get the GEB×0173 variant. The types of mutations are summarized in Table 12. The GEB×0173 variant PAM determination assay was performed as described in Example 5 and the related nucleic acid sequences (Human Codon Optimized sequence) of Cas12 GEB×0173-variant are shown in Table 13. The result of PAM is shown in FIG. 16, demonstrated a greatly change in −1 to −4 position comparing with the wildtype GEB×0173. GEB×0173-variant recognize a PAM having a sequence TNYN (Y is C or T, N is A, T, G or C).

TABLE 12
Types of mutations in GEBx0173-v1 variant-

| | | Mutation |
|---|---|---|
| Name | Site | type |
| GEBx0173-variant | I182 | S |
| | K532 | V |
| | E535 | N |
| | N536 | R |
| | K586 | R |

TABLE 13
The nucleic acid sequences of LbCpf1, Cas12 GEBx0173 and the variant

| | SEQ ID | |
|---|---|---|
| Name | NO: | 5′-3′ |
| LbCas12a | 90 | atggctcctaagaagaagcgcaaagtgATGAGCAAGCTGGAGAAGTTTACAAACTGCTACTCCCTGTCTAAGA |
| | | CCCTGAGGTTCAAGGCCATCCCTGTGGGCAAGACCCAGGAGAACATCGACAATAAGCGGCTGCTGGTGGAGGA |
| | | GACGAAGAGAGCCGAGGATTATAAGGGCGTGAAGAAGCTGCTGGATCGCTACTATCTGTCTTTTATCAACG |
| | | ACGTGCTGCACAGCATCAAGCTGAAGAATCTGAACAATTACATCAGCCTGTTCCGGAAGAAAACCAGAA |
| | | CCGAGAAGGAGAATAAGGAGCTGGAGAACCTGGAGATCAATCTGCGGAAGGAGATCGCCAAGGCCTTC |
| | | AAGGGCAACGAGGGCTACAAGTCCCTGTTTAAGAAGGATATCATCGAGACAATCCTGCCAGAGTTCCTG |
| | | GACGATAAGGACGAGATCGCCCTGGTGAACAGCTTCAATGGCTTTACCACAGCCTTCACCGGCTTCTTTG |
| | | ATAACAGAGAGAATATGTTTTCCGAGGAGGCCAAGAGCACATCCATCGCCTTCAGGTGTATCAACGAGAA |
| | | TCTGACCCGCTACATCTCTAATATGGACATCTTCGAGAAGGTGGACGCCATCTTTGATAAGCACGAGGTG |
| | | CAGGAGATCAAGGAGAAGATCCTGAACAGCGACTATGATGTGGAGGATTTCTTTGAGGGCGAGTTCTTTA |
| | | ACTTTGTGCTGACACAGGAGGGCATCGACGTGTATAACGCCATCATCGGCGGCTTCGTGACCGAGAGCG |
| | | GCGAGAAGATCAAGGGCCTGAACGAGTACATCAACCTGTATAATCAGAAAACCAAGCAGAAGCTGCCTA |
| | | AGTTTAAGCCACTGTATAAGCAGGTGCTGAGCGATCGGGAGTCTCTGAGCTTCTACGGCGAGGGCTATAC |
| | | ATCCGATGAGGAGGTGCTGGAGGTGTTTAGAAACACCCTGAACAAGAACAGCGAGATCTTCAGCTCCAT |
| | | CAAGAAGCTGGAGAAGCTGTTCAAGAATTTTGACGAGTACTCTAGCGCCGGCATCTTTGTGAAGAACGG |
| | | CCCCGCCATCAGCACAATCTCCAAGGATATCTTCGGCGAGTGGAACGTGATCCGGGACAAGTGGAATGC |
| | | CGAGTATGACGATATCCACCTGAAGAAGAAGGCCGTGGTGACCGAGAAGTACGAGGACGATCGGAGAA |
| | | AGTCCTTCAAGAAGATCGGCTCCTTTTCTCTGGAGCAGCTGCAGGAGTACGCCGACGCCGATCTGTCTGT |
| | | GGTGGAGAAGCTGAAGGAGATCATCATCCAGAAGGTGGATGAGATCTACAAGGTGTATGGCTCCTCTGA |
| | | GAAGCTGTTCGACGCCGATTTTGTGCTGGAGAAGAGCCTGAAGAAGAACGACGCCGTGGTGGCCATCAT |
| | | GAAGGACCTGCTGGATTCTGTGAAGAGCTTCGAGAATTACATCAAGGCCTTCTTTGGCGAGGGCAAGGA |
| | | GACAAACAGGGACGAGTCCTTCTATGGCGATTTTGTGCTGGCCTACGACATCCTGCTGAAGGTGGACCAC |
| | | ATCTACGATGCCATCCGCAATTATGTGACCCAGAAGCCCTACTCTAAGGATAAGTTCAAGCTGTATTTTCA |
| | | GAACCCTCAGTTCATGGGCGGCTGGGACAAGGATAAGGAGACAGACTATCGGGCCACCATCCTGAGATA |
| | | CGGCTCCAAGTACTATCTGGCCATCATGGATAAGAAGTACGCCAAGTGCCTGCAGAAGATCGACAAGGA |
| | | CGATGTGAACGGCAATTACGAGAAGATCAACTATAAGCTGCTGCCCGGCCCTAATAAGATGCTGCCAAAG |
| | | GTGTTCTTTTCTAAGAAGTGGATGGCCTACTATAACCCCAGCGAGGACATCCAGAAGATCTACAAGAATG |
| | | GCACATTCAAGAAGGGCGATATGTTTAACCTGAATGACTGTCACAAGCTGATCGACTTCTTTAAGGATAG |
| | | CATCTCCCGGTATCCAAAGTGGTCCAATGCCTACGATTTCAACTTTTCTGAGACAGAGAAGTATAAGGAC |
| | | ATCGCCGGCTTTTACAGAGAGGTGGAGGAGCAGGGCTATAAGGTGAGCTTCGAGTCTGCCAGCAAGAAG |
| | | GAGGTGGATAAGCTGGTGGAGGAGGGCAAGCTGTATATGTTCCAGATCTATAACAAGGACTTTTCCGATA |
| | | AGTCTCACGGCACACCCAATCTGCACACCATGTACTTCAAGCTGCTGTTTGACGAGAACAATCACGGAC |
| | | AGATCAGGCTGAGCGGAGGAGCAGAGCTGTTCATGAGGCGCGCCTCCCTGAAGAAGGAGGAGCTGGTG |
| | | GTGCACCCAGCCAACTCCCCTATCGCCAACAAGAATCCAGATAATCCCAAGAAAACCACAACCCTGTCC |
| | | TACGACGTGTATAAGGATAAGAGGTTTTCTGAGGACCAGTACGAGCTGCACATCCCAATCGCCATCAATA |
| | | AGTGCCCCAAGAACATCTTCAAGATCAATACAGAGGTGCGCGTGCTGCTGAAGCACGACGATAACCCCT |
| | | ATGTGATCGGCATCGATAGGGGCGAGCGCAATCTGCTGTATATCGTGGTGGTGGACGGCAAGGGCAACAT |
| | | CGTGGAGCAGTATTCCCTGAACGAGATCATCAACAACTTCAACGGCATCAGGATCAAGACAGATTACCAC |
| | | TCTCTGCTGGACAAGAAGGAGAAGGAGAGGTTCGAGGCCCGCCAGAACTGGACCTCCATCGAGAATATC |
| | | AAGGAGCTGAAGGCCGGCTATATCTCTCAGGTGGTGCACAAGATCTGCGAGCTGGTGGAGAAGTACGAT |
| | | GCCGTGATCGCCCTGGAGGACCTGAACTCTGGCTTTAAGAATAGCCGCGTGAAGGTGGAGAAGCAGGTG |
| | | TATCAGAAGTTCGAGAAGATGCTGATCGATAAGCTGAACTACATGGTGGACAAGAAGTCTAATCCTTGTG |
| | | CAACAGGCGGCGCCCTGAAGGGCTATCAGATCACCAATAAGTTCGAGAGCTTTAAGTCCATGTCTACCCA |
| | | GAACGGCTTCATCTTTTACATCCCTGCCTGGCTGACATCCAAGATCGATCCATCTACCGGCTTTGTGAACC |
| | | TGCTGAAAACCAAGTATACCAGCATCGCCGATTCCAAGAAGTTCATCAGCTCCTTTGACAGGATCATGTA |
| | | CGTGCCCGAGGAGGATCTGTTCGAGTTTGCCCTGGACTATAAGAACTTCTCTCGCACAGACGCCGATTAC |
| | | ATCAAGAAGTGGAAGCTGTACTCCTACGGCAACCGGATCAGAATCTTCCGGAATCCTAAGAAGAACAAC |
| | | GTGTTCGACTGGGAGGAGGTGTGCCTGACCAGCGCCTATAAGGAGCTGTTCAACAAGTACGGCATCAAT |
| | | TATCAGCAGGGCGATATCAGAGCCCTGCTGTGCGAGCAGTCCGACAAGGCCTTCTACTCTAGCTTTATGG |
| | | CCCTGATGAGCCTGATGCTGCAGATGCGGAACAGCATCACAGGCCGCACCGACGTGGATTTTCTGATCAG |
| | | CCCTGTGAAGAACTCCGACGGCATCTTCTACGATAGCCGGAACTATGAGGCCCAGGAGAATGCCATCCTG |
| | | CCAAAGAACGCCGACGCCAATGGCGCCTATAACATCGCCAGAAAGGTGCTGTGGGCCATCGGCCAGTTC |
| | | AAGAAGGCCGAGGACGAGAAGCTGGATAAGGTGAAGATCGCCATCTCTAACAAGGAGTGGCTGGAGTA |
| | | CGCCCAGACCAGCGTGAAGCACaaacgaccagctgcaacaaagaaggctggccaagcaaaaaagaagaaagat |
| | | tataaagatgatgacgataagtga |
| GEBx0173 | 91 | GCCTTGTTTTCCGAGAACTTTGTTGGGTGTTATCCAGTTTCTAAGACTTTGAAGTTTGAACTGCGCCCAGT |
| | | GCCCGAAACGAAAGCATACCTGGATGATGATAACTGTCCACTGTTGGCTGGTGATCTGGAGAGAGATAAA |
| | | GCCTACCCGGTGGTGAAGGAATTGTTGGACAAGTACTATAGGTATTTCATTGACATTTGTCTTAAAGATAA |
| | | AAAGTTGTCCCCCGAAACCATTGACGAGGCCTACCAGGCCTACAGTAGCCACGACGAGAAATCTCAGAA |
| | | GGCACTGAAGGCCGCCAGTCAGAAGCTGAGGAAGGAGGTCGCCGGATTTTTCGATAAAAAAACCCGCG |
| | | AAACCTACTGTCTCGACAAGTACAGTAATTTGTTGGTGCTCGACAAGAAAAACGATAATAAAGAGAAGG |
| | | GAACGCCCAAGACGATCCCGTCAGTTCTCAAGAAATGGCTTGAGAATGAGTCAGGATTCTCCGAGAAAG |
| | | AGGTGGAAAAATACTCCCAGGCTGTGCAGACGTTTAACGGTTTTACCACATACTTCATTTCCTACAAAGA |
| | | AAACAGGGAGAACATGTTCAGCAGTGAGGAGAAAGCCTCTGCCATCTCTTACAGAGTTGTTAATCAGAA |
| | | CATGCTGAGATTTTTCTCCAATGTGGAAAATTTCGAAAACATAAAAAAGAAGTACCCTGACCTTTATCAG |
| | | ACACTGAAAGAATTTGAGGAATCCTTTGTGCCTGCCGCCTACTCAAGCCTCCTTTCTCAGGCCGGTATAG |
| | | ATGACTATAATTACAGAGTTATCGGACGACCTGCAGAAGACGAGGATTTCGCCGGAGTCAATCGCCTGAT |
| | | AAATGAGTATAGGCAGAAAAATGCCGTGCGAAACCGGAACCTGCCAGTGATGGTTACTTTGTACAAACA |
| | | GATTCTCAGCGATCGGGAAAATGGTTTCGGGCGGGACATCTTGGCTTCCGACTCAGAAGCAAAAGAGTA |
| | | TATTCGGGGGAAGTACTCCAAGGCTTATGACACCGCCTGCCAACTGACTGTATTTGTCAATAATTACATAC |
| | | TGACAGAAGATCTGGACACTGTGTACGTTAAGACCCAGCACCTGAAGGATATCTCTCAAAGCATTTTCGG |
| | | AGAATGGTCCGTCCTTGATGACGCTATCAACAACGGAGGGATCGACAGCAAGATTATCTCATTGAAGGCT |
| | | CTGGCTGAGGCATTCGAGAAATATTGTTCAGCCCTTGATCCCGATATGGCCCAAAAATACCGGGACGCCT |
| | | TCAGTTTGAAGGAGTATCTCAAGAATCCGCCTGCTGTGGAACCCAGACTGCCTGAAACGGCCGAGAAGG |
| | | TTACCGCTTATAAGCCCGCTATGGATGAGTTGATCTCTCTGATAAGGTTTTACAAGCCTTTCTATTTGTACG |
| | | ACGGGAAGAAGAGACTGCAGGTGCCTGCCGAGGGAATGGATTTCTCTAATGAGTTCGACAAATATTTCG |
| | | AGGACTTGAAGGAATTTAGTCACGCATACGATAAAGTTCGAAATTTTGCAACCAAGAAACCCTACAGCGT |
| | | AGAGAAGATAAAACTCAATTTCAACCAGCCAACACTCCTGGACGGGTGGGACGTGAACAAGGAGAAAG |
| | | AGAATGCATCATTTCTCTTTGTCAAAGAGGGCAAGTACTACCTGGGAATTGCCGACAAGTCCGATAAAAA |
| | | TCTGTTCGATCGGAGTCGGGATGCCGTCAAAGAGGCACTCACTCTTACTTGCGGCGAAGATTACTATGAG |
| | | AAGGTGCAGTATAAGCAGCTTCCGAACATACGGATGAACCTGCCTCGCATAGTTTTTAAGCCTAAATGGG |
| | | AGAAGGTCTTTGCTCCTATAATCACTGAGAGAATCAGCGCCATTGAGAAAGAAAAGCTTTACACCGCAG |
| | | GTGCCAACGATAGGCAGGCGGTGATCGAGTGGATAGACTTCGTCAAGTCTGTCCTTGCGCTGCACTCCGA |
| | | GTGGAACAACTATTTCCAGTTTAAGTTTAAGGAGACGTCCGCATATAAAAATACAAAAGAGTTCTACGAC |
| | | GATCTTGATAACCAGGCTTACATCTTGAGTAAGATCAAAATCAACGCCTCATATATCCACAAACTGGTTCG |
| | | GGAGCGCAAATTGTATCTGTTTCAGATCTATTGTAAAGACTTTTCTGATAAGAAAAAAAAGACCGGCGCC |
| | | GACAATCTCCATACCATGTATTGGAGATGCCTGTTCTCCGATGAAAACTTGGCAGCTCTCGCACAGGGAC |
| | | AGGCCCCAATCATAAAGTTGAACGGGAAAGCTGAAATCTTCCTCCGCGAGCCTTCTCTCAAATATGAGGC |
| | | GACGCATCCGAAGAACCAGCCAGTGGCCAACAAGAATCCCCTGAACTCAAAACGGGAGAGCCTGTTCA |
| | | GCTACGATCTGGCAAAAAATCGCCGCTTTACCGAGAGGAAGTTGTTCTTCCACTGCCCCCTGACCATCAA |
| | | CTTTAGGACAGCTGCGGAAGGTGCGCGGAGCTTCAATGAGAGAGTTAATAGGTTCGTCGAGAACAACCC |
| | | CGATATCAGAATAATAGGCATTGATAGGGGCGAGAGACACCTGCTGTACTACACTGTGATCAACCAGAGC |
| | | GGCAAGATTCTGGAGCAGGGAAGTCTGAACAAAATTAGCTCAGATTACCGAACTCCTGAGGGCGCCGAG |
| | | GTGGAAAAAGTCACTGACTATCACGAGCTCTTGAACCGGAAAGAAGGAGATAGACGAGCTGCCCGACA |
| | | GACATGGGACACCATAGAAAATATCAAGGAGCTTAAAGAGGGATACCTCAGCCAGGTGGTGCATAAGCT |
| | | GACTACACTGATGATTAAACACAATGCTGTGGTCGTTTTGGAGAACCTTAATCAAAACTTCAAACGCTCC |
| | | CGCATTAAGGTCGAGAAGCAGGTCTATCAGAAGTTCGAGAAGGCTCTGATCGATAAACTGAATTATTTGG |
| | | TTTTCAAGGATAGACCTTACGGAGCTCCCGGTTCCTATGCCAATGGGCTGCAGCTTACGGCTCCTTTCGA |
| | | ATCCTTTGATAGACTCGGCAAGCAGACAGGGGCTCTCTATTACGTGGTGCCCAGCTACACATCCCACATT |
| | | GATCCTAAGACTGGGTTTGTGAACCTCCTGGGCGCAAAGCTCCGGTACGAAAGCGTGGTGAAAGCTCAG |
| | | GAAACAATGAAGAAGATCGGGAGTTTCAAGTTCAACCCCGAGAGGAATTGGTTTGAAATCAGCTTCGAC |
| | | TACGGCGACTTCGGGATCGAGATGGCACATCCCCAGTGGACCGCATGTACTCACGGCGATGCTTATTGGG |
| | | GATACAATCCCAAGGAAAAAAGGGCCGTTAGCTACGACGTGACGGAAGAGATTAAAAAGCTGCTGACA |
| | | GAATACGGAATTGATTATGGGACTGGAGCAGATATCAAGGAGGCCGCTATTCGACAGACCAAGCCCGCTT |
| | | TTTGGAAGACCCTGTTTTACATGCTCAAGCTGGCATTGCAGTTGAGGAATACCGTGGCTGGCACTGATAA |
| | | CGAGAACGACTACATTCTGTCACCAGTCGAGTATGAGGCCGGGAAGTTCTTTGATTCTAGAAAGGCTGG |
| | | AGAGGACGAACCCCAGAATGCAGACGCCAATGGAGCGTACCACATCGCGATGAAAGGATTGCAGGCATT |
| | | GCGGCGCATCGAGGACGGCAAAGTGAACAAATTCCCGAAGAACGGTGAGCGAAAAGCTTGGCTGGCCT |
| | | TTATGCAAAATAGGGAATATTTGAAA |
| GEBx0173 | 92 | atggccccaaagaagaagcggaaggtcGCCTTGTTTTCCGAGAACTTTGTTGGGTGTTATCCAGTTTCTAAGA |
| with | | CTTTGAAGTTTGAACTGCGCCCAGTGCCCGAAACGAAAGCATACCTGGATGATGATAACTGTCCACTGTTGGC |
| NLS and | | TGGTGATCTGGAGAGAGATAAAGCCTACCCGGTGGTGAAGGAATTGTTGGACAAGTACTATAGGTATTTCATT |
| FLAG | | GACATTTGTCTTAAAGATAAAAAGTTGTCCCCCGAAACCATTGACGAGGCCTACCAGGCCTACAGTAGCCA |
| | | CGACGAGAAATCTCAGAAGGCACTGAAGGCCGCCAGTCAGAAGCTGAGGAAGGAGGTCGCCGGATTTT |
| | | TCGATAAAAAAACCCGCGAAACCTACTGTCTCGACAAGTACAGTAATTTGTTGGTGCTCGACAAGAAAA |
| | | ACGATAATAAAGAGAAGGGAACGCCCAAGACGATCCCGTCAGTTCTCAAGAAATGGCTTGAGAATGAGT |
| | | CAGGATTCTCCGAGAAAGAGGTGGAAAAATACTCCCAGGCTGTGCAGACGTTTAACGGTTTTACCACAT |
| | | ACTTCATTTCCTACAAAGAAAACAGGGAGAACATGTTCAGCAGTGAGGAGAAAGCCTCTGCCATCTCTT |
| | | ACAGAGTTGTTAATCAGAACATGCTGAGATTTTTCTCCAATGTGGAAAATTTCGAAAACATAAAAAAGAA |
| | | GTACCCTGACCTTTATCAGACACTGAAAGAATTTGAGGAATCCTTTGTGCCTGCCGCCTACTCAAGCCTC |
| | | CTTTCTCAGGCCGGTATAGATGACTATAATTACAGAGTTATCGGACGACCTGCAGAAGACGAGGATTTCG |
| | | CCGGAGTCAATCGCCTGATAAATGAGTATAGGCAGAAAAATGCCGTGCGAAACCGGAACCTGCCAGTGA |
| | | TGGTTACTTTGTACAAACAGATTCTCAGCGATCGGGAAAATGGTTTCGGGCGGGACATCTTGGCTTCCGA |
| | | CTCAGAAGCAAAAGAGTATATTCGGGGGAAGTACTCCAAGGCTTATGACACCGCCTGCCAACTGACTGT |
| | | ATTTGTCAATAATTACATACTGACAGAAGATCTGGACACTGTGTACGTTAAGACCCAGCACCTGAAGGAT |
| | | ATCTCTCAAAGCATTTTCGGAGAATGGTCCGTCCTTGATGACGCTATCAACAACGGAGGGATCGACAGCA |
| | | AGATTATCTCATTGAAGGCTCTGGCTGAGGCATTCGAGAAATATTGTTCAGCCCTTGATCCCGATATGGCC |
| | | CAAAAATACCGGGACGCCTTCAGTTTGAAGGAGTATCTCAAGAATCCGCCTGCTGTGGAACCCAGACTG |
| | | CCTGAAACGGCCGAGAAGGTTACCGCTTATAAGCCCGCTATGGATGAGTTGATCTCTCTGATAAGGTTTTA |
| | | CAAGCCTTTCTATTTGTACGACGGGAAGAAGAGACTGCAGGTGCCTGCCGAGGGAATGGATTTCTCTAAT |
| | | GAGTTCGACAAATATTTCGAGGACTTGAAGGAATTTAGTCACGCATACGATAAAGTTCGAAATTTTGCAA |
| | | CCAAGAAACCCTACAGCGTAGAGAAGATAAAACTCAATTTCAACCAGCCAACACTCCTGGACGGGTGGG |
| | | ACGTGAACAAGGAGAAAGAGAATGCATCATTTCTCTTTGTCAAAGAGGGCAAGTACTACCTGGGAATTG |
| | | CCGACAAGTCCGATAAAAATCTGTTCGATCGGAGTCGGGATGCCGTCAAAGAGGCACTCACTCTTACTTG |
| | | CGGCGAAGATTACTATGAGAAGGTGCAGTATAAGCAGCTTCCGAACATACGGATGAACCTGCCTCGCATA |
| | | GTTTTTAAGCCTAAATGGGAGAAGGTCTTTGCTCCTATAATCACTGAGAGAATCAGCGCCATTGAGAAAG |
| | | AAAAGCTTTACACCGCAGGTGCCAACGATAGGCAGGCGGTGATCGAGTGGATAGACTTCGTCAAGTCTG |
| | | TCCTTGCGCTGCACTCCGAGTGGAACAACTATTTCCAGTTTAAGTTTAAGGAGACGTCCGCATATAAAAA |
| | | TACAAAAGAGTTCTACGACGATCTTGATAACCAGGCTTACATCTTGAGTAAGATCAAAATCAACGCCTCA |
| | | TATATCCACAAACTGGTTCGGGAGCGCAAATTGTATCTGTTTCAGATCTATTGTAAAGACTTTTCTGATAA |
| | | GAAAAAAAAGACCGGCGCCGACAATCTCCATACCATGTATTGGAGATGCCTGTTCTCCGATGAAAACTTG |
| | | GCAGCTCTCGCACAGGGACAGGCCCCAATCATAAAGTTGAACGGGAAAGCTGAAATCTTCCTCCGCGAG |
| | | CCTTCTCTCAAATATGAGGCGACGCATCCGAAGAACCAGCCAGTGGCCAACAAGAATCCCCTGAACTCA |
| | | AAACGGGAGAGCCTGTTCAGCTACGATCTGGCAAAAAATCGCCGCTTTACCGAGAGGAAGTTGTTCTTC |
| | | CACTGCCCCCTGACCATCAACTTTAGGACAGCTGCGGAAGGTGCGCGGAGCTTCAATGAGAGAGTTAAT |
| | | AGGTTCGTCGAGAACAACCCCGATATCAGAATAATAGGCATTGATAGGGGCGAGAGACACCTGCTGTACT |
| | | ACACTGTGATCAACCAGAGCGGCAAGATTCTGGAGCAGGGAAGTCTGAACAAAATTAGCTCAGATTACC |
| | | GAACTCCTGAGGGCGCCGAGGTGGAAAAAGTCACTGACTATCACGAGCTCTTGAACCGGAAAGAAGGA |
| | | GATAGACGAGCTGCCCGACAGACATGGGACACCATAGAAAATATCAAGGAGCTTAAAGAGGGATACCTC |
| | | AGCCAGGTGGTGCATAAGCTGACTACACTGATGATTAAACACAATGCTGTGGTCGTTTTGGAGAACCTTA |
| | | ATCAAAACTTCAAACGCTCCCGCATTAAGGTCGAGAAGCAGGTCTATCAGAAGTTCGAGAAGGCTCTGA |
| | | TCGATAAACTGAATTATTTGGTTTTCAAGGATAGACCTTACGGAGCTCCCGGTTCCTATGCCAATGGGCTG |
| | | CAGCTTACGGCTCCTTTCGAATCCTTTGATAGACTCGGCAAGCAGACAGGGGCTCTCTATTACGTGGTGC |
| | | CCAGCTACACATCCCACATTGATCCTAAGACTGGGTTTGTGAACCTCCTGGGCGCAAAGCTCCGGTACGA |
| | | AAGCGTGGTGAAAGCTCAGGAAACAATGAAGAAGATCGGGAGTTTCAAGTTCAACCCCGAGAGGAATT |
| | | GGTTTGAAATCAGCTTCGACTACGGCGACTTCGGGATCGAGATGGCACATCCCCAGTGGACCGCATGTAC |
| | | TCACGGCGATGCTTATTGGGGATACAATCCCAAGGAAAAAAGGGCCGTTAGCTACGACGTGACGGAAGA |
| | | GATTAAAAAGCTGCTGACAGAATACGGAATTGATTATGGGACTGGAGCAGATATCAAGGAGGCCGCTATT |
| | | CGACAGACCAAGCCCGCTTTTTGGAAGACCCTGTTTTACATGCTCAAGCTGGCATTGCAGTTGAGGAATA |
| | | CCGTGGCTGGCACTGATAACGAGAACGACTACATTCTGTCACCAGTCGAGTATGAGGCCGGGAAGTTCTT |
| | | TGATTCTAGAAAGGCTGGAGAGGACGAACCCCAGAATGCAGACGCCAATGGAGCGTACCACATCGCGAT |
| | | GAAAGGATTGCAGGCATTGCGGCGCATCGAGGACGGCAAAGTGAACAAATTCCCGAAGAACGGTGAGC |
| | | GAAAAGCTTGGCTGGCCTTTATGCAAAATAGGGAATATTTGAAAaaacgacccgccgccacaaaaaaggccgg |
| | | gcaggcaaagaaaaaaaaggattacaaagacgatgacgacaagtaa |
| GEBx0173- | 93 | GCCTTGTTTTCCGAGAACTTTGTTGGGTGTTATCCAGTTTCTAAGACTTTGAAGTTTGAACTGCGCCCAGT |
| variant1 | | GCCCGAAACGAAAGCATACCTGGATGATGATAACTGTCCACTGTTGGCTGGTGATCTGGAGAGAGATAAA |
| (GEBx0173- | | GCCTACCCGGTGGTGAAGGAATTGTTGGACAAGTACTATAGGTATTTCATTGACATTTGTCTTAAAGATAA |
| v1) | | AAAGTTGTCCCCCGAAACCATTGACGAGGCCTACCAGGCCTACAGTAGCCACGACGAGAAATCTCAGAA |
| | | GGCACTGAAGGCCGCCAGTCAGAAGCTGAGGAAGGAGGTCGCCGGATTTTTCGATAAAAAAACCCGCG |
| | | AAACCTACTGTCTCGACAAGTACAGTAATTTGTTGGTGCTCGACAAGAAAAACGATAATAAAGAGAAGG |
| | | GAACGCCCAAGACGATCCCGTCAGTTCTCAAGAAATGGCTTGAGAATGAGTCAGGATTCTCCGAGAAAG |
| | | AGGTGGAAAAATACTCCCAGGCTGTGCAGACGTTTAACGGTTTTACCACATACTTCTCCTCCTACAAACG |
| | | AAACAGGGAGAACATGTTCAGCAGTGAGGAGAAAGCCTCTGCCATCTCTTACAGAGTTGTTAATCAGAA |
| | | CATGCTGAGATTTTTCTCCAATGTGGAAAATTTCGAAAACATAAAAAAGAAGTACCCTGACCTTTATCAG |
| | | ACACTGAAAGAATTTGAGGAATCCTTTGTGCCTGCCGCCTACTCAAGCCTCCTTTCTCAGGCCGGTATAG |
| | | ATGACTATAATTACAGAGTTATCGGACGACCTGCAGAAGACGAGGATTTCGCCGGAGTCAATCGCCTGAT |
| | | AAATGAGTATAGGCAGAAAAATGCCGTGCGAAACCGGAACCTGCCAGTGATGGTTACTTTGTACAAACA |
| | | GATTCTCAGCGATCGGGAAAATGGTTTCGGGCGGGACATCTTGGCTTCCGACTCAGAAGCAAAAGAGTA |
| | | TATTCGGGGGAAGTACTCCAAGGCTTATGACACCGCCTGCCAACTGACTGTATTTGTCAATAATTACATAC |
| | | TGACAGAAGATCTGGACACTGTGTACGTTAAGACCCAGCACCTGAAGGATATCTCTCAAAGCATTTTCGG |
| | | AGAATGGTCCGTCCTTGATGACGCTATCAACAACGGAGGGATCGACAGCAAGATTATCTCATTGAAGGCT |
| | | CTGGCTGAGGCATTCGAGAAATATTGTTCAGCCCTTGATCCCGATATGGCCCAAAAATACCGGGACGCCT |
| | | TCAGTTTGAAGGAGTATCTCAAGAATCCGCCTGCTGTGGAACCCAGACTGCCTGAAACGGCCGAGAAGG |
| | | TTACCGCTTATAAGCCCGCTATGGATGAGTTGATCTCTCTGATAAGGTTTTACAAGCCTTTCTATTTGTACG |
| | | ACGGGAAGAAGAGACTGCAGGTGCCTGCCGAGGGAATGGATTTCTCTAATGAGTTCGACAAATATTTCG |
| | | AGGACTTGAAGGAATTTAGTCACGCATACGATAAAGTTCGAAATTTTGCAACCAAGAAACCCTACAGCGT |
| | | AGAGAAGATAAAACTCAATTTCAACCAGCCAACACTCCTGCGCGGGTGGGACGTGAACGTGGAGAAAA |
| | | ATCGGGCATCATTTCTCTTTGTCAAAGAGGGCAAGTACTACCTGGGAATTGCCGACAAGTCCGATAAAAA |
| | | TCTGTTCGATCGGAGTCGGGATGCCGTCAAAGAGGCACTCACTCTTACTTGCGGCGAAGATTACTATGAG |
| | | AAGGTGCAGTATCGGCAGCTTCCGAACATACGGATGAACCTGCCTCGCATAGTTTTTAAGCCTAAATGGG |
| | | AGAAGGTCTTTGCTCCTATAATCACTGAGAGAATCAGCGCCATTGAGAAAGAAAAGCTTTACACCGCAG |
| | | GTGCCAACGATAGGCAGGCGGTGATCGAGTGGATAGACTTCGTCAAGTCTGTCCTTGCGCTGCACTCCGA |
| | | GTGGAACAACTATTTCCAGTTTAAGTTTAAGGAGACGTCCGCATATAAAAATACAAAAGAGTTCTACGAC |
| | | GATCTTGATAACCAGGCTTACATCTTGAGTAAGATCAAAATCAACGCCTCATATATCCACAAACTGGTTCG |
| | | GGAGCGCAAATTGTATCTGTTTCAGATCTATTGTAAAGACTTTTCTGATAAGAAAAAAAAGACCGGCGCC |
| | | GACAATCTCCATACCATGTATTGGAGATGCCTGTTCTCCGATGAAAACTTGGCAGCTCTCGCACAGGGAC |
| | | AGGCCCCAATCATAAAGTTGAACGGGAAAGCTGAAATCTTCCTCCGCGAGCCTTCTCTCAAATATGAGGC |
| | | GACGCATCCGAAGAACCAGCCAGTGGCCAACAAGAATCCCCTGAACTCAAAACGGGAGAGCCTGTTCA |
| | | GCTACGATCTGGCAAAAAATCGCCGCTTTACCGAGAGGAAGTTGTTCTTCCACTGCCCCCTGACCATCAA |
| | | CTTTAGGACAGCTGCGGAAGGTGCGCGGAGCTTCAATGAGAGAGTTAATAGGTTCGTCGAGAACAACCC |
| | | CGATATCAGAATAATAGGCATTGATAGGGGCGAGAGACACCTGCTGTACTACACTGTGATCAACCAGAGC |
| | | GGCAAGATTCTGGAGCAGGGAAGTCTGAACAAAATTAGCTCAGATTACCGAACTCCTGAGGGCGCCGAG |
| | | GTGGAAAAAGTCACTGACTATCACGAGCTCTTGAACCGGAAAGAAGGAGATAGACGAGCTGCCCGACA |
| | | GACATGGGACACCATAGAAAATATCAAGGAGCTTAAAGAGGGATACCTCAGCCAGGTGGTGCATAAGCT |
| | | GACTACACTGATGATTAAACACAATGCTGTGGTCGTTTTGGAGAACCTTAATCAAAACTTCAAACGCTCC |
| | | CGCATTAAGGTCGAGAAGCAGGTCTATCAGAAGTTCGAGAAGGCTCTGATCGATAAACTGAATTATTTGG |
| | | TTTTCAAGGATAGACCTTACGGAGCTCCCGGTTCCTATGCCAATGGGCTGCAGCTTACGGCTCCTTTCGA |
| | | ATCCTTTGATAGACTCGGCAAGCAGACAGGGGCTCTCTATTACGTGGTGCCCAGCTACACATCCCACATT |
| | | GATCCTAAGACTGGGTTTGTGAACCTCCTGGGCGCAAAGCTCCGGTACGAAAGCGTGGTGAAAGCTCAG |
| | | GAAACAATGAAGAAGATCGGGAGTTTCAAGTTCAACCCCGAGAGGAATTGGTTTGAAATCAGCTTCGAC |
| | | TACGGCGACTTCGGGATCGAGATGGCACATCCCCAGTGGACCGCATGTACTCACGGCGATGCTTATTGGG |
| | | GATACAATCCCAAGGAAAAAAGGGCCGTTAGCTACGACGTGACGGAAGAGATTAAAAAGCTGCTGACA |
| | | GAATACGGAATTGATTATGGGACTGGAGCAGATATCAAGGAGGCCGCTATTCGACAGACCAAGCCCGCTT |
| | | TTTGGAAGACCCTGTTTTACATGCTCAAGCTGGCATTGCAGTTGAGGAATACCGTGGCTGGCACTGATAA |
| | | CGAGAACGACTACATTCTGTCACCAGTCGAGTATGAGGCCGGGAAGTTCTTTGATTCTAGAAAGGCTGG |
| | | AGAGGACGAACCCCAGAATGCAGACGCCAATGGAGCGTACCACATCGCGATGAAAGGATTGCAGGCATT |
| | | GCGGCGCATCGAGGACGGCAAAGTGAACAAATTCCCGAAGAACGGTGAGCGAAAAGCTTGGCTGGCCT |
| | | TTATGCAAAATAGGGAATATTTGAAA |
| GEBx0173- | 94 | ATGGCCCCAAAGAAGAAGCGGAAGGTCGCCTTGTTTTCCGAGAACTTTGTTGGGTGTTATCCAGTTTCTA |
| variant1 | | AGACTTTGAAGTTTGAACTGCGCCCAGTGCCCGAAACGAAAGCATACCTGGATGATGATAACTGTCCACT |
| with | | GTTGGCTGGTGATCTGGAGAGAGATAAAGCCTACCCGGTGGTGAAGGAATTGTTGGACAAGTACTATAG |
| NLS and | | GTATTTCATTGACATTTGTCTTAAAGATAAAAAGTTGTCCCCCGAAACCATTGACGAGGCCTACCAGGCCT |
| FLAG | | ACAGTAGCCACGACGAGAAATCTCAGAAGGCACTGAAGGCCGCCAGTCAGAAGCTGAGGAAGGAGGTC |
| | | GCCGGATTTTTCGATAAAAAAACCCGCGAAACCTACTGTCTCGACAAGTACAGTAATTTGTTGGTGCTCG |
| | | ACAAGAAAAACGATAATAAAGAGAAGGGAACGCCCAAGACGATCCCGTCAGTTCTCAAGAAATGGCTT |
| | | GAGAATGAGTCAGGATTCTCCGAGAAAGAGGTGGAAAAATACTCCCAGGCTGTGCAGACGTTTAACGGT |
| | | TTTACCACATACTTCTCCTCCTACAAACGAAACAGGGAGAACATGTTCAGCAGTGAGGAGAAAGCCTCT |
| | | GCCATCTCTTACAGAGTTGTTAATCAGAACATGCTGAGATTTTTCTCCAATGTGGAAAATTTCGAAAACAT |
| | | AAAAAAGAAGTACCCTGACCTTTATCAGACACTGAAAGAATTTGAGGAATCCTTTGTGCCTGCCGCCTAC |
| | | TCAAGCCTCCTTTCTCAGGCCGGTATAGATGACTATAATTACAGAGTTATCGGACGACCTGCAGAAGACG |
| | | AGGATTTCGCCGGAGTCAATCGCCTGATAAATGAGTATAGGCAGAAAAATGCCGTGCGAAACCGGAACC |
| | | TGCCAGTGATGGTTACTTTGTACAAACAGATTCTCAGCGATCGGGAAAATGGTTTCGGGCGGGACATCTT |
| | | GGCTTCCGACTCAGAAGCAAAAGAGTATATTCGGGGGAAGTACTCCAAGGCTTATGACACCGCCTGCCA |
| | | ACTGACTGTATTTGTCAATAATTACATACTGACAGAAGATCTGGACACTGTGTACGTTAAGACCCAGCACC |
| | | TGAAGGATATCTCTCAAAGCATTTTCGGAGAATGGTCCGTCCTTGATGACGCTATCAACAACGGAGGGAT |
| | | CGACAGCAAGATTATCTCATTGAAGGCTCTGGCTGAGGCATTCGAGAAATATTGTTCAGCCCTTGATCCCG |
| | | ATATGGCCCAAAAATACCGGGACGCCTTCAGTTTGAAGGAGTATCTCAAGAATCCGCCTGCTGTGGAACC |
| | | CAGACTGCCTGAAACGGCCGAGAAGGTTACCGCTTATAAGCCCGCTATGGATGAGTTGATCTCTCTGATA |
| | | AGGTTTTACAAGCCTTTCTATTTGTACGACGGGAAGAAGAGACTGCAGGTGCCTGCCGAGGGAATGGAT |
| | | TTCTCTAATGAGTTCGACAAATATTTCGAGGACTTGAAGGAATTTAGTCACGCATACGATAAAGTTCGAAA |
| | | TTTTGCAACCAAGAAACCCTACAGCGTAGAGAAGATAAAACTCAATTTCAACCAGCCAACACTCCTGCG |
| | | CGGGTGGGACGTGAACGTGGAGAAAAATCGGGCATCATTTCTCTTTGTCAAAGAGGGCAAGTACTACCT |
| | | GGGAATTGCCGACAAGTCCGATAAAAATCTGTTCGATCGGAGTCGGGATGCCGTCAAAGAGGCACTCAC |
| | | TCTTACTTGCGGCGAAGATTACTATGAGAAGGTGCAGTATCGGCAGCTTCCGAACATACGGATGAACCTG |
| | | CCTCGCATAGTTTTTAAGCCTAAATGGGAGAAGGTCTTTGCTCCTATAATCACTGAGAGAATCAGCGCCAT |
| | | TGAGAAAGAAAAGCTTTACACCGCAGGTGCCAACGATAGGCAGGCGGTGATCGAGTGGATAGACTTCGT |
| | | CAAGTCTGTCCTTGCGCTGCACTCCGAGTGGAACAACTATTTCCAGTTTAAGTTTAAGGAGACGTCCGCA |
| | | TATAAAAATACAAAAGAGTTCTACGACGATCTTGATAACCAGGCTTACATCTTGAGTAAGATCAAAATCAA |
| | | CGCCTCATATATCCACAAACTGGTTCGGGAGCGCAAATTGTATCTGTTTCAGATCTATTGTAAAGACTTTTC |
| | | TGATAAGAAAAAAAAGACCGGCGCCGACAATCTCCATACCATGTATTGGAGATGCCTGTTCTCCGATGAA |
| | | AACTTGGCAGCTCTCGCACAGGGACAGGCCCCAATCATAAAGTTGAACGGGAAAGCTGAAATCTTCCTC |
| | | CGCGAGCCTTCTCTCAAATATGAGGCGACGCATCCGAAGAACCAGCCAGTGGCCAACAAGAATCCCCTG |
| | | AACTCAAAACGGGAGAGCCTGTTCAGCTACGATCTGGCAAAAAATCGCCGCTTTACCGAGAGGAAGTTG |
| | | TTCTTCCACTGCCCCCTGACCATCAACTTTAGGACAGCTGCGGAAGGTGCGCGGAGCTTCAATGAGAGA |
| | | GTTAATAGGTTCGTCGAGAACAACCCCGATATCAGAATAATAGGCATTGATAGGGGCGAGAGACACCTGC |
| | | TGTACTACACTGTGATCAACCAGAGCGGCAAGATTCTGGAGCAGGGAAGTCTGAACAAAATTAGCTCAG |
| | | ATTACCGAACTCCTGAGGGCGCCGAGGTGGAAAAAGTCACTGACTATCACGAGCTCTTGAACCGGAAAG |
| | | AAGGAGATAGACGAGCTGCCCGACAGACATGGGACACCATAGAAAATATCAAGGAGCTTAAAGAGGGAT |
| | | ACCTCAGCCAGGTGGTGCATAAGCTGACTACACTGATGATTAAACACAATGCTGTGGTCGTTTTGGAGAA |
| | | CCTTAATCAAAACTTCAAACGCTCCCGCATTAAGGTCGAGAAGCAGGTCTATCAGAAGTTCGAGAAGGC |
| | | TCTGATCGATAAACTGAATTATTTGGTTTTCAAGGATAGACCTTACGGAGCTCCCGGTTCCTATGCCAATG |
| | | GGCTGCAGCTTACGGCTCCTTTCGAATCCTTTGATAGACTCGGCAAGCAGACAGGGGCTCTCTATTACGT |
| | | GGTGCCCAGCTACACATCCCACATTGATCCTAAGACTGGGTTTGTGAACCTCCTGGGCGCAAAGCTCCGG |
| | | TACGAAAGCGTGGTGAAAGCTCAGGAAACAATGAAGAAGATCGGGAGTTTCAAGTTCAACCCCGAGAG |
| | | GAATTGGTTTGAAATCAGCTTCGACTACGGCGACTTCGGGATCGAGATGGCACATCCCCAGTGGACCGCA |
| | | TGTACTCACGGCGATGCTTATTGGGGATACAATCCCAAGGAAAAAAGGGCCGTTAGCTACGACGTGACG |
| | | GAAGAGATTAAAAAGCTGCTGACAGAATACGGAATTGATTATGGGACTGGAGCAGATATCAAGGAGGCC |
| | | GCTATTCGACAGACCAAGCCCGCTTTTTGGAAGACCCTGTTTTACATGCTCAAGCTGGCATTGCAGTTGA |
| | | GGAATACCGTGGCTGGCACTGATAACGAGAACGACTACATTCTGTCACCAGTCGAGTATGAGGCCGGGA |
| | | AGTTCTTTGATTCTAGAAAGGCTGGAGAGGACGAACCCCAGAATGCAGACGCCAATGGAGCGTACCACA |
| | | TCGCGATGAAAGGATTGCAGGCATTGCGGCGCATCGAGGACGGCAAAGTGAACAAATTCCCGAAGAAC |
| | | GGTGAGCGAAAAGCTTGGCTGGCCTTTATGCAAAATAGGGAATATTTGAAAAAACGACCCGCCGCCACA |
| | | AAAAAGGCCGGGCAGGCAAAGAAAAAAAAGGATTACAAAGACGATGACGACAAGTAA |
| GEBx0173- | 156 | atggccccaaagaagaagcggaaggtcGCCTTGTTTTCCGAGAACTTTGTTGGGTGTTATCCAGTTTCTAAGA |
| variant | | CTTTGAAGTTTGAACTGCGCCCAGTGCCCGAAACGAAAGCATACCTGGATGATGATAACTGTCCACTGTTGGC |
| 2 | | TGGTGATCTGGAGAGAGATAAAGCCTACCCGGTGGTGAAGGAATTGTTGGACAAGTACTATAGGTATTTCATT |
| with NLS | | GACATTTGTCTTAAAGATAAAAAGTTGTCCCCCGAAACCATTGACGAGGCCTACCAGGCCTACAGTAGCCA |
| and | | CGACGAGAAATCTCAGAAGGCACTGAAGGCCGCCAGTCAGAAGCTGAGGAAGGAGGTCGCCGGATTTT |
| FLAG | | TCGATAAAAAAACCCGCGAAACCTACTGTCTCGACAAGTACAGTAATTTGTTGGTGCTCGACAAGAAAA |
| | | ACGATAATAAAGAGAAGGGAACGCCCAAGACGATCCCGTCAGTTCTCAAGAAATGGCTTGAGAATGAGT |
| | | CAGGATTCTCCGAGAAAGAGGTGGAAAAATACTCCCAGGCTGTGCAGACGTTTAACGGTTTTACCACAT |
| | | ACTTCATTTCCTACAAAcgAAACAGGGAGAACATGTTCAGCAGTGAGGAGAAAGCCTCTGCCATCTCTTA |
| | | CAGAGTTGTTAATCAGAACATGCTGAGATTTTTCTCCAATGTGGAAAATTTCGAAAACATAAAAAAGAAG |
| | | TACCCTGACCTTTATCAGACACTGAAAGAATTTGAGGAATCCTTTGTGCCTGCCGCCTACTCAAGCCTCCT |
| | | TTCTCAGGCCGGTATAGATGACTATAATTACAGAGTTATCGGACGACCTGCAGAAGACGAGGATTTCGCC |
| | | GGAGTCAATCGCCTGATAAATGAGTATAGGCAGAAAAATGCCGTGCGAAACCGGAACCTGCCAGTGATG |
| | | GTTACTTTGTACAAACAGATTCTCAGCGATCGGGAAAATGGTTTCGGGCGGGACATCTTGGCTTCCGACT |
| | | CAGAAGCAAAAGAGTATATTCGGGGGAAGTACTCCAAGGCTTATGACACCGCCTGCCAACTGACTGTATT |
| | | TGTCAATAATTACATACTGACAGAAGATCTGGACACTGTGTACGTTAAGACCCAGCACCTGAAGGATATC |
| | | TCTCAAAGCATTTTCGGAGAATGGTCCGTCCTTGATGACGCTATCAACAACGGAGGGATCGACAGCAAG |
| | | ATTATCTCATTGAAGGCTCTGGCTGAGGCATTCGAGAAATATTGTTCAGCCCTTGATCCCGATATGGCCCA |
| | | AAAATACCGGGACGCCTTCAGTTTGAAGGAGTATCTCAAGAATCCGCCTGCTGTGGAACCCAGACTGCC |
| | | TGAAACGGCCGAGAAGGTTACCGCTTATAAGCCCGCTATGGATGAGTTGATCTCTCTGATAAGGTTTTACA |
| | | AGCCTTTCTATTTGTACGACGGGAAGAAGAGACTGCAGGTGCCTGCCGAGGGAATGGATTTCTCTAATGA |
| | | GTTCGACAAATATTTCGAGGACTTGAAGGAATTTAGTCACGCATACGATAAAGTTCGAAATTTTGCAACC |
| | | AAGAAACCCTACAGCGTAGAGAAGATAAAACTCAATTTCAACCAGCCAACACTCCTGcgCGGGTGGGAC |
| | | GTGAACcgGGAGAAAGAGAATGCATCATTTCTCTTTGTCAAAGAGGGCAAGTACTACCTGGGAATTGCCG |
| | | ACAAGTCCGATAAAAATCTGTTCGATCGGAGTCGGGATGCCGTCAAAGAGGCACTCACTCTTACTTGCGG |
| | | CGAAGATTACTATGAGAAGGTGCAGTATAAGCAGCTTCCGAACATACGGATGAACCTGCCTCGCATAGTT |
| | | TTTAAGCCTAAATGGGAGAAGGTCTTTGCTCCTATAATCACTGAGAGAATCAGCGCCATTGAGAAAGAAA |
| | | AGCTTTACACCGCAGGTGCCAACGATAGGCAGGCGGTGATCGAGTGGATAGACTTCGTCAAGTCTGTCCT |
| | | TGCGCTGCACTCCGAGTGGAACAACTATTTCCAGTTTAAGTTTAAGGAGACGTCCGCATATAAAAATACA |
| | | AAAGAGTTCTACGACGATCTTGATAACCAGGCTTACATCTTGAGTAAGATCAAAATCAACGCCTCATATAT |
| | | CCACAAACTGGTTCGGGAGCGCAAATTGTATCTGTTTCAGATCTATTGTAAAGACTTTTCTGATAAGAAA |
| | | AAAAAGACCGGCGCCGACAATCTCCATACCATGTATTGGAGATGCCTGTTCTCCGATGAAAACTTGGCAG |
| | | CTCTCGCACAGGGACAGGCCCCAATCATAAAGTTGAACGGGAAAGCTGAAATCTTCCTCCGCGAGCCTT |
| | | CTCTCAAATATGAGGCGACGCATCCGAAGAACCAGCCAGTGGCCAACAAGAATCCCCTGAACTCAAAAC |
| | | GGGAGAGCCTGTTCAGCTACGATCTGGCAAAAAATCGCCGCTTTACCGAGAGGAAGTTGTTCTTCCACT |
| | | GCCCCCTGACCATCAACTTTAGGACAGCTGCGGAAGGTGCGCGGAGCTTCAATGAGAGAGTTAATAGGT |
| | | TCGTCGAGAACAACCCCGATATCAGAATAATAGGCATTGATAGGGGCGAGAGACACCTGCTGTACTACAC |
| | | TGTGATCAACCAGAGCGGCAAGATTCTGGAGCAGGGAAGTCTGAACAAAATTAGCTCAGATTACCGAAC |
| | | TCCTGAGGGCGCCGAGGTGGAAAAAGTCACTGACTATCACGAGCTCTTGAACCGGAAAGAAGGAGATA |
| | | GACGAGCTGCCCGACAGACATGGGACACCATAGAAAATATCAAGGAGCTTAAAGAGGGATACCTCAGCC |
| | | AGGTGGTGCATAAGCTGACTACACTGATGATTAAACACAATGCTGTGGTCGTTTTGGAGAACCTTAATCA |
| | | AAACTTCAAACGCTCCCGCATTAAGGTCGAGAAGCAGGTCTATCAGAAGTTCGAGAAGGCTCTGATCGA |
| | | TAAACTGAATTATTTGGTTTTCAAGGATAGACCTTACGGAGCTCCCGGTTCCTATGCCAATGGGCTGCAGC |
| | | TTACGGCTCCTTTCGAATCCTTTGATAGACTCGGCAAGCAGACAGGGGCTCTCTATTACGTGGTGCCCAG |
| | | CTACACATCCCACATTGATCCTAAGACTGGGTTTGTGAACCTCCTGGGCGCAAAGCTCCGGTACGAAAGC |
| | | GTGGTGAAAGCTCAGGAAACAATGAAGAAGATCGGGAGTTTCAAGTTCAACCCCGAGAGGAATTGGTTT |
| | | GAAATCAGCTTCGACTACGGCGACTTCGGGATCGAGATGGCACATCCCCAGTGGACCGCATGTACTCACG |
| | | GCGATGCTTATTGGGGATACAATCCCAAGGAAAAAAGGGCCGTTAGCTACGACGTGACGGAAGAGATTA |
| | | AAAAGCTGCTGACAGAATACGGAATTGATTATGGGACTGGAGCAGATATCAAGGAGGCCGCTATTCGACA |
| | | GACCAAGCCCGCTTTTTGGAAGACCCTGTTTTACATGCTCAAGCTGGCATTGCAGTTGAGGAATACCGTG |
| | | GCTGGCACTGATAACGAGAACGACTACATTCTGTCACCAGTCGAGTATGAGGCCGGGAAGTTCTTTGATT |
| | | CTAGAAAGGCTGGAGAGGACGAACCCCAGAATGCAGACGCCAATGGAGCGTACCACATCGCGATGAAA |
| | | GGATTGCAGGCATTGCGGCGCATCGAGGACGGCAAAGTGAACAAATTCCCGAAGAACGGTGAGCGAAA |
| | | AGCTTGGCTGGCCTTTATGCAAAATAGGGAATATTTGAAAaaacgacccgccgccacaaaaaaggccgggcag |
| | | gcaaagaaaaaaaaggattacaaagacgatgacgacaagtaa |
| GEBx0173- | 157 | atggccccaaagaagaagcggaaggtcGCCTTGTTTTCCGAGAACTTTGTTGGGTGTTATCCAGTTTCTAAGA |
| variant | | CTTTGAAGTTTGAACTGCGCCCAGTGCCCGAAACGAAAGCATACCTGGATGATGATAACTGTCCACTGTTGGC |
| 3 | | TGGTGATCTGGAGAGAGATAAAGCCTACCCGGTGGTGAAGGAATTGTTGGACAAGTACTATAGGTATTTCATT |
| with NLS | | GACATTTGTCTTAAAGATAAAAAGTTGTCCCCCGAAACCATTGACGAGGCCTACCAGGCCTACAGTAGCCA |
| and | | CGACGAGAAATCTCAGAAGGCACTGAAGGCCGCCAGTCAGAAGCTGAGGAAGGAGGTCGCCGGATTTT |
| FLAG | | TCGATAAAAAAACCCGCGAAACCTACTGTCTCGACAAGTACAGTAATTTGTTGGTGCTCGACAAGAAAA |
| | | ACGATAATAAAGAGAAGGGAACGCCCAAGACGATCCCGTCAGTTCTCAAGAAATGGCTTGAGAATGAGT |
| | | CAGGATTCTCCGAGAAAGAGGTGGAAAAATACTCCCAGGCTGTGCAGACGTTTAACGGTTTTACCACAT |
| | | ACTTCATTTCCTACAAAcgAAACAGGGAGAACATGTTCAGCAGTGAGGAGAAAGCCTCTGCCATCTCTTA |
| | | CAGAGTTGTTAATCAGAACATGCTGAGATTTTTCTCCAATGTGGAAAATTTCGAAAACATAAAAAAGAAG |
| | | TACCCTGACCTTTATCAGACACTGAAAGAATTTGAGGAATCCTTTGTGCCTGCCGCCTACTCAAGCCTCCT |
| | | TTCTCAGGCCGGTATAGATGACTATAATTACAGAGTTATCGGACGACCTGCAGAAGACGAGGATTTCGCC |
| | | GGAGTCAATCGCCTGATAAATGAGTATAGGCAGAAAAATGCCGTGCGAAACCGGAACCTGCCAGTGATG |
| | | GTTACTTTGTACAAACAGATTCTCAGCGATCGGGAAAATGGTTTCGGGGGGACATCTTGGCTTCCGACT |
| | | CAGAAGCAAAAGAGTATATTCGGGGGAAGTACTCCAAGGCTTATGACACCGCCTGCCAACTGACTGTATT |
| | | TGTCAATAATTACATACTGACAGAAGATCTGGACACTGTGTACGTTAAGACCCAGCACCTGAAGGATATC |
| | | TCTCAAAGCATTTTCGGAGAATGGTCCGTCCTTGATGACGCTATCAACAACGGAGGGATCGACAGCAAG |
| | | ATTATCTCATTGAAGGCTCTGGCTGAGGCATTCGAGAAATATTGTTCAGCCCTTGATCCCGATATGGCCCA |
| | | AAAATACCGGGACGCCTTCAGTTTGAAGGAGTATCTCAAGAATCCGCCTGCTGTGGAACCCAGACTGCC |
| | | TGAAACGGCCGAGAAGGTTACCGCTTATAAGCCCGCTATGGATGAGTTGATCTCTCTGATAAGGTTTTACA |
| | | AGCCTTTCTATTTGTACGACGGGAAGAAGAGACTGCAGGTGCCTGCCGAGGGAATGGATTTCTCTAATGA |
| | | GTTCGACAAATATTTCGAGGACTTGAAGGAATTTAGTCACGCATACGATAAAGTTCGAAATTTTGCAACC |
| | | AAGAAACCCTACAGCGTAGAGAAGATAAAACTCAATTTCAACCAGCCAACACTCCTGcgCGGGTGGGAC |
| | | GTGAACcgGGAGAAAGAGAATGCATCATTTCTCTTTGTCAAAGAGGGCAAGTACTACCTGGGAATTGCCG |
| | | ACAAGTCCGATAAAAATCTGTTCGATCGGAGTCGGGATGCCGTCAAAGAGGCACTCACTCTTACTTGCGG |
| | | CGAAGATTACTATGAGAAGGTGCAGTATAAGCAGCTTCCGAACATACGGATGAACCTGCCTCGCATAGTT |
| | | TTTAAGCCTAAATGGGAGAAGGTCTTTGCTCCTATAATCACTGAGAGAATCAGCGCCATTGAGAAAGAAA |
| | | AGCTTTACACCGCAGGTGCCAACGATAGGCAGGCGGTGATCGAGTGGATAGACTTCGTCAAGTCTGTCCT |
| | | TGCGCTGCACTCCGAGTGGAACAACTATTTCCAGTTTAAGTTTAAGGAGACGTCCGCATATAAAAATACA |
| | | AAAGAGTTCTACGACGATCTTGATAACCAGGCTTACATCTTGAGTAAGATCAAAATCAACGCCTCATATAT |
| | | CCACAAACTGGTTCGGGAGCGCAAATTGTATCTGTTTCAGATCTATTGTAAAGACTTTTCTGATAAGAAA |
| | | AAAAAGACCGGCGCCGACAATCTCCATACCATGTATTGGAGATGCCTGTTCTCCGATGAAAACTTGGCAG |
| | | CTCTCGCACAGGGACAGGCCCCAATCATAAAGTTGAACGGGAAAGCTGAAATCTTCCTCCGCGAGCCTT |
| | | CTCTCAAATATGAGGCGACGCATCCGAAGAACCAGCCAGTGGCCAACAAGAATCCCCTGAACTCAAAAC |
| | | GGGAGAGCCTGTTCAGCTACGATCTGGCAAAAAATCGCCGCTTTACCGAGAGGAAGTTGTTCTTCCACT |
| | | GCCCCCTGACCATCAACTTTAGGACAGCTGCGGAAGGTGCGCGGAGCTTCAATGAGAGAGTTAATAGGT |
| | | TCGTCGAGAACAACCCCGATATCAGAATAATAGGCATTGATAGGGGCGAGAGACACCTGCTGTACTACAC |
| | | TGTGATCAACCAGAGCGGCAAGATTCTGGAGCAGGGAAGTCTGAACAAAATTAGCTCAGATTACCGAAC |
| | | TCCTGAGGGCGCCGAGGTGGAAAAAGTCACTGACTATCACGAGCTCTTGAACCGGAAAGAAGGAGATA |
| | | GACGAGCTGCCCGACAGACATGGGACACCATAGAAAATATCAAGGAGCTTAAAGAGGGATACCTCAGCC |
| | | AGGTGGTGCATAAGCTGACTACACTGATGATTAAACACAATGCTGTGGTCGTTTTGGAGAACCTTAATCA |
| | | AAACTTCAAACGCTCCCGCATTAAGGTCGAGAAGCAGGTCTATCAGAAGTTCGAGAAGGCTCTGATCGA |
| | | TAAACTGAATTATTTGGTTTTCAAGGATAGACCTTACGGAGCTCCCGGTTCCTATGCCAATGGGCTGCAGC |
| | | TTACGGCTCCTTTCGAATCCTTTGATAGACTCGGCAAGCAGACAGGGGCTCTCTATTACGTGGTGCCCAG |
| | | CTACACATCCCACATTGATCCTAAGACTGGGTTTGTGAACCTCCTGGGCGCAAAGCTCCGGTACGAAAGC |
| | | GTGGTGAAAGCTCAGGAAACAATGAAGAAGATCGGGAGTTTCAAGTTCAACCCCGAGAGGAATTGGTTT |
| | | GAAATCAGCTTCGACTACGGCGACTTCGGGATCGAGATGGCACATCCCCAGTGGACCGCATGTACTCACG |
| | | GCGATGCTTATTGGGGATACAATCCCAAGGAAAAAAGGGCCGTTAGCTACGACGTGACGGAAGAGATTA |
| | | AAAAGCTGCTGACAGAATACGGAATTGATTATGGGACTGGAGCAGATATCAAGGAGGCCGCTATTCGACA |
| | | GACCAAGCCCGCTTTTTGGAAGACCCTGTTTTACATGCTCAAGCTGGCATTGCAGTTGAGGAATACCGTG |
| | | GCTGGCACTGATAACGAGAACGACTACATTCTGTCACCAGTCGAGTATGAGGCCGGGAAGTTCTTTGATT |
| | | CTAGAAAGGCTGGAGAGGACGAACCCCAGAATGCAGACGCCAATGGAGCGTACCACATCGCGATGAAA |
| | | GGATTGCAGGCATTGCGGCGCATCGAGGACGGCAAAGTGAACAAATTCCCGAAGAACGGTGAGCGAAA |
| | | AGCTTGGCTGGCCTTTATGCAAAATAGGGAATATTTGAAAaaacgacccgccgccacaaaaaaggccgggcag |
| | | gcaaagaaaaaaaaggattacaaagacgatgacgacaagtaa |

### Example 11. Gaussia Luciferase Reporter Assay

In set of experiments, the HEK293T was cultured in DMEM media supplemented with 10% fetal bovine serum (Gibco™). A volume of 100 μL of cells with a density of 20,000 cells/well were seeded in 96-well plates 24 hours pre-transfection. Cells were transfected with a lipoplex containing Lipofectamine™ 3000 (Invitrogen™), Gaussia reporter plasmid (100 ng/well), pCasX plasmid (100 ng/well), pgRNA plasmid (harbored MYOD1-TTTG spacer with NNTG PAM, 100 ng/well) and Opti-Mem up to L/well per the manufacturer's protocol. Plated cells were allowed to settle and adhere for 48 hours in a tissue culture incubator at 37° C. and 5% CO₂ atmosphere. Three parallel controls were set up for each sample. The nucleotide sequence of the pgRNA of GEB×0173-wt and GEB×0173-v1 variant is composed of the Cas protein DR (SEQ ID NO: 64) and the corresponding spacer (SEQ ID NO: 84) arranged from 5′-3′ direction. The structure of the corresponding gRNA with is shown in table 10 (SEQ ID NO:144.

Gaussia-Lumi™ Gaussia Luciferase Reporter Gene Assay Kit (Beyotime, Cat.RG072S) was used to measure the luciferase activity, which also indicated the editing efficiency. In brief, Gaussia-luciferase assay substrate(100×) and Gaussia-luciferase assay buffer were mixed at the ratio of 1:100 to prepare working solution. 25 μL working solution was added to each well of 96-Well white plates. The supernatant of cell culture was incubated at room temperature for 5 min. Add 25 μL supernatant from each hole to the 96-well white plate (working solution added) and incubated at room temperature for 5-10 min. The luminescence signal was read on an Infinite 200 pro plate reader (TECAN).

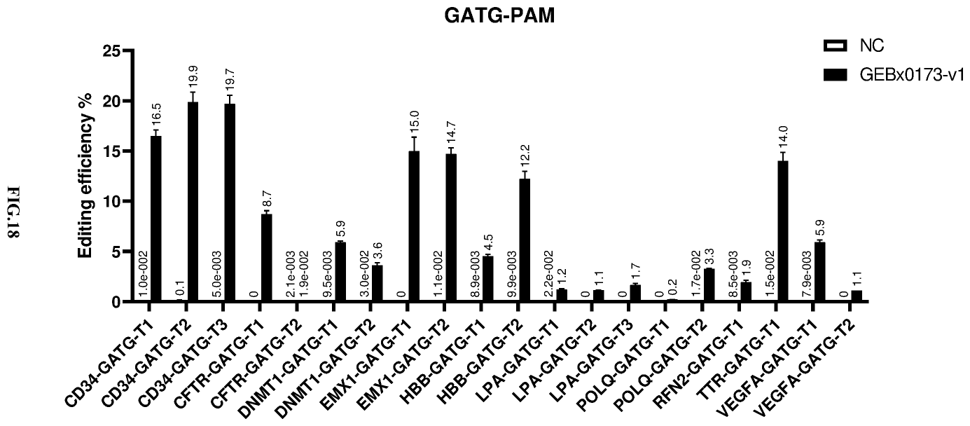

FIG. 17 shows the Luciferase reporter assay result of GEB×0173-wt and GEB×0173-v1 variant on NNTG PAM. Notably, GEB×0173-v1 indicated indel activity on NRTG PAM (R stand in for A and G) while GEB×0173-wt show no indel activity on those PAM.

### Example 12 In Vitro Gene Editing Activity Screening of GEB×0173-v1 Variant

In set of experiments, the HEK293T was cultured in DMEM media supplemented with 10% fetal bovine serum (Gibco™). For lipoplex transfection. A volume of 200 μL of cells with a density of 50,000 cells/well were seeded 24 hours pre-transfection. Cells were transfected with a lipoplex containing Lipofectamine™ 3000 (0.4 μL/well), P3000 (2 μL/well), pgRNA/pCasX (SEQ ID NO: 94) plasmid (125 ng/well and 375 ng/well, respectively) and Opti-Mem up to 50 μL/well per the manufacturer's protocol. Plated cells were allowed to settle and adhere for 72 hours in a tissue culture incubator at 37° C. and 5% CO₂ atmosphere. The nucleotide sequences of the pgRNA of GEB×0173-v1 variant are composed of the Cas protein DR (SEQ ID NO: 64) and the corresponding spacers (SEQ ID NO: 117-136, Table 8) arranged from 5′-3′ direction. The structure of the corresponding gRNA is shown in table 10 (SEQ ID NO:144).

Method of genomic DNA extraction and NGS is identical to that described in Example 7.

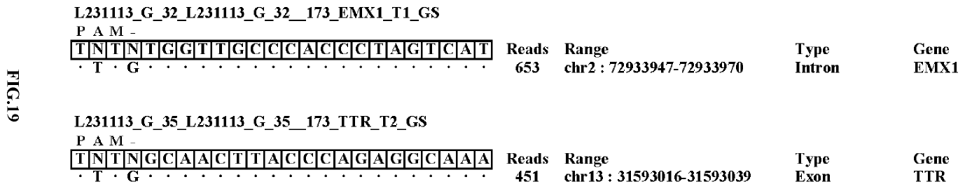

FIG. 18 shows in vitro human cell genome editing efficiency of GEB×0173-v1 on 20 targets with GATG PAM. 7 targets indicate over 10% indel.

In this disclosure, the editing efficiency (e.g., the “editing percentage” or “percent editing” or “indel frequency”) is defined as the total number of sequences reads with insertions/deletions (“indels”) or substitutions over the total number of sequences reads, including wild type.

### Example 13 Off-Target Profiling in Cell Lines Using GUIDE-Seq

GUIDE-Seq leverages a dsODN to insert into the double-strand break site generated by CRISPR/Cas. The HEK293T was cultured in advanced DMEM media supplemented with 5% fetal bovine serum (Gibco™). Cells were seeded at a density of 100,000 cells/well in a 24-well plate 24 hours prior to transfection. Cells were transfected with 400 ng of pCasX plasmid, 150 ng of pgRNA plasmid, and 10 pmol of dsODN using Lipofectamine 3000 (Invitrogen™) per the manufacturer's protocol, cultured at 37° C. and 5% CO₂, and harvested on day three post-transfection.

For GUIDE-Seq library construction, an amount of 500 ng genomic DNA was used for GUIDE-Seq library construction. Briefly, DNA was fragmented by KAPA Frag Kit (KAPA Biosystems), followed by adaptor ligation and two rounds of hemi-nested PCR enrichment for dsODN-integrated fragments. Final sequencing libraries were quantified by KAPA Library Quantification Kits and sequenced on a Illumina NextSeq 1000 System. Data demultiplexing of Index 1 was performed by bcl2fq (version 2.19), followed by custom scripts for Index 2 demultiplexing, adaptor trimming using the BBduk tool, and analyzed by the GUIDE-seq software. Briefly, unique molecular index (UMI)-tagged FASTQ file was consolidated to generate UMI-consensus sequence, and aligned to human reference genome (hg19) using BWA MEM. High-quality alignments (MAPQ≥50) were used for identifying genomic loci harboring the dsODN as the potential off-target sites. Candidate loci with up to 6 mismatches against the corresponding on-target protospacer were identified as bona fide off-target sites.

For the GEB×0173 guides targeting TTR and EMX1 gene, no off-target site could be detected at EXM1-TTTG-T1 and TTR-TTTG-T2 sites (FIG. 19).

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. An engineered, non-naturally occurring Cas12 protein comprising an amino acid sequence having at least 70% sequence identity to SEQ ID NO: 12.

2. (canceled)

3. The Cas12 protein of claim 1, comprising one or more mutations in REC.1 domain, and/or WED.2 domain of SEQ ID NO: 12.

4-5. (canceled)

6. The Cas12 protein of claim 1 comprising the mutations at positions corresponding to: I182, K532, E535, N536, and K586 of SEQ ID NO: 12.

7. The Cas12 protein of claim 3, wherein the Cas12 protein recognizes at least one PAM sequence selected from; AATG, ACTG, AGTG, ATTG, CATG, CCTG, CGTG, CTTG, GATG, GCTG, GGTG, GTTG, TATG, TCTG or TGTG.

8-12. (canceled)

13. An engineered, non-naturally occurring Cas12 polynucleotide encoding the Cas12 protein of claim 1.

14-17. (canceled)

18. An engineered, non-naturally occurring Clustered Regularly Interspersed Short Palindromic Repeat (CRISPR)-CRISPR associated (Cas) (CRISPR-Cas) system comprising:
(a) the Cas12 protein of claim 1, or a polynucleotide encoding said Cas12 protein; and
(b) a guide RNA which comprises a spacer sequence linked to a direct repeat sequence, or a polynucleotide encoding the guide RNA;
wherein the spacer sequence is capable of hybridizing to a target sequence.

19-20. (canceled)

21. The system of claim 18, wherein the direct repeat sequence comprises a stem-loop structure and the direct repeat sequence comprises a nucleotide sequence having at least 90% identity to SEQ ID NO: 29.

22. The system of claim 18, wherein the spacer sequence is between 18 and 23 nucleotides in length.

23-34. (canceled)

35. A method of modifying or targeting a target DNA locus, the method comprising
delivering to the locus the Cas12 protein of claim 1, a polynucleotide of encoding said Cas12 protein, or a CRISPR-Cas system;
wherein the CRISPR-Cas system comprises: (a) the Cas12 protein or the polynucleotide; and (b) a guide RNA comprising a spacer sequence linked to a direct repeat sequence, or a polynucleotide encoding the guide RNA; and wherein the spacer sequence is capable of hybridizing to a target sequence.

36-45. (canceled)

46. The Cas12 protein of claim 6, comprising the following mutations: I182S, K532V, E535N, N536R, and K586R of SEQ ID NO: 12.


SEQUENCE LISTING (187 sequences)

SEQ ID NO: 1 - protein, 1250 aa, synthetic construct
KTMTDLTGLYPLSKTLRFELKPIGKTLEHIERKGLIAQDEQRAEDYERVKDLIDKYHKKFISLCLNKCRLKVESTGEYDSLEEYVEFAGIAKRTQEEENAFTKVKEMLRKTVADSFKKDPDQFKELFKKEIITEHLPNMLLTDGSEEDKKKVESFALFTSYFTGFHQNRENMYSAEEKSTAIAYRLIHQNLPMFLDNIKSFEKIQATIDGKDMEELENAFRECLNVEHIADMFQLAYFTEILTQEQIDVYNYIIGGKTEEDGTKIKGINEYVNLYNQQHKDSRLPLLKPLYKMILSDRTTLSWLPEQFKSDEEMLTAINEAIKKLNDEVLNTANQDNLQQLLSHLSDYDTRHIYITNNHCLTDISQQIFGQYDRYTTAIKEEIKSSLKPTAKEKKDPDLLNDRTRKLFDARKSFSIAELNEYAQAEANQTIEVYFAQLGSYDRNGEQQISLLSRLNMAYNEAKEVLDGKYTNLNQSKEATEKIKNLLDAYKAIQFFAQPLAGSGDEAEKDNEFDAKLRNLTNTLSEVITPLYDKVRNWLTRKPYSTEKIKLNFENAQLLGGWDVNKEPDCTAILLRKHKQYYLGIMDKKSNHSFDIDELPSEGDCYEKIDYKLLPGANKMLPKVFFSKSRIDEFNPSDAIKKAYEKGTHKKGPDFSLADCHRLIDFFKASIAKHEDWKRFPFSFSDTGSYEDINGFYQEVEQQGYMIGFRPVSEAYINQLVEEGKLYLFQIWNKDFSEYSKGRPNMHTMYWNMLFDEKNLADVIYKLNGQAEVFFRQKSLDIAKTCVHAAQQPINNKNKSNQKQQSTFDYDIIKDRRYTMDKFQFHVPITMNFKSKGRENINSQVREIIRNNGIQHVIGIDRGERHLLYLSLIDLKGNIVKQMTLNDIINEYKGNTYATNYKQLLEYREGERTEARRSWQTIENIKELKEGYLSQVVHVIAKMMVEYNAIVVLEDLNGGFMRGRQKIERNVYEQFEKKLIDKLNYYVDKQKEATEAGGVLHALQLTSKFESFQKLGKQSGCLFYIPAWNTSKIDPVTGFTNQLDTRYQNVEKALQFFGRFDSIRYNKEKDWFEFMLDYDKFTTKAEGTQTRWTLCTYGTRIRSFRNDINKWVPKDVVLTEEFKQAFAAANIDVNGNLKDAICNLNEKAHLEELMHLVGLLVQLRNSKTNTEIDYMLSPVADKNGVFYDSRTCPDTLPKDADANGAYNIARKGIWVIRKIQEDQTNDKVDLKITNKEWLAFAQQKPYLKHL

SEQ ID NO: 2 - protein, 1244 aa, synthetic construct
EPIGKTADYIKNYQYLETDERLAKESKKVKELADEYHKEFIGDVLSSLELPLSKINELWDIYMSNDTDREIKFKKLQENLRKVIAEAFSKDKRFGNLFKKEIITDILPKFLQDKDDDIKIVNRFKGFTTYFYAFHKNRENMYVSEEKSTAIPYRIVNQNLVKYFDNYKTFKEKVMHLLKDKNIVESIERDFKDILNEKSIEDVFGLANFTHTLCQADIEKYNTLIGGLVVKNEKKEIKGINQYINEHNQTSKKGNGIPKLKPLFNQILSDRKSLSFTLDDIKKTSEAIRTIKDEYENLRDKLATIERLIKSIKEYDLAGIYIKMGEDTSTISQHWFGAYYKIIEAIADAWERRNPKKNRESKAYSKYVSSLKSISLQEIDDLKIGEPIENYFATFGTTCSDRTSGVSSLNRIKAAYTEFVNKFPEGFEDGDDCNDAYFKANVEVVKNLLDSIKDFQRFVKPLLGNEDERDKDEAFYGEFVPTYTDMDNIITPLYNRVRNFATKKPYSTDKIKINFENVVLLKGWDKNKESDYASIILMKDGQYFLGVLRNGSKSTLKTILPNTGDCYQKMVYKYFKDIKSNLPRCTTQRKDVKAHFAESSDDYTLLDTKAFVSALTISREVFELYNAPDKEKKFKKEYLKNTNDSIGYANAVSVCKRFCLEFLKKYRSTAIYDLSDVETSVDSFDDLSSFYQEIDKRLYSISFENVSVDSVNELVDNGNMLLFRIANKDFSPNSKGRPNLHTIYWRMLFDPANLKDVVYQLNGNAEIFFRKASVTRTEPTHPANVAIKNKSEYNKQNKPYSTFKYGLIKDRRYTTDQFEFHVPITMNFKQPESSKLQDKLNKQVLDFLKQDGVRHIIGIDRGERNLLYLVMVDMEGKIKKQISLNEIAGNPKNPEFKQDFLALLHEREGDRLESRRSWNTIQSIKELKEGYMSLVVHEIANMMLENDAIVVLENLNRSFMQKRGEKSVYQKFEKMLIDKLGYIVDKTKDVSDNGGALHAVQLADTFENFNKTQKGAIRQCGFIFYIPAWRTSKIDPVTGFVPMLRCQYESIVESKKFFGKFDSIYYDATGKYFVFQTDFTKFNTESKGGIQKWDICTYGDRIYAPRTKDRNNNPVSERVNLTEEMKSLFVSHNINIQGDIKAGIMQQTDKEFFESLHRLLRLTLQIRNSKKSTGKDYEDYIISPVMGKDGRFFDSRNADATQPKDADANGAYNIARKGLMLLRQIQAQEKQDLSNGKWLEFAQR

SEQ ID NO: 3 - protein, 1301 aa, synthetic construct
TRKFDDFIHQYSLSKTLRFEARPIGETLHNFIKNGLLERDEHRAESYVKVKKLIDEYHKAFIDRVLNDGCLNYEDKGEYDSLTEYYALYSAPRKDEAEREHFKETQQHLRDQVVKKLTGDNAYKHLFGKELIESYKDKEDKKKLNQADLVQFINTATPKQRLNFSKEEAIDLVQEFCGFTTYFGDFYQNRENMYSAEEKSTGIAYRLINENLPKFIDNMETFKKIAAIPEMEDNLKEIHDNLAEHLNVENIQDMFQLNYYNQLLTQKQIDVYNAIIGGKTDDEHKEKIKGINEYVNLFNQAHKDAKLPKLKTLFKQILSDKNAISWLPEEFDNDQEVLNAIKDCYARLSENILGKENLKRLLCSLSEYDTKGIFLRNDLQLTSISKKMSGSWTDIPNAIKNDLKDGVPAKKHKESEEDYEKRIDNLFKRQDSFSIGYIDDCLNKFDDNNTFTIEGYFKKLGAKDTQSEDIIKQIANAYTDVKPLLNSTYPKSKNLSQDKENVVKIKRFLDTLMSLVHFVKPLLGKGDESNKDEKFYGELSLLWTELETIVPLYNMVRNYMTRKPYSNSKIKLNFDNSQLLGGWDANKESDYASILLRRDGKYYLAIFDKDSKKLLGKSMPSDGECYEKMVYKFFKDVKTMIPKCSTQLKKVVKHFSISDTDFVLSDENFKVPLTITKEVYDLNNTMYGKYKKFHKGYLTTTGDIDGYTSAVKTWINFCMDFLGAYGSTMSYDLSSLATKEYSSLESFYQEANRLLYTVNFVKVSTSYVSRLVTEGKMYLFQIYNKDFSEYSKGTPNMHTLYWKALFDDRNLKDVVYKLNGQAEMFFRKKSIDCNRPTHPANQPIQNKNKDNKKKESVFEYDLIKDRRYTVDKFMFHVPITMNFKSTDKENINLPVMEYLQTSNDTHIIGIDRGERHLLYLVVIDLHGNIVEQYTLNDIVNEYNGNTYRTNYNDLLEARGNEQQKASQNWQTIENIKELKEGYLSQVIHKITQLMIKYHAIIVLEDLNMGFKRSRQKVEKQVYQKFEKMLIDKLNYLVDKKADIESTGGLLNAYQLTNKFVSFKKLGKQSGFLFYIPAWNTSKIDPVTGFVNLLDTRCQNKNKAKAFFAKFDSIRYNKDKDWFEFALDYDKFGSKAEGTRTKWILCTQGKRIKTFRNAEKNFQWYNQVIDLTKDLKQLFAQYDIDINGNLKEAISKQTKKTFFDDLMGLLKLTLQMRNSITRTETDYLISPVADEIGNFYDSRTCGPSLPENADANGAFNIARKGLMIIEQIKASEDLSKLKFDISNKSWLNFAQQKPYKHE

SEQ ID NO: 4 - protein, 1282 aa, synthetic construct
NTSKFIFQNLTHQFQLQKTLRFELIPQGRTLEHIEKNGLLEADELKANRYKQVKEWIDLYHKAFIEKVLREPKLEGLSEYRVFFENKQRNEKDEEAFKKLKSDLRKKISSFFKDEPNKTFYKRLFDKELIKADLPDFLREQGMHEEATMLLEEFGNFTTYFKGFHENRKNLYVAEDKSTAIAFRLIDENLPRYMQNLLLVEKHREVFEQFFPQIQERLGKELDGLRVEEFFSEEGFNLCLNGSGIHQYNTILGGVKGEEEEGIPGLNMLINLYRQKHKLKSRELPTLFPLHKQILSDRESSSFRPEPYEDGDENTVVEDLLKLGDQWFNPEGFVPQLVAHLTKLKDEEQDQLFVNNKANALSAVSQATLGHWAVLGEALSSYWNHVHDVKENALKGKALTAKEKWLKQPYFSFQEIELALRHYMGAEGKENASKWNVWLEKPLVQYFNYGLTKEAEKTKVDTAKAAWGHAENILRELQNHENPKLKTKETKDAIKAYFDACLDLLHFIKPLVVDLEQCEASPTFYGDFLSLYHEMDDLVPLYNKVRNYMTKKPYSYEKFKLNFNKGTLLDGWSQSKETDNLCVLFREGNNFFLGIMDRKHNKSFVKPPMAKEEEPVMQKVVYNLLPDPSKMLPKCFTAPYFRRIAKEKTKDPEQIVSEYSKEKGLIKPKLDVLDAYAHKKHTKENFQRNGFEQVVEYFKEGAQKHPDWHVFDFKFKPTSAYKDVSDFYQEVTEQGYCLDFVDIPKRYINDLVESGKLYLFQIYNKDFSKYSKGKPNLHTMYWKAIFDPQNLKAPIYKLNGEAEVFFRKKSLEYTQEQLDTGHHREELKGKFDYPIVKDRRFAFDKFQFHVPITLNHARKNQAVGPQVLRYIQKHKEELHYIGLDRGERHLLYLSVVNAKGEIIEQRSLNKIGNGFVKDGKPYTHDYQGKLLEKERNRDSARKDWDAVESIKELKEGYMSIIVHEIAQLVVKYNAVVILEDLNFGFKRGRFKVERQVYQKFERKLIEKLNHLVFKEVAPQEPGGLLKALQLTDQFTSFTKLGKQSGILFYLPAAYTSKIDPLTGFINQVPWKYTNLEAAQKWIGNFKRISYHAELDLFEWVLDQRDFLSNPAGCKHQWVLRMDNQERWHWNSKAEGTNGKGAYSPIRLKEQFEDLLARQNISYNGNSKLLEDIQAVKDASFFKTLFFFLRTLTQLRYNNGKSGAEEEDFILSAVANPGMEPFDTRKAKQGLPLDADANGAYHIAMKGLMTLGQLKEKPNGKLEIPKLDKSAWLCFMQQRSNQG

SEQ ID NO: 5 - protein, 1262 aa, synthetic construct
QTKNFMSFSDFTNLYALSKTLRFELRPIGRTEEFFKKNKVFEKDKTINDSYNQAKLYFDEMHRRLIRDALNTQNTSALDYEKYAKHWKELQKDKGKYIQNWKDAQKEFRECISNLLDQQAEQWKSKYASVGLNKADIKQKGTDFLSSAGMVKILKKEFPKEAENQLVKEGFPSLFIQKAEAPEQKQYIFDSFDRFSTYLAKFNQTRDNLYKDDGTPTAIATRVVENFVAFLQNQAIFNNHYISLKSELDFSREEIDCFTANYYVRCLLQEDIDEYNKTIGQINARIKELRDRKQKESDFKKSGYPLLKSLNKQILGKIEKKRQFIENKADVFPAFSELIEQANARLTALRQLINNLCEEVFEEAYGGIYIKNTGINTISRRWFASWYDFEIALPQKSKNEEESKVVTFVSLQDIKDTLENKLSGDIFKEEYYKKKIINKEQSNWKQILAILQNEWNELFSEYQKALENTKKLDLNSVFSNSKEQVEIIKSFADASLRVYQVVKYFALEHKKSDEIPSSYSTDFYEEFDKQYNNFEFIPYYNALRNFLTKKVVGENKIKINFEKGNLLNGWAESPEGNAQFCGYILKKENKYFLGITDQPTILDTKDNPSLTNITDKEYYEKMAYRQLKSTTIYGSGYEGLYNSKYANDKKYISESKIIERVKKLLQEKYLTRFPELKQIIEKEYKDANELAKNLGNFNLYSLEFIPVNKNYIEKLTHDKNGSIKYLYIFEIANKDLGKITPRKYNIHSLYFKNLFSEDNLEKPVFKLSGGAEIFFRPKTVNLPKKKDKAGKDIIDHKRYAQDKLFFHLPIVINFGAGKVGKFNQELNKKLLAGNKNINIIGIDRGEKHLAYYSVINQKGEIIEQDSLNEINGVNYYGLLQKREKERLENRKSWLPLQQIKDLKRGYISQVVYKIVRLAIEHNAIIVLEDLNMRFKQIRGGIEKSIYQQLEKALIDKLGYLVFKDRNAKEVGGVLQGYQLSAPFESFQKIGKQTGIIFYTQASYTSVTDPLTGFRKNIYVSNSAPIEKIKVAINNFEEIGWDDVVQSYFFNYDTKNFSNGNDIQSKKWKIYAKVPRIKREKVNGYWQYNDVDPNDMLEKLFRLYEFKNLRSNMLEQIHAKETNGELRGERDFDNKPRNFYQSLVYILNLILQLRNSTSKQIKLNEKEDVYETGEDVDFIASPAYPFFTTLSSKSNSNLAGFENRFTGLNHDQEQKKFNGDANGAYNIARKGIIILERITKNSDKPNLYISNEDWDKFVSQSYP

SEQ ID NO: 6 - protein, 1277 aa, synthetic construct
ESKTLDRFIGKYQLSKTLRFELIPQGKTKEHIEQKGLLQQDEHRAKAYKMVKKIIDRYHQHFIATALNNLALDGLQSYEELYHKKEKEEADKKNFESLKAGMRKQIGDAFNKSEASKVQEIWKVLFDKELIRKALPEFVTSEEDKKLLAEFKDFTTYFTGFHQNRANIYTHEEKATAVAFRLVHENLPKFLDNSRVFDWIQTEYPDLDLSAIEKELAPELQGTTLPEIFALSFFNHTLTQNGIDLYNAVLGGKTTEQQNKIRGLNEYINLYNQRRTAEAKKEGEKFRRVPQFKELYKQILSDRESISFLPETFENDSEVLSAIENFFATQLVHFEKDNRSISVFAELQKLLQELKEDDRSKIFVRNDLALTNISQNIFGDWQVIKNALEHYYTTTVNPLAVGKKLTKAYENGKEKWQKQSYYSIYEIEEALLAYSSEADVLKEKNLTAASRPLCNYFATFSKDEEGNNLFQRIETAYTAVKDLLNTDYPEEKNLAQDKTNVAAIKTFLDAIKNLLHFIKPLHPARLEGEKDERFYGAFIPLFDQLNLLTPLYNKTRNYLTRKPYSTEKVKLNFENSTLLDGWDANKETANTSVLLRKDGLYYLAVMDKKYNKVFEQMPEVAEGEKVFEKVVYKLLPGANKMLPKVFFSAKNIQYYNPSTELLENYKNETHKKGATFNLVHCHQLIDFFKASIARHQDWKNFNFQFSDTGSYADLSGFYREVEQQGYKITFHNLPESYINQLVDQGKLYLFQIYNKDFSPFSKGRPNLHTMYWKMVFHEGNLTNVVYKLNGQAEIFFRKKSLNYDEKTLREGHHAAALKDKFAYPIISNRRFAFDKYQFHVPITINFKSTGRDNVNAEVLSFLQHNPSVNIIGIDRGERHLLYLTLIDQKGNIKKQMSLNQIINSYRNKEGDMVNVKTDYQNKLHLKEEERLKARQGWGLIENIKELKEGYLSHVVHEIAKMMVEHNAIVVMEDLNFGFKRGRQKVEKQVYQKFEKMLIDKLNYLVFKDRTATEPGGLLHALQLTGSYTDFMKYKQKQCGFLFYVPAWNTSKIDPLTGFVDFLKPKYESIEKAQNFLAKFKSIRYNPAKQWFEFTFDYKDFTDKAEGSRTRWTLCSTNQPRYRWNPKAANGKGAQEAVNVTQALEDLFSAHNIAYGSADDLVTAITAQSSADFYKALLRAMVTLLALRHNNGKAGTDEQDYILSPVEPFFDSRLADDSRPKDADANGAYHIAKKGLWVLQQINELQGDDWKNLKLAISNKQWLQFAQQLNADISPKVS

SEQ ID NO: 7 - protein, 1342 aa, synthetic construct
SSSIVSVSTEKKQAEVLSNKSVFDQFTRKYALSKTLRFELRPVGKTRENIEKANPNFIRDKEIEDAYQILKPVFDKLHEEFITESLESDEAKHLSFEDYFSVYKNLRNEQDREEKKKIEKSLETKEKNLRAAFSDIYKAEGEKFKSKVGSDKKGKPILKEDSFKILTEVGILKYVKAKIEEFVVMNLKTHEGKEVKKEDLEKALGTADSKGVFEGFFTYLTGFNQNRENYYSTEEKATAVASRIIDENLPKFADNVLEFQKRKDEYLKVYDFLKENGIETIGKNQKGEKVELEKIIADIFEIGYFTNCLSQREIEKYNLNIGNANNLINRYNQQQSEKNKKLRIFKTLYKQIGCGEKGDFIPAIKNDDGLRAVLKNVVENGEKYFGQFDGKIEDKVDTVGEFVHYIINHESFEGIFWSDKALNTISGKYFANWFILKKALKEGGVFGGKKKDDEDIKIPQAVELKNLFEILDKTEKWKNKEDGLFKTSLFDGEGNKKGIIERAEKPSKALLGMIFADVEKMAKDFSELSPKVLSITTDFKKPEHIENIKMLLDSALYVNQIVKYWKVKDKFAVDPILAESLKAILFGDNNPTKFYDIIRNYLTKRPQEGLNKLKLNFDNSVLGGGWDVNKEPERWCLILKDGEKKYLAILTKASQKLFGEKAKYKEVVTDENYLKMDYKLLPGPNKMLPKVLLPKRNRKEFGATDDILSIYDEGGFKKNQPSFTKEKLWKIIDFYKEGLKVYPSAEKSWQDLFGFNFSPTEKFESIDQFYNEVEKQGYKLSWNIISKKLINEAVENGEIYFFEIRNKDNNLKEGKEKTSEKNLHTIYWNEIFGDVANKPKLNGEAEIFYRKTILKKKENETDKEFEIRLKEKLGKKKDKSGKEVLEHKRFANEKFVFHCPITLNFCLRSLKFNDEINESMVKLNDDITTIGIDRGEKHLAYYFVVKQTKDGKIETLDQGSFNEINGQNYAEKLEKMAGNRDEARKNWKTIGTIKELKDGYISQVVRKIVDLAIYEDVEKKVFRETPAFIVLEDLNIGFKRGRQKIEKQVYQKLELALAKKLNFLVDKHAKEGEIGSVTSALQLTPLVNNFGDIEGKKQFGIMFYTKADYTSQTDPVTGWRKTIYLKKGSEESIKKQILDNFDDIKFDGKDYYFKYTDRNTNKPWELYSGKDGESLDRYYRELVHENSDKKWVPKEQNLKKILDGLFEGFDKEKSLLEQLRQGKNPNKINDRTSWESLRFVIDLIQQIRNSGKTEEDADFILSPVRDKNGNHFDSRKINLNLPTSGDANGAYNIARKGIIMCEHIKRDLSLFISDEEWSAWLAGREIWENWIKKNEKNLKRKQ

SEQ ID NO: 8 - protein, 1289 aa, synthetic construct
KSIEELIGQYSLTKTLRFELKPIGKTLEQIERKGVLSADQTRADQYKRVKESIDRYHKAFITESLDSLVLKVKSTGNTSDSLEEYASLLDNSADDEALDKVKKGLRKQVAERFDKQIRLKKLFKNDLITKILPEFLKVNGNEDERKAVESFRNFTTYFSGLNENRKNMYSKEAKSTAIAYRLIDENFPRFHDNMLSFAKIAAVNEVSNHFAEIAEAFKSEMVVASVAEMFGLEYFNNTLTQQKIDLYNYVIGGKTEGDKVKIQGINEYVNLYNQNHPNDKLPLLKPLYKMILSDRVALSWLPEEFNDDEAMVCAIKEFYNAIQPVLWGEVEDDDNSVCHLLKNIGKYDIEHIFISNGAALNDVSRQMFGQYDFLANAIKKDIRQNTKRSSRETDEKFEGKVNTLFKKAKSYSIAHLNALIKENRSETIESFYSNLGACDRDGEQKINLLTQIELAHEAAQDVLDGKYADINRDEKATMLIKELLDAFKALQYFIKPLLGSGEEASKDNVFCTKLLNAWQALDAVTPLYDKVRNRLTRKPYSTDKIKLYFENKGKFLDGWVDSKTDNSDNGTQFGGYILRKKNEIGEYDYFLGISSDTKLLRANPDIQYEHGMYERLDYYQIQSKTFLGSSYKGEYNDDAMRLMSAFVEYGKKSRFDSCVLPTDKEKSLPTYLKRIYTDNNSFYNEAMTDTVVAEAYNNIKSHLLLTLAGIKRVEAAVNLSKHNEYNLIQIFDEIGKITSSSKIFSFFPVDTSLVEKLNERDNKPLFLFKISNKDLSYAETSSKNIRKSRGKENLHTMYFKALMEQSHNVFDIGTAEVFFRKATPGLDKSTTIHKANEALCNKNPHSKKTSSTFGYDIIKDRRYVENKFQLHLSMVINYQGKNPNTDIISPQVLDIIRNNGIKHIIGIDRGERNLLYLSLIDLKGNIIKQISLNEIKPLGNDGCVTNYRDILADREKDRKEARRSWKKLENIKDLKSGYLSQVVHVIAKMMVDYQAIVVLEDLNSAFMQSRQKIERSVYEQFEHKLINKLNYYVDKSVSDDLPGGLLYGLQLASKKTHDVQCGCIFYVSAWNTSKIDPVTGFVNMLDTRFENVSSSRSFFSHFDSIRYNVAKDWFEFTFDYNNFHKKLEGTKTRWTLCTYGTRILTQRNDRNQWESREVDITDEFKKAFVAAGIDIHGNLKDAICALDKRVHLEQLMRLMKLLLQLRNSIINQDTDYILSPVAENGVFYDSRRHADNLPIDADANGAYNIARKGLYLLRKIKADRSAKPELCISNKEWLRFVQDKSNPGD

SEQ ID NO: 9 - protein, 1326 aa, synthetic construct
VTIDDFCGQKNGYSRSITLRNKLIPVGKTQENLEKANMLEEDFSRAKAYVEVKKIIDDFHRSFIEDVLSKANLNWKELYEKIDSFQRETDKAKKAKNQKDLQPVQVKMRKAIVKIFKDDERFDKLFKKELLSDLLPEIIKSAPENEISNKQTALDVFKGFATYFTGFHENRKNMYSEEDKATAISYRIVNENFPKFYANIKLFEKIQKEFPVIIEETEKSLASFLAGKKLKDIFTPEAFNQVLTQSGIDFYNTVIGGIAGEAGTEKVQGLNEKINLARQQLPDEEKSKLRGKMVVLYKQILSDRETASFIPVGFENKDEVYKTLQEFKKSTVEKSIEIIRETFKSPENFDLSKIYVPAKELTAFSLSVFGRWNILSDGLFAIVEEKSKKALSEKQTEKLANEIAKKDYSLSELQAAYEKYLEKNPVEDEGRCRRLSAKNYFELIELRKIDDSAEKEFVNVLSNLDELWSKIDFENPRNLQQEKEAATPIKNYLDEVQNLYHFLKLVDYRGEEEKDSEFYSKLTEILDLLSEIVPLYNKVRNFVTKKPGEVKKIKLNFDCSSFLKGWGTEFGTKDAFIFIDEKKYYLGVINKKLDSDTVKYLQTPGEHIIKQVFVNFQKPDFKNFPRLFIRSKKDRFAPAVEQFNLPVNDILDIYDKGLYKTEYRKENPIIYKESLIKMIDYFKLGISQHESFKGFKLTWKESSEYENISDFYADVTASCYQVNFTNVNFDRLLSLANEIEDDNKKIFLFEIYNKDFSTGKNGGNGSTGKKNLHTMYFENLFSKENLKDIVLKLSGEAEIFLKSENTELKKIVHKQGTTLVNRTTKDKKLIPENIYQEIYQFKNSMIDNISDEAKAWLNSGNVECKIAKFDLIKDKHLIEKSYYLHCSIIQNFKAPEKVFNFNEKVQRYLKDNPDVKIIGLDRGERHLIYLSLINQKGEIELQKTLNLVEQVRNDKTVKVNYQEKLVQKEGDRDKARRNWQTIGNIKELKEGYLSNVVHEIAKLMVEHNAIVVMEELNFGFKRGRFPVERQVYQKFENMLIGKLNYLVFKDKSVTEPGGVLNAYQLTDKSTNVSDVGKQCGWLFYVPAAYTSKIDPKTGFANLLNMKDLTNVAKKRMFFSKFKDIHFDSGSNDFAFTFDYKDFDGKGAAEMTQTEWTVWSNGKRIFYNSKHHSTEEINVSEKLKELFDASKIDYKTENNIFESIMEVGADLKEGEKPSKEIANFWDSLLWYFKLILQMRNSIPNSETDYLISPVKAEDGTFFDSRVQKELGKDSKLPIDADANGAYHIALKGLYLLKNDFNLNDKGYIENISNAEWFRFVQERDYLN

SEQ ID NO: 10 - protein, 1301 aa, synthetic construct
PNLFDNFTQQYSLTKTLRFELKPEGKTLENMQNAFKFDKDLQTFLVDQDIEDAYQTLKPYIDFIHEEFITESLESDIAKGIDFSEYFENFKIKKDINQKDFQNLEHKLRKEFDQLYIQIANKWKEEKYKQYKWKKGSKVANGSEILSSEDILKLVVDLNQDNNQLKDIVQNTFKGFFTYFSGFNDNRRNYYQVEKEAKSSIATRIVNENLPKFCDNFLLFENRKNEYLGVYNTLKLLGKDLVDKDRKTLVTIEEIIFSIKHFNFCLSQKEIENYNKQIGNANLLINLYNQNTSNESDYKRLPLFKTLYKQIGCGKRDSLFFEITDYTKLEAEGKRILGKSAISVEEVLIQVKESGIKYFTEKLDSLLNYILEKETDNYQGLYWSNIAVNSISSKYLANWNNLKELFKGKKEVASYDKGREEEIKINEAVDLSILFSILNNTEDWKVKEVFFKENLNEESETKKIIETSKTPSQALLRIVIGDLKKNASNFLNLSDNIEQLLESKEKIKESMDYALSVTRMLKYFLVKESKTKGAPLDSKLVEDLNLLLLSDDAPWFMWYDGLRNYLTKKPHDDAKENKLKLNFENSTLAGGWDINKEPANYCVILTNSANDQYLAINAKRNNKIFCKELVDGKGKDKKLLKNPLYESDEQHWKKMEYKLLPGPNKMLPKCLLPKSNLRKFGASEEVLSIYENGSFKKNTPDFSTQDLHKLIDFYKSALLKYEDWKCFHFKFNDTRSYGDISQFYSDLEKQGYLLSFIDINKSVLNQLVDSGKIYLFQIRNQDSNISKKINHQDNLHTIYWRAVFEDVFNKPKLNGQAEVFYRKALPQEDLQLLKDKDNKEIIANYRFSKEKFLFHVPITLNFCLKNKKINDLANREILSNDNIYFLGIDRGEKHLAYYSLVDQKGNIVIDQNGKSIQGTLNIPFLDSNGNQRLVKATKRVMKDGKEDAQVVECKDYNELLEARASDRDYARKNWQTIGAIKDLKDGYISQVVRKIADLAVEYNAYIVLEDLNTGFKRGRQKIEKSVYQKLELALAKKLNFLVDKSAKIGEVGSVTNALQLTPPVNNYSDIENRKQFGVVLYTRANYTSQTDPVTGWRKTIYLKTGSEDIIKKQIFESFSNIYFDGQDYIFTYTDKNIDRKWELYSGKNGKSLDRYRGRKVNNKGWVIDKINIQEILDKIFINFEICYGEKNNIFEKLKAGVELEKYNEYTAWESLRFAIDIIQQIRNTGIESSDSDFLLSPIRVNNLHFDSRLASENQPNSGDANGAYNIARKGIIMANYIKQGVSPFVSDIDWDNFVTKN

SEQ ID NO: 11 - protein, 1404 aa, synthetic construct
NKFLDSFTHQYSLSKTLRFELKPVGKTAEWIQKRDIIGTKKQENGEAQLTGKDAVRAENYEYAKKLLNEMHRLFIEDSLAAIKEEQKVELNKLLVKIAESGSLDGFRKPMSNICKELFDSATQDWLLAYSEEMPCFWDEDILELTNKAETTENKKNIKFFLKKAEDIRKFKDDPLKAFKKKKHEALNSNTEALKLLEWKIRRGEVLISGKDLGRDESEEILSTKELILILRSFYSFVTYFTGFNENRANIYNLDDKKYISTSIAFRSFEQNLFFHIENIKKWHTITNSIEEHTDQLNASDYPIKDKLRELELAFDCKLADLFAVDSFSQFLSQSGIDRYNSIMGGIAQKTGKTKVQGLNEIINQARQQTGAKRHKFPPMKLLYKQILSKGEGIFVESYVDDADMLTSIAGFHENHLNESLCSNWLHELEELIDSVQESKDTIFIARDKLNRISQEITGSWSAIQDWIVAHQKDQKEVDALTKKKVFSIQELEDYFSKSSDDELFTAKIPEDRSAYLPGEGEGILFQFFKVRAKEILAELQSGWYALDKAQLLQEKQLDKNRENEGSKGFEQVGLLKGYLDACNSLSRFIADWNIQKDIKQERCSLWYEFLQKHLDTVPIFDLYNQVRNHVSKKAYSTDKLKVNFEKSTLLDGWDRNKESQNFGVLFEKDGAYYLGIMTPDSNGIFEYDVNPDENTKKKAIKEELRKQTTPENGEDRFQKINYKLLPGPNKMLPKVFFAKSNEDLFQPSARITEIKENKKYAKAYTDKHGKAELHEYIDFCQKSLVRHPEWSVAYGFDQTTFRKATEYISIDEFYREVELLGYKVTFDSIKATYIEEKVANGELYLFQIYNKDFSTAKKGGGKDNLHTLYWKSLFSEDNLKDVVFKLNGQAEVFFRPSSIVWSEKVMKQGHHYDDDKMLKDKFKYPIIKDFRFTQDKFFFHCPISLNFKAPAKPYGFNYKVHDFLRKNGEINVIGIDRGEKHLLYYSITNQQGDIIEQGSLNSIESSYEKGSSSVPHSTNYLEKLQEKEKNRDSARKSWSRIENIKELKAGYLSHVVHKLAKLIIKHNAVVVLEDLNAGFKRGRFKVERQVYQKFEKALIDKLNYLVFKDQEKPSAPGHPLRAYQLTDPFESFEKLGKQSGILFYTTAAYTSTTDPVTGFLKNVYKPYANVVDSSDFWKSFDEIHYDVKKERFEFTYDLRKIKNKQVDRDKDEAKVSRSVWTVCSCVERSRYVKRKANEEQKQGAASKSIGKMGEYEVFVVTDKLKALFEKESISYQSGEDIRSQLSNRIDKKDAKLHRELIYYFGTLLNMRVTDKQSESGTDENDYILSPIEPFFDSRKGYEKLPVNGDANGAYNIARKGIAILNRIHESDSGDKVDLMIRKKDWQDFVQNEEVVQKQMGKLDL

SEQ ID NO: 12 - protein, 1238 aa, synthetic construct
ALFSENFVGCYPVSKTLKFELRPVPETKAYLDDDNCPLLAGDLERDKAYPVVKELLDKYYRYFIDICLKDKKLSPETIDEAYQAYSSHDEKSQKALKAASQKLRKEVAGFFDKKTRETYCLDKYSNLLVLDKKNDNKEKGTPKTIPSVLKKWLENESGFSEKEVEKYSQAVQTFNGFTTYFISYKENRENMFSSEEKASAISYRVVNQNMLRFFSNVENFENIKKKYPDLYQTLKEFEESFVPAAYSSLLSQAGIDDYNYRVIGRPAEDEDFAGVNRLINEYRQKNAVRNRNLPVMVTLYKQILSDRENGFGRDILASDSEAKEYIRGKYSKAYDTACQLTVFVNNYILTEDLDTVYVKTQHLKDISQSIFGEWSVLDDAINNGGIDSKIISLKALAEAFEKYCSALDPDMAQKYRDAFSLKEYLKNPPAVEPRLPETAEKVTAYKPAMDELISLIRFYKPFYLYDGKKRLQVPAEGMDFSNEFDKYFEDLKEFSHAYDKVRNFATKKPYSVEKIKLNFNQPTLLDGWDVNKEKENASFLFVKEGKYYLGIADKSDKNLFDRSRDAVKEALTLTCGEDYYEKVQYKQLPNIRMNLPRIVFKPKWEKVFAPIITERISAIEKEKLYTAGANDRQAVIEWIDFVKSVLALHSEWNNYFQFKFKETSAYKNTKEFYDDLDNQAYILSKIKINASYIHKLVRERKLYLFQIYCKDFSDKKKKTGADNLHTMYWRCLFSDENLAALAQGQAPIIKLNGKAEIFLREPSLKYEATHPKNQPVANKNPLNSKRESLFSYDLAKNRRFTERKLFFHCPLTINFRTAAEGARSFNERVNRFVENNPDIRIIGIDRGERHLLYYTVINQSGKILEQGSLNKISSDYRTPEGAEVEKVTDYHELLNRKEGDRRAARQTWDTIENIKELKEGYLSQVVHKLTTLMIKHNAVVVLENLNQNFKRSRIKVEKQVYQKFEKALIDKLNYLVFKDRPYGAPGSYANGLQLTAPFESFDRLGKQTGALYYVVPSYTSHIDPKTGFVNLLGAKLRYESVVKAQETMKKIGSFKFNPERNWFEISFDYGDFGIEMAHPQWTACTHGDAYWGYNPKEKRAVSYDVTEEIKKLLTEYGIDYGTGADIKEAAIRQTKPAFWKTLFYMLKLALQLRNTVAGTDNENDYILSPVEYEAGKFFDSRKAGEDEPQNADANGAYHIAMKGLQALRRIEDGKVNKFPKNGERKAWLAFMQNREYLK

SEQ ID NO: 13 - protein, 1301 aa, synthetic construct
SKLSTFNEHFQKTITLRNELVPVGKTLENIISSNVLINDEKRSEDYKKAKEIIDSYHQEFIEKSLSKVDIEWDDLAYCLTKKEPENFDKKKKYQEEINKLQNEKRKKSVEIFKKFSFGVLCDNNGQEVQIKFDDLFKSTLFKYVLPNFLKNDEDKTVVRDFSEFTSYFTGFHENRKNLYKSAPMPTAVAYRIVNDNFPKFISNQKIFCAWKDNVPHFVEIAKTKLSEVGISDLDIESKFELSNFNSCLNQTGIDYYNDLRGQLNNVISHICQQDKNLGDLLRKKRCLKMVPLYKQILSDKDSSFSIDEFDNDESAIQDVISFYKKMVGERCPQRKLSELLRELSSHDREKIFIQGKNLNSISRILFGGKNWSLLREAIIEEKSKDKSFKKAIKSNSTDEALEKILSKDEFSISFLSRICHENLSEKIDKFIKNQDEQLININSQTWPKTLKNSEEKKIIKEHLDFLLNFYRFAKSFSSNTIDKDMSFYADFDESLSSLENVIGLYNKVRNYVTKKPYSLEKIKLNFEIPNLAEGWSKTKELANLTVIFLKDNEYYLGIINKNSKIDFSLIKEKTSSENCFRKMEYSLFGKIAQNINRCVFTKAVKNHFSKSPDNFILSDKKKFLKEIEITKEIYDLVKSDESDKKYLKEYKKINEKEFRSALSKWINFCIEFLKSYKNTSQFDFSCIKKAESYDDINDFYDDIEKVTYKIEFVDISTSLIDALVDKGQLFLFKIRNKDFAKKASGTPNLHTLYFKSIFDTKNFENGIVKLNGEAEIFYRKKSLNVDDTTVHQKGSYIVNKVVVDPSSGKSEQIPDNVYNNIYAFVNGKLKTLSKEDEVYYAKATIKKATHEIVKDRRFTEDKFFFHCPITINYKSKEKPSKFNDKVLDFLRKNDDINIIGIDRGERNLIYATVINKNGEIIDCKSFNTIKHQSSSVNFDVDYHNKLQEREKNRKEEKRSWNSISKIADLKEGYLSAVIHEIALMMVKYNAIVVMENLNQGFKKIRGGIAERSVYQKFEKMLIDKLNYFVIKNENWTNPGGVLNGYQLTNKVSTIKDIGNQCGFLFYVPAAYTSKIDPSTGFVNLINFNKYKNSNDRRKLICSFDKICFVQNENLFKFSIDYEKLCPNSKMPVKKWDIFSYGNRIVREDLKHIPMKENPKYDPTEELKTLFSSKEIEYQKGQNLLEIISTKDMTEDKEFWNSLFNIFKAILQMRNSLTNSPVDRLLSPVKGKDGSFFDTDKVEGTKFEKLKDADANGAYNIALKGLLVLKKNDSVKTDKDMKNVKNVSLEDWLKFVQVTLRV

SEQ ID NO: 14 - DNA, 3750 bp, synthetic construct
  source 1..3750: mol_type = other DNA
aagaccatgaccgacctgaccggcctgtaccccctgagcaagaccctgaggttcgagctgaagcccatcggcaagaccctggagcacatcgagaggaagggcctgatcgcccaggacgagcagagggccgaggactacgagagggtgaaggacctgatcgacaagtaccacaagaagttcatcagcctgtgcctgaacaagtgcaggctgaaggtggagagcaccggcgagtacgacagcctggaggagtacgtggagttcgccggcatcgccaagaggacccaggaggaggagaacgccttcaccaaggtgaaggagatgctgaggaagaccgtggccgacagcttcaagaaggaccccgaccagttcaaggagctgttcaagaaggagatcatcaccgagcacctgcccaacatgctgctgaccgacggcagcgaggaggacaagaagaaggtggagagcttcgccctgttcaccagctacttcaccggcttccaccagaacagggagaacatgtacagcgccgaggagaagagcaccgccatcgcctacaggctgatccaccagaacctgcccatgttcctggacaacatcaagagcttcgagaagatccaggccaccatcgacggcaaggacatggaggagctggagaacgccttcagggagtgcctgaacgtggagcacatcgccgacatgttccagctggcctacttcaccgagatcctgacccaggagcagatcgacgtgtacaactacatcatcggcggcaagaccgaggaggacggcaccaagatcaagggcatcaacgagtacgtgaacctgtacaaccagcagcacaaggacagcaggctgcccctgctgaagcccctgtacaagatgatcctgagcgacaggaccaccctgagctggctgcccgagcagttcaagagcgacgaggagatgctgaccgccatcaacgaggccatcaagaagctgaacgacgaggtgctgaacaccgccaaccaggacaacctgcagcagctgctgagccacctgagcgactacgacaccaggcacatctacatcaccaacaaccactgcctgaccgacatcagccagcagatcttcggccagtacgacaggtacaccaccgccatcaaggaggagatcaagagcagcctgaagcccaccgccaaggagaagaaggaccccgacctgctgaacgacaggaccaggaagctgttcgacgccaggaagagcttcagcatcgccgagctgaacgagtacgcccaggccgaggccaaccagaccatcgaggtgtacttcgcccagctgggcagctacgacaggaacggcgagcagcagatcagcctgctgagcaggctgaacatggcctacaacgaggccaaggaggtgctggacggcaagtacaccaacctgaaccagagcaaggaggccaccgagaagatcaagaacctgctggacgcctacaaggccatccagttcttcgcccagcccctggccggcagcggcgacgaggccgagaaggacaacgagttcgacgccaagctgaggaacctgaccaacaccctgagcgaggtgatcacccccctgtacgacaaggtgaggaactggctgaccaggaagccctacagcaccgagaagatcaagctgaacttcgagaacgcccagctgctgggcggctgggacgtgaacaaggagcccgactgcaccgccatcctgctgaggaagcacaagcagtactacctgggcatcatggacaagaagagcaaccacagcttcgacatcgacgagctgcccagcgagggcgactgctacgagaagatcgactacaagctgctgcccggcgccaacaagatgctgcccaaggtgttcttcagcaagagcaggatcgacgagttcaaccccagcgacgccatcaagaaggcctacgagaagggcacccacaagaagggccccgacttcagcctggccgactgccacaggctgatcgacttcttcaaggccagcatcgccaagcacgaggactggaagaggttccccttcagcttcagcgacaccggcagctacgaggacatcaacggcttctaccaggaggtggagcagcagggctacatgatcggcttcaggcccgtgagcgaggcctacatcaaccagctggtggaggagggcaagctgtacctgttccagatctggaacaaggacttcagcgagtacagcaagggcaggcccaacatgcacaccatgtactggaacatgctgttcgacgagaagaacctggccgacgtgatctacaagctgaacggccaggccgaggtgttcttcaggcagaagagcctggacatcgccaagacctgcgtgcacgccgcccagcagcccatcaacaacaagaacaagagcaaccagaagcagcagagcaccttcgactacgacatcatcaaggacaggaggtacaccatggacaagttccagttccacgtgcccatcaccatgaacttcaagagcaagggcagggagaacatcaacagccaggtgagggagatcatcaggaacaacggcatccagcacgtgatcggcatcgacaggggcgagaggcacctgctgtacctgagcctgatcgacctgaagggcaacatcgtgaagcagatgaccctgaacgacatcatcaacgagtacaagggcaacacctacgccaccaactacaagcagctgctggagtacagggagggcgagaggaccgaggccaggaggagctggcagaccatcgagaacatcaaggagctgaaggagggctacctgagccaggtggtgcacgtgatcgccaagatgatggtggagtacaacgccatcgtggtgctggaggacctgaacggcggcttcatgaggggcaggcagaagatcgagaggaacgtgtacgagcagttcgagaagaagctgatcgacaagctgaactactacgtggacaagcagaaggaggccaccgaggccggcggcgtgctgcacgccctgcagctgaccagcaagttcgagagcttccagaagctgggcaagcagagcggctgcctgttctacatccccgcctggaacaccagcaagatcgaccccgtgaccggcttcaccaaccagctggacaccaggtaccagaacgtggagaaggccctgcagttcttcggcaggttcgacagcatcaggtacaacaaggagaaggactggttcgagttcatgctggactacgacaagttcaccaccaaggccgagggcacccagaccaggtggaccctgtgcacctacggcaccaggatcaggagcttcaggaacgacatcaacaagtgggtgcccaaggacgtggtgctgaccgaggagttcaagcaggccttcgccgccgccaacatcgacgtgaacggcaacctgaaggacgccatctgcaacctgaacgagaaggcccacctggaggagctgatgcacctggtgggcctgctggtgcagctgaggaacagcaagaccaacaccgagatcgactacatgctgagccccgtggccgacaagaacggcgtgttctacgacagcaggacctgccccgacaccctgcccaaggacgccgacgccaacggcgcctacaacatcgccaggaagggcatctgggtgatcaggaagatccaggaggaccagaccaacgacaaggtggacctgaagatcaccaacaaggagtggctggccttcgcccagcagaagccctacctgaagcacctg

SEQ ID NO: 15 - DNA, 3732 bp, synthetic construct
  source 1..3732: mol_type = other DNA
gagcccatcggcaagaccgccgactacatcaagaactaccagtacctggagaccgacgagaggctggccaaggagagcaagaaggtgaaggagctggccgacgagtaccacaaggagttcatcggcgacgtgctgagcagcctggagctgcccctgagcaagatcaacgagctgtgggacatctacatgagcaacgacaccgacagggagatcaagttcaagaagctgcaggagaacctgaggaaggtgatcgccgaggccttcagcaaggacaagaggttcggcaacctgttcaagaaggagatcatcaccgacatcctgcccaagttcctgcaggacaaggacgacgacatcaagatcgtgaacaggttcaagggcttcaccacctacttctacgccttccacaagaacagggagaacatgtacgtgagcgaggagaagagcaccgccatcccctacaggatcgtgaaccagaacctggtgaagtacttcgacaactacaagaccttcaaggagaaggtgatgcacctgctgaaggacaagaacatcgtggagagcatcgagagggacttcaaggacatcctgaacgagaagagcatcgaggacgtgttcggcctggccaacttcacccacaccctgtgccaggccgacatcgagaagtacaacaccctgatcggcggcctggtggtgaagaacgagaagaaggagatcaagggcatcaaccagtacatcaacgagcacaaccagaccagcaagaagggcaacggcatccccaagctgaagcccctgttcaaccagatcctgagcgacaggaagagcctgagcttcaccctggacgacatcaagaagaccagcgaggccatcaggaccatcaaggacgagtacgagaacctgagggacaagctggccaccatcgagaggctgatcaagagcatcaaggagtacgacctggccggcatctacatcaagatgggcgaggacaccagcaccatcagccagcactggttcggcgcctactacaagatcatcgaggccatcgccgacgcctgggagaggaggaaccccaagaagaacagggagagcaaggcctacagcaagtacgtgagcagcctgaagagcatcagcctgcaggagatcgacgacctgaagatcggcgagcccatcgagaactacttcgccaccttcggcaccacctgcagcgacaggaccagcggcgtgagcagcctgaacaggatcaaggccgcctacaccgagttcgtgaacaagttccccgagggcttcgaggacggcgacgactgcaacgacgcctacttcaaggccaacgtggaggtggtgaagaacctgctggacagcatcaaggacttccagaggttcgtgaagcccctgctgggcaacgaggacgagagggacaaggacgaggccttctacggcgagttcgtgcccacctacaccgacatggacaacatcatcacccccctgtacaacagggtgaggaacttcgccaccaagaagccctacagcaccgacaagatcaagatcaacttcgagaacgtggtgctgctgaagggctgggacaagaacaaggagagcgactacgccagcatcatcctgatgaaggacggccagtacttcctgggcgtgctgaggaacggcagcaagagcaccctgaagaccatcctgcccaacaccggcgactgctaccagaagatggtgtacaagtacttcaaggacatcaagagcaacctgcccaggtgcaccacccagaggaaggacgtgaaggcccacttcgccgagagcagcgacgactacaccctgctggacaccaaggccttcgtgagcgccctgaccatcagcagggaggtgttcgagctgtacaacgcccccgacaaggagaagaagttcaagaaggagtacctgaagaacaccaacgacagcatcggctacgccaacgccgtgagcgtgtgcaagaggttctgcctggagttcctgaagaagtacaggagcaccgccatctacgacctgagcgacgtggagaccagcgtggacagcttcgacgacctgagcagcttctaccaggagatcgacaagaggctgtacagcatcagcttcgagaacgtgagcgtggacagcgtgaacgagctggtggacaacggcaacatgctgctgttcaggatcgccaacaaggacttcagccccaacagcaagggcaggcccaacctgcacaccatctactggaggatgctgttcgaccccgccaacctgaaggacgtggtgtaccagctgaacggcaacgccgagatcttcttcaggaaggccagcgtgaccaggaccgagcccacccaccccgccaacgtggccatcaagaacaagagcgagtacaacaagcagaacaagccctacagcaccttcaagtacggcctgatcaaggacaggaggtacaccaccgaccagttcgagttccacgtgcccatcaccatgaacttcaagcagcccgagagcagcaagctgcaggacaagctgaacaagcaggtgctggacttcctgaagcaggacggcgtgaggcacatcatcggcatcgacaggggcgagaggaacctgctgtacctggtgatggtggacatggagggcaagatcaagaagcagatcagcctgaacgagatcgccggcaaccccaagaaccccgagttcaagcaggacttcctggccctgctgcacgagagggagggcgacaggctggagagcaggaggagctggaacaccatccagagcatcaaggagctgaaggagggctacatgagcctggtggtgcacgagatcgccaacatgatgctggagaacgacgccatcgtggtgctggagaacctgaacaggagcttcatgcagaagaggggcgagaagagcgtgtaccagaagttcgagaagatgctgatcgacaagctgggctacatcgtggacaagaccaaggacgtgagcgacaacggcggcgccctgcacgccgtgcagctggccgacaccttcgagaacttcaacaagacccagaagggcgccatcaggcagtgcggcttcatcttctacatccccgcctggaggaccagcaagatcgaccccgtgaccggcttcgtgcccatgctgaggtgccagtacgagagcatcgtggagagcaagaagttcttcggcaagttcgacagcatctactacgacgccaccggcaagtacttcgtgttccagaccgacttcaccaagttcaacaccgagagcaagggcggcatccagaagtgggacatctgcacctacggcgacaggatctacgcccccaggaccaaggacaggaacaacaaccccgtgagcgagagggtgaacctgaccgaggagatgaagagcctgttcgtgagccacaacatcaacatccagggcgacatcaaggccggcatcatgcagcagaccgacaaggagttcttcgagagcctgcacaggctgctgaggctgaccctgcagatcaggaacagcaagaagagcaccggcaaggactacgaggactacatcatcagccccgtgatgggcaaggacggcaggttcttcgacagcaggaacgccgacgccacccagcccaaggacgccgacgccaacggcgcctacaacatcgccaggaagggcctgatgctgctgaggcagatccaggcccaggagaagcaggacctgagcaacggcaagtggctggagttcgcccagagg

SEQ ID NO: 16 - DNA, 3903 bp, synthetic construct
  source 1..3903: mol_type = other DNA
accaggaagttcgacgacttcatccaccagtacagcctgagcaagaccctgaggttcgaggccaggcccatcggcgagaccctgcacaacttcatcaagaacggcctgctggagagggacgagcacagggccgagagctacgtgaaggtgaagaagctgatcgacgagtaccacaaggccttcatcgacagggtgctgaacgacggctgcctgaactacgaggacaagggcgagtacgacagcctgaccgagtactacgccctgtacagcgcccccaggaaggacgaggccgagagggagcacttcaaggagacccagcagcacctgagggaccaggtggtgaagaagctgaccggcgacaacgcctacaagcacctgttcggcaaggagctgatcgagagctacaaggacaaggaggacaagaagaagctgaaccaggccgacctggtgcagttcatcaacaccgccacccccaagcagaggctgaacttcagcaaggaggaggccatcgacctggtgcaggagttctgcggcttcaccacctacttcggcgacttctaccagaacagggagaacatgtacagcgccgaggagaagagcaccggcatcgcctacaggctgatcaacgagaacctgcccaagttcatcgacaacatggagaccttcaagaagatcgccgccatccccgagatggaggacaacctgaaggagatccacgacaacctggccgagcacctgaacgtggagaacatccaggacatgttccagctgaactactacaaccagctgctgacccagaagcagatcgacgtgtacaacgccatcatcggcggcaagaccgacgacgagcacaaggagaagatcaagggcatcaacgagtacgtgaacctgttcaaccaggcccacaaggacgccaagctgcccaagctgaagaccctgttcaagcagatcctgagcgacaagaacgccatcagctggctgcccgaggagttcgacaacgaccaggaggtgctgaacgccatcaaggactgctacgccaggctgagcgagaacatcctgggcaaggagaacctgaagaggctgctgtgcagcctgagcgagtacgacaccaagggcatcttcctgaggaacgacctgcagctgaccagcatcagcaagaagatgagcggcagctggaccgacatccccaacgccatcaagaacgacctgaaggacggcgtgcccgccaagaagcacaaggagagcgaggaggactacgagaagaggatcgacaacctgttcaagaggcaggacagcttcagcatcggctacatcgacgactgcctgaacaagttcgacgacaacaacaccttcaccatcgagggctacttcaagaagctgggcgccaaggacacccagagcgaggacatcatcaagcagatcgccaacgcctacaccgacgtgaagcccctgctgaacagcacctaccccaagagcaagaacctgagccaggacaaggagaacgtggtgaagatcaagaggttcctggacaccctgatgagcctggtgcacttcgtgaagcccctgctgggcaagggcgacgagagcaacaaggacgagaagttctacggcgagctgagcctgctgtggaccgagctggagaccatcgtgcccctgtacaacatggtgaggaactacatgaccaggaagccctacagcaacagcaagatcaagctgaacttcgacaacagccagctgctgggcggctgggacgccaacaaggagagcgactacgccagcatcctgctgaggagggacggcaagtactacctggccatcttcgacaaggacagcaagaagctgctgggcaagagcatgcccagcgacggcgagtgctacgagaagatggtgtacaagttcttcaaggacgtgaagaccatgatccccaagtgcagcacccagctgaagaaggtggtgaagcacttcagcatcagcgacaccgacttcgtgctgagcgacgagaacttcaaggtgcccctgaccatcaccaaggaggtgtacgacctgaacaacaccatgtacggcaagtacaagaagttccacaagggctacctgaccaccaccggcgacatcgacggctacaccagcgccgtgaagacctggatcaacttctgcatggacttcctgggcgcctacggcagcaccatgagctacgacctgagcagcctggccaccaaggagtacagcagcctggagagcttctaccaggaggccaacaggctgctgtacaccgtgaacttcgtgaaggtgagcaccagctacgtgagcaggctggtgaccgagggcaagatgtacctgttccagatctacaacaaggacttcagcgagtacagcaagggcacccccaacatgcacaccctgtactggaaggccctgttcgacgacaggaacctgaaggacgtggtgtacaagctgaacggccaggccgagatgttcttcaggaagaagagcatcgactgcaacaggcccacccaccccgccaaccagcccatccagaacaagaacaaggacaacaagaagaaggagagcgtgttcgagtacgacctgatcaaggacaggaggtacaccgtggacaagttcatgttccacgtgcccatcaccatgaacttcaagagcaccgacaaggagaacatcaacctgcccgtgatggagtacctgcagaccagcaacgacacccacatcatcggcatcgacaggggcgagaggcacctgctgtacctggtggtgatcgacctgcacggcaacatcgtggagcagtacaccctgaacgacatcgtgaacgagtacaacggcaacacctacaggaccaactacaacgacctgctggaggccaggggcaacgagcagcagaaggccagccagaactggcagaccatcgagaacatcaaggagctgaaggagggctacctgagccaggtgatccacaagatcacccagctgatgatcaagtaccacgccatcatcgtgctggaggacctgaacatgggcttcaagaggagcaggcagaaggtggagaagcaggtgtaccagaagttcgagaagatgctgatcgacaagctgaactacctggtggacaagaaggccgacatcgagagcaccggcggcctgctgaacgcctaccagctgaccaacaagttcgtgagcttcaagaagctgggcaagcagagcggcttcctgttctacatccccgcctggaacaccagcaagatcgaccccgtgaccggcttcgtgaacctgctggacaccaggtgccagaacaagaacaaggccaaggccttcttcgccaagttcgacagcatcaggtacaacaaggacaaggactggttcgagttcgccctggactacgacaagttcggcagcaaggccgagggcaccaggaccaagtggatcctgtgcacccagggcaagaggatcaagaccttcaggaacgccgagaagaacttccagtggtacaaccaggtgatcgacctgaccaaggacctgaagcagctgttcgcccagtacgacatcgacatcaacggcaacctgaaggaggccatcagcaagcagaccaagaagaccttcttcgacgacctgatgggcctgctgaagctgaccctgcagatgaggaacagcatcaccaggaccgagaccgactacctgatcagccccgtggccgacgagatcggcaacttctacgacagcaggacctgcggccccagcctgcccgagaacgccgacgccaacggcgccttcaacatcgccaggaagggcctgatgatcatcgagcagatcaaggccagcgaggacctgagcaagctgaagttcgacatcagcaacaagagctggctgaacttcgcccagcagaagccctacaagcacgag

SEQ ID NO: 17 - DNA, 3846 bp, synthetic construct
  source 1..3846: mol_type = other DNA
aacaccagcaagttcatcttccagaacctgacccaccagttccagctgcagaagaccctgaggttcgagctgatcccccagggcaggaccctggagcacatcgagaagaacggcctgctggaggccgacgagctgaaggccaacaggtacaagcaggtgaaggagtggatcgacctgtaccacaaggccttcatcgagaaggtgctgagggagcccaagctggagggcctgagcgagtacagggtgttcttcgagaacaagcagaggaacgagaaggacgaggaggccttcaagaagctgaagagcgacctgaggaagaagatcagcagcttcttcaaggacgagcccaacaagaccttctacaagaggctgttcgacaaggagctgatcaaggccgacctgcccgacttcctgagggagcagggcatgcacgaggaggccaccatgctgctggaggagttcggcaacttcaccacctacttcaagggcttccacgagaacaggaagaacctgtacgtggccgaggacaagagcaccgccatcgccttcaggctgatcgacgagaacctgcccaggtacatgcagaacctgctgctggtggagaagcacagggaggtgttcgagcagttcttcccccagatccaggagaggctgggcaaggagctggacggcctgagggtggaggagttcttcagcgaggagggcttcaacctgtgcctgaacggcagcggcatccaccagtacaacaccatcctgggcggcgtgaagggcgaggaggaggagggcatccccggcctgaacatgctgatcaacctgtacaggcagaagcacaagctgaagagcagggagctgcccaccctgttccccctgcacaagcagatcctgagcgacagggagagcagcagcttcaggcccgagccctacgaggacggcgacgagaacaccgtggtggaggacctgctgaagctgggcgaccagtggttcaaccccgagggcttcgtgccccagctggtggcccacctgaccaagctgaaggacgaggagcaggaccagctgttcgtgaacaacaaggccaacgccctgagcgccgtgagccaggccaccctgggccactgggccgtgctgggcgaggccctgagcagctactggaaccacgtgcacgacgtgaaggagaacgccctgaagggcaaggccctgaccgccaaggagaagtggctgaagcagccctacttcagcttccaggagatcgagctggccctgaggcactacatgggcgccgagggcaaggagaacgccagcaagtggaacgtgtggctggagaagcccctggtgcagtacttcaactacggcctgaccaaggaggccgagaagaccaaggtggacaccgccaaggccgcctggggccacgccgagaacatcctgagggagctgcagaaccacgagaaccccaagctgaagaccaaggagaccaaggacgccatcaaggcctacttcgacgcctgcctggacctgctgcacttcatcaagcccctggtggtggacctggagcagtgcgaggccagccccaccttctacggcgacttcctgagcctgtaccacgagatggacgacctggtgcccctgtacaacaaggtgaggaactacatgaccaagaagccctacagctacgagaagttcaagctgaacttcaacaagggcaccctgctggacggctggagccagagcaaggagaccgacaacctgtgcgtgctgttcagggagggcaacaacttcttcctgggcatcatggacaggaagcacaacaagagcttcgtgaagccccccatggccaaggaggaggagcccgtgatgcagaaggtggtgtacaacctgctgcccgaccccagcaagatgctgcccaagtgcttcaccgccccctacttcaggaggatcgccaaggagaagaccaaggaccccgagcagatcgtgagcgagtacagcaaggagaagggcctgatcaagcccaagctggacgtgctggacgcctacgcccacaagaagcacaccaaggagaacttccagaggaacggcttcgagcaggtggtggagtacttcaaggagggcgcccagaagcaccccgactggcacgtgttcgacttcaagttcaagcccaccagcgcctacaaggacgtgagcgacttctaccaggaggtgaccgagcagggctactgcctggacttcgtggacatccccaagaggtacatcaacgacctggtggagagcggcaagctgtacctgttccagatctacaacaaggacttcagcaagtacagcaagggcaagcccaacctgcacaccatgtactggaaggccatcttcgacccccagaacctgaaggcccccatctacaagctgaacggcgaggccgaggtgttcttcaggaagaagagcctggagtacacccaggagcagctggacaccggccaccacagggaggagctgaagggcaagttcgactaccccatcgtgaaggacaggaggttcgccttcgacaagttccagttccacgtgcccatcaccctgaaccacgccaggaagaaccaggccgtgggcccccaggtgctgaggtacatccagaagcacaaggaggagctgcactacatcggcctggacaggggcgagaggcacctgctgtacctgagcgtggtgaacgccaagggcgagatcatcgagcagaggagcctgaacaagatcggcaacggcttcgtgaaggacggcaagccctacacccacgactaccagggcaagctgctggagaaggagaggaacagggacagcgccaggaaggactgggacgccgtggagagcatcaaggagctgaaggagggctacatgagcatcatcgtgcacgagatcgcccagctggtggtgaagtacaacgccgtggtgatcctggaggacctgaacttcggcttcaagaggggcaggttcaaggtggagaggcaggtgtaccagaagttcgagaggaagctgatcgagaagctgaaccacctggtgttcaaggaggtggccccccaggagcccggcggcctgctgaaggccctgcagctgaccgaccagttcaccagcttcaccaagctgggcaagcagagcggcatcctgttctacctgcccgccgcctacaccagcaagatcgaccccctgaccggcttcatcaaccaggtgccctggaagtacaccaacctggaggccgcccagaagtggatcggcaacttcaagaggatcagctaccacgccgagctggacctgttcgagtgggtgctggaccagagggacttcctgagcaaccccgccggctgcaagcaccagtgggtgctgaggatggacaaccaggagaggtggcactggaacagcaaggccgagggcaccaacggcaagggcgcctacagccccatcaggctgaaggagcagttcgaggacctgctggccaggcagaacatcagctacaacggcaacagcaagctgctggaggacatccaggccgtgaaggacgccagcttcttcaagaccctgttcttcttcctgaggaccctgacccagctgaggtacaacaacggcaagagcggcgccgaggaggaggacttcatcctgagcgccgtggccaaccccggcatggagcccttcgacaccaggaaggccaagcagggcctgcccctggacgccgacgccaacggcgcctaccacatcgccatgaagggcctgatgaccctgggccagctgaaggagaagcccaacggcaagctggagatccccaagctggacaagagcgcctggctgtgcttcatgcagcagaggagcaaccagggc

SEQ ID NO: 18 - DNA, 3786 bp, synthetic construct
  source 1..3786: mol_type = other DNA
cagaccaagaacttcatgagcttcagcgacttcaccaacctgtacgccctgagcaagaccctgaggttcgagctgaggcccatcggcaggaccgaggagttcttcaagaagaacaaggtgttcgagaaggacaagaccatcaacgacagctacaaccaggccaagctgtacttcgacgagatgcacaggaggctgatcagggacgccctgaacacccagaacaccagcgccctggactacgagaagtacgccaagcactggaaggagctgcagaaggacaagggcaagtacatccagaactggaaggacgcccagaaggagttcagggagtgcatcagcaacctgctggaccagcaggccgagcagtggaagagcaagtacgccagcgtgggcctgaacaaggccgacatcaagcagaagggcaccgacttcctgagcagcgccggcatggtgaagatcctgaagaaggagttccccaaggaggccgagaaccagctggtgaaggagggcttccccagcctgttcatccagaaggccgaggcccccgagcagaagcagtacatcttcgacagcttcgacaggttcagcacctacctggccaagttcaaccagaccagggacaacctgtacaaggacgacggcacccccaccgccatcgccaccagggtggtggagaacttcgtggccttcctgcagaaccaggccatcttcaacaaccactacatcagcctgaagagcgagctggacttcagcagggaggagatcgactgcttcaccgccaactactacgtgaggtgcctgctgcaggaggacatcgacgagtacaacaagaccatcggccagatcaacgccaggatcaaggagctgagggacaggaagcagaaggagagcgacttcaagaagagcggctaccccctgctgaagagcctgaacaagcagatcctgggcaagatcgagaagaagaggcagttcatcgagaacaaggccgacgtgttccccgccttcagcgagctgatcgagcaggccaacgccaggctgaccgccctgaggcagctgatcaacaacctgtgcgaggaggtgttcgaggaggcctacggcggcatctacatcaagaacaccggcatcaacaccatcagcaggaggtggttcgccagctggtacgacttcgagatcgccctgccccagaagagcaagaacgaggaggagagcaaggtggtgaccttcgtgagcctgcaggacatcaaggacaccctggagaacaagctgagcggcgacatcttcaaggaggagtactacaagaagaagatcatcaacaaggagcagagcaactggaagcagatcctggccatcctgcagaacgagtggaacgagctgttcagcgagtaccagaaggccctggagaacaccaagaagctggacctgaacagcgtgttcagcaacagcaaggagcaggtggagatcatcaagagcttcgccgacgccagcctgagggtgtaccaggtggtgaagtacttcgccctggagcacaagaagagcgacgagatccccagcagctacagcaccgacttctacgaggagttcgacaagcagtacaacaacttcgagttcatcccctactacaacgccctgaggaacttcctgaccaagaaggtggtgggcgagaacaagatcaagatcaacttcgagaagggcaacctgctgaacggctgggccgagagccccgagggcaacgcccagttctgcggctacatcctgaagaaggagaacaagtacttcctgggcatcaccgaccagcccaccatcctggacaccaaggacaaccccagcctgaccaacatcaccgacaaggagtactacgagaagatggcctacaggcagctgaagagcaccaccatctacggcagcggctacgagggcctgtacaacagcaagtacgccaacgacaagaagtacatcagcgagagcaagatcatcgagagggtgaagaagctgctgcaggagaagtacctgaccaggttccccgagctgaagcagatcatcgagaaggagtacaaggacgccaacgagctggccaagaacctgggcaacttcaacctgtacagcctggagttcatccccgtgaacaagaactacatcgagaagctgacccacgacaagaacggcagcatcaagtacctgtacatcttcgagatcgccaacaaggacctgggcaagatcacccccaggaagtacaacatccacagcctgtacttcaagaacctgttcagcgaggacaacctggagaagcccgtgttcaagctgagcggcggcgccgagatcttcttcaggcccaagaccgtgaacctgcccaagaagaaggacaaggccggcaaggacatcatcgaccacaagaggtacgcccaggacaagctgttcttccacctgcccatcgtgatcaacttcggcgccggcaaggtgggcaagttcaaccaggagctgaacaagaagctgctggccggcaacaagaacatcaacatcatcggcatcgacaggggcgagaagcacctggcctactacagcgtgatcaaccagaagggcgagatcatcgagcaggacagcctgaacgagatcaacggcgtgaactactacggcctgctgcagaagagggagaaggagaggctggagaacaggaagagctggctgcccctgcagcagatcaaggacctgaagaggggctacatcagccaggtggtgtacaagatcgtgaggctggccatcgagcacaacgccatcatcgtgctggaggacctgaacatgaggttcaagcagatcaggggcggcatcgagaagagcatctaccagcagctggagaaggccctgatcgacaagctgggctacctggtgttcaaggacaggaacgccaaggaggtgggcggcgtgctgcagggctaccagctgagcgcccccttcgagagcttccagaagatcggcaagcagaccggcatcatcttctacacccaggccagctacaccagcgtgaccgaccccctgaccggcttcaggaagaacatctacgtgagcaacagcgcccccatcgagaagatcaaggtggccatcaacaacttcgaggagatcggctgggacgacgtggtgcagagctacttcttcaactacgacaccaagaacttcagcaacggcaacgacatccagagcaagaagtggaagatctacgccaaggtgcccaggatcaagagggagaaggtgaacggctactggcagtacaacgacgtggaccccaacgacatgctggagaagctgttcaggctgtacgagttcaagaacctgaggagcaacatgctggagcagatccacgccaaggagaccaacggcgagctgaggggcgagagggacttcgacaacaagcccaggaacttctaccagagcctggtgtacatcctgaacctgatcctgcagctgaggaacagcaccagcaagcagatcaagctgaacgagaaggaggacgtgtacgagaccggcgaggacgtggacttcatcgccagccccgcctaccccttcttcaccaccctgagcagcaagagcaacagcaacctggccggcttcgagaacaggttcaccggcctgaaccacgaccaggagcagaagaagttcaacggcgacgccaacggcgcctacaacatcgccaggaagggcatcatcatcctggagaggatcaccaagaacagcgacaagcccaacctgtacatcagcaacgaggactgggacaagttcgtgagccagagctacccc

SEQ ID NO: 19 - DNA, 3831 bp, synthetic construct
  source 1..3831: mol_type = other DNA
gagagcaagaccctggacaggttcatcggcaagtaccagctgagcaagaccctgaggttcgagctgatcccccagggcaagaccaaggagcacatcgagcagaagggcctgctgcagcaggacgagcacagggccaaggcctacaagatggtgaagaagatcatcgacaggtaccaccagcacttcatcgccaccgccctgaacaacctggccctggacggcctgcagagctacgaggagctgtaccacaagaaggagaaggaggaggccgacaagaagaacttcgagagcctgaaggccggcatgaggaagcagatcggcgacgccttcaacaagagcgaggccagcaaggtgcaggagatctggaaggtgctgttcgacaaggagctgatcaggaaggccctgcccgagttcgtgaccagcgaggaggacaagaagctgctggccgagttcaaggacttcaccacctacttcaccggcttccaccagaacagggccaacatctacacccacgaggagaaggccaccgccgtggccttcaggctggtgcacgagaacctgcccaagttcctggacaacagcagggtgttcgactggatccagaccgagtaccccgacctggacctgagcgccatcgagaaggagctggcccccgagctgcagggcaccaccctgcccgagatcttcgccctgagcttcttcaaccacaccctgacccagaacggcatcgacctgtacaacgccgtgctgggcggcaagaccaccgagcagcagaacaagatcaggggcctgaacgagtacatcaacctgtacaaccagaggaggaccgccgaggccaagaaggagggcgagaagttcaggagggtgccccagttcaaggagctgtacaagcagatcctgagcgacagggagagcatcagcttcctgcccgagaccttcgagaacgacagcgaggtgctgagcgccatcgagaacttcttcgccacccagctggtgcacttcgagaaggacaacaggagcatcagcgtgttcgccgagctgcagaagctgctgcaggagctgaaggaggacgacaggagcaagatcttcgtgaggaacgacctggccctgaccaacatcagccagaacatcttcggcgactggcaggtgatcaagaacgccctggagcactactacaccaccaccgtgaaccccctggccgtgggcaagaagctgaccaaggcctacgagaacggcaaggagaagtggcagaagcagagctactacagcatctacgagatcgaggaggccctgctggcctacagcagcgaggccgacgtgctgaaggagaagaacctgaccgccgccagcaggcccctgtgcaactacttcgccaccttcagcaaggacgaggagggcaacaacctgttccagaggatcgagaccgcctacaccgccgtgaaggacctgctgaacaccgactaccccgaggagaagaacctggcccaggacaagaccaacgtggccgccatcaagaccttcctggacgccatcaagaacctgctgcacttcatcaagcccctgcaccccgccaggctggagggcgagaaggacgagaggttctacggcgccttcatccccctgttcgaccagctgaacctgctgacccccctgtacaacaagaccaggaactacctgaccaggaagccctacagcaccgagaaggtgaagctgaacttcgagaacagcaccctgctggacggctgggacgccaacaaggagaccgccaacaccagcgtgctgctgaggaaggacggcctgtactacctggccgtgatggacaagaagtacaacaaggtgttcgagcagatgcccgaggtggccgagggcgagaaggtgttcgagaaggtggtgtacaagctgctgcccggcgccaacaagatgctgcccaaggtgttcttcagcgccaagaacatccagtactacaaccccagcaccgagctgctggagaactacaagaacgagacccacaagaagggcgccaccttcaacctggtgcactgccaccagctgatcgacttcttcaaggccagcatcgccaggcaccaggactggaagaacttcaacttccagttcagcgacaccggcagctacgccgacctgagcggcttctacagggaggtggagcagcagggctacaagatcaccttccacaacctgcccgagagctacatcaaccagctggtggaccagggcaagctgtacctgttccagatctacaacaaggacttcagccccttcagcaagggcaggcccaacctgcacaccatgtactggaagatggtgttccacgagggcaacctgaccaacgtggtgtacaagctgaacggccaggccgagatcttcttcaggaagaagagcctgaactacgacgagaagaccctgagggagggccaccacgccgccgccctgaaggacaagttcgcctaccccatcatcagcaacaggaggttcgccttcgacaagtaccagttccacgtgcccatcaccatcaacttcaagagcaccggcagggacaacgtgaacgccgaggtgctgagcttcctgcagcacaaccccagcgtgaacatcatcggcatcgacaggggcgagaggcacctgctgtacctgaccctgatcgaccagaagggcaacatcaagaagcagatgagcctgaaccagatcatcaacagctacaggaacaaggagggcgacatggtgaacgtgaagaccgactaccagaacaagctgcacctgaaggaggaggagaggctgaaggccaggcagggctggggcctgatcgagaacatcaaggagctgaaggagggctacctgagccacgtggtgcacgagatcgccaagatgatggtggagcacaacgccatcgtggtgatggaggacctgaacttcggcttcaagaggggcaggcagaaggtggagaagcaggtgtaccagaagttcgagaagatgctgatcgacaagctgaactacctggtgttcaaggacaggaccgccaccgagcccggcggcctgctgcacgccctgcagctgaccggcagctacaccgacttcatgaagtacaagcagaagcagtgcggcttcctgttctacgtgcccgcctggaacaccagcaagatcgaccccctgaccggcttcgtggacttcctgaagcccaagtacgagagcatcgagaaggcccagaacttcctggccaagttcaagagcatcaggtacaaccccgccaagcagtggttcgagttcaccttcgactacaaggacttcaccgacaaggccgagggcagcaggaccaggtggaccctgtgcagcaccaaccagcccaggtacaggtggaaccccaaggccgccaacggcaagggcgcccaggaggccgtgaacgtgacccaggccctggaggacctgttcagcgcccacaacatcgcctacggcagcgccgacgacctggtgaccgccatcaccgcccagagcagcgccgacttctacaaggccctgctgagggccatggtgaccctgctggccctgaggcacaacaacggcaaggccggcaccgacgagcaggactacatcctgagccccgtggagcccttcttcgacagcaggctggccgacgacagcaggcccaaggacgccgacgccaacggcgcctaccacatcgccaagaagggcctgtgggtgctgcagcagatcaacgagctgcagggcgacgactggaagaacctgaagctggccatcagcaacaagcagtggctgcagttcgcccagcagctgaacgccgacatcagccccaaggtgagc

SEQ ID NO: 20 - DNA, 4026 bp, synthetic construct
  source 1..4026: mol_type = other DNA
agcagcagcatcgtgagcgtgagcaccgagaagaagcaggccgaggtgctgagcaacaagagcgtgttcgaccagttcaccaggaagtacgccctgagcaagaccctgaggttcgagctgaggcccgtgggcaagaccagggagaacatcgagaaggccaaccccaacttcatcagggacaaggagatcgaggacgcctaccagatcctgaagcccgtgttcgacaagctgcacgaggagttcatcaccgagagcctggagagcgacgaggccaagcacctgagcttcgaggactacttcagcgtgtacaagaacctgaggaacgagcaggacagggaggagaagaagaagatcgagaagagcctggagaccaaggagaagaacctgagggccgccttcagcgacatctacaaggccgagggcgagaagttcaagagcaaggtgggcagcgacaagaagggcaagcccatcctgaaggaggacagcttcaagatcctgaccgaggtgggcatcctgaagtacgtgaaggccaagatcgaggagttcgtggtgatgaacctgaagacccacgagggcaaggaggtgaagaaggaggacctggagaaggccctgggcaccgccgacagcaagggcgtgttcgagggcttcttcacctacctgaccggcttcaaccagaacagggagaactactacagcaccgaggagaaggccaccgccgtggccagcaggatcatcgacgagaacctgcccaagttcgccgacaacgtgctggagttccagaagaggaaggacgagtacctgaaggtgtacgacttcctgaaggagaacggcatcgagaccatcggcaagaaccagaagggcgagaaggtggagctggagaagatcatcgccgacatcttcgagatcggctacttcaccaactgcctgagccagagggagatcgagaagtacaacctgaacatcggcaacgccaacaacctgatcaacaggtacaaccagcagcagagcgagaagaacaagaagctgaggatcttcaagaccctgtacaagcagatcggctgcggcgagaagggcgacttcatccccgccatcaagaacgacgacggcctgagggccgtgctgaagaacgtggtggagaacggcgagaagtacttcggccagttcgacggcaagatcgaggacaaggtggacaccgtgggcgagttcgtgcactacatcatcaaccacgagagcttcgagggcatcttctggagcgacaaggccctgaacaccatcagcggcaagtacttcgccaactggttcatcctgaagaaggccctgaaggagggcggcgtgttcggcggcaagaagaaggacgacgaggacatcaagatcccccaggccgtggagctgaagaacctgttcgagatcctggacaagaccgagaagtggaagaacaaggaggacggcctgttcaagaccagcctgttcgacggcgagggcaacaagaagggcatcatcgagagggccgagaagcccagcaaggccctgctgggcatgatcttcgccgacgtggagaagatggccaaggacttcagcgagctgagccccaaggtgctgagcatcaccaccgacttcaagaagcccgagcacatcgagaacatcaagatgctgctggacagcgccctgtacgtgaaccagatcgtgaagtactggaaggtgaaggacaagttcgccgtggaccccatcctggccgagagcctgaaggccatcctgttcggcgacaacaaccccaccaagttctacgacatcatcaggaactacctgaccaagaggccccaggagggcctgaacaagctgaagctgaacttcgacaacagcgtgctgggcggcggctgggacgtgaacaaggagcccgagaggtggtgcctgatcctgaaggacggcgagaagaagtacctggccatcctgaccaaggccagccagaagctgttcggcgagaaggccaagtacaaggaggtggtgaccgacgagaactacctgaagatggactacaagctgctgcccggccccaacaagatgctgcccaaggtgctgctgcccaagaggaacaggaaggagttcggcgccaccgacgacatcctgagcatctacgacgagggcggcttcaagaagaaccagcccagcttcaccaaggagaagctgtggaagatcatcgacttctacaaggagggcctgaaggtgtaccccagcgccgagaagagctggcaggacctgttcggcttcaacttcagccccaccgagaagttcgagagcatcgaccagttctacaacgaggtggagaagcagggctacaagctgagctggaacatcatcagcaagaagctgatcaacgaggccgtggagaacggcgagatctacttcttcgagatcaggaacaaggacaacaacctgaaggagggcaaggagaagaccagcgagaagaacctgcacaccatctactggaacgagatcttcggcgacgtggccaacaagcccaagctgaacggcgaggccgagatcttctacaggaagaccatcctgaagaagaaggagaacgagaccgacaaggagttcgagatcaggctgaaggagaagctgggcaagaagaaggacaagagcggcaaggaggtgctggagcacaagaggttcgccaacgagaagttcgtgttccactgccccatcaccctgaacttctgcctgaggagcctgaagttcaacgacgagatcaacgagagcatggtgaagctgaacgacgacatcaccaccatcggcatcgacaggggcgagaagcacctggcctactacttcgtggtgaagcagaccaaggacggcaagatcgagaccctggaccagggcagcttcaacgagatcaacggccagaactacgccgagaagctggagaagatggccggcaacagggacgaggccaggaagaactggaagaccatcggcaccatcaaggagctgaaggacggctacatcagccaggtggtgaggaagatcgtggacctggccatctacgaggacgtggagaagaaggtgttcagggagacccccgccttcatcgtgctggaggacctgaacatcggcttcaagaggggcaggcagaagatcgagaagcaggtgtaccagaagctggagctggccctggccaagaagctgaacttcctggtggacaagcacgccaaggagggcgagatcggcagcgtgaccagcgccctgcagctgacccccctggtgaacaacttcggcgacatcgagggcaagaagcagttcggcatcatgttctacaccaaggccgactacaccagccagaccgaccccgtgaccggctggaggaagaccatctacctgaagaagggcagcgaggagagcatcaagaagcagatcctggacaacttcgacgacatcaagttcgacggcaaggactactacttcaagtacaccgacaggaacaccaacaagccctgggagctgtacagcggcaaggacggcgagagcctggacaggtactacagggagctggtgcacgagaacagcgacaagaagtgggtgcccaaggagcagaacctgaagaagatcctggacggcctgttcgagggcttcgacaaggagaagagcctgctggagcagctgaggcagggcaagaaccccaacaagatcaacgacaggaccagctgggagagcctgaggttcgtgatcgacctgatccagcagatcaggaacagcggcaagaccgaggaggacgccgacttcatcctgagccccgtgagggacaagaacggcaaccacttcgacagcaggaagatcaacctgaacctgcccaccagcggcgacgccaacggcgcctacaacatcgccaggaagggcatcatcatgtgcgagcacatcaagagggacctgagcctgttcatcagcgacgaggagtggagcgcctggctggccggcagggagatctgggagaactggatcaagaagaacgagaagaacctgaagaggaagcag

SEQ ID NO: 21 - DNA, 3867 bp, synthetic construct
  source 1..3867: mol_type = other DNA
aagagcatcgaggagctgatcggccagtacagcctgaccaagaccctgaggttcgagctgaagcccatcggcaagaccctggagcagatcgagaggaagggcgtgctgagcgccgaccagaccagggccgaccagtacaagagggtgaaggagagcatcgacaggtaccacaaggccttcatcaccgagagcctggacagcctggtgctgaaggtgaagagcaccggcaacaccagcgacagcctggaggagtacgccagcctgctggacaacagcgccgacgacgaggccctggacaaggtgaagaagggcctgaggaagcaggtggccgagaggttcgacaagcagatcaggctgaagaagctgttcaagaacgacctgatcaccaagatcctgcccgagttcctgaaggtgaacggcaacgaggacgagaggaaggccgtggagagcttcaggaacttcaccacctacttcagcggcctgaacgagaacaggaagaacatgtacagcaaggaggccaagagcaccgccatcgcctacaggctgatcgacgagaacttccccaggttccacgacaacatgctgagcttcgccaagatcgccgccgtgaacgaggtgagcaaccacttcgccgagatcgccgaggccttcaagagcgagatggtggtggccagcgtggccgagatgttcggcctggagtacttcaacaacaccctgacccagcagaagatcgacctgtacaactacgtgatcggcggcaagaccgagggcgacaaggtgaagatccagggcatcaacgagtacgtgaacctgtacaaccagaaccaccccaacgacaagctgcccctgctgaagcccctgtacaagatgatcctgagcgacagggtggccctgagctggctgcccgaggagttcaacgacgacgaggccatggtgtgcgccatcaaggagttctacaacgccatccagcccgtgctgtggggcgaggtggaggacgacgacaacagcgtgtgccacctgctgaagaacatcggcaagtacgacatcgagcacatcttcatcagcaacggcgccgccctgaacgacgtgagcaggcagatgttcggccagtacgacttcctggccaacgccatcaagaaggacatcaggcagaacaccaagaggagcagcagggagaccgacgagaagttcgagggcaaggtgaacaccctgttcaagaaggccaagagctacagcatcgcccacctgaacgccctgatcaaggagaacaggagcgagaccatcgagagcttctacagcaacctgggcgcctgcgacagggacggcgagcagaagatcaacctgctgacccagatcgagctggcccacgaggccgcccaggacgtgctggacggcaagtacgccgacatcaacagggacgagaaggccaccatgctgatcaaggagctgctggacgccttcaaggccctgcagtacttcatcaagcccctgctgggcagcggcgaggaggccagcaaggacaacgtgttctgcaccaagctgctgaacgcctggcaggccctggacgccgtgacccccctgtacgacaaggtgaggaacaggctgaccaggaagccctacagcaccgacaagatcaagctgtacttcgagaacaagggcaagttcctggacggctgggtggacagcaagaccgacaacagcgacaacggcacccagttcggcggctacatcctgaggaagaagaacgagatcggcgagtacgactacttcctgggcatcagcagcgacaccaagctgctgagggccaaccccgacatccagtacgagcacggcatgtacgagaggctggactactaccagatccagagcaagaccttcctgggcagcagctacaagggcgagtacaacgacgacgccatgaggctgatgagcgccttcgtggagtacggcaagaagagcaggttcgacagctgcgtgctgcccaccgacaaggagaagagcctgcccacctacctgaagaggatctacaccgacaacaacagcttctacaacgaggccatgaccgacaccgtggtggccgaggcctacaacaacatcaagagccacctgctgctgaccctggccggcatcaagagggtggaggccgccgtgaacctgagcaagcacaacgagtacaacctgatccagatcttcgacgagatcggcaagatcaccagcagcagcaagatcttcagcttcttccccgtggacaccagcctggtggagaagctgaacgagagggacaacaagcccctgttcctgttcaagatcagcaacaaggacctgagctacgccgagaccagcagcaagaacatcaggaagagcaggggcaaggagaacctgcacaccatgtacttcaaggccctgatggagcagagccacaacgtgttcgacatcggcaccgccgaggtgttcttcaggaaggccacccccggcctggacaagagcaccaccatccacaaggccaacgaggccctgtgcaacaagaacccccacagcaagaagaccagcagcaccttcggctacgacatcatcaaggacaggaggtacgtggagaacaagttccagctgcacctgagcatggtgatcaactaccagggcaagaaccccaacaccgacatcatcagcccccaggtgctggacatcatcaggaacaacggcatcaagcacatcatcggcatcgacaggggcgagaggaacctgctgtacctgagcctgatcgacctgaagggcaacatcatcaagcagatcagcctgaacgagatcaagcccctgggcaacgacggctgcgtgaccaactacagggacatcctggccgacagggagaaggacaggaaggaggccaggaggagctggaagaagctggagaacatcaaggacctgaagagcggctacctgagccaggtggtgcacgtgatcgccaagatgatggtggactaccaggccatcgtggtgctggaggacctgaacagcgccttcatgcagagcaggcagaagatcgagaggagcgtgtacgagcagttcgagcacaagctgatcaacaagctgaactactacgtggacaagagcgtgagcgacgacctgcccggcggcctgctgtacggcctgcagctggccagcaagaagacccacgacgtgcagtgcggctgcatcttctacgtgagcgcctggaacaccagcaagatcgaccccgtgaccggcttcgtgaacatgctggacaccaggttcgagaacgtgagcagcagcaggagcttcttcagccacttcgacagcatcaggtacaacgtggccaaggactggttcgagttcaccttcgactacaacaacttccacaagaagctggagggcaccaagaccaggtggaccctgtgcacctacggcaccaggatcctgacccagaggaacgacaggaaccagtgggagagcagggaggtggacatcaccgacgagttcaagaaggccttcgtggccgccggcatcgacatccacggcaacctgaaggacgccatctgcgccctggacaagagggtgcacctggagcagctgatgaggctgatgaagctgctgctgcagctgaggaacagcatcatcaaccaggacaccgactacatcctgagccccgtggccgagaacggcgtgttctacgacagcaggaggcacgccgacaacctgcccatcgacgccgacgccaacggcgcctacaacatcgccaggaagggcctgtacctgctgaggaagatcaaggccgacaggagcgccaagcccgagctgtgcatcagcaacaaggagtggctgaggttcgtgcaggacaagagcaaccccggcgac

SEQ ID NO: 22 - DNA, 3978 bp, synthetic construct
  source 1..3978: mol_type = other DNA
gtgaccatcgacgacttctgcggccagaagaacggctacagcaggagcatcaccctgaggaacaagctgatccccgtgggcaagacccaggagaacctggagaaggccaacatgctggaggaggacttcagcagggccaaggcctacgtggaggtgaagaagatcatcgacgacttccacaggagcttcatcgaggacgtgctgagcaaggccaacctgaactggaaggagctgtacgagaagatcgacagcttccagagggagaccgacaaggccaagaaggccaagaaccagaaggacctgcagcccgtgcaggtgaagatgaggaaggccatcgtgaagatcttcaaggacgacgagaggttcgacaagctgttcaagaaggagctgctgagcgacctgctgcccgagatcatcaagagcgcccccgagaacgagatcagcaacaagcagaccgccctggacgtgttcaagggcttcgccacctacttcaccggcttccacgagaacaggaagaacatgtacagcgaggaggacaaggccaccgccatcagctacaggatcgtgaacgagaacttccccaagttctacgccaacatcaagctgttcgagaagatccagaaggagttccccgtgatcatcgaggagaccgagaagagcctggccagcttcctggccggcaagaagctgaaggacatcttcacccccgaggccttcaaccaggtgctgacccagagcggcatcgacttctacaacaccgtgatcggcggcatcgccggcgaggccggcaccgagaaggtgcagggcctgaacgagaagatcaacctggccaggcagcagctgcccgacgaggagaagagcaagctgaggggcaagatggtggtgctgtacaagcagatcctgagcgacagggagaccgccagcttcatccccgtgggcttcgagaacaaggacgaggtgtacaagaccctgcaggagttcaagaagagcaccgtggagaagagcatcgagatcatcagggagaccttcaagagccccgagaacttcgacctgagcaagatctacgtgcccgccaaggagctgaccgccttcagcctgagcgtgttcggcaggtggaacatcctgagcgacggcctgttcgccatcgtggaggagaagagcaagaaggccctgagcgagaagcagaccgagaagctggccaacgagatcgccaagaaggactacagcctgagcgagctgcaggccgcctacgagaagtacctggagaagaaccccgtggaggacgagggcaggtgcaggaggctgagcgccaagaactacttcgagctgatcgagctgaggaagatcgacgacagcgccgagaaggagttcgtgaacgtgctgagcaacctggacgagctgtggagcaagatcgacttcgagaaccccaggaacctgcagcaggagaaggaggccgccacccccatcaagaactacctggacgaggtgcagaacctgtaccacttcctgaagctggtggactacaggggcgaggaggagaaggacagcgagttctacagcaagctgaccgagatcctggacctgctgagcgagatcgtgcccctgtacaacaaggtgaggaacttcgtgaccaagaagcccggcgaggtgaagaagatcaagctgaacttcgactgcagcagcttcctgaagggctggggcaccgagttcggcaccaaggacgccttcatcttcatcgacgagaagaagtactacctgggcgtgatcaacaagaagctggacagcgacaccgtgaagtacctgcagacccccggcgagcacatcatcaagcaggtgttcgtgaacttccagaagcccgacttcaagaacttccccaggctgttcatcaggagcaagaaggacaggttcgcccccgccgtggagcagttcaacctgcccgtgaacgacatcctggacatctacgacaagggcctgtacaagaccgagtacaggaaggagaaccccatcatctacaaggagagcctgatcaagatgatcgactacttcaagctgggcatcagccagcacgagagcttcaagggcttcaagctgacctggaaggagagcagcgagtacgagaacatcagcgacttctacgccgacgtgaccgccagctgctaccaggtgaacttcaccaacgtgaacttcgacaggctgctgagcctggccaacgagatcgaggacgacaacaagaagatcttcctgttcgagatctacaacaaggacttcagcaccggcaagaacggcggcaacggcagcaccggcaagaagaacctgcacaccatgtacttcgagaacctgttcagcaaggagaacctgaaggacatcgtgctgaagctgagcggcgaggccgagatcttcctgaagagcgagaacaccgagctgaagaagatcgtgcacaagcagggcaccaccctggtgaacaggaccaccaaggacaagaagctgatccccgagaacatctaccaggagatctaccagttcaagaacagcatgatcgacaacatcagcgacgaggccaaggcctggctgaacagcggcaacgtggagtgcaagatcgccaagttcgacctgatcaaggacaagcacctgatcgagaagagctactacctgcactgcagcatcatccagaacttcaaggcccccgagaaggtgttcaacttcaacgagaaggtgcagaggtacctgaaggacaaccccgacgtgaagatcatcggcctggacaggggcgagaggcacctgatctacctgagcctgatcaaccagaagggcgagatcgagctgcagaagaccctgaacctggtggagcaggtgaggaacgacaagaccgtgaaggtgaactaccaggagaagctggtgcagaaggagggcgacagggacaaggccaggaggaactggcagaccatcggcaacatcaaggagctgaaggagggctacctgagcaacgtggtgcacgagatcgccaagctgatggtggagcacaacgccatcgtggtgatggaggagctgaacttcggcttcaagaggggcaggttccccgtggagaggcaggtgtaccagaagttcgagaacatgctgatcggcaagctgaactacctggtgttcaaggacaagagcgtgaccgagcccggcggcgtgctgaacgcctaccagctgaccgacaagagcaccaacgtgagcgacgtgggcaagcagtgcggctggctgttctacgtgcccgccgcctacaccagcaagatcgaccccaagaccggcttcgccaacctgctgaacatgaaggacctgaccaacgtggccaagaagaggatgttcttcagcaagttcaaggacatccacttcgacagcggcagcaacgacttcgccttcaccttcgactacaaggacttcgacggcaagggcgccgccgagatgacccagaccgagtggaccgtgtggagcaacggcaagaggatcttctacaacagcaagcaccacagcaccgaggagatcaacgtgagcgagaagctgaaggagctgttcgacgccagcaagatcgactacaagaccgagaacaacatcttcgagagcatcatggaggtgggcgccgacctgaaggagggcgagaagcccagcaaggagatcgccaacttctgggacagcctgctgtggtacttcaagctgatcctgcagatgaggaacagcatccccaacagcgagaccgactacctgatcagccccgtgaaggccgaggacggcaccttcttcgacagcagggtgcagaaggagctgggcaaggacagcaagctgcccatcgacgccgacgccaacggcgcctaccacatcgccctgaagggcctgtacctgctgaagaacgacttcaacctgaacgacaagggctacatcgagaacatcagcaacgccgagtggttcaggttcgtgcaggagagggactacctgaac

SEQ ID NO: 23 - DNA, 3903 bp, Synthetic construct
  source 1..3903: mol_type = other DNA
cccaacctgttcgacaacttcacccagcagtacagcctgaccaagaccctgaggttcgagctgaagcccgagggcaagaccctggagaacatgcagaacgccttcaagttcgacaaggacctgcagaccttcctggtggaccaggacatcgaggacgcctaccagaccctgaagccctacatcgacttcatccacgaggagttcatcaccgagagcctggagagcgacatcgccaagggcatcgacttcagcgagtacttcgagaacttcaagatcaagaaggacatcaaccagaaggacttccagaacctggagcacaagctgaggaaggagttcgaccagctgtacatccagatcgccaacaagtggaaggaggagaagtacaagcagtacaagtggaagaagggcagcaaggtggccaacggcagcgagatcctgagcagcgaggacatcctgaagctggtggtggacctgaaccaggacaacaaccagctgaaggacatcgtgcagaacaccttcaagggcttcttcacctacttcagcggcttcaacgacaacaggaggaactactaccaggtggagaaggaggccaagagcagcatcgccaccaggatcgtgaacgagaacctgcccaagttctgcgacaacttcctgctgttcgagaacaggaagaacgagtacctgggcgtgtacaacaccctgaagctgctgggcaaggacctggtggacaaggacaggaagaccctggtgaccatcgaggagatcatcttcagcatcaagcacttcaacttctgcctgagccagaaggagatcgagaactacaacaagcagatcggcaacgccaacctgctgatcaacctgtacaaccagaacaccagcaacgagagcgactacaagaggctgcccctgttcaagaccctgtacaagcagatcggctgcggcaagagggacagcctgttcttcgagatcaccgactacaccaagctggaggccgagggcaagaggatcctgggcaagagcgccatcagcgtggaggaggtgctgatccaggtgaaggagagcggcatcaagtacttcaccgagaagctggacagcctgctgaactacatcctggagaaggagaccgacaactaccagggcctgtactggagcaacatcgccgtgaacagcatcagcagcaagtacctggccaactggaacaacctgaaggagctgttcaagggcaagaaggaggtggccagctacgacaagggcagggaggaggagatcaagatcaacgaggccgtggacctgagcatcctgttcagcatcctgaacaacaccgaggactggaaggtgaaggaggtgttcttcaaggagaacctgaacgaggagagcgagaccaagaagatcatcgagaccagcaagacccccagccaggccctgctgaggatcgtgatcggcgacctgaagaagaacgccagcaacttcctgaacctgagcgacaacatcgagcagctgctggagagcaaggagaagatcaaggagagcatggactacgccctgagcgtgaccaggatgctgaagtacttcctggtgaaggagagcaagaccaagggcgcccccctggacagcaagctggtggaggacctgaacctgctgctgctgagcgacgacgccccctggttcatgtggtacgacggcctgaggaactacctgaccaagaagccccacgacgacgccaaggagaacaagctgaagctgaacttcgagaacagcaccctggccggcggctgggacatcaacaaggagcccgccaactactgcgtgatcctgaccaacagcgccaacgaccagtacctggccatcaacgccaagaggaacaacaagatcttctgcaaggagctggtggacggcaagggcaaggacaagaagctgctgaagaaccccctgtacgagagcgacgagcagcactggaagaagatggagtacaagctgctgcccggccccaacaagatgctgcccaagtgcctgctgcccaagagcaacctgaggaagttcggcgccagcgaggaggtgctgagcatctacgagaacggcagcttcaagaagaacacccccgacttcagcacccaggacctgcacaagctgatcgacttctacaagagcgccctgctgaagtacgaggactggaagtgcttccacttcaagttcaacgacaccaggagctacggcgacatcagccagttctacagcgacctggagaagcagggctacctgctgagcttcatcgacatcaacaagagcgtgctgaaccagctggtggacagcggcaagatctacctgttccagatcaggaaccaggacagcaacatcagcaagaagatcaaccaccaggacaacctgcacaccatctactggagggccgtgttcgaggacgtgttcaacaagcccaagctgaacggccaggccgaggtgttctacaggaaggccctgccccaggaggacctgcagctgctgaaggacaaggacaacaaggagatcatcgccaactacaggttcagcaaggagaagttcctgttccacgtgcccatcaccctgaacttctgcctgaagaacaagaagatcaacgacctggccaacagggagatcctgagcaacgacaacatctacttcctgggcatcgacaggggcgagaagcacctggcctactacagcctggtggaccagaagggcaacatcgtgatcgaccagaacggcaagagcatccagggcaccctgaacatccccttcctggacagcaacggcaaccagaggctggtgaaggccaccaagagggtgatgaaggacggcaaggaggacgcccaggtggtggagtgcaaggactacaacgagctgctggaggccagggccagcgacagggactacgccaggaagaactggcagaccatcggcgccatcaaggacctgaaggacggctacatcagccaggtggtgaggaagatcgccgacctggccgtggagtacaacgcctacatcgtgctggaggacctgaacaccggcttcaagaggggcaggcagaagatcgagaagagcgtgtaccagaagctggagctggccctggccaagaagctgaacttcctggtggacaagagcgccaagatcggcgaggtgggcagcgtgaccaacgccctgcagctgaccccccccgtgaacaactacagcgacatcgagaacaggaagcagttcggcgtggtgctgtacaccagggccaactacaccagccagaccgaccccgtgaccggctggaggaagaccatctacctgaagaccggcagcgaggacatcatcaagaagcagatcttcgagagcttcagcaacatctacttcgacggccaggactacatcttcacctacaccgacaagaacatcgacaggaagtgggagctgtacagcggcaagaacggcaagagcctggacaggtacaggggcaggaaggtgaacaacaagggctgggtgatcgacaagatcaacatccaggagatcctggacaagatcttcatcaacttcgagatctgctacggcgagaagaacaacatcttcgagaagctgaaggccggcgtggagctggagaagtacaacgagtacaccgcctgggagagcctgaggttcgccatcgacatcatccagcagatcaggaacaccggcatcgagagcagcgacagcgacttcctgctgagccccatcagggtgaacaacctgcacttcgacagcaggctggccagcgagaaccagcccaacagcggcgacgccaacggcgcctacaacatcgccaggaagggcatcatcatggccaactacatcaagcagggcgtgagccccttcgtgagcgacatcgactgggacaacttcgtgaccaagaac

SEQ ID NO: 24 - DNA, 4212 bp, synthetic construct
  source 1..4212: mol_type = other DNA
aacaagttcctggacagcttcacccaccagtacagcctgagcaagaccctgaggttcgagctgaagcccgtgggcaagaccgccgagtggatccagaagagggacatcatcggcaccaagaagcaggagaacggcgaggcccagctgaccggcaaggacgccgtgagggccgagaactacgagtacgccaagaagctgctgaacgagatgcacaggctgttcatcgaggacagcctggccgccatcaaggaggagcagaaggtggagctgaacaagctgctggtgaagatcgccgagagcggcagcctggacggcttcaggaagcccatgagcaacatctgcaaggagctgttcgacagcgccacccaggactggctgctggcctacagcgaggagatgccctgcttctgggacgaggacatcctggagctgaccaacaaggccgagaccaccgagaacaagaagaacatcaagttcttcctgaagaaggccgaggacatcaggaagttcaaggacgaccccctgaaggccttcaagaagaagaagcacgaggccctgaacagcaacaccgaggccctgaagctgctggagtggaagatcaggaggggcgaggtgctgatcagcggcaaggacctgggcagggacgagagcgaggagatcctgagcaccaaggagctgatcctgatcctgaggagcttctacagcttcgtgacctacttcaccggcttcaacgagaacagggccaacatctacaacctggacgacaagaagtacatcagcaccagcatcgccttcaggagcttcgagcagaacctgttcttccacatcgagaacatcaagaagtggcacaccatcaccaacagcatcgaggagcacaccgaccagctgaacgccagcgactaccccatcaaggacaagctgagggagctggagctggccttcgactgcaagctggccgacctgttcgccgtggacagcttcagccagttcctgagccagagcggcatcgacaggtacaacagcatcatgggcggcatcgcccagaagaccggcaagaccaaggtgcagggcctgaacgagatcatcaaccaggccaggcagcagaccggcgccaagaggcacaagttcccccccatgaagctgctgtacaagcagatcctgagcaagggcgagggcatcttcgtggagagctacgtggacgacgccgacatgctgaccagcatcgccggcttccacgagaaccacctgaacgagagcctgtgcagcaactggctgcacgagctggaggagctgatcgacagcgtgcaggagagcaaggacaccatcttcatcgccagggacaagctgaacaggatcagccaggagatcaccggcagctggagcgccatccaggactggatcgtggcccaccagaaggaccagaaggaggtggacgccctgaccaagaagaaggtgttcagcatccaggagctggaggactacttcagcaagagcagcgacgacgagctgttcaccgccaagatccccgaggacaggagcgcctacctgcccggcgagggcgagggcatcctgttccagttcttcaaggtgagggccaaggagatcctggccgagctgcagagcggctggtacgccctggacaaggcccagctgctgcaggagaagcagctggacaagaacagggagaacgagggcagcaagggcttcgagcaggtgggcctgctgaagggctacctggacgcctgcaacagcctgagcaggttcatcgccgactggaacatccagaaggacatcaagcaggagaggtgcagcctgtggtacgagttcctgcagaagcacctggacaccgtgcccatcttcgacctgtacaaccaggtgaggaaccacgtgagcaagaaggcctacagcaccgacaagctgaaggtgaacttcgagaagagcaccctgctggacggctgggacaggaacaaggagagccagaacttcggcgtgctgttcgagaaggacggcgcctactacctgggcatcatgacccccgacagcaacggcatcttcgagtacgacgtgaaccccgacgagaacaccaagaagaaggccatcaaggaggagctgaggaagcagaccacccccgagaacggcgaggacaggttccagaagatcaactacaagctgctgcccggccccaacaagatgctgcccaaggtgttcttcgccaagagcaacgaggacctgttccagcccagcgccaggatcaccgagatcaaggagaacaagaagtacgccaaggcctacaccgacaagcacggcaaggccgagctgcacgagtacatcgacttctgccagaagagcctggtgaggcaccccgagtggagcgtggcctacggcttcgaccagaccaccttcaggaaggccaccgagtacatcagcatcgacgagttctacagggaggtggagctgctgggctacaaggtgaccttcgacagcatcaaggccacctacatcgaggagaaggtggccaacggcgagctgtacctgttccagatctacaacaaggacttcagcaccgccaagaagggcggcggcaaggacaacctgcacaccctgtactggaagagcctgttcagcgaggacaacctgaaggacgtggtgttcaagctgaacggccaggccgaggtgttcttcaggcccagcagcatcgtgtggagcgagaaggtgatgaagcagggccaccactacgacgacgacaagatgctgaaggacaagttcaagtaccccatcatcaaggacttcaggttcacccaggacaagttcttcttccactgccccatcagcctgaacttcaaggcccccgccaagccctacggcttcaactacaaggtgcacgacttcctgaggaagaacggcgagatcaacgtgatcggcatcgacaggggcgagaagcacctgctgtactacagcatcaccaaccagcagggcgacatcatcgagcagggcagcctgaacagcatcgagagcagctacgagaagggcagcagcagcgtgccccacagcaccaactacctggagaagctgcaggagaaggagaagaacagggacagcgccaggaagagctggagcaggatcgagaacatcaaggagctgaaggccggctacctgagccacgtggtgcacaagctggccaagctgatcatcaagcacaacgccgtggtggtgctggaggacctgaacgccggcttcaagaggggcaggttcaaggtggagaggcaggtgtaccagaagttcgagaaggccctgatcgacaagctgaactacctggtgttcaaggaccaggagaagcccagcgcccccggccaccccctgagggcctaccagctgaccgaccccttcgagagcttcgagaagctgggcaagcagagcggcatcctgttctacaccaccgccgcctacaccagcaccaccgaccccgtgaccggcttcctgaagaacgtgtacaagccctacgccaacgtggtggacagcagcgacttctggaagagcttcgacgagatccactacgacgtgaagaaggagaggttcgagttcacctacgacctgaggaagatcaagaacaagcaggtggacagggacaaggacgaggccaaggtgagcaggagcgtgtggaccgtgtgcagctgcgtggagaggagcaggtacgtgaagaggaaggccaacgaggagcagaagcagggcgccgccagcaagagcatcggcaagatgggcgagtacgaggtgttcgtggtgaccgacaagctgaaggccctgttcgagaaggagagcatcagctaccagagcggcgaggacatcaggagccagctgagcaacaggatcgacaagaaggacgccaagctgcacagggagctgatctactacttcggcaccctgctgaacatgagggtgaccgacaagcagagcgagagcggcaccgacgagaacgactacatcctgagccccatcgagcccttcttcgacagcaggaagggctacgagaagctgcccgtgaacggcgacgccaacggcgcctacaacatcgccaggaagggcatcgccatcctgaacaggatccacgagagcgacagcggcgacaaggtggacctgatgatcaggaagaaggactggcaggacttcgtgcagaacgaggaggtggtgcagaagcagatgggcaagctggacctg

SEQ ID NO: 25 - DNA, 3714 bp, synthetic construct
  source 1..3714: mol_type = other DNA
gccctgttcagcgagaacttcgtgggctgctaccccgtgagcaagaccctgaagttcgagctgaggcccgtgcccgagaccaaggcctacctggacgacgacaactgccccctgctggccggcgacctggagagggacaaggcctaccccgtggtgaaggagctgctggacaagtactacaggtacttcatcgacatctgcctgaaggacaagaagctgagccccgagaccatcgacgaggcctaccaggcctacagcagccacgacgagaagagccagaaggccctgaaggccgccagccagaagctgaggaaggaggtggccggcttcttcgacaagaagaccagggagacctactgcctggacaagtacagcaacctgctggtgctggacaagaagaacgacaacaaggagaagggcacccccaagaccatccccagcgtgctgaagaagtggctggagaacgagagcggcttcagcgagaaggaggtggagaagtacagccaggccgtgcagaccttcaacggcttcaccacctacttcatcagctacaaggagaacagggagaacatgttcagcagcgaggagaaggccagcgccatcagctacagggtggtgaaccagaacatgctgaggttcttcagcaacgtggagaacttcgagaacatcaagaagaagtaccccgacctgtaccagaccctgaaggagttcgaggagagcttcgtgcccgccgcctacagcagcctgctgagccaggccggcatcgacgactacaactacagggtgatcggcaggcccgccgaggacgaggacttcgccggcgtgaacaggctgatcaacgagtacaggcagaagaacgccgtgaggaacaggaacctgcccgtgatggtgaccctgtacaagcagatcctgagcgacagggagaacggcttcggcagggacatcctggccagcgacagcgaggccaaggagtacatcaggggcaagtacagcaaggcctacgacaccgcctgccagctgaccgtgttcgtgaacaactacatcctgaccgaggacctggacaccgtgtacgtgaagacccagcacctgaaggacatcagccagagcatcttcggcgagtggagcgtgctggacgacgccatcaacaacggcggcatcgacagcaagatcatcagcctgaaggccctggccgaggccttcgagaagtactgcagcgccctggaccccgacatggcccagaagtacagggacgccttcagcctgaaggagtacctgaagaacccccccgccgtggagcccaggctgcccgagaccgccgagaaggtgaccgcctacaagcccgccatggacgagctgatcagcctgatcaggttctacaagcccttctacctgtacgacggcaagaagaggctgcaggtgcccgccgagggcatggacttcagcaacgagttcgacaagtacttcgaggacctgaaggagttcagccacgcctacgacaaggtgaggaacttcgccaccaagaagccctacagcgtggagaagatcaagctgaacttcaaccagcccaccctgctggacggctgggacgtgaacaaggagaaggagaacgccagcttcctgttcgtgaaggagggcaagtactacctgggcatcgccgacaagagcgacaagaacctgttcgacaggagcagggacgccgtgaaggaggccctgaccctgacctgcggcgaggactactacgagaaggtgcagtacaagcagctgcccaacatcaggatgaacctgcccaggatcgtgttcaagcccaagtgggagaaggtgttcgcccccatcatcaccgagaggatcagcgccatcgagaaggagaagctgtacaccgccggcgccaacgacaggcaggccgtgatcgagtggatcgacttcgtgaagagcgtgctggccctgcacagcgagtggaacaactacttccagttcaagttcaaggagaccagcgcctacaagaacaccaaggagttctacgacgacctggacaaccaggcctacatcctgagcaagatcaagatcaacgccagctacatccacaagctggtgagggagaggaagctgtacctgttccagatctactgcaaggacttcagcgacaagaagaagaagaccggcgccgacaacctgcacaccatgtactggaggtgcctgttcagcgacgagaacctggccgccctggcccagggccaggcccccatcatcaagctgaacggcaaggccgagatcttcctgagggagcccagcctgaagtacgaggccacccaccccaagaaccagcccgtggccaacaagaaccccctgaacagcaagagggagagcctgttcagctacgacctggccaagaacaggaggttcaccgagaggaagctgttcttccactgccccctgaccatcaacttcaggaccgccgccgagggcgccaggagcttcaacgagagggtgaacaggttcgtggagaacaaccccgacatcaggatcatcggcatcgacaggggcgagaggcacctgctgtactacaccgtgatcaaccagagcggcaagatcctggagcagggcagcctgaacaagatcagcagcgactacaggacccccgagggcgccgaggtggagaaggtgaccgactaccacgagctgctgaacaggaaggagggcgacaggagggccgccaggcagacctgggacaccatcgagaacatcaaggagctgaaggagggctacctgagccaggtggtgcacaagctgaccaccctgatgatcaagcacaacgccgtggtggtgctggagaacctgaaccagaacttcaagaggagcaggatcaaggtggagaagcaggtgtaccagaagttcgagaaggccctgatcgacaagctgaactacctggtgttcaaggacaggccctacggcgcccccggcagctacgccaacggcctgcagctgaccgcccccttcgagagcttcgacaggctgggcaagcagaccggcgccctgtactacgtggtgcccagctacaccagccacatcgaccccaagaccggcttcgtgaacctgctgggcgccaagctgaggtacgagagcgtggtgaaggcccaggagaccatgaagaagatcggcagcttcaagttcaaccccgagaggaactggttcgagatcagcttcgactacggcgacttcggcatcgagatggcccacccccagtggaccgcctgcacccacggcgacgcctactggggctacaaccccaaggagaagagggccgtgagctacgacgtgaccgaggagatcaagaagctgctgaccgagtacggcatcgactacggcaccggcgccgacatcaaggaggccgccatcaggcagaccaagcccgccttctggaagaccctgttctacatgctgaagctggccctgcagctgaggaacaccgtggccggcaccgacaacgagaacgactacatcctgagccccgtggagtacgaggccggcaagttcttcgacagcaggaaggccggcgaggacgagccccagaacgccgacgccaacggcgcctaccacatcgccatgaagggcctgcaggccctgaggaggatcgaggacggcaaggtgaacaagttccccaagaacggcgagaggaaggcctggctggccttcatgcagaacagggagtacctgaag

SEQ ID NO: 26 - DNA, 3903 bp, synthetic construct
  source 1..3903: mol_type = other DNA
agcaagctgagcaccttcaacgagcacttccagaagaccatcaccctgaggaacgagctggtgcccgtgggcaagaccctggagaacatcatcagcagcaacgtgctgatcaacgacgagaagaggagcgaggactacaagaaggccaaggagatcatcgacagctaccaccaggagttcatcgagaagagcctgagcaaggtggacatcgagtgggacgacctggcctactgcctgaccaagaaggagcccgagaacttcgacaagaagaagaagtaccaggaggagatcaacaagctgcagaacgagaagaggaagaagagcgtggagatcttcaagaagttcagcttcggcgtgctgtgcgacaacaacggccaggaggtgcagatcaagttcgacgacctgttcaagagcaccctgttcaagtacgtgctgcccaacttcctgaagaacgacgaggacaagaccgtggtgagggacttcagcgagttcaccagctacttcaccggcttccacgagaacaggaagaacctgtacaagagcgcccccatgcccaccgccgtggcctacaggatcgtgaacgacaacttccccaagttcatcagcaaccagaagatcttctgcgcctggaaggacaacgtgccccacttcgtggagatcgccaagaccaagctgagcgaggtgggcatcagcgacctggacatcgagagcaagttcgagctgagcaacttcaacagctgcctgaaccagaccggcatcgactactacaacgacctgaggggccagctgaacaacgtgatcagccacatctgccagcaggacaagaacctgggcgacctgctgaggaagaagaggtgcctgaagatggtgcccctgtacaagcagatcctgagcgacaaggacagcagcttcagcatcgacgagttcgacaacgacgagagcgccatccaggacgtgatcagcttctacaagaagatggtgggcgagaggtgcccccagaggaagctgagcgagctgctgagggagctgagcagccacgacagggagaagatcttcatccagggcaagaacctgaacagcatcagcaggatcctgttcggcggcaagaactggagcctgctgagggaggccatcatcgaggagaagagcaaggacaagagcttcaagaaggccatcaagagcaacagcaccgacgaggccctggagaagatcctgagcaaggacgagttcagcatcagcttcctgagcaggatctgccacgagaacctgagcgagaagatcgacaagttcatcaagaaccaggacgagcagctgatcaacatcaacagccagacctggcccaagaccctgaagaacagcgaggagaagaagatcatcaaggagcacctggacttcctgctgaacttctacaggttcgccaagagcttcagcagcaacaccatcgacaaggacatgagcttctacgccgacttcgacgagagcctgagcagcctggagaacgtgatcggcctgtacaacaaggtgaggaactacgtgaccaagaagccctacagcctggagaagatcaagctgaacttcgagatccccaacctggccgagggctggagcaagaccaaggagctggccaacctgaccgtgatcttcctgaaggacaacgagtactacctgggcatcatcaacaagaacagcaagatcgacttcagcctgatcaaggagaagaccagcagcgagaactgcttcaggaagatggagtacagcctgttcggcaagatcgcccagaacatcaacaggtgcgtgttcaccaaggccgtgaagaaccacttcagcaagagccccgacaacttcatcctgagcgacaagaagaagttcctgaaggagatcgagatcaccaaggagatctacgacctggtgaagagcgacgagagcgacaagaagtacctgaaggagtacaagaagatcaacgagaaggagttcaggagcgccctgagcaagtggatcaacttctgcatcgagttcctgaagagctacaagaacaccagccagttcgacttcagctgcatcaagaaggccgagagctacgacgacatcaacgacttctacgacgacatcgagaaggtgacctacaagatcgagttcgtggacatcagcaccagcctgatcgacgccctggtggacaagggccagctgttcctgttcaagatcaggaacaaggacttcgccaagaaggccagcggcacccccaacctgcacaccctgtacttcaagagcatcttcgacaccaagaacttcgagaacggcatcgtgaagctgaacggcgaggccgagatcttctacaggaagaagagcctgaacgtggacgacaccaccgtgcaccagaagggcagctacatcgtgaacaaggtggtggtggaccccagcagcggcaagagcgagcagatccccgacaacgtgtacaacaacatctacgccttcgtgaacggcaagctgaagaccctgagcaaggaggacgaggtgtactacgccaaggccaccatcaagaaggccacccacgagatcgtgaaggacaggaggttcaccgaggacaagttcttcttccactgccccatcaccatcaactacaagagcaaggagaagcccagcaagttcaacgacaaggtgctggacttcctgaggaagaacgacgacatcaacatcatcggcatcgacaggggcgagaggaacctgatctacgccaccgtgatcaacaagaacggcgagatcatcgactgcaagagcttcaacaccatcaagcaccagagcagcagcgtgaacttcgacgtggactaccacaacaagctgcaggagagggagaagaacaggaaggaggagaagaggagctggaacagcatcagcaagatcgccgacctgaaggagggctacctgagcgccgtgatccacgagatcgccctgatgatggtgaagtacaacgccatcgtggtgatggagaacctgaaccagggcttcaagaagatcaggggcggcatcgccgagaggagcgtgtaccagaagttcgagaagatgctgatcgacaagctgaactacttcgtgatcaagaacgagaactggaccaaccccggcggcgtgctgaacggctaccagctgaccaacaaggtgagcaccatcaaggacatcggcaaccagtgcggcttcctgttctacgtgcccgccgcctacaccagcaagatcgaccccagcaccggcttcgtgaacctgatcaacttcaacaagtacaagaacagcaacgacaggaggaagctgatctgcagcttcgacaagatctgcttcgtgcagaacgagaacctgttcaagttcagcatcgactacgagaagctgtgccccaacagcaagatgcccgtgaagaagtgggacatcttcagctacggcaacaggatcgtgagggaggacctgaagcacatccccatgaaggagaaccccaagtacgaccccaccgaggagctgaagaccctgttcagcagcaaggagatcgagtaccagaagggccagaacctgctggagatcatcagcaccaaggacatgaccgaggacaaggagttctggaacagcctgttcaacatcttcaaggccatcctgcagatgaggaacagcctgaccaacagccccgtggacaggctgctgagccccgtgaagggcaaggacggcagcttcttcgacaccgacaaggtggagggcaccaagttcgagaagctgaaggacgccgacgccaacggcgcctacaacatcgccctgaagggcctgctggtgctgaagaagaacgacagcgtgaagaccgacaaggacatgaagaacgtgaagaacgtgagcctggaggactggctgaagttcgtgcaggtgaccctgagggtg

SEQ ID NO: 27 - RNA, 19 nt, synthetic construct
  source 1..19: mol_type = other RNA
aatttctactattgtagat

SEQ ID NO: 28 - RNA, 19 nt, synthetic construct
  source 1..19: mol_type = other RNA
aatttctactatcgtagat

SEQ ID NO: 29 - RNA, 19 nt, synthetic construct
  source 1..19: mol_type = other RNA
aatttctactgttgtagat

SEQ ID NO: 30 - RNA, 19 nt, synthetic construct
  source 1..19: mol_type = other RNA
catttctacttttgtagat

SEQ ID NO: 31 - RNA, 19 nt, synthetic construct
  source 1..19: mol_type = other RNA
aatttctacttttgtagat

SEQ ID NO: 32 - RNA, 19 nt, synthetic construct
  source 1..19: mol_type = other RNA
gatttctacttttgtagat

SEQ ID NO: 33 - RNA, 19 nt, synthetic construct
  source 1..19: mol_type = other RNA
aatttctactttcgtagat

SEQ ID NO: 34 - protein, 1283 aa, synthetic construct
MAPKKKRKVKTMTDLTGLYPLSKTLRFELKPIGKTLEHIERKGLIAQDEQRAEDYERVKDLIDKYHKKFISLCLNKCRLKVESTGEYDSLEEYVEFAGIAKRTQEEENAFTKVKEMLRKTVADSFKKDPDQFKELFKKEIITEHLPNMLLTDGSEEDKKKVESFALFTSYFTGFHQNRENMYSAEEKSTAIAYRLIHQNLPMFLDNIKSFEKIQATIDGKDMEELENAFRECLNVEHIADMFQLAYFTEILTQEQIDVYNYIIGGKTEEDGTKIKGINEYVNLYNQQHKDSRLPLLKPLYKMILSDRTTLSWLPEQFKSDEEMLTAINEAIKKLNDEVLNTANQDNLQQLLSHLSDYDTRHIYITNNHCLTDISQQIFGQYDRYTTAIKEEIKSSLKPTAKEKKDPDLLNDRTRKLFDARKSFSIAELNEYAQAEANQTIEVYFAQLGSYDRNGEQQISLLSRLNMAYNEAKEVLDGKYTNLNQSKEATEKIKNLLDAYKAIQFFAQPLAGSGDEAEKDNEFDAKLRNLTNTLSEVITPLYDKVRNWLTRKPYSTEKIKLNFENAQLLGGWDVNKEPDCTAILLRKHKQYYLGIMDKKSNHSFDIDELPSEGDCYEKIDYKLLPGANKMLPKVFFSKSRIDEFNPSDAIKKAYEKGTHKKGPDFSLADCHRLIDFFKASIAKHEDWKRFPFSFSDTGSYEDINGFYQEVEQQGYMIGFRPVSEAYINQLVEEGKLYLFQIWNKDFSEYSKGRPNMHTMYWNMLFDEKNLADVIYKLNGQAEVFFRQKSLDIAKTCVHAAQQPINNKNKSNQKQQSTFDYDIIKDRRYTMDKFQFHVPITMNFKSKGRENINSQVREIIRNNGIQHVIGIDRGERHLLYLSLIDLKGNIVKQMTLNDIINEYKGNTYATNYKQLLEYREGERTEARRSWQTIENIKELKEGYLSQVVHVIAKMMVEYNAIVVLEDLNGGFMRGRQKIERNVYEQFEKKLIDKLNYYVDKQKEATEAGGVLHALQLTSKFESFQKLGKQSGCLFYIPAWNTSKIDPVTGFTNQLDTRYQNVEKALQFFGRFDSIRYNKEKDWFEFMLDYDKFTTKAEGTQTRWTLCTYGTRIRSFRNDINKWVPKDVVLTEEFKQAFAAANIDVNGNLKDAICNLNEKAHLEELMHLVGLLVQLRNSKTNTEIDYMLSPVADKNGVFYDSRTCPDTLPKDADANGAYNIARKGIWVIRKIQEDQTNDKVDLKITNKEWLAFAQQKPYLKHLKRPAATKKAGQAKKKKDYKDDDDK

SEQ ID NO: 35 - protein, 1277 aa, synthetic construct
MAPKKKRKVEPIGKTADYIKNYQYLETDERLAKESKKVKELADEYHKEFIGDVLSSLELPLSKINELWDIYMSNDTDREIKFKKLQENLRKVIAEAFSKDKRFGNLFKKEIITDILPKFLQDKDDDIKIVNRFKGFTTYFYAFHKNRENMYVSEEKSTAIPYRIVNQNLVKYFDNYKTFKEKVMHLLKDKNIVESIERDFKDILNEKSIEDVFGLANFTHTLCQADIEKYNTLIGGLVVKNEKKEIKGINQYINEHNQTSKKGNGIPKLKPLFNQILSDRKSLSFTLDDIKKTSEAIRTIKDEYENLRDKLATIERLIKSIKEYDLAGIYIKMGEDTSTISQHWFGAYYKIIEAIADAWERRNPKKNRESKAYSKYVSSLKSISLQEIDDLKIGEPIENYFATFGTTCSDRTSGVSSLNRIKAAYTEFVNKFPEGFEDGDDCNDAYFKANVEVVKNLLDSIKDFQRFVKPLLGNEDERDKDEAFYGEFVPTYTDMDNIITPLYNRVRNFATKKPYSTDKIKINFENVVLLKGWDKNKESDYASIILMKDGQYFLGVLRNGSKSTLKTILPNTGDCYQKMVYKYFKDIKSNLPRCTTQRKDVKAHFAESSDDYTLLDTKAFVSALTISREVFELYNAPDKEKKFKKEYLKNTNDSIGYANAVSVCKRFCLEFLKKYRSTAIYDLSDVETSVDSFDDLSSFYQEIDKRLYSISFENVSVDSVNELVDNGNMLLFRIANKDFSPNSKGRPNLHTIYWRMLFDPANLKDVVYQLNGNAEIFFRKASVTRTEPTHPANVAIKNKSEYNKQNKPYSTFKYGLIKDRRYTTDQFEFHVPITMNFKQPESSKLQDKLNKQVLDFLKQDGVRHIIGIDRGERNLLYLVMVDMEGKIKKQISLNEIAGNPKNPEFKQDFLALLHEREGDRLESRRSWNTIQSIKELKEGYMSLVVHEIANMMLENDAIVVLENLNRSFMQKRGEKSVYQKFEKMLIDKLGYIVDKTKDVSDNGGALHAVQLADTFENFNKTQKGAIRQCGFIFYIPAWRTSKIDPVTGFVPMLRCQYESIVESKKFFGKFDSIYYDATGKYFVFQTDFTKFNTESKGGIQKWDICTYGDRIYAPRTKDRNNNPVSERVNLTEEMKSLFVSHNINIQGDIKAGIMQQTDKEFFESLHRLLRLTLQIRNSKKSTGKDYEDYIISPVMGKDGRFFDSRNADATQPKDADANGAYNIARKGLMLLRQIQAQEKQDLSNGKWLEFAQRKRPAATKKAGQAKKKKDYKDDDDK

SEQ ID NO: 36 - protein, 1334 aa, synthetic construct
MAPKKKRKVTRKFDDFIHQYSLSKTLRFEARPIGETLHNFIKNGLLERDEHRAESYVKVKKLIDEYHKAFIDRVLNDGCLNYEDKGEYDSLTEYYALYSAPRKDEAEREHFKETQQHLRDQVVKKLTGDNAYKHLFGKELIESYKDKEDKKKLNQADLVQFINTATPKQRLNFSKEEAIDLVQEFCGFTTYFGDFYQNRENMYSAEEKSTGIAYRLINENLPKFIDNMETFKKIAAIPEMEDNLKEIHDNLAEHLNVENIQDMFQLNYYNQLLTQKQIDVYNAIIGGKTDDEHKEKIKGINEYVNLFNQAHKDAKLPKLKTLFKQILSDKNAISWLPEEFDNDQEVLNAIKDCYARLSENILGKENLKRLLCSLSEYDTKGIFLRNDLQLTSISKKMSGSWTDIPNAIKNDLKDGVPAKKHKESEEDYEKRIDNLFKRQDSFSIGYIDDCLNKFDDNNTFTIEGYFKKLGAKDTQSEDIIKQIANAYTDVKPLLNSTYPKSKNLSQDKENVVKIKRFLDTLMSLVHFVKPLLGKGDESNKDEKFYGELSLLWTELETIVPLYNMVRNYMTRKPYSNSKIKLNFDNSQLLGGWDANKESDYASILLRRDGKYYLAIFDKDSKKLLGKSMPSDGECYEKMVYKFFKDVKTMIPKCSTQLKKVVKHFSISDTDFVLSDENFKVPLTITKEVYDLNNTMYGKYKKFHKGYLTTTGDIDGYTSAVKTWINFCMDFLGAYGSTMSYDLSSLATKEYSSLESFYQEANRLLYTVNFVKVSTSYVSRLVTEGKMYLFQIYNKDFSEYSKGTPNMHTLYWKALFDDRNLKDVVYKLNGQAEMFFRKKSIDCNRPTHPANQPIQNKNKDNKKKESVFEYDLIKDRRYTVDKFMFHVPITMNFKSTDKENINLPVMEYLQTSNDTHIIGIDRGERHLLYLVVIDLHGNIVEQYTLNDIVNEYNGNTYRTNYNDLLEARGNEQQKASQNWQTIENIKELKEGYLSQVIHKITQLMIKYHAIIVLEDLNMGFKRSRQKVEKQVYQKFEKMLIDKLNYLVDKKADIESTGGLLNAYQLTNKFVSFKKLGKQSGFLFYIPAWNTSKIDPVTGFVNLLDTRCQNKNKAKAFFAKFDSIRYNKDKDWFEFALDYDKFGSKAEGTRTKWILCTQGKRIKTFRNAEKNFQWYNQVIDLTKDLKQLFAQYDIDINGNLKEAISKQTKKTFFDDLMGLLKLTLQMRNSITRTETDYLISPVADEIGNFYDSRTCGPSLPENADANGAFNIARKGLMIIEQIKASEDLSKLKFDISNKSWLNFAQQKPYKHEKRPAATKKAGQAKKKKDYKDDDDK

SEQ ID NO: 37 - protein, 1315 aa, synthetic construct
MAPKKKRKVNTSKFIFQNLTHQFQLQKTLRFELIPQGRTLEHIEKNGLLEADELKANRYKQVKEWIDLYHKAFIEKVLREPKLEGLSEYRVFFENKQRNEKDEEAFKKLKSDLRKKISSFFKDEPNKTFYKRLFDKELIKADLPDFLREQGMHEEATMLLEEFGNFTTYFKGFHENRKNLYVAEDKSTAIAFRLIDENLPRYMQNLLLVEKHREVFEQFFPQIQERLGKELDGLRVEEFFSEEGFNLCLNGSGIHQYNTILGGVKGEEEEGIPGLNMLINLYRQKHKLKSRELPTLFPLHKQILSDRESSSFRPEPYEDGDENTVVEDLLKLGDQWFNPEGFVPQLVAHLTKLKDEEQDQLFVNNKANALSAVSQATLGHWAVLGEALSSYWNHVHDVKENALKGKALTAKEKWLKQPYFSFQEIELALRHYMGAEGKENASKWNVWLEKPLVQYFNYGLTKEAEKTKVDTAKAAWGHAENILRELQNHENPKLKTKETKDAIKAYFDACLDLLHFIKPLVVDLEQCEASPTFYGDFLSLYHEMDDLVPLYNKVRNYMTKKPYSYEKFKLNFNKGTLLDGWSQSKETDNLCVLFREGNNFFLGIMDRKHNKSFVKPPMAKEEEPVMQKVVYNLLPDPSKMLPKCFTAPYFRRIAKEKTKDPEQIVSEYSKEKGLIKPKLDVLDAYAHKKHTKENFQRNGFEQVVEYFKEGAQKHPDWHVFDFKFKPTSAYKDVSDFYQEVTEQGYCLDFVDIPKRYINDLVESGKLYLFQIYNKDFSKYSKGKPNLHTMYWKAIFDPQNLKAPIYKLNGEAEVFFRKKSLEYTQEQLDTGHHREELKGKFDYPIVKDRRFAFDKFQFHVPITLNHARKNQAVGPQVLRYIQKHKEELHYIGLDRGERHLLYLSVVNAKGEIIEQRSLNKIGNGFVKDGKPYTHDYQGKLLEKERNRDSARKDWDAVESIKELKEGYMSIIVHEIAQLVVKYNAVVILEDLNFGFKRGRFKVERQVYQKFERKLIEKLNHLVFKEVAPQEPGGLLKALQLTDQFTSFTKLGKQSGILFYLPAAYTSKIDPLTGFINQVPWKYTNLEAAQKWIGNFKRISYHAELDLFEWVLDQRDFLSNPAGCKHQWVLRMDNQERWHWNSKAEGTNGKGAYSPIRLKEQFEDLLARQNISYNGNSKLLEDIQAVKDASFFKTLFFFLRTLTQLRYNNGKSGAEEEDFILSAVANPGMEPFDTRKAKQGLPLDADANGAYHIAMKGLMTLGQLKEKPNGKLEIPKLDKSAWLCFMQQRSNQGKRPAATKKAGQAKKKKDYKDDDDK

SEQ ID NO: 38 - protein, 1295 aa, synthetic construct
MAPKKKRKVQTKNFMSFSDFTNLYALSKTLRFELRPIGRTEEFFKKNKVFEKDKTINDSYNQAKLYFDEMHRRLIRDALNTQNTSALDYEKYAKHWKELQKDKGKYIQNWKDAQKEFRECISNLLDQQAEQWKSKYASVGLNKADIKQKGTDFLSSAGMVKILKKEFPKEAENQLVKEGFPSLFIQKAEAPEQKQYIFDSFDRFSTYLAKFNQTRDNLYKDDGTPTAIATRVVENFVAFLQNQAIFNNHYISLKSELDFSREEIDCFTANYYVRCLLQEDIDEYNKTIGQINARIKELRDRKQKESDFKKSGYPLLKSLNKQILGKIEKKRQFIENKADVFPAFSELIEQANARLTALRQLINNLCEEVFEEAYGGIYIKNTGINTISRRWFASWYDFEIALPQKSKNEEESKVVTFVSLQDIKDTLENKLSGDIFKEEYYKKKIINKEQSNWKQILAILQNEWNELFSEYQKALENTKKLDLNSVFSNSKEQVEIIKSFADASLRVYQVVKYFALEHKKSDEIPSSYSTDFYEEFDKQYNNFEFIPYYNALRNFLTKKVVGENKIKINFEKGNLLNGWAESPEGNAQFCGYILKKENKYFLGITDQPTILDTKDNPSLTNITDKEYYEKMAYRQLKSTTIYGSGYEGLYNSKYANDKKYISESKIIERVKKLLQEKYLTRFPELKQIIEKEYKDANELAKNLGNFNLYSLEFIPVNKNYIEKLTHDKNGSIKYLYIFEIANKDLGKITPRKYNIHSLYFKNLFSEDNLEKPVFKLSGGAEIFFRPKTVNLPKKKDKAGKDIIDHKRYAQDKLFFHLPIVINFGAGKVGKFNQELNKKLLAGNKNINIIGIDRGEKHLAYYSVINQKGEIIEQDSLNEINGVNYYGLLQKREKERLENRKSWLPLQQIKDLKRGYISQVVYKIVRLAIEHNAIIVLEDLNMRFKQIRGGIEKSIYQQLEKALIDKLGYLVFKDRNAKEVGGVLQGYQLSAPFESFQKIGKQTGIIFYTQASYTSVTDPLTGFRKNIYVSNSAPIEKIKVAINNFEEIGWDDVVQSYFFNYDTKNFSNGNDIQSKKWKIYAKVPRIKREKVNGYWQYNDVDPNDMLEKLFRLYEFKNLRSNMLEQIHAKETNGELRGERDFDNKPRNFYQSLVYILNLILQLRNSTSKQIKLNEKEDVYETGEDVDFIASPAYPFFTTLSSKSNSNLAGFENRFTGLNHDQEQKKFNGDANGAYNIARKGIIILERITKNSDKPNLYISNEDWDKFVSQSYPKRPAATKKAGQAKKKKDYKDDDDK

SEQ ID NO: 39 - protein, 1310 aa, synthetic construct
MAPKKKRKVESKTLDRFIGKYQLSKTLRFELIPQGKTKEHIEQKGLLQQDEHRAKAYKMVKKIIDRYHQHFIATALNNLALDGLQSYEELYHKKEKEEADKKNFESLKAGMRKQIGDAFNKSEASKVQEIWKVLFDKELIRKALPEFVTSEEDKKLLAEFKDFTTYFTGFHQNRANIYTHEEKATAVAFRLVHENLPKFLDNSRVFDWIQTEYPDLDLSAIEKELAPELQGTTLPEIFALSFFNHTLTQNGIDLYNAVLGGKTTEQQNKIRGLNEYINLYNQRRTAEAKKEGEKFRRVPQFKELYKQILSDRESISFLPETFENDSEVLSAIENFFATQLVHFEKDNRSISVFAELQKLLQELKEDDRSKIFVRNDLALTNISQNIFGDWQVIKNALEHYYTTTVNPLAVGKKLTKAYENGKEKWQKQSYYSIYEIEEALLAYSSEADVLKEKNLTAASRPLCNYFATFSKDEEGNNLFQRIETAYTAVKDLLNTDYPEEKNLAQDKTNVAAIKTFLDAIKNLLHFIKPLHPARLEGEKDERFYGAFIPLFDQLNLLTPLYNKTRNYLTRKPYSTEKVKLNFENSTLLDGWDANKETANTSVLLRKDGLYYLAVMDKKYNKVFEQMPEVAEGEKVFEKVVYKLLPGANKMLPKVFFSAKNIQYYNPSTELLENYKNETHKKGATFNLVHCHQLIDFFKASIARHQDWKNFNFQFSDTGSYADLSGFYREVEQQGYKITFHNLPESYINQLVDQGKLYLFQIYNKDFSPFSKGRPNLHTMYWKMVFHEGNLTNVVYKLNGQAEIFFRKKSLNYDEKTLREGHHAAALKDKFAYPIISNRRFAFDKYQFHVPITINFKSTGRDNVNAEVLSFLQHNPSVNIIGIDRGERHLLYLTLIDQKGNIKKQMSLNQIINSYRNKEGDMVNVKTDYQNKLHLKEEERLKARQGWGLIENIKELKEGYLSHVVHEIAKMMVEHNAIVVMEDLNFGFKRGRQKVEKQVYQKFEKMLIDKLNYLVFKDRTATEPGGLLHALQLTGSYTDFMKYKQKQCGFLFYVPAWNTSKIDPLTGFVDFLKPKYESIEKAQNFLAKFKSIRYNPAKQWFEFTFDYKDFTDKAEGSRTRWTLCSTNQPRYRWNPKAANGKGAQEAVNVTQALEDLFSAHNIAYGSADDLVTAITAQSSADFYKALLRAMVTLLALRHNNGKAGTDEQDYILSPVEPFFDSRLADDSRPKDADANGAYHIAKKGLWVLQQINELQGDDWKNLKLAISNKQWLQFAQQLNADISPKVSKRPAATKKAGQAKKKKDYKDDDDK

SEQ ID NO: 40 - protein, 1375 aa, synthetic construct
MAPKKKRKVSSSIVSVSTEKKQAEVLSNKSVFDQFTRKYALSKTLRFELRPVGKTRENIEKANPNFIRDKEIEDAYQILKPVFDKLHEEFITESLESDEAKHLSFEDYFSVYKNLRNEQDREEKKKIEKSLETKEKNLRAAFSDIYKAEGEKFKSKVGSDKKGKPILKEDSFKILTEVGILKYVKAKIEEFVVMNLKTHEGKEVKKEDLEKALGTADSKGVFEGFFTYLTGFNQNRENYYSTEEKATAVASRIIDENLPKFADNVLEFQKRKDEYLKVYDFLKENGIETIGKNQKGEKVELEKIIADIFEIGYFTNCLSQREIEKYNLNIGNANNLINRYNQQQSEKNKKLRIFKTLYKQIGCGEKGDFIPAIKNDDGLRAVLKNVVENGEKYFGQFDGKIEDKVDTVGEFVHYIINHESFEGIFWSDKALNTISGKYFANWFILKKALKEGGVFGGKKKDDEDIKIPQAVELKNLFEILDKTEKWKNKEDGLFKTSLFDGEGNKKGIIERAEKPSKALLGMIFADVEKMAKDFSELSPKVLSITTDFKKPEHIENIKMLLDSALYVNQIVKYWKVKDKFAVDPILAESLKAILFGDNNPTKFYDIIRNYLTKRPQEGLNKLKLNFDNSVLGGGWDVNKEPERWCLILKDGEKKYLAILTKASQKLFGEKAKYKEVVTDENYLKMDYKLLPGPNKMLPKVLLPKRNRKEFGATDDILSIYDEGGFKKNQPSFTKEKLWKIIDFYKEGLKVYPSAEKSWQDLFGFNFSPTEKFESIDQFYNEVEKQGYKLSWNIISKKLINEAVENGEIYFFEIRNKDNNLKEGKEKTSEKNLHTIYWNEIFGDVANKPKLNGEAEIFYRKTILKKKENETDKEFEIRLKEKLGKKKDKSGKEVLEHKRFANEKFVFHCPITLNFCLRSLKFNDEINESMVKLNDDITTIGIDRGEKHLAYYFVVKQTKDGKIETLDQGSFNEINGQNYAEKLEKMAGNRDEARKNWKTIGTIKELKDGYISQVVRKIVDLAIYEDVEKKVFRETPAFIVLEDLNIGFKRGRQKIEKQVYQKLELALAKKLNFLVDKHAKEGEIGSVTSALQLTPLVNNFGDIEGKKQFGIMFYTKADYTSQTDPVTGWRKTIYLKKGSEESIKKQILDNFDDIKFDGKDYYFKYTDRNTNKPWELYSGKDGESLDRYYRELVHENSDKKWVPKEQNLKKILDGLFEGFDKEKSLLEQLRQGKNPNKINDRTSWESLRFVIDLIQQIRNSGKTEEDADFILSPVRDKNGNHFDSRKINLNLPTSGDANGAYNIARKGIIMCEHIKRDLSLFISDEEWSAWLAGREIWENWIKKNEKNLKRKQKRPAATKKAGQAKKKKDYKDDDDK

SEQ ID NO: 41 - protein, 1322 aa, synthetic construct
MAPKKKRKVKSIEELIGQYSLTKTLRFELKPIGKTLEQIERKGVLSADQTRADQYKRVKESIDRYHKAFITESLDSLVLKVKSTGNTSDSLEEYASLLDNSADDEALDKVKKGLRKQVAERFDKQIRLKKLFKNDLITKILPEFLKVNGNEDERKAVESFRNFTTYFSGLNENRKNMYSKEAKSTAIAYRLIDENFPRFHDNMLSFAKIAAVNEVSNHFAEIAEAFKSEMVVASVAEMFGLEYFNNTLTQQKIDLYNYVIGGKTEGDKVKIQGINEYVNLYNQNHPNDKLPLLKPLYKMILSDRVALSWLPEEFNDDEAMVCAIKEFYNAIQPVLWGEVEDDDNSVCHLLKNIGKYDIEHIFISNGAALNDVSRQMFGQYDFLANAIKKDIRQNTKRSSRETDEKFEGKVNTLFKKAKSYSIAHLNALIKENRSETIESFYSNLGACDRDGEQKINLLTQIELAHEAAQDVLDGKYADINRDEKATMLIKELLDAFKALQYFIKPLLGSGEEASKDNVFCTKLLNAWQALDAVTPLYDKVRNRLTRKPYSTDKIKLYFENKGKFLDGWVDSKTDNSDNGTQFGGYILRKKNEIGEYDYFLGISSDTKLLRANPDIQYEHGMYERLDYYQIQSKTFLGSSYKGEYNDDAMRLMSAFVEYGKKSRFDSCVLPTDKEKSLPTYLKRIYTDNNSFYNEAMTDTVVAEAYNNIKSHLLLTLAGIKRVEAAVNLSKHNEYNLIQIFDEIGKITSSSKIFSFFPVDTSLVEKLNERDNKPLFLFKISNKDLSYAETSSKNIRKSRGKENLHTMYFKALMEQSHNVFDIGTAEVFFRKATPGLDKSTTIHKANEALCNKNPHSKKTSSTFGYDIIKDRRYVENKFQLHLSMVINYQGKNPNTDIISPQVLDIIRNNGIKHIIGIDRGERNLLYLSLIDLKGNIIKQISLNEIKPLGNDGCVTNYRDILADREKDRKEARRSWKKLENIKDLKSGYLSQVVHVIAKMMVDYQAIVVLEDLNSAFMQSRQKIERSVYEQFEHKLINKLNYYVDKSVSDDLPGGLLYGLQLASKKTHDVQCGCIFYVSAWNTSKIDPVTGFVNMLDTRFENVSSSRSFFSHFDSIRYNVAKDWFEFTFDYNNFHKKLEGTKTRWTLCTYGTRILTQRNDRNQWESREVDITDEFKKAFVAAGIDIHGNLKDAICALDKRVHLEQLMRLMKLLLQLRNSIINQDTDYILSPVAENGVFYDSRRHADNLPIDADANGAYNIARKGLYLLRKIKADRSAKPELCISNKEWLRFVQDKSNPGDKRPAATKKAGQAKKKKDYKDDDDK

SEQ ID NO: 42 - protein, 1359 aa, synthetic construct
MAPKKKRKVVTIDDFCGQKNGYSRSITLRNKLIPVGKTQENLEKANMLEEDFSRAKAYVEVKKIIDDFHRSFIEDVLSKANLNWKELYEKIDSFQRETDKAKKAKNQKDLQPVQVKMRKAIVKIFKDDERFDKLFKKELLSDLLPEIIKSAPENEISNKQTALDVFKGFATYFTGFHENRKNMYSEEDKATAISYRIVNENFPKFYANIKLFEKIQKEFPVIIEETEKSLASFLAGKKLKDIFTPEAFNQVLTQSGIDFYNTVIGGIAGEAGTEKVQGLNEKINLARQQLPDEEKSKLRGKMVVLYKQILSDRETASFIPVGFENKDEVYKTLQEFKKSTVEKSIEIIRETFKSPENFDLSKIYVPAKELTAFSLSVFGRWNILSDGLFAIVEEKSKKALSEKQTEKLANEIAKKDYSLSELQAAYEKYLEKNPVEDEGRCRRLSAKNYFELIELRKIDDSAEKEFVNVLSNLDELWSKIDFENPRNLQQEKEAATPIKNYLDEVQNLYHFLKLVDYRGEEEKDSEFYSKLTEILDLLSEIVPLYNKVRNFVTKKPGEVKKIKLNFDCSSFLKGWGTEFGTKDAFIFIDEKKYYLGVINKKLDSDTVKYLQTPGEHIIKQVFVNFQKPDFKNFPRLFIRSKKDRFAPAVEQFNLPVNDILDIYDKGLYKTEYRKENPIIYKESLIKMIDYFKLGISQHESFKGFKLTWKESSEYENISDFYADVTASCYQVNFTNVNFDRLLSLANEIEDDNKKIFLFEIYNKDFSTGKNGGNGSTGKKNLHTMYFENLFSKENLKDIVLKLSGEAEIFLKSENTELKKIVHKQGTTLVNRTTKDKKLIPENIYQEIYQFKNSMIDNISDEAKAWLNSGNVECKIAKFDLIKDKHLIEKSYYLHCSIIQNFKAPEKVFNFNEKVQRYLKDNPDVKIIGLDRGERHLIYLSLINQKGEIELQKTLNLVEQVRNDKTVKVNYQEKLVQKEGDRDKARRNWQTIGNIKELKEGYLSNVVHEIAKLMVEHNAIVVMEELNFGFKRGRFPVERQVYQKFENMLIGKLNYLVFKDKSVTEPGGVLNAYQLTDKSTNVSDVGKQCGWLFYVPAAYTSKIDPKTGFANLLNMKDLTNVAKKRMFFSKFKDIHFDSGSNDFAFTFDYKDFDGKGAAEMTQTEWTVWSNGKRIFYNSKHHSTEEINVSEKLKELFDASKIDYKTENNIFESIMEVGADLKEGEKPSKEIANFWDSLLWYFKLILQMRNSIPNSETDYLISPVKAEDGTFFDSRVQKELGKDSKLPIDADANGAYHIALKGLYLLKNDFNLNDKGYIENISNAEWFRFVQERDYLNKRPAATKKAGQAKKKKDYKDDDDK

SEQ ID NO: 43 - protein, 1334 aa, synthetic construct
MAPKKKRKVPNLFDNFTQQYSLTKTLRFELKPEGKTLENMQNAFKFDKDLQTFLVDQDIEDAYQTLKPYIDFIHEEFITESLESDIAKGIDFSEYFENFKIKKDINQKDFQNLEHKLRKEFDQLYIQIANKWKEEKYKQYKWKKGSKVANGSEILSSEDILKLVVDLNQDNNQLKDIVQNTFKGFFTYFSGFNDNRRNYYQVEKEAKSSIATRIVNENLPKFCDNFLLFENRKNEYLGVYNTLKLLGKDLVDKDRKTLVTIEEIIFSIKHFNFCLSQKEIENYNKQIGNANLLINLYNQNTSNESDYKRLPLFKTLYKQIGCGKRDSLFFEITDYTKLEAEGKRILGKSAISVEEVLIQVKESGIKYFTEKLDSLLNYILEKETDNYQGLYWSNIAVNSISSKYLANWNNLKELFKGKKEVASYDKGREEEIKINEAVDLSILFSILNNTEDWKVKEVFFKENLNEESETKKIIETSKTPSQALLRIVIGDLKKNASNFLNLSDNIEQLLESKEKIKESMDYALSVTRMLKYFLVKESKTKGAPLDSKLVEDLNLLLLSDDAPWFMWYDGLRNYLTKKPHDDAKENKLKLNFENSTLAGGWDINKEPANYCVILTNSANDQYLAINAKRNNKIFCKELVDGKGKDKKLLKNPLYESDEQHWKKMEYKLLPGPNKMLPKCLLPKSNLRKFGASEEVLSIYENGSFKKNTPDFSTQDLHKLIDFYKSALLKYEDWKCFHFKFNDTRSYGDISQFYSDLEKQGYLLSFIDINKSVLNQLVDSGKIYLFQIRNQDSNISKKINHQDNLHTIYWRAVFEDVFNKPKLNGQAEVFYRKALPQEDLQLLKDKDNKEIIANYRFSKEKFLFHVPITLNFCLKNKKINDLANREILSNDNIYFLGIDRGEKHLAYYSLVDQKGNIVIDQNGKSIQGTLNIPFLDSNGNQRLVKATKRVMKDGKEDAQVVECKDYNELLEARASDRDYARKNWQTIGAIKDLKDGYISQVVRKIADLAVEYNAYIVLEDLNTGFKRGRQKIEKSVYQKLELALAKKLNFLVDKSAKIGEVGSVTNALQLTPPVNNYSDIENRKQFGVVLYTRANYTSQTDPVTGWRKTIYLKTGSEDIIKKQIFESFSNIYFDGQDYIFTYTDKNIDRKWELYSGKNGKSLDRYRGRKVNNKGWVIDKINIQEILDKIFINFEICYGEKNNIFEKLKAGVELEKYNEYTAWESLRFAIDIIQQIRNTGIESSDSDFLLSPIRVNNLHFDSRLASENQPNSGDANGAYNIARKGIIMANYIKQGVSPFVSDIDWDNFVTKNKRPAATKKAGQAKKKKDYKDDDDK

SEQ ID NO: 44 - protein, 1437 aa, synthetic construct
MAPKKKRKVNKFLDSFTHQYSLSKTLRFELKPVGKTAEWIQKRDIIGTKKQENGEAQLTGKDAVRAENYEYAKKLLNEMHRLFIEDSLAAIKEEQKVELNKLLVKIAESGSLDGFRKPMSNICKELFDSATQDWLLAYSEEMPCFWDEDILELTNKAETTENKKNIKFFLKKAEDIRKFKDDPLKAFKKKKHEALNSNTEALKLLEWKIRRGEVLISGKDLGRDESEEILSTKELILILRSFYSFVTYFTGFNENRANIYNLDDKKYISTSIAFRSFEQNLFFHIENIKKWHTITNSIEEHTDQLNASDYPIKDKLRELELAFDCKLADLFAVDSFSQFLSQSGIDRYNSIMGGIAQKTGKTKVQGLNEIINQARQQTGAKRHKFPPMKLLYKQILSKGEGIFVESYVDDADMLTSIAGFHENHLNESLCSNWLHELEELIDSVQESKDTIFIARDKLNRISQEITGSWSAIQDWIVAHQKDQKEVDALTKKKVFSIQELEDYFSKSSDDELFTAKIPEDRSAYLPGEGEGILFQFFKVRAKEILAELQSGWYALDKAQLLQEKQLDKNRENEGSKGFEQVGLLKGYLDACNSLSRFIADWNIQKDIKQERCSLWYEFLQKHLDTVPIFDLYNQVRNHVSKKAYSTDKLKVNFEKSTLLDGWDRNKESQNFGVLFEKDGAYYLGIMTPDSNGIFEYDVNPDENTKKKAIKEELRKQTTPENGEDRFQKINYKLLPGPNKMLPKVFFAKSNEDLFQPSARITEIKENKKYAKAYTDKHGKAELHEYIDFCQKSLVRHPEWSVAYGFDQTTFRKATEYISIDEFYREVELLGYKVTFDSIKATYIEEKVANGELYLFQIYNKDFSTAKKGGGKDNLHTLYWKSLFSEDNLKDVVFKLNGQAEVFFRPSSIVWSEKVMKQGHHYDDDKMLKDKFKYPIIKDFRFTQDKFFFHCPISLNFKAPAKPYGFNYKVHDFLRKNGEINVIGIDRGEKHLLYYSITNQQGDIIEQGSLNSIESSYEKGSSSVPHSTNYLEKLQEKEKNRDSARKSWSRIENIKELKAGYLSHVVHKLAKLIIKHNAVVVLEDLNAGFKRGRFKVERQVYQKFEKALIDKLNYLVFKDQEKPSAPGHPLRAYQLTDPFESFEKLGKQSGILFYTTAAYTSTTDPVTGFLKNVYKPYANVVDSSDFWKSFDEIHYDVKKERFEFTYDLRKIKNKQVDRDKDEAKVSRSVWTVCSCVERSRYVKRKANEEQKQGAASKSIGKMGEYEVFVVTDKLKALFEKESISYQSGEDIRSQLSNRIDKKDAKLHRELIYYFGTLLNMRVTDKQSESGTDENDYILSPIEPFFDSRKGYEKLPVNGDANGAYNIARKGIAILNRIHESDSGDKVDLMIRKKDWQDFVQNEEVVQKQMGKLDLKRPAATKKAGQAKKKKDYKDDDDK

SEQ ID NO: 45 - protein, 1271 aa, synthetic construct
MAPKKKRKVALFSENFVGCYPVSKTLKFELRPVPETKAYLDDDNCPLLAGDLERDKAYPVVKELLDKYYRYFIDICLKDKKLSPETIDEAYQAYSSHDEKSQKALKAASQKLRKEVAGFFDKKTRETYCLDKYSNLLVLDKKNDNKEKGTPKTIPSVLKKWLENESGFSEKEVEKYSQAVQTFNGFTTYFISYKENRENMFSSEEKASAISYRVVNQNMLRFFSNVENFENIKKKYPDLYQTLKEFEESFVPAAYSSLLSQAGIDDYNYRVIGRPAEDEDFAGVNRLINEYRQKNAVRNRNLPVMVTLYKQILSDRENGFGRDILASDSEAKEYIRGKYSKAYDTACQLTVFVNNYILTEDLDTVYVKTQHLKDISQSIFGEWSVLDDAINNGGIDSKIISLKALAEAFEKYCSALDPDMAQKYRDAFSLKEYLKNPPAVEPRLPETAEKVTAYKPAMDELISLIRFYKPFYLYDGKKRLQVPAEGMDFSNEFDKYFEDLKEFSHAYDKVRNFATKKPYSVEKIKLNFNQPTLLDGWDVNKEKENASFLFVKEGKYYLGIADKSDKNLFDRSRDAVKEALTLTCGEDYYEKVQYKQLPNIRMNLPRIVFKPKWEKVFAPIITERISAIEKEKLYTAGANDRQAVIEWIDFVKSVLALHSEWNNYFQFKFKETSAYKNTKEFYDDLDNQAYILSKIKINASYIHKLVRERKLYLFQIYCKDFSDKKKKTGADNLHTMYWRCLFSDENLAALAQGQAPIIKLNGKAEIFLREPSLKYEATHPKNQPVANKNPLNSKRESLFSYDLAKNRRFTERKLFFHCPLTINFRTAAEGARSFNERVNRFVENNPDIRIIGIDRGERHLLYYTVINQSGKILEQGSLNKISSDYRTPEGAEVEKVTDYHELLNRKEGDRRAARQTWDTIENIKELKEGYLSQVVHKLTTLMIKHNAVVVLENLNQNFKRSRIKVEKQVYQKFEKALIDKLNYLVFKDRPYGAPGSYANGLQLTAPFESFDRLGKQTGALYYVVPSYTSHIDPKTGFVNLLGAKLRYESVVKAQETMKKIGSFKFNPERNWFEISFDYGDFGIEMAHPQWTACTHGDAYWGYNPKEKRAVSYDVTEEIKKLLTEYGIDYGTGADIKEAAIRQTKPAFWKTLFYMLKLALQLRNTVAGTDNENDYILSPVEYEAGKFFDSRKAGEDEPQNADANGAYHIAMKGLQALRRIEDGKVNKFPKNGERKAWLAFMQNREYLKKRPAATKKAGQAKKKKDYKDDDDK

SEQ ID NO: 46 - protein, 1334 aa, synthetic construct
MAPKKKRKVSKLSTFNEHFQKTITLRNELVPVGKTLENIISSNVLINDEKRSEDYKKAKEIIDSYHQEFIEKSLSKVDIEWDDLAYCLTKKEPENFDKKKKYQEEINKLQNEKRKKSVEIFKKFSFGVLCDNNGQEVQIKFDDLFKSTLFKYVLPNFLKNDEDKTVVRDFSEFTSYFTGFHENRKNLYKSAPMPTAVAYRIVNDNFPKFISNQKIFCAWKDNVPHFVEIAKTKLSEVGISDLDIESKFELSNFNSCLNQTGIDYYNDLRGQLNNVISHICQQDKNLGDLLRKKRCLKMVPLYKQILSDKDSSFSIDEFDNDESAIQDVISFYKKMVGERCPQRKLSELLRELSSHDREKIFIQGKNLNSISRILFGGKNWSLLREAIIEEKSKDKSFKKAIKSNSTDEALEKILSKDEFSISFLSRICHENLSEKIDKFIKNQDEQLININSQTWPKTLKNSEEKKIIKEHLDFLLNFYRFAKSFSSNTIDKDMSFYADFDESLSSLENVIGLYNKVRNYVTKKPYSLEKIKLNFEIPNLAEGWSKTKELANLTVIFLKDNEYYLGIINKNSKIDFSLIKEKTSSENCFRKMEYSLFGKIAQNINRCVFTKAVKNHFSKSPDNFILSDKKKFLKEIEITKEIYDLVKSDESDKKYLKEYKKINEKEFRSALSKWINFCIEFLKSYKNTSQFDFSCIKKAESYDDINDFYDDIEKVTYKIEFVDISTSLIDALVDKGQLFLFKIRNKDFAKKASGTPNLHTLYFKSIFDTKNFENGIVKLNGEAEIFYRKKSLNVDDTTVHQKGSYIVNKVVVDPSSGKSEQIPDNVYNNIYAFVNGKLKTLSKEDEVYYAKATIKKATHEIVKDRRFTEDKFFFHCPITINYKSKEKPSKFNDKVLDFLRKNDDINIIGIDRGERNLIYATVINKNGEIIDCKSFNTIKHQSSSVNFDVDYHNKLQEREKNRKEEKRSWNSISKIADLKEGYLSAVIHEIALMMVKYNAIVVMENLNQGFKKIRGGIAERSVYQKFEKMLIDKLNYFVIKNENWTNPGGVLNGYQLTNKVSTIKDIGNQCGFLFYVPAAYTSKIDPSTGFVNLINFNKYKNSNDRRKLICSFDKICFVQNENLFKFSIDYEKLCPNSKMPVKKWDIFSYGNRIVREDLKHIPMKENPKYDPTEELKTLFSSKEIEYQKGQNLLEIISTKDMTEDKEFWNSLFNIFKAILQMRNSLTNSPVDRLLSPVKGKDGSFFDTDKVEGTKFEKLKDADANGAYNIALKGLLVLKKNDSVKTDKDMKNVKNVSLEDWLKFVQVTLRVKRPAATKKAGQAKKKKDYKDDDDK

SEQ ID NO: 47 - DNA, 3852 bp, Synthetic construct
  source 1..3852: mol_type = other DNA
atggcccccaagaaaaagcggaaggtgaagaccatgaccgacctgaccggcctgtaccccctgagcaagaccctgaggttcgagctgaagcccatcggcaagaccctggagcacatcgagaggaagggcctgatcgcccaggacgagcagagggccgaggactacgagagggtgaaggacctgatcgacaagtaccacaagaagttcatcagcctgtgcctgaacaagtgcaggctgaaggtggagagcaccggcgagtacgacagcctggaggagtacgtggagttcgccggcatcgccaagaggacccaggaggaggagaacgccttcaccaaggtgaaggagatgctgaggaagaccgtggccgacagcttcaagaaggaccccgaccagttcaaggagctgttcaagaaggagatcatcaccgagcacctgcccaacatgctgctgaccgacggcagcgaggaggacaagaagaaggtggagagcttcgccctgttcaccagctacttcaccggcttccaccagaacagggagaacatgtacagcgccgaggagaagagcaccgccatcgcctacaggctgatccaccagaacctgcccatgttcctggacaacatcaagagcttcgagaagatccaggccaccatcgacggcaaggacatggaggagctggagaacgccttcagggagtgcctgaacgtggagcacatcgccgacatgttccagctggcctacttcaccgagatcctgacccaggagcagatcgacgtgtacaactacatcatcggcggcaagaccgaggaggacggcaccaagatcaagggcatcaacgagtacgtgaacctgtacaaccagcagcacaaggacagcaggctgcccctgctgaagcccctgtacaagatgatcctgagcgacaggaccaccctgagctggctgcccgagcagttcaagagcgacgaggagatgctgaccgccatcaacgaggccatcaagaagctgaacgacgaggtgctgaacaccgccaaccaggacaacctgcagcagctgctgagccacctgagcgactacgacaccaggcacatctacatcaccaacaaccactgcctgaccgacatcagccagcagatcttcggccagtacgacaggtacaccaccgccatcaaggaggagatcaagagcagcctgaagcccaccgccaaggagaagaaggaccccgacctgctgaacgacaggaccaggaagctgttcgacgccaggaagagcttcagcatcgccgagctgaacgagtacgcccaggccgaggccaaccagaccatcgaggtgtacttcgcccagctgggcagctacgacaggaacggcgagcagcagatcagcctgctgagcaggctgaacatggcctacaacgaggccaaggaggtgctggacggcaagtacaccaacctgaaccagagcaaggaggccaccgagaagatcaagaacctgctggacgcctacaaggccatccagttcttcgcccagcccctggccggcagcggcgacgaggccgagaaggacaacgagttcgacgccaagctgaggaacctgaccaacaccctgagcgaggtgatcacccccctgtacgacaaggtgaggaactggctgaccaggaagccctacagcaccgagaagatcaagctgaacttcgagaacgcccagctgctgggcggctgggacgtgaacaaggagcccgactgcaccgccatcctgctgaggaagcacaagcagtactacctgggcatcatggacaagaagagcaaccacagcttcgacatcgacgagctgcccagcgagggcgactgctacgagaagatcgactacaagctgctgcccggcgccaacaagatgctgcccaaggtgttcttcagcaagagcaggatcgacgagttcaaccccagcgacgccatcaagaaggcctacgagaagggcacccacaagaagggccccgacttcagcctggccgactgccacaggctgatcgacttcttcaaggccagcatcgccaagcacgaggactggaagaggttccccttcagcttcagcgacaccggcagctacgaggacatcaacggcttctaccaggaggtggagcagcagggctacatgatcggcttcaggcccgtgagcgaggcctacatcaaccagctggtggaggagggcaagctgtacctgttccagatctggaacaaggacttcagcgagtacagcaagggcaggcccaacatgcacaccatgtactggaacatgctgttcgacgagaagaacctggccgacgtgatctacaagctgaacggccaggccgaggtgttcttcaggcagaagagcctggacatcgccaagacctgcgtgcacgccgcccagcagcccatcaacaacaagaacaagagcaaccagaagcagcagagcaccttcgactacgacatcatcaaggacaggaggtacaccatggacaagttccagttccacgtgcccatcaccatgaacttcaagagcaagggcagggagaacatcaacagccaggtgagggagatcatcaggaacaacggcatccagcacgtgatcggcatcgacaggggcgagaggcacctgctgtacctgagcctgatcgacctgaagggcaacatcgtgaagcagatgaccctgaacgacatcatcaacgagtacaagggcaacacctacgccaccaactacaagcagctgctggagtacagggagggcgagaggaccgaggccaggaggagctggcagaccatcgagaacatcaaggagctgaaggagggctacctgagccaggtggtgcacgtgatcgccaagatgatggtggagtacaacgccatcgtggtgctggaggacctgaacggcggcttcatgaggggcaggcagaagatcgagaggaacgtgtacgagcagttcgagaagaagctgatcgacaagctgaactactacgtggacaagcagaaggaggccaccgaggccggcggcgtgctgcacgccctgcagctgaccagcaagttcgagagcttccagaagctgggcaagcagagcggctgcctgttctacatccccgcctggaacaccagcaagatcgaccccgtgaccggcttcaccaaccagctggacaccaggtaccagaacgtggagaaggccctgcagttcttcggcaggttcgacagcatcaggtacaacaaggagaaggactggttcgagttcatgctggactacgacaagttcaccaccaaggccgagggcacccagaccaggtggaccctgtgcacctacggcaccaggatcaggagcttcaggaacgacatcaacaagtgggtgcccaaggacgtggtgctgaccgaggagttcaagcaggccttcgccgccgccaacatcgacgtgaacggcaacctgaaggacgccatctgcaacctgaacgagaaggcccacctggaggagctgatgcacctggtgggcctgctggtgcagctgaggaacagcaagaccaacaccgagatcgactacatgctgagccccgtggccgacaagaacggcgtgttctacgacagcaggacctgccccgacaccctgcccaaggacgccgacgccaacggcgcctacaacatcgccaggaagggcatctgggtgatcaggaagatccaggaggaccagaccaacgacaaggtggacctgaagatcaccaacaaggagtggctggccttcgcccagcagaagccctacctgaagcacctgaaaagacctgccgccacaaagaaagccggacaggccaagaagaaaaaggattacaaagacgatgacgataagtga

SEQ ID NO: 48 - DNA, 3834 bp, synthetic construct
  source 1..3834: mol_type = other DNA
atggcccccaagaaaaagcggaaggtggagcccatcggcaagaccgccgactacatcaagaactaccagtacctggagaccgacgagaggctggccaaggagagcaagaaggtgaaggagctggccgacgagtaccacaaggagttcatcggcgacgtgctgagcagcctggagctgcccctgagcaagatcaacgagctgtgggacatctacatgagcaacgacaccgacagggagatcaagttcaagaagctgcaggagaacctgaggaaggtgatcgccgaggccttcagcaaggacaagaggttcggcaacctgttcaagaaggagatcatcaccgacatcctgcccaagttcctgcaggacaaggacgacgacatcaagatcgtgaacaggttcaagggcttcaccacctacttctacgccttccacaagaacagggagaacatgtacgtgagcgaggagaagagcaccgccatcccctacaggatcgtgaaccagaacctggtgaagtacttcgacaactacaagaccttcaaggagaaggtgatgcacctgctgaaggacaagaacatcgtggagagcatcgagagggacttcaaggacatcctgaacgagaagagcatcgaggacgtgttcggcctggccaacttcacccacaccctgtgccaggccgacatcgagaagtacaacaccctgatcggcggcctggtggtgaagaacgagaagaaggagatcaagggcatcaaccagtacatcaacgagcacaaccagaccagcaagaagggcaacggcatccccaagctgaagcccctgttcaaccagatcctgagcgacaggaagagcctgagcttcaccctggacgacatcaagaagaccagcgaggccatcaggaccatcaaggacgagtacgagaacctgagggacaagctggccaccatcgagaggctgatcaagagcatcaaggagtacgacctggccggcatctacatcaagatgggcgaggacaccagcaccatcagccagcactggttcggcgcctactacaagatcatcgaggccatcgccgacgcctgggagaggaggaaccccaagaagaacagggagagcaaggcctacagcaagtacgtgagcagcctgaagagcatcagcctgcaggagatcgacgacctgaagatcggcgagcccatcgagaactacttcgccaccttcggcaccacctgcagcgacaggaccagcggcgtgagcagcctgaacaggatcaaggccgcctacaccgagttcgtgaacaagttccccgagggcttcgaggacggcgacgactgcaacgacgcctacttcaaggccaacgtggaggtggtgaagaacctgctggacagcatcaaggacttccagaggttcgtgaagcccctgctgggcaacgaggacgagagggacaaggacgaggccttctacggcgagttcgtgcccacctacaccgacatggacaacatcatcacccccctgtacaacagggtgaggaacttcgccaccaagaagccctacagcaccgacaagatcaagatcaacttcgagaacgtggtgctgctgaagggctgggacaagaacaaggagagcgactacgccagcatcatcctgatgaaggacggccagtacttcctgggcgtgctgaggaacggcagcaagagcaccctgaagaccatcctgcccaacaccggcgactgctaccagaagatggtgtacaagtacttcaaggacatcaagagcaacctgcccaggtgcaccacccagaggaaggacgtgaaggcccacttcgccgagagcagcgacgactacaccctgctggacaccaaggccttcgtgagcgccctgaccatcagcagggaggtgttcgagctgtacaacgcccccgacaaggagaagaagttcaagaaggagtacctgaagaacaccaacgacagcatcggctacgccaacgccgtgagcgtgtgcaagaggttctgcctggagttcctgaagaagtacaggagcaccgccatctacgacctgagcgacgtggagaccagcgtggacagcttcgacgacctgagcagcttctaccaggagatcgacaagaggctgtacagcatcagcttcgagaacgtgagcgtggacagcgtgaacgagctggtggacaacggcaacatgctgctgttcaggatcgccaacaaggacttcagccccaacagcaagggcaggcccaacctgcacaccatctactggaggatgctgttcgaccccgccaacctgaaggacgtggtgtaccagctgaacggcaacgccgagatcttcttcaggaaggccagcgtgaccaggaccgagcccacccaccccgccaacgtggccatcaagaacaagagcgagtacaacaagcagaacaagccctacagcaccttcaagtacggcctgatcaaggacaggaggtacaccaccgaccagttcgagttccacgtgcccatcaccatgaacttcaagcagcccgagagcagcaagctgcaggacaagctgaacaagcaggtgctggacttcctgaagcaggacggcgtgaggcacatcatcggcatcgacaggggcgagaggaacctgctgtacctggtgatggtggacatggagggcaagatcaagaagcagatcagcctgaacgagatcgccggcaaccccaagaaccccgagttcaagcaggacttcctggccctgctgcacgagagggagggcgacaggctggagagcaggaggagctggaacaccatccagagcatcaaggagctgaaggagggctacatgagcctggtggtgcacgagatcgccaacatgatgctggagaacgacgccatcgtggtgctggagaacctgaacaggagcttcatgcagaagaggggcgagaagagcgtgtaccagaagttcgagaagatgctgatcgacaagctgggctacatcgtggacaagaccaaggacgtgagcgacaacggcggcgccctgcacgccgtgcagctggccgacaccttcgagaacttcaacaagacccagaagggcgccatcaggcagtgcggcttcatcttctacatccccgcctggaggaccagcaagatcgaccccgtgaccggcttcgtgcccatgctgaggtgccagtacgagagcatcgtggagagcaagaagttcttcggcaagttcgacagcatctactacgacgccaccggcaagtacttcgtgttccagaccgacttcaccaagttcaacaccgagagcaagggcggcatccagaagtgggacatctgcacctacggcgacaggatctacgcccccaggaccaaggacaggaacaacaaccccgtgagcgagagggtgaacctgaccgaggagatgaagagcctgttcgtgagccacaacatcaacatccagggcgacatcaaggccggcatcatgcagcagaccgacaaggagttcttcgagagcctgcacaggctgctgaggctgaccctgcagatcaggaacagcaagaagagcaccggcaaggactacgaggactacatcatcagccccgtgatgggcaaggacggcaggttcttcgacagcaggaacgccgacgccacccagcccaaggacgccgacgccaacggcgcctacaacatcgccaggaagggcctgatgctgctgaggcagatccaggcccaggagaagcaggacctgagcaacggcaagtggctggagttcgcccagaggaaaagacctgccgccacaaagaaagccggacaggccaagaagaaaaaggattacaaagacgatgacgataagtga

SEQ ID NO: 49 - DNA, 4005 bp, synthetic construct
  source 1..4005: mol_type = other DNA
atggcccccaagaaaaagcggaaggtgaccaggaagttcgacgacttcatccaccagtacagcctgagcaagaccctgaggttcgaggccaggcccatcggcgagaccctgcacaacttcatcaagaacggcctgctggagagggacgagcacagggccgagagctacgtgaaggtgaagaagctgatcgacgagtaccacaaggccttcatcgacagggtgctgaacgacggctgcctgaactacgaggacaagggcgagtacgacagcctgaccgagtactacgccctgtacagcgcccccaggaaggacgaggccgagagggagcacttcaaggagacccagcagcacctgagggaccaggtggtgaagaagctgaccggcgacaacgcctacaagcacctgttcggcaaggagctgatcgagagctacaaggacaaggaggacaagaagaagctgaaccaggccgacctggtgcagttcatcaacaccgccacccccaagcagaggctgaacttcagcaaggaggaggccatcgacctggtgcaggagttctgcggcttcaccacctacttcggcgacttctaccagaacagggagaacatgtacagcgccgaggagaagagcaccggcatcgcctacaggctgatcaacgagaacctgcccaagttcatcgacaacatggagaccttcaagaagatcgccgccatccccgagatggaggacaacctgaaggagatccacgacaacctggccgagcacctgaacgtggagaacatccaggacatgttccagctgaactactacaaccagctgctgacccagaagcagatcgacgtgtacaacgccatcatcggcggcaagaccgacgacgagcacaaggagaagatcaagggcatcaacgagtacgtgaacctgttcaaccaggcccacaaggacgccaagctgcccaagctgaagaccctgttcaagcagatcctgagcgacaagaacgccatcagctggctgcccgaggagttcgacaacgaccaggaggtgctgaacgccatcaaggactgctacgccaggctgagcgagaacatcctgggcaaggagaacctgaagaggctgctgtgcagcctgagcgagtacgacaccaagggcatcttcctgaggaacgacctgcagctgaccagcatcagcaagaagatgagcggcagctggaccgacatccccaacgccatcaagaacgacctgaaggacggcgtgcccgccaagaagcacaaggagagcgaggaggactacgagaagaggatcgacaacctgttcaagaggcaggacagcttcagcatcggctacatcgacgactgcctgaacaagttcgacgacaacaacaccttcaccatcgagggctacttcaagaagctgggcgccaaggacacccagagcgaggacatcatcaagcagatcgccaacgcctacaccgacgtgaagcccctgctgaacagcacctaccccaagagcaagaacctgagccaggacaaggagaacgtggtgaagatcaagaggttcctggacaccctgatgagcctggtgcacttcgtgaagcccctgctgggcaagggcgacgagagcaacaaggacgagaagttctacggcgagctgagcctgctgtggaccgagctggagaccatcgtgcccctgtacaacatggtgaggaactacatgaccaggaagccctacagcaacagcaagatcaagctgaacttcgacaacagccagctgctgggcggctgggacgccaacaaggagagcgactacgccagcatcctgctgaggagggacggcaagtactacctggccatcttcgacaaggacagcaagaagctgctgggcaagagcatgcccagcgacggcgagtgctacgagaagatggtgtacaagttcttcaaggacgtgaagaccatgatccccaagtgcagcacccagctgaagaaggtggtgaagcacttcagcatcagcgacaccgacttcgtgctgagcgacgagaacttcaaggtgcccctgaccatcaccaaggaggtgtacgacctgaacaacaccatgtacggcaagtacaagaagttccacaagggctacctgaccaccaccggcgacatcgacggctacaccagcgccgtgaagacctggatcaacttctgcatggacttcctgggcgcctacggcagcaccatgagctacgacctgagcagcctggccaccaaggagtacagcagcctggagagcttctaccaggaggccaacaggctgctgtacaccgtgaacttcgtgaaggtgagcaccagctacgtgagcaggctggtgaccgagggcaagatgtacctgttccagatctacaacaaggacttcagcgagtacagcaagggcacccccaacatgcacaccctgtactggaaggccctgttcgacgacaggaacctgaaggacgtggtgtacaagctgaacggccaggccgagatgttcttcaggaagaagagcatcgactgcaacaggcccacccaccccgccaaccagcccatccagaacaagaacaaggacaacaagaagaaggagagcgtgttcgagtacgacctgatcaaggacaggaggtacaccgtggacaagttcatgttccacgtgcccatcaccatgaacttcaagagcaccgacaaggagaacatcaacctgcccgtgatggagtacctgcagaccagcaacgacacccacatcatcggcatcgacaggggcgagaggcacctgctgtacctggtggtgatcgacctgcacggcaacatcgtggagcagtacaccctgaacgacatcgtgaacgagtacaacggcaacacctacaggaccaactacaacgacctgctggaggccaggggcaacgagcagcagaaggccagccagaactggcagaccatcgagaacatcaaggagctgaaggagggctacctgagccaggtgatccacaagatcacccagctgatgatcaagtaccacgccatcatcgtgctggaggacctgaacatgggcttcaagaggagcaggcagaaggtggagaagcaggtgtaccagaagttcgagaagatgctgatcgacaagctgaactacctggtggacaagaaggccgacatcgagagcaccggcggcctgctgaacgcctaccagctgaccaacaagttcgtgagcttcaagaagctgggcaagcagagcggcttcctgttctacatccccgcctggaacaccagcaagatcgaccccgtgaccggcttcgtgaacctgctggacaccaggtgccagaacaagaacaaggccaaggccttcttcgccaagttcgacagcatcaggtacaacaaggacaaggactggttcgagttcgccctggactacgacaagttcggcagcaaggccgagggcaccaggaccaagtggatcctgtgcacccagggcaagaggatcaagaccttcaggaacgccgagaagaacttccagtggtacaaccaggtgatcgacctgaccaaggacctgaagcagctgttcgcccagtacgacatcgacatcaacggcaacctgaaggaggccatcagcaagcagaccaagaagaccttcttcgacgacctgatgggcctgctgaagctgaccctgcagatgaggaacagcatcaccaggaccgagaccgactacctgatcagccccgtggccgacgagatcggcaacttctacgacagcaggacctgcggccccagcctgcccgagaacgccgacgccaacggcgccttcaacatcgccaggaagggcctgatgatcatcgagcagatcaaggccagcgaggacctgagcaagctgaagttcgacatcagcaacaagagctggctgaacttcgcccagcagaagccctacaagcacgagaaaagacctgccgccacaaagaaagccggacaggccaagaagaaaaaggattacaaagacgatgacgataagtga

SEQ ID NO: 50 - DNA, 3948 bp, synthetic construct
  source 1..3948: mol_type = other DNA
atggcccccaagaaaaagcggaaggtgaacaccagcaagttcatcttccagaacctgacccaccagttccagctgcagaagaccctgaggttcgagctgatcccccagggcaggaccctggagcacatcgagaagaacggcctgctggaggccgacgagctgaaggccaacaggtacaagcaggtgaaggagtggatcgacctgtaccacaaggccttcatcgagaaggtgctgagggagcccaagctggagggcctgagcgagtacagggtgttcttcgagaacaagcagaggaacgagaaggacgaggaggccttcaagaagctgaagagcgacctgaggaagaagatcagcagcttcttcaaggacgagcccaacaagaccttctacaagaggctgttcgacaaggagctgatcaaggccgacctgcccgacttcctgagggagcagggcatgcacgaggaggccaccatgctgctggaggagttcggcaacttcaccacctacttcaagggcttccacgagaacaggaagaacctgtacgtggccgaggacaagagcaccgccatcgccttcaggctgatcgacgagaacctgcccaggtacatgcagaacctgctgctggtggagaagcacagggaggtgttcgagcagttcttcccccagatccaggagaggctgggcaaggagctggacggcctgagggtggaggagttcttcagcgaggagggcttcaacctgtgcctgaacggcagcggcatccaccagtacaacaccatcctgggcggcgtgaagggcgaggaggaggagggcatccccggcctgaacatgctgatcaacctgtacaggcagaagcacaagctgaagagcagggagctgcccaccctgttccccctgcacaagcagatcctgagcgacagggagagcagcagcttcaggcccgagccctacgaggacggcgacgagaacaccgtggtggaggacctgctgaagctgggcgaccagtggttcaaccccgagggcttcgtgccccagctggtggcccacctgaccaagctgaaggacgaggagcaggaccagctgttcgtgaacaacaaggccaacgccctgagcgccgtgagccaggccaccctgggccactgggccgtgctgggcgaggccctgagcagctactggaaccacgtgcacgacgtgaaggagaacgccctgaagggcaaggccctgaccgccaaggagaagtggctgaagcagccctacttcagcttccaggagatcgagctggccctgaggcactacatgggcgccgagggcaaggagaacgccagcaagtggaacgtgtggctggagaagcccctggtgcagtacttcaactacggcctgaccaaggaggccgagaagaccaaggtggacaccgccaaggccgcctggggccacgccgagaacatcctgagggagctgcagaaccacgagaaccccaagctgaagaccaaggagaccaaggacgccatcaaggcctacttcgacgcctgcctggacctgctgcacttcatcaagcccctggtggtggacctggagcagtgcgaggccagccccaccttctacggcgacttcctgagcctgtaccacgagatggacgacctggtgcccctgtacaacaaggtgaggaactacatgaccaagaagccctacagctacgagaagttcaagctgaacttcaacaagggcaccctgctggacggctggagccagagcaaggagaccgacaacctgtgcgtgctgttcagggagggcaacaacttcttcctgggcatcatggacaggaagcacaacaagagcttcgtgaagccccccatggccaaggaggaggagcccgtgatgcagaaggtggtgtacaacctgctgcccgaccccagcaagatgctgcccaagtgcttcaccgccccctacttcaggaggatcgccaaggagaagaccaaggaccccgagcagatcgtgagcgagtacagcaaggagaagggcctgatcaagcccaagctggacgtgctggacgcctacgcccacaagaagcacaccaaggagaacttccagaggaacggcttcgagcaggtggtggagtacttcaaggagggcgcccagaagcaccccgactggcacgtgttcgacttcaagttcaagcccaccagcgcctacaaggacgtgagcgacttctaccaggaggtgaccgagcagggctactgcctggacttcgtggacatccccaagaggtacatcaacgacctggtggagagcggcaagctgtacctgttccagatctacaacaaggacttcagcaagtacagcaagggcaagcccaacctgcacaccatgtactggaaggccatcttcgacccccagaacctgaaggcccccatctacaagctgaacggcgaggccgaggtgttcttcaggaagaagagcctggagtacacccaggagcagctggacaccggccaccacagggaggagctgaagggcaagttcgactaccccatcgtgaaggacaggaggttcgccttcgacaagttccagttccacgtgcccatcaccctgaaccacgccaggaagaaccaggccgtgggcccccaggtgctgaggtacatccagaagcacaaggaggagctgcactacatcggcctggacaggggcgagaggcacctgctgtacctgagcgtggtgaacgccaagggcgagatcatcgagcagaggagcctgaacaagatcggcaacggcttcgtgaaggacggcaagccctacacccacgactaccagggcaagctgctggagaaggagaggaacagggacagcgccaggaaggactgggacgccgtggagagcatcaaggagctgaaggagggctacatgagcatcatcgtgcacgagatcgcccagctggtggtgaagtacaacgccgtggtgatcctggaggacctgaacttcggcttcaagaggggcaggttcaaggtggagaggcaggtgtaccagaagttcgagaggaagctgatcgagaagctgaaccacctggtgttcaaggaggtggccccccaggagcccggcggcctgctgaaggccctgcagctgaccgaccagttcaccagcttcaccaagctgggcaagcagagcggcatcctgttctacctgcccgccgcctacaccagcaagatcgaccccctgaccggcttcatcaaccaggtgccctggaagtacaccaacctggaggccgcccagaagtggatcggcaacttcaagaggatcagctaccacgccgagctggacctgttcgagtgggtgctggaccagagggacttcctgagcaaccccgccggctgcaagcaccagtgggtgctgaggatggacaaccaggagaggtggcactggaacagcaaggccgagggcaccaacggcaagggcgcctacagccccatcaggctgaaggagcagttcgaggacctgctggccaggcagaacatcagctacaacggcaacagcaagctgctggaggacatccaggccgtgaaggacgccagcttcttcaagaccctgttcttcttcctgaggaccctgacccagctgaggtacaacaacggcaagagcggcgccgaggaggaggacttcatcctgagcgccgtggccaaccccggcatggagcccttcgacaccaggaaggccaagcagggcctgcccctggacgccgacgccaacggcgcctaccacatcgccatgaagggcctgatgaccctgggccagctgaaggagaagcccaacggcaagctggagatccccaagctggacaagagcgcctggctgtgcttcatgcagcagaggagcaaccagggcaaaagacctgccgccacaaagaaagccggacaggccaagaagaaaaaggattacaaagacgatgacgataagtga

SEQ ID NO: 51 - DNA, 3888 bp, synthetic construct
  source 1..3888: mol_type = other DNA
atggcccccaagaaaaagcggaaggtgcagaccaagaacttcatgagcttcagcgacttcaccaacctgtacgccctgagcaagaccctgaggttcgagctgaggcccatcggcaggaccgaggagttcttcaagaagaacaaggtgttcgagaaggacaagaccatcaacgacagctacaaccaggccaagctgtacttcgacgagatgcacaggaggctgatcagggacgccctgaacacccagaacaccagcgccctggactacgagaagtacgccaagcactggaaggagctgcagaaggacaagggcaagtacatccagaactggaaggacgcccagaaggagttcagggagtgcatcagcaacctgctggaccagcaggccgagcagtggaagagcaagtacgccagcgtgggcctgaacaaggccgacatcaagcagaagggcaccgacttcctgagcagcgccggcatggtgaagatcctgaagaaggagttccccaaggaggccgagaaccagctggtgaaggagggcttccccagcctgttcatccagaaggccgaggcccccgagcagaagcagtacatcttcgacagcttcgacaggttcagcacctacctggccaagttcaaccagaccagggacaacctgtacaaggacgacggcacccccaccgccatcgccaccagggtggtggagaacttcgtggccttcctgcagaaccaggccatcttcaacaaccactacatcagcctgaagagcgagctggacttcagcagggaggagatcgactgcttcaccgccaactactacgtgaggtgcctgctgcaggaggacatcgacgagtacaacaagaccatcggccagatcaacgccaggatcaaggagctgagggacaggaagcagaaggagagcgacttcaagaagagcggctaccccctgctgaagagcctgaacaagcagatcctgggcaagatcgagaagaagaggcagttcatcgagaacaaggccgacgtgttccccgccttcagcgagctgatcgagcaggccaacgccaggctgaccgccctgaggcagctgatcaacaacctgtgcgaggaggtgttcgaggaggcctacggcggcatctacatcaagaacaccggcatcaacaccatcagcaggaggtggttcgccagctggtacgacttcgagatcgccctgccccagaagagcaagaacgaggaggagagcaaggtggtgaccttcgtgagcctgcaggacatcaaggacaccctggagaacaagctgagcggcgacatcttcaaggaggagtactacaagaagaagatcatcaacaaggagcagagcaactggaagcagatcctggccatcctgcagaacgagtggaacgagctgttcagcgagtaccagaaggccctggagaacaccaagaagctggacctgaacagcgtgttcagcaacagcaaggagcaggtggagatcatcaagagcttcgccgacgccagcctgagggtgtaccaggtggtgaagtacttcgccctggagcacaagaagagcgacgagatccccagcagctacagcaccgacttctacgaggagttcgacaagcagtacaacaacttcgagttcatcccctactacaacgccctgaggaacttcctgaccaagaaggtggtgggcgagaacaagatcaagatcaacttcgagaagggcaacctgctgaacggctgggccgagagccccgagggcaacgcccagttctgcggctacatcctgaagaaggagaacaagtacttcctgggcatcaccgaccagcccaccatcctggacaccaaggacaaccccagcctgaccaacatcaccgacaaggagtactacgagaagatggcctacaggcagctgaagagcaccaccatctacggcagcggctacgagggcctgtacaacagcaagtacgccaacgacaagaagtacatcagcgagagcaagatcatcgagagggtgaagaagctgctgcaggagaagtacctgaccaggttccccgagctgaagcagatcatcgagaaggagtacaaggacgccaacgagctggccaagaacctgggcaacttcaacctgtacagcctggagttcatccccgtgaacaagaactacatcgagaagctgacccacgacaagaacggcagcatcaagtacctgtacatcttcgagatcgccaacaaggacctgggcaagatcacccccaggaagtacaacatccacagcctgtacttcaagaacctgttcagcgaggacaacctggagaagcccgtgttcaagctgagcggcggcgccgagatcttcttcaggcccaagaccgtgaacctgcccaagaagaaggacaaggccggcaaggacatcatcgaccacaagaggtacgcccaggacaagctgttcttccacctgcccatcgtgatcaacttcggcgccggcaaggtgggcaagttcaaccaggagctgaacaagaagctgctggccggcaacaagaacatcaacatcatcggcatcgacaggggcgagaagcacctggcctactacagcgtgatcaaccagaagggcgagatcatcgagcaggacagcctgaacgagatcaacggcgtgaactactacggcctgctgcagaagagggagaaggagaggctggagaacaggaagagctggctgcccctgcagcagatcaaggacctgaagaggggctacatcagccaggtggtgtacaagatcgtgaggctggccatcgagcacaacgccatcatcgtgctggaggacctgaacatgaggttcaagcagatcaggggcggcatcgagaagagcatctaccagcagctggagaaggccctgatcgacaagctgggctacctggtgttcaaggacaggaacgccaaggaggtgggcggcgtgctgcagggctaccagctgagcgcccccttcgagagcttccagaagatcggcaagcagaccggcatcatcttctacacccaggccagctacaccagcgtgaccgaccccctgaccggcttcaggaagaacatctacgtgagcaacagcgcccccatcgagaagatcaaggtggccatcaacaacttcgaggagatcggctgggacgacgtggtgcagagctacttcttcaactacgacaccaagaacttcagcaacggcaacgacatccagagcaagaagtggaagatctacgccaaggtgcccaggatcaagagggagaaggtgaacggctactggcagtacaacgacgtggaccccaacgacatgctggagaagctgttcaggctgtacgagttcaagaacctgaggagcaacatgctggagcagatccacgccaaggagaccaacggcgagctgaggggcgagagggacttcgacaacaagcccaggaacttctaccagagcctggtgtacatcctgaacctgatcctgcagctgaggaacagcaccagcaagcagatcaagctgaacgagaaggaggacgtgtacgagaccggcgaggacgtggacttcatcgccagccccgcctaccccttcttcaccaccctgagcagcaagagcaacagcaacctggccggcttcgagaacaggttcaccggcctgaaccacgaccaggagcagaagaagttcaacggcgacgccaacggcgcctacaacatcgccaggaagggcatcatcatcctggagaggatcaccaagaacagcgacaagcccaacctgtacatcagcaacgaggactgggacaagttcgtgagccagagctaccccaaaagacctgccgccacaaagaaagccggacaggccaagaagaaaaaggattacaaagacgatgacgataagtga

SEQ ID NO: 52 - DNA, 3933 bp, synthetic construct
  source 1..3933: mol_type = other DNA
atggcccccaagaaaaagcggaaggtggagagcaagaccctggacaggttcatcggcaagtaccagctgagcaagaccctgaggttcgagctgatcccccagggcaagaccaaggagcacatcgagcagaagggcctgctgcagcaggacgagcacagggccaaggcctacaagatggtgaagaagatcatcgacaggtaccaccagcacttcatcgccaccgccctgaacaacctggccctggacggcctgcagagctacgaggagctgtaccacaagaaggagaaggaggaggccgacaagaagaacttcgagagcctgaaggccggcatgaggaagcagatcggcgacgccttcaacaagagcgaggccagcaaggtgcaggagatctggaaggtgctgttcgacaaggagctgatcaggaaggccctgcccgagttcgtgaccagcgaggaggacaagaagctgctggccgagttcaaggacttcaccacctacttcaccggcttccaccagaacagggccaacatctacacccacgaggagaaggccaccgccgtggccttcaggctggtgcacgagaacctgcccaagttcctggacaacagcagggtgttcgactggatccagaccgagtaccccgacctggacctgagcgccatcgagaaggagctggcccccgagctgcagggcaccaccctgcccgagatcttcgccctgagcttcttcaaccacaccctgacccagaacggcatcgacctgtacaacgccgtgctgggcggcaagaccaccgagcagcagaacaagatcaggggcctgaacgagtacatcaacctgtacaaccagaggaggaccgccgaggccaagaaggagggcgagaagttcaggagggtgccccagttcaaggagctgtacaagcagatcctgagcgacagggagagcatcagcttcctgcccgagaccttcgagaacgacagcgaggtgctgagcgccatcgagaacttcttcgccacccagctggtgcacttcgagaaggacaacaggagcatcagcgtgttcgccgagctgcagaagctgctgcaggagctgaaggaggacgacaggagcaagatcttcgtgaggaacgacctggccctgaccaacatcagccagaacatcttcggcgactggcaggtgatcaagaacgccctggagcactactacaccaccaccgtgaaccccctggccgtgggcaagaagctgaccaaggcctacgagaacggcaaggagaagtggcagaagcagagctactacagcatctacgagatcgaggaggccctgctggcctacagcagcgaggccgacgtgctgaaggagaagaacctgaccgccgccagcaggcccctgtgcaactacttcgccaccttcagcaaggacgaggagggcaacaacctgttccagaggatcgagaccgcctacaccgccgtgaaggacctgctgaacaccgactaccccgaggagaagaacctggcccaggacaagaccaacgtggccgccatcaagaccttcctggacgccatcaagaacctgctgcacttcatcaagcccctgcaccccgccaggctggagggcgagaaggacgagaggttctacggcgccttcatccccctgttcgaccagctgaacctgctgacccccctgtacaacaagaccaggaactacctgaccaggaagccctacagcaccgagaaggtgaagctgaacttcgagaacagcaccctgctggacggctgggacgccaacaaggagaccgccaacaccagcgtgctgctgaggaaggacggcctgtactacctggccgtgatggacaagaagtacaacaaggtgttcgagcagatgcccgaggtggccgagggcgagaaggtgttcgagaaggtggtgtacaagctgctgcccggcgccaacaagatgctgcccaaggtgttcttcagcgccaagaacatccagtactacaaccccagcaccgagctgctggagaactacaagaacgagacccacaagaagggcgccaccttcaacctggtgcactgccaccagctgatcgacttcttcaaggccagcatcgccaggcaccaggactggaagaacttcaacttccagttcagcgacaccggcagctacgccgacctgagcggcttctacagggaggtggagcagcagggctacaagatcaccttccacaacctgcccgagagctacatcaaccagctggtggaccagggcaagctgtacctgttccagatctacaacaaggacttcagccccttcagcaagggcaggcccaacctgcacaccatgtactggaagatggtgttccacgagggcaacctgaccaacgtggtgtacaagctgaacggccaggccgagatcttcttcaggaagaagagcctgaactacgacgagaagaccctgagggagggccaccacgccgccgccctgaaggacaagttcgcctaccccatcatcagcaacaggaggttcgccttcgacaagtaccagttccacgtgcccatcaccatcaacttcaagagcaccggcagggacaacgtgaacgccgaggtgctgagcttcctgcagcacaaccccagcgtgaacatcatcggcatcgacaggggcgagaggcacctgctgtacctgaccctgatcgaccagaagggcaacatcaagaagcagatgagcctgaaccagatcatcaacagctacaggaacaaggagggcgacatggtgaacgtgaagaccgactaccagaacaagctgcacctgaaggaggaggagaggctgaaggccaggcagggctggggcctgatcgagaacatcaaggagctgaaggagggctacctgagccacgtggtgcacgagatcgccaagatgatggtggagcacaacgccatcgtggtgatggaggacctgaacttcggcttcaagaggggcaggcagaaggtggagaagcaggtgtaccagaagttcgagaagatgctgatcgacaagctgaactacctggtgttcaaggacaggaccgccaccgagcccggcggcctgctgcacgccctgcagctgaccggcagctacaccgacttcatgaagtacaagcagaagcagtgcggcttcctgttctacgtgcccgcctggaacaccagcaagatcgaccccctgaccggcttcgtggacttcctgaagcccaagtacgagagcatcgagaaggcccagaacttcctggccaagttcaagagcatcaggtacaaccccgccaagcagtggttcgagttcaccttcgactacaaggacttcaccgacaaggccgagggcagcaggaccaggtggaccctgtgcagcaccaaccagcccaggtacaggtggaaccccaaggccgccaacggcaagggcgcccaggaggccgtgaacgtgacccaggccctggaggacctgttcagcgcccacaacatcgcctacggcagcgccgacgacctggtgaccgccatcaccgcccagagcagcgccgacttctacaaggccctgctgagggccatggtgaccctgctggccctgaggcacaacaacggcaaggccggcaccgacgagcaggactacatcctgagccccgtggagcccttcttcgacagcaggctggccgacgacagcaggcccaaggacgccgacgccaacggcgcctaccacatcgccaagaagggcctgtgggtgctgcagcagatcaacgagctgcagggcgacgactggaagaacctgaagctggccatcagcaacaagcagtggctgcagttcgcccagcagctgaacgccgacatcagccccaaggtgagcaaaagacctgccgccacaaagaaagccggacaggccaagaagaaaaaggattacaaagacgatgacgataagtga

SEQ ID NO: 53 - DNA, 4128 bp, synthetic construct
  source 1..4128: mol_type = other DNA
atggcccccaagaaaaagcggaaggtgagcagcagcatcgtgagcgtgagcaccgagaagaagcaggccgaggtgctgagcaacaagagcgtgttcgaccagttcaccaggaagtacgccctgagcaagaccctgaggttcgagctgaggcccgtgggcaagaccagggagaacatcgagaaggccaaccccaacttcatcagggacaaggagatcgaggacgcctaccagatcctgaagcccgtgttcgacaagctgcacgaggagttcatcaccgagagcctggagagcgacgaggccaagcacctgagcttcgaggactacttcagcgtgtacaagaacctgaggaacgagcaggacagggaggagaagaagaagatcgagaagagcctggagaccaaggagaagaacctgagggccgccttcagcgacatctacaaggccgagggcgagaagttcaagagcaaggtgggcagcgacaagaagggcaagcccatcctgaaggaggacagcttcaagatcctgaccgaggtgggcatcctgaagtacgtgaaggccaagatcgaggagttcgtggtgatgaacctgaagacccacgagggcaaggaggtgaagaaggaggacctggagaaggccctgggcaccgccgacagcaagggcgtgttcgagggcttcttcacctacctgaccggcttcaaccagaacagggagaactactacagcaccgaggagaaggccaccgccgtggccagcaggatcatcgacgagaacctgcccaagttcgccgacaacgtgctggagttccagaagaggaaggacgagtacctgaaggtgtacgacttcctgaaggagaacggcatcgagaccatcggcaagaaccagaagggcgagaaggtggagctggagaagatcatcgccgacatcttcgagatcggctacttcaccaactgcctgagccagagggagatcgagaagtacaacctgaacatcggcaacgccaacaacctgatcaacaggtacaaccagcagcagagcgagaagaacaagaagctgaggatcttcaagaccctgtacaagcagatcggctgcggcgagaagggcgacttcatccccgccatcaagaacgacgacggcctgagggccgtgctgaagaacgtggtggagaacggcgagaagtacttcggccagttcgacggcaagatcgaggacaaggtggacaccgtgggcgagttcgtgcactacatcatcaaccacgagagcttcgagggcatcttctggagcgacaaggccctgaacaccatcagcggcaagtacttcgccaactggttcatcctgaagaaggccctgaaggagggcggcgtgttcggcggcaagaagaaggacgacgaggacatcaagatcccccaggccgtggagctgaagaacctgttcgagatcctggacaagaccgagaagtggaagaacaaggaggacggcctgttcaagaccagcctgttcgacggcgagggcaacaagaagggcatcatcgagagggccgagaagcccagcaaggccctgctgggcatgatcttcgccgacgtggagaagatggccaaggacttcagcgagctgagccccaaggtgctgagcatcaccaccgacttcaagaagcccgagcacatcgagaacatcaagatgctgctggacagcgccctgtacgtgaaccagatcgtgaagtactggaaggtgaaggacaagttcgccgtggaccccatcctggccgagagcctgaaggccatcctgttcggcgacaacaaccccaccaagttctacgacatcatcaggaactacctgaccaagaggccccaggagggcctgaacaagctgaagctgaacttcgacaacagcgtgctgggcggcggctgggacgtgaacaaggagcccgagaggtggtgcctgatcctgaaggacggcgagaagaagtacctggccatcctgaccaaggccagccagaagctgttcggcgagaaggccaagtacaaggaggtggtgaccgacgagaactacctgaagatggactacaagctgctgcccggccccaacaagatgctgcccaaggtgctgctgcccaagaggaacaggaaggagttcggcgccaccgacgacatcctgagcatctacgacgagggcggcttcaagaagaaccagcccagcttcaccaaggagaagctgtggaagatcatcgacttctacaaggagggcctgaaggtgtaccccagcgccgagaagagctggcaggacctgttcggcttcaacttcagccccaccgagaagttcgagagcatcgaccagttctacaacgaggtggagaagcagggctacaagctgagctggaacatcatcagcaagaagctgatcaacgaggccgtggagaacggcgagatctacttcttcgagatcaggaacaaggacaacaacctgaaggagggcaaggagaagaccagcgagaagaacctgcacaccatctactggaacgagatcttcggcgacgtggccaacaagcccaagctgaacggcgaggccgagatcttctacaggaagaccatcctgaagaagaaggagaacgagaccgacaaggagttcgagatcaggctgaaggagaagctgggcaagaagaaggacaagagcggcaaggaggtgctggagcacaagaggttcgccaacgagaagttcgtgttccactgccccatcaccctgaacttctgcctgaggagcctgaagttcaacgacgagatcaacgagagcatggtgaagctgaacgacgacatcaccaccatcggcatcgacaggggcgagaagcacctggcctactacttcgtggtgaagcagaccaaggacggcaagatcgagaccctggaccagggcagcttcaacgagatcaacggccagaactacgccgagaagctggagaagatggccggcaacagggacgaggccaggaagaactggaagaccatcggcaccatcaaggagctgaaggacggctacatcagccaggtggtgaggaagatcgtggacctggccatctacgaggacgtggagaagaaggtgttcagggagacccccgccttcatcgtgctggaggacctgaacatcggcttcaagaggggcaggcagaagatcgagaagcaggtgtaccagaagctggagctggccctggccaagaagctgaacttcctggtggacaagcacgccaaggagggcgagatcggcagcgtgaccagcgccctgcagctgacccccctggtgaacaacttcggcgacatcgagggcaagaagcagttcggcatcatgttctacaccaaggccgactacaccagccagaccgaccccgtgaccggctggaggaagaccatctacctgaagaagggcagcgaggagagcatcaagaagcagatcctggacaacttcgacgacatcaagttcgacggcaaggactactacttcaagtacaccgacaggaacaccaacaagccctgggagctgtacagcggcaaggacggcgagagcctggacaggtactacagggagctggtgcacgagaacagcgacaagaagtgggtgcccaaggagcagaacctgaagaagatcctggacggcctgttcgagggcttcgacaaggagaagagcctgctggagcagctgaggcagggcaagaaccccaacaagatcaacgacaggaccagctgggagagcctgaggttcgtgatcgacctgatccagcagatcaggaacagcggcaagaccgaggaggacgccgacttcatcctgagccccgtgagggacaagaacggcaaccacttcgacagcaggaagatcaacctgaacctgcccaccagcggcgacgccaacggcgcctacaacatcgccaggaagggcatcatcatgtgcgagcacatcaagagggacctgagcctgttcatcagcgacgaggagtggagcgcctggctggccggcagggagatctgggagaactggatcaagaagaacgagaagaacctgaagaggaagcagaaaagacctgccgccacaaagaaagccggacaggccaagaagaaaaaggattacaaagacgatgacgataagtga

SEQ ID NO: 54 - DNA, 3969 bp, synthetic construct
  source 1..3969: mol_type = other DNA
atggcccccaagaaaaagcggaaggtgaagagcatcgaggagctgatcggccagtacagcctgaccaagaccctgaggttcgagctgaagcccatcggcaagaccctggagcagatcgagaggaagggcgtgctgagcgccgaccagaccagggccgaccagtacaagagggtgaaggagagcatcgacaggtaccacaaggccttcatcaccgagagcctggacagcctggtgctgaaggtgaagagcaccggcaacaccagcgacagcctggaggagtacgccagcctgctggacaacagcgccgacgacgaggccctggacaaggtgaagaagggcctgaggaagcaggtggccgagaggttcgacaagcagatcaggctgaagaagctgttcaagaacgacctgatcaccaagatcctgcccgagttcctgaaggtgaacggcaacgaggacgagaggaaggccgtggagagcttcaggaacttcaccacctacttcagcggcctgaacgagaacaggaagaacatgtacagcaaggaggccaagagcaccgccatcgcctacaggctgatcgacgagaacttccccaggttccacgacaacatgctgagcttcgccaagatcgccgccgtgaacgaggtgagcaaccacttcgccgagatcgccgaggccttcaagagcgagatggtggtggccagcgtggccgagatgttcggcctggagtacttcaacaacaccctgacccagcagaagatcgacctgtacaactacgtgatcggcggcaagaccgagggcgacaaggtgaagatccagggcatcaacgagtacgtgaacctgtacaaccagaaccaccccaacgacaagctgcccctgctgaagcccctgtacaagatgatcctgagcgacagggtggccctgagctggctgcccgaggagttcaacgacgacgaggccatggtgtgcgccatcaaggagttctacaacgccatccagcccgtgctgtggggcgaggtggaggacgacgacaacagcgtgtgccacctgctgaagaacatcggcaagtacgacatcgagcacatcttcatcagcaacggcgccgccctgaacgacgtgagcaggcagatgttcggccagtacgacttcctggccaacgccatcaagaaggacatcaggcagaacaccaagaggagcagcagggagaccgacgagaagttcgagggcaaggtgaacaccctgttcaagaaggccaagagctacagcatcgcccacctgaacgccctgatcaaggagaacaggagcgagaccatcgagagcttctacagcaacctgggcgcctgcgacagggacggcgagcagaagatcaacctgctgacccagatcgagctggcccacgaggccgcccaggacgtgctggacggcaagtacgccgacatcaacagggacgagaaggccaccatgctgatcaaggagctgctggacgccttcaaggccctgcagtacttcatcaagcccctgctgggcagcggcgaggaggccagcaaggacaacgtgttctgcaccaagctgctgaacgcctggcaggccctggacgccgtgacccccctgtacgacaaggtgaggaacaggctgaccaggaagccctacagcaccgacaagatcaagctgtacttcgagaacaagggcaagttcctggacggctgggtggacagcaagaccgacaacagcgacaacggcacccagttcggcggctacatcctgaggaagaagaacgagatcggcgagtacgactacttcctgggcatcagcagcgacaccaagctgctgagggccaaccccgacatccagtacgagcacggcatgtacgagaggctggactactaccagatccagagcaagaccttcctgggcagcagctacaagggcgagtacaacgacgacgccatgaggctgatgagcgccttcgtggagtacggcaagaagagcaggttcgacagctgcgtgctgcccaccgacaaggagaagagcctgcccacctacctgaagaggatctacaccgacaacaacagcttctacaacgaggccatgaccgacaccgtggtggccgaggcctacaacaacatcaagagccacctgctgctgaccctggccggcatcaagagggtggaggccgccgtgaacctgagcaagcacaacgagtacaacctgatccagatcttcgacgagatcggcaagatcaccagcagcagcaagatcttcagcttcttccccgtggacaccagcctggtggagaagctgaacgagagggacaacaagcccctgttcctgttcaagatcagcaacaaggacctgagctacgccgagaccagcagcaagaacatcaggaagagcaggggcaaggagaacctgcacaccatgtacttcaaggccctgatggagcagagccacaacgtgttcgacatcggcaccgccgaggtgttcttcaggaaggccacccccggcctggacaagagcaccaccatccacaaggccaacgaggccctgtgcaacaagaacccccacagcaagaagaccagcagcaccttcggctacgacatcatcaaggacaggaggtacgtggagaacaagttccagctgcacctgagcatggtgatcaactaccagggcaagaaccccaacaccgacatcatcagcccccaggtgctggacatcatcaggaacaacggcatcaagcacatcatcggcatcgacaggggcgagaggaacctgctgtacctgagcctgatcgacctgaagggcaacatcatcaagcagatcagcctgaacgagatcaagcccctgggcaacgacggctgcgtgaccaactacagggacatcctggccgacagggagaaggacaggaaggaggccaggaggagctggaagaagctggagaacatcaaggacctgaagagcggctacctgagccaggtggtgcacgtgatcgccaagatgatggtggactaccaggccatcgtggtgctggaggacctgaacagcgccttcatgcagagcaggcagaagatcgagaggagcgtgtacgagcagttcgagcacaagctgatcaacaagctgaactactacgtggacaagagcgtgagcgacgacctgcccggcggcctgctgtacggcctgcagctggccagcaagaagacccacgacgtgcagtgcggctgcatcttctacgtgagcgcctggaacaccagcaagatcgaccccgtgaccggcttcgtgaacatgctggacaccaggttcgagaacgtgagcagcagcaggagcttcttcagccacttcgacagcatcaggtacaacgtggccaaggactggttcgagttcaccttcgactacaacaacttccacaagaagctggagggcaccaagaccaggtggaccctgtgcacctacggcaccaggatcctgacccagaggaacgacaggaaccagtgggagagcagggaggtggacatcaccgacgagttcaagaaggccttcgtggccgccggcatcgacatccacggcaacctgaaggacgccatctgcgccctggacaagagggtgcacctggagcagctgatgaggctgatgaagctgctgctgcagctgaggaacagcatcatcaaccaggacaccgactacatcctgagccccgtggccgagaacggcgtgttctacgacagcaggaggcacgccgacaacctgcccatcgacgccgacgccaacggcgcctacaacatcgccaggaagggcctgtacctgctgaggaagatcaaggccgacaggagcgccaagcccgagctgtgcatcagcaacaaggagtggctgaggttcgtgcaggacaagagcaaccccggcgacaaaagacctgccgccacaaagaaagccggacaggccaagaagaaaaaggattacaaagacgatgacgataagtga

SEQ ID NO: 55 - DNA, 4080 bp, synthetic construct
  source 1..4080: mol_type = other DNA
atggcccccaagaaaaagcggaaggtggtgaccatcgacgacttctgcggccagaagaacggctacagcaggagcatcaccctgaggaacaagctgatccccgtgggcaagacccaggagaacctggagaaggccaacatgctggaggaggacttcagcagggccaaggcctacgtggaggtgaagaagatcatcgacgacttccacaggagcttcatcgaggacgtgctgagcaaggccaacctgaactggaaggagctgtacgagaagatcgacagcttccagagggagaccgacaaggccaagaaggccaagaaccagaaggacctgcagcccgtgcaggtgaagatgaggaaggccatcgtgaagatcttcaaggacgacgagaggttcgacaagctgttcaagaaggagctgctgagcgacctgctgcccgagatcatcaagagcgcccccgagaacgagatcagcaacaagcagaccgccctggacgtgttcaagggcttcgccacctacttcaccggcttccacgagaacaggaagaacatgtacagcgaggaggacaaggccaccgccatcagctacaggatcgtgaacgagaacttccccaagttctacgccaacatcaagctgttcgagaagatccagaaggagttccccgtgatcatcgaggagaccgagaagagcctggccagcttcctggccggcaagaagctgaaggacatcttcacccccgaggccttcaaccaggtgctgacccagagcggcatcgacttctacaacaccgtgatcggcggcatcgccggcgaggccggcaccgagaaggtgcagggcctgaacgagaagatcaacctggccaggcagcagctgcccgacgaggagaagagcaagctgaggggcaagatggtggtgctgtacaagcagatcctgagcgacagggagaccgccagcttcatccccgtgggcttcgagaacaaggacgaggtgtacaagaccctgcaggagttcaagaagagcaccgtggagaagagcatcgagatcatcagggagaccttcaagagccccgagaacttcgacctgagcaagatctacgtgcccgccaaggagctgaccgccttcagcctgagcgtgttcggcaggtggaacatcctgagcgacggcctgttcgccatcgtggaggagaagagcaagaaggccctgagcgagaagcagaccgagaagctggccaacgagatcgccaagaaggactacagcctgagcgagctgcaggccgcctacgagaagtacctggagaagaaccccgtggaggacgagggcaggtgcaggaggctgagcgccaagaactacttcgagctgatcgagctgaggaagatcgacgacagcgccgagaaggagttcgtgaacgtgctgagcaacctggacgagctgtggagcaagatcgacttcgagaaccccaggaacctgcagcaggagaaggaggccgccacccccatcaagaactacctggacgaggtgcagaacctgtaccacttcctgaagctggtggactacaggggcgaggaggagaaggacagcgagttctacagcaagctgaccgagatcctggacctgctgagcgagatcgtgcccctgtacaacaaggtgaggaacttcgtgaccaagaagcccggcgaggtgaagaagatcaagctgaacttcgactgcagcagcttcctgaagggctggggcaccgagttcggcaccaaggacgccttcatcttcatcgacgagaagaagtactacctgggcgtgatcaacaagaagctggacagcgacaccgtgaagtacctgcagacccccggcgagcacatcatcaagcaggtgttcgtgaacttccagaagcccgacttcaagaacttccccaggctgttcatcaggagcaagaaggacaggttcgcccccgccgtggagcagttcaacctgcccgtgaacgacatcctggacatctacgacaagggcctgtacaagaccgagtacaggaaggagaaccccatcatctacaaggagagcctgatcaagatgatcgactacttcaagctgggcatcagccagcacgagagcttcaagggcttcaagctgacctggaaggagagcagcgagtacgagaacatcagcgacttctacgccgacgtgaccgccagctgctaccaggtgaacttcaccaacgtgaacttcgacaggctgctgagcctggccaacgagatcgaggacgacaacaagaagatcttcctgttcgagatctacaacaaggacttcagcaccggcaagaacggcggcaacggcagcaccggcaagaagaacctgcacaccatgtacttcgagaacctgttcagcaaggagaacctgaaggacatcgtgctgaagctgagcggcgaggccgagatcttcctgaagagcgagaacaccgagctgaagaagatcgtgcacaagcagggcaccaccctggtgaacaggaccaccaaggacaagaagctgatccccgagaacatctaccaggagatctaccagttcaagaacagcatgatcgacaacatcagcgacgaggccaaggcctggctgaacagcggcaacgtggagtgcaagatcgccaagttcgacctgatcaaggacaagcacctgatcgagaagagctactacctgcactgcagcatcatccagaacttcaaggcccccgagaaggtgttcaacttcaacgagaaggtgcagaggtacctgaaggacaaccccgacgtgaagatcatcggcctggacaggggcgagaggcacctgatctacctgagcctgatcaaccagaagggcgagatcgagctgcagaagaccctgaacctggtggagcaggtgaggaacgacaagaccgtgaaggtgaactaccaggagaagctggtgcagaaggagggcgacagggacaaggccaggaggaactggcagaccatcggcaacatcaaggagctgaaggagggctacctgagcaacgtggtgcacgagatcgccaagctgatggtggagcacaacgccatcgtggtgatggaggagctgaacttcggcttcaagaggggcaggttccccgtggagaggcaggtgtaccagaagttcgagaacatgctgatcggcaagctgaactacctggtgttcaaggacaagagcgtgaccgagcccggcggcgtgctgaacgcctaccagctgaccgacaagagcaccaacgtgagcgacgtgggcaagcagtgcggctggctgttctacgtgcccgccgcctacaccagcaagatcgaccccaagaccggcttcgccaacctgctgaacatgaaggacctgaccaacgtggccaagaagaggatgttcttcagcaagttcaaggacatccacttcgacagcggcagcaacgacttcgccttcaccttcgactacaaggacttcgacggcaagggcgccgccgagatgacccagaccgagtggaccgtgtggagcaacggcaagaggatcttctacaacagcaagcaccacagcaccgaggagatcaacgtgagcgagaagctgaaggagctgttcgacgccagcaagatcgactacaagaccgagaacaacatcttcgagagcatcatggaggtgggcgccgacctgaaggagggcgagaagcccagcaaggagatcgccaacttctgggacagcctgctgtggtacttcaagctgatcctgcagatgaggaacagcatccccaacagcgagaccgactacctgatcagccccgtgaaggccgaggacggcaccttcttcgacagcagggtgcagaaggagctgggcaaggacagcaagctgcccatcgacgccgacgccaacggcgcctaccacatcgccctgaagggcctgtacctgctgaagaacgacttcaacctgaacgacaagggctacatcgagaacatcagcaacgccgagtggttcaggttcgtgcaggagagggactacctgaacaaaagacctgccgccacaaagaaagccggacaggccaagaagaaaaaggattacaaagacgatgacgataagtga

SEQ ID NO: 56 - DNA, 4005 bp, synthetic construct
  source 1..4005: mol_type = other DNA
atggcccccaagaaaaagcggaaggtgcccaacctgttcgacaacttcacccagcagtacagcctgaccaagaccctgaggttcgagctgaagcccgagggcaagaccctggagaacatgcagaacgccttcaagttcgacaaggacctgcagaccttcctggtggaccaggacatcgaggacgcctaccagaccctgaagccctacatcgacttcatccacgaggagttcatcaccgagagcctggagagcgacatcgccaagggcatcgacttcagcgagtacttcgagaacttcaagatcaagaaggacatcaaccagaaggacttccagaacctggagcacaagctgaggaaggagttcgaccagctgtacatccagatcgccaacaagtggaaggaggagaagtacaagcagtacaagtggaagaagggcagcaaggtggccaacggcagcgagatcctgagcagcgaggacatcctgaagctggtggtggacctgaaccaggacaacaaccagctgaaggacatcgtgcagaacaccttcaagggcttcttcacctacttcagcggcttcaacgacaacaggaggaactactaccaggtggagaaggaggccaagagcagcatcgccaccaggatcgtgaacgagaacctgcccaagttctgcgacaacttcctgctgttcgagaacaggaagaacgagtacctgggcgtgtacaacaccctgaagctgctgggcaaggacctggtggacaaggacaggaagaccctggtgaccatcgaggagatcatcttcagcatcaagcacttcaacttctgcctgagccagaaggagatcgagaactacaacaagcagatcggcaacgccaacctgctgatcaacctgtacaaccagaacaccagcaacgagagcgactacaagaggctgcccctgttcaagaccctgtacaagcagatcggctgcggcaagagggacagcctgttcttcgagatcaccgactacaccaagctggaggccgagggcaagaggatcctgggcaagagcgccatcagcgtggaggaggtgctgatccaggtgaaggagagcggcatcaagtacttcaccgagaagctggacagcctgctgaactacatcctggagaaggagaccgacaactaccagggcctgtactggagcaacatcgccgtgaacagcatcagcagcaagtacctggccaactggaacaacctgaaggagctgttcaagggcaagaaggaggtggccagctacgacaagggcagggaggaggagatcaagatcaacgaggccgtggacctgagcatcctgttcagcatcctgaacaacaccgaggactggaaggtgaaggaggtgttcttcaaggagaacctgaacgaggagagcgagaccaagaagatcatcgagaccagcaagacccccagccaggccctgctgaggatcgtgatcggcgacctgaagaagaacgccagcaacttcctgaacctgagcgacaacatcgagcagctgctggagagcaaggagaagatcaaggagagcatggactacgccctgagcgtgaccaggatgctgaagtacttcctggtgaaggagagcaagaccaagggcgcccccctggacagcaagctggtggaggacctgaacctgctgctgctgagcgacgacgccccctggttcatgtggtacgacggcctgaggaactacctgaccaagaagccccacgacgacgccaaggagaacaagctgaagctgaacttcgagaacagcaccctggccggcggctgggacatcaacaaggagcccgccaactactgcgtgatcctgaccaacagcgccaacgaccagtacctggccatcaacgccaagaggaacaacaagatcttctgcaaggagctggtggacggcaagggcaaggacaagaagctgctgaagaaccccctgtacgagagcgacgagcagcactggaagaagatggagtacaagctgctgcccggccccaacaagatgctgcccaagtgcctgctgcccaagagcaacctgaggaagttcggcgccagcgaggaggtgctgagcatctacgagaacggcagcttcaagaagaacacccccgacttcagcacccaggacctgcacaagctgatcgacttctacaagagcgccctgctgaagtacgaggactggaagtgcttccacttcaagttcaacgacaccaggagctacggcgacatcagccagttctacagcgacctggagaagcagggctacctgctgagcttcatcgacatcaacaagagcgtgctgaaccagctggtggacagcggcaagatctacctgttccagatcaggaaccaggacagcaacatcagcaagaagatcaaccaccaggacaacctgcacaccatctactggagggccgtgttcgaggacgtgttcaacaagcccaagctgaacggccaggccgaggtgttctacaggaaggccctgccccaggaggacctgcagctgctgaaggacaaggacaacaaggagatcatcgccaactacaggttcagcaaggagaagttcctgttccacgtgcccatcaccctgaacttctgcctgaagaacaagaagatcaacgacctggccaacagggagatcctgagcaacgacaacatctacttcctgggcatcgacaggggcgagaagcacctggcctactacagcctggtggaccagaagggcaacatcgtgatcgaccagaacggcaagagcatccagggcaccctgaacatccccttcctggacagcaacggcaaccagaggctggtgaaggccaccaagagggtgatgaaggacggcaaggaggacgcccaggtggtggagtgcaaggactacaacgagctgctggaggccagggccagcgacagggactacgccaggaagaactggcagaccatcggcgccatcaaggacctgaaggacggctacatcagccaggtggtgaggaagatcgccgacctggccgtggagtacaacgcctacatcgtgctggaggacctgaacaccggcttcaagaggggcaggcagaagatcgagaagagcgtgtaccagaagctggagctggccctggccaagaagctgaacttcctggtggacaagagcgccaagatcggcgaggtgggcagcgtgaccaacgccctgcagctgaccccccccgtgaacaactacagcgacatcgagaacaggaagcagttcggcgtggtgctgtacaccagggccaactacaccagccagaccgaccccgtgaccggctggaggaagaccatctacctgaagaccggcagcgaggacatcatcaagaagcagatcttcgagagcttcagcaacatctacttcgacggccaggactacatcttcacctacaccgacaagaacatcgacaggaagtgggagctgtacagcggcaagaacggcaagagcctggacaggtacaggggcaggaaggtgaacaacaagggctgggtgatcgacaagatcaacatccaggagatcctggacaagatcttcatcaacttcgagatctgctacggcgagaagaacaacatcttcgagaagctgaaggccggcgtggagctggagaagtacaacgagtacaccgcctgggagagcctgaggttcgccatcgacatcatccagcagatcaggaacaccggcatcgagagcagcgacagcgacttcctgctgagccccatcagggtgaacaacctgcacttcgacagcaggctggccagcgagaaccagcccaacagcggcgacgccaacggcgcctacaacatcgccaggaagggcatcatcatggccaactacatcaagcagggcgtgagccccttcgtgagcgacatcgactgggacaacttcgtgaccaagaacaaaagacctgccgccacaaagaaagccggacaggccaagaagaaaaaggattacaaagacgatgacgataagtga

SEQ ID NO: 57 - DNA, 4314 bp, synthetic construct
  source 1..4314: mol_type = other DNA
atggcccccaagaaaaagcggaaggtgaacaagttcctggacagcttcacccaccagtacagcctgagcaagaccctgaggttcgagctgaagcccgtgggcaagaccgccgagtggatccagaagagggacatcatcggcaccaagaagcaggagaacggcgaggcccagctgaccggcaaggacgccgtgagggccgagaactacgagtacgccaagaagctgctgaacgagatgcacaggctgttcatcgaggacagcctggccgccatcaaggaggagcagaaggtggagctgaacaagctgctggtgaagatcgccgagagcggcagcctggacggcttcaggaagcccatgagcaacatctgcaaggagctgttcgacagcgccacccaggactggctgctggcctacagcgaggagatgccctgcttctgggacgaggacatcctggagctgaccaacaaggccgagaccaccgagaacaagaagaacatcaagttcttcctgaagaaggccgaggacatcaggaagttcaaggacgaccccctgaaggccttcaagaagaagaagcacgaggccctgaacagcaacaccgaggccctgaagctgctggagtggaagatcaggaggggcgaggtgctgatcagcggcaaggacctgggcagggacgagagcgaggagatcctgagcaccaaggagctgatcctgatcctgaggagcttctacagcttcgtgacctacttcaccggcttcaacgagaacagggccaacatctacaacctggacgacaagaagtacatcagcaccagcatcgccttcaggagcttcgagcagaacctgttcttccacatcgagaacatcaagaagtggcacaccatcaccaacagcatcgaggagcacaccgaccagctgaacgccagcgactaccccatcaaggacaagctgagggagctggagctggccttcgactgcaagctggccgacctgttcgccgtggacagcttcagccagttcctgagccagagcggcatcgacaggtacaacagcatcatgggcggcatcgcccagaagaccggcaagaccaaggtgcagggcctgaacgagatcatcaaccaggccaggcagcagaccggcgccaagaggcacaagttcccccccatgaagctgctgtacaagcagatcctgagcaagggcgagggcatcttcgtggagagctacgtggacgacgccgacatgctgaccagcatcgccggcttccacgagaaccacctgaacgagagcctgtgcagcaactggctgcacgagctggaggagctgatcgacagcgtgcaggagagcaaggacaccatcttcatcgccagggacaagctgaacaggatcagccaggagatcaccggcagctggagcgccatccaggactggatcgtggcccaccagaaggaccagaaggaggtggacgccctgaccaagaagaaggtgttcagcatccaggagctggaggactacttcagcaagagcagcgacgacgagctgttcaccgccaagatccccgaggacaggagcgcctacctgcccggcgagggcgagggcatcctgttccagttcttcaaggtgagggccaaggagatcctggccgagctgcagagcggctggtacgccctggacaaggcccagctgctgcaggagaagcagctggacaagaacagggagaacgagggcagcaagggcttcgagcaggtgggcctgctgaagggctacctggacgcctgcaacagcctgagcaggttcatcgccgactggaacatccagaaggacatcaagcaggagaggtgcagcctgtggtacgagttcctgcagaagcacctggacaccgtgcccatcttcgacctgtacaaccaggtgaggaaccacgtgagcaagaaggcctacagcaccgacaagctgaaggtgaacttcgagaagagcaccctgctggacggctgggacaggaacaaggagagccagaacttcggcgtgctgttcgagaaggacggcgcctactacctgggcatcatgacccccgacagcaacggcatcttcgagtacgacgtgaaccccgacgagaacaccaagaagaaggccatcaaggaggagctgaggaagcagaccacccccgagaacggcgaggacaggttccagaagatcaactacaagctgctgcccggccccaacaagatgctgcccaaggtgttcttcgccaagagcaacgaggacctgttccagcccagcgccaggatcaccgagatcaaggagaacaagaagtacgccaaggcctacaccgacaagcacggcaaggccgagctgcacgagtacatcgacttctgccagaagagcctggtgaggcaccccgagtggagcgtggcctacggcttcgaccagaccaccttcaggaaggccaccgagtacatcagcatcgacgagttctacagggaggtggagctgctgggctacaaggtgaccttcgacagcatcaaggccacctacatcgaggagaaggtggccaacggcgagctgtacctgttccagatctacaacaaggacttcagcaccgccaagaagggcggcggcaaggacaacctgcacaccctgtactggaagagcctgttcagcgaggacaacctgaaggacgtggtgttcaagctgaacggccaggccgaggtgttcttcaggcccagcagcatcgtgtggagcgagaaggtgatgaagcagggccaccactacgacgacgacaagatgctgaaggacaagttcaagtaccccatcatcaaggacttcaggttcacccaggacaagttcttcttccactgccccatcagcctgaacttcaaggcccccgccaagccctacggcttcaactacaaggtgcacgacttcctgaggaagaacggcgagatcaacgtgatcggcatcgacaggggcgagaagcacctgctgtactacagcatcaccaaccagcagggcgacatcatcgagcagggcagcctgaacagcatcgagagcagctacgagaagggcagcagcagcgtgccccacagcaccaactacctggagaagctgcaggagaaggagaagaacagggacagcgccaggaagagctggagcaggatcgagaacatcaaggagctgaaggccggctacctgagccacgtggtgcacaagctggccaagctgatcatcaagcacaacgccgtggtggtgctggaggacctgaacgccggcttcaagaggggcaggttcaaggtggagaggcaggtgtaccagaagttcgagaaggccctgatcgacaagctgaactacctggtgttcaaggaccaggagaagcccagcgcccccggccaccccctgagggcctaccagctgaccgaccccttcgagagcttcgagaagctgggcaagcagagcggcatcctgttctacaccaccgccgcctacaccagcaccaccgaccccgtgaccggcttcctgaagaacgtgtacaagccctacgccaacgtggtggacagcagcgacttctggaagagcttcgacgagatccactacgacgtgaagaaggagaggttcgagttcacctacgacctgaggaagatcaagaacaagcaggtggacagggacaaggacgaggccaaggtgagcaggagcgtgtggaccgtgtgcagctgcgtggagaggagcaggtacgtgaagaggaaggccaacgaggagcagaagcagggcgccgccagcaagagcatcggcaagatgggcgagtacgaggtgttcgtggtgaccgacaagctgaaggccctgttcgagaaggagagcatcagctaccagagcggcgaggacatcaggagccagctgagcaacaggatcgacaagaaggacgccaagctgcacagggagctgatctactacttcggcaccctgctgaacatgagggtgaccgacaagcagagcgagagcggcaccgacgagaacgactacatcctgagccccatcgagcccttcttcgacagcaggaagggctacgagaagctgcccgtgaacggcgacgccaacggcgcctacaacatcgccaggaagggcatcgccatcctgaacaggatccacgagagcgacagcggcgacaaggtggacctgatgatcaggaagaaggactggcaggacttcgtgcagaacgaggaggtggtgcagaagcagatgggcaagctggacctgaaaagacctgccgccacaaagaaagccggacaggccaagaagaaaaaggattacaaagacgatgacgataagtga

SEQ ID NO: 58 - DNA, 3816 bp, synthetic construct
  source 1..3816: mol_type = other DNA
atggcccccaagaaaaagcggaaggtggccctgttcagcgagaacttcgtgggctgctaccccgtgagcaagaccctgaagttcgagctgaggcccgtgcccgagaccaaggcctacctggacgacgacaactgccccctgctggccggcgacctggagagggacaaggcctaccccgtggtgaaggagctgctggacaagtactacaggtacttcatcgacatctgcctgaaggacaagaagctgagccccgagaccatcgacgaggcctaccaggcctacagcagccacgacgagaagagccagaaggccctgaaggccgccagccagaagctgaggaaggaggtggccggcttcttcgacaagaagaccagggagacctactgcctggacaagtacagcaacctgctggtgctggacaagaagaacgacaacaaggagaagggcacccccaagaccatccccagcgtgctgaagaagtggctggagaacgagagcggcttcagcgagaaggaggtggagaagtacagccaggccgtgcagaccttcaacggcttcaccacctacttcatcagctacaaggagaacagggagaacatgttcagcagcgaggagaaggccagcgccatcagctacagggtggtgaaccagaacatgctgaggttcttcagcaacgtggagaacttcgagaacatcaagaagaagtaccccgacctgtaccagaccctgaaggagttcgaggagagcttcgtgcccgccgcctacagcagcctgctgagccaggccggcatcgacgactacaactacagggtgatcggcaggcccgccgaggacgaggacttcgccggcgtgaacaggctgatcaacgagtacaggcagaagaacgccgtgaggaacaggaacctgcccgtgatggtgaccctgtacaagcagatcctgagcgacagggagaacggcttcggcagggacatcctggccagcgacagcgaggccaaggagtacatcaggggcaagtacagcaaggcctacgacaccgcctgccagctgaccgtgttcgtgaacaactacatcctgaccgaggacctggacaccgtgtacgtgaagacccagcacctgaaggacatcagccagagcatcttcggcgagtggagcgtgctggacgacgccatcaacaacggcggcatcgacagcaagatcatcagcctgaaggccctggccgaggccttcgagaagtactgcagcgccctggaccccgacatggcccagaagtacagggacgccttcagcctgaaggagtacctgaagaacccccccgccgtggagcccaggctgcccgagaccgccgagaaggtgaccgcctacaagcccgccatggacgagctgatcagcctgatcaggttctacaagcccttctacctgtacgacggcaagaagaggctgcaggtgcccgccgagggcatggacttcagcaacgagttcgacaagtacttcgaggacctgaaggagttcagccacgcctacgacaaggtgaggaacttcgccaccaagaagccctacagcgtggagaagatcaagctgaacttcaaccagcccaccctgctggacggctgggacgtgaacaaggagaaggagaacgccagcttcctgttcgtgaaggagggcaagtactacctgggcatcgccgacaagagcgacaagaacctgttcgacaggagcagggacgccgtgaaggaggccctgaccctgacctgcggcgaggactactacgagaaggtgcagtacaagcagctgcccaacatcaggatgaacctgcccaggatcgtgttcaagcccaagtgggagaaggtgttcgcccccatcatcaccgagaggatcagcgccatcgagaaggagaagctgtacaccgccggcgccaacgacaggcaggccgtgatcgagtggatcgacttcgtgaagagcgtgctggccctgcacagcgagtggaacaactacttccagttcaagttcaaggagaccagcgcctacaagaacaccaaggagttctacgacgacctggacaaccaggcctacatcctgagcaagatcaagatcaacgccagctacatccacaagctggtgagggagaggaagctgtacctgttccagatctactgcaaggacttcagcgacaagaagaagaagaccggcgccgacaacctgcacaccatgtactggaggtgcctgttcagcgacgagaacctggccgccctggcccagggccaggcccccatcatcaagctgaacggcaaggccgagatcttcctgagggagcccagcctgaagtacgaggccacccaccccaagaaccagcccgtggccaacaagaaccccctgaacagcaagagggagagcctgttcagctacgacctggccaagaacaggaggttcaccgagaggaagctgttcttccactgccccctgaccatcaacttcaggaccgccgccgagggcgccaggagcttcaacgagagggtgaacaggttcgtggagaacaaccccgacatcaggatcatcggcatcgacaggggcgagaggcacctgctgtactacaccgtgatcaaccagagcggcaagatcctggagcagggcagcctgaacaagatcagcagcgactacaggacccccgagggcgccgaggtggagaaggtgaccgactaccacgagctgctgaacaggaaggagggcgacaggagggccgccaggcagacctgggacaccatcgagaacatcaaggagctgaaggagggctacctgagccaggtggtgcacaagctgaccaccctgatgatcaagcacaacgccgtggtggtgctggagaacctgaaccagaacttcaagaggagcaggatcaaggtggagaagcaggtgtaccagaagttcgagaaggccctgatcgacaagctgaactacctggtgttcaaggacaggccctacggcgcccccggcagctacgccaacggcctgcagctgaccgcccccttcgagagcttcgacaggctgggcaagcagaccggcgccctgtactacgtggtgcccagctacaccagccacatcgaccccaagaccggcttcgtgaacctgctgggcgccaagctgaggtacgagagcgtggtgaaggcccaggagaccatgaagaagatcggcagcttcaagttcaaccccgagaggaactggttcgagatcagcttcgactacggcgacttcggcatcgagatggcccacccccagtggaccgcctgcacccacggcgacgcctactggggctacaaccccaaggagaagagggccgtgagctacgacgtgaccgaggagatcaagaagctgctgaccgagtacggcatcgactacggcaccggcgccgacatcaaggaggccgccatcaggcagaccaagcccgccttctggaagaccctgttctacatgctgaagctggccctgcagctgaggaacaccgtggccggcaccgacaacgagaacgactacatcctgagccccgtggagtacgaggccggcaagttcttcgacagcaggaaggccggcgaggacgagccccagaacgccgacgccaacggcgcctaccacatcgccatgaagggcctgcaggccctgaggaggatcgaggacggcaaggtgaacaagttccccaagaacggcgagaggaaggcctggctggccttcatgcagaacagggagtacctgaagaaaagacctgccgccacaaagaaagccggacaggccaagaagaaaaaggattacaaagacgatgacgataagtga

SEQ ID NO: 59 - DNA, 4005 bp, synthetic construct
  source 1..4005: mol_type = other DNA
atggcccccaagaaaaagcggaaggtgagcaagctgagcaccttcaacgagcacttccagaagaccatcaccctgaggaacgagctggtgcccgtgggcaagaccctggagaacatcatcagcagcaacgtgctgatcaacgacgagaagaggagcgaggactacaagaaggccaaggagatcatcgacagctaccaccaggagttcatcgagaagagcctgagcaaggtggacatcgagtgggacgacctggcctactgcctgaccaagaaggagcccgagaacttcgacaagaagaagaagtaccaggaggagatcaacaagctgcagaacgagaagaggaagaagagcgtggagatcttcaagaagttcagcttcggcgtgctgtgcgacaacaacggccaggaggtgcagatcaagttcgacgacctgttcaagagcaccctgttcaagtacgtgctgcccaacttcctgaagaacgacgaggacaagaccgtggtgagggacttcagcgagttcaccagctacttcaccggcttccacgagaacaggaagaacctgtacaagagcgcccccatgcccaccgccgtggcctacaggatcgtgaacgacaacttccccaagttcatcagcaaccagaagatcttctgcgcctggaaggacaacgtgccccacttcgtggagatcgccaagaccaagctgagcgaggtgggcatcagcgacctggacatcgagagcaagttcgagctgagcaacttcaacagctgcctgaaccagaccggcatcgactactacaacgacctgaggggccagctgaacaacgtgatcagccacatctgccagcaggacaagaacctgggcgacctgctgaggaagaagaggtgcctgaagatggtgcccctgtacaagcagatcctgagcgacaaggacagcagcttcagcatcgacgagttcgacaacgacgagagcgccatccaggacgtgatcagcttctacaagaagatggtgggcgagaggtgcccccagaggaagctgagcgagctgctgagggagctgagcagccacgacagggagaagatcttcatccagggcaagaacctgaacagcatcagcaggatcctgttcggcggcaagaactggagcctgctgagggaggccatcatcgaggagaagagcaaggacaagagcttcaagaaggccatcaagagcaacagcaccgacgaggccctggagaagatcctgagcaaggacgagttcagcatcagcttcctgagcaggatctgccacgagaacctgagcgagaagatcgacaagttcatcaagaaccaggacgagcagctgatcaacatcaacagccagacctggcccaagaccctgaagaacagcgaggagaagaagatcatcaaggagcacctggacttcctgctgaacttctacaggttcgccaagagcttcagcagcaacaccatcgacaaggacatgagcttctacgccgacttcgacgagagcctgagcagcctggagaacgtgatcggcctgtacaacaaggtgaggaactacgtgaccaagaagccctacagcctggagaagatcaagctgaacttcgagatccccaacctggccgagggctggagcaagaccaaggagctggccaacctgaccgtgatcttcctgaaggacaacgagtactacctgggcatcatcaacaagaacagcaagatcgacttcagcctgatcaaggagaagaccagcagcgagaactgcttcaggaagatggagtacagcctgttcggcaagatcgcccagaacatcaacaggtgcgtgttcaccaaggccgtgaagaaccacttcagcaagagccccgacaacttcatcctgagcgacaagaagaagttcctgaaggagatcgagatcaccaaggagatctacgacctggtgaagagcgacgagagcgacaagaagtacctgaaggagtacaagaagatcaacgagaaggagttcaggagcgccctgagcaagtggatcaacttctgcatcgagttcctgaagagctacaagaacaccagccagttcgacttcagctgcatcaagaaggccgagagctacgacgacatcaacgacttctacgacgacatcgagaaggtgacctacaagatcgagttcgtggacatcagcaccagcctgatcgacgccctggtggacaagggccagctgttcctgttcaagatcaggaacaaggacttcgccaagaaggccagcggcacccccaacctgcacaccctgtacttcaagagcatcttcgacaccaagaacttcgagaacggcatcgtgaagctgaacggcgaggccgagatcttctacaggaagaagagcctgaacgtggacgacaccaccgtgcaccagaagggcagctacatcgtgaacaaggtggtggtggaccccagcagcggcaagagcgagcagatccccgacaacgtgtacaacaacatctacgccttcgtgaacggcaagctgaagaccctgagcaaggaggacgaggtgtactacgccaaggccaccatcaagaaggccacccacgagatcgtgaaggacaggaggttcaccgaggacaagttcttcttccactgccccatcaccatcaactacaagagcaaggagaagcccagcaagttcaacgacaaggtgctggacttcctgaggaagaacgacgacatcaacatcatcggcatcgacaggggcgagaggaacctgatctacgccaccgtgatcaacaagaacggcgagatcatcgactgcaagagcttcaacaccatcaagcaccagagcagcagcgtgaacttcgacgtggactaccacaacaagctgcaggagagggagaagaacaggaaggaggagaagaggagctggaacagcatcagcaagatcgccgacctgaaggagggctacctgagcgccgtgatccacgagatcgccctgatgatggtgaagtacaacgccatcgtggtgatggagaacctgaaccagggcttcaagaagatcaggggcggcatcgccgagaggagcgtgtaccagaagttcgagaagatgctgatcgacaagctgaactacttcgtgatcaagaacgagaactggaccaaccccggcggcgtgctgaacggctaccagctgaccaacaaggtgagcaccatcaaggacatcggcaaccagtgcggcttcctgttctacgtgcccgccgcctacaccagcaagatcgaccccagcaccggcttcgtgaacctgatcaacttcaacaagtacaagaacagcaacgacaggaggaagctgatctgcagcttcgacaagatctgcttcgtgcagaacgagaacctgttcaagttcagcatcgactacgagaagctgtgccccaacagcaagatgcccgtgaagaagtgggacatcttcagctacggcaacaggatcgtgagggaggacctgaagcacatccccatgaaggagaaccccaagtacgaccccaccgaggagctgaagaccctgttcagcagcaaggagatcgagtaccagaagggccagaacctgctggagatcatcagcaccaaggacatgaccgaggacaaggagttctggaacagcctgttcaacatcttcaaggccatcctgcagatgaggaacagcctgaccaacagccccgtggacaggctgctgagccccgtgaagggcaaggacggcagcttcttcgacaccgacaaggtggagggcaccaagttcgagaagctgaaggacgccgacgccaacggcgcctacaacatcgccctgaagggcctgctggtgctgaagaagaacgacagcgtgaagaccgacaaggacatgaagaacgtgaagaacgtgagcctggaggactggctgaagttcgtgcaggtgaccctgagggtgaaaagacctgccgccacaaagaaagccggacaggccaagaagaaaaaggattacaaagacgatgacgataagtga

SEQ ID NO: 60 - protein, 1307 aa, synthetic construct
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 61 - protein, 1300 aa, synthetic construct
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 62 - protein, 1228 aa, synthetic construct
AASKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGAAGYKSLFKKDIIETILPEAADDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNAKTKQALPKFKPLYKQVLSDRESLSFYGEGYTSDEEVLEVFRNTLNKNSEIFSSIKKLEKLFKNFDEYSSAGIFVKNGPAISTISKDIFGEWNLIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFAAAKKNNVFAWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 63 - DNA, 23 bp, synthetic construct
  source 1..23: mol_type = other DNA
gagaagtcattcaataaggccac

SEQ ID NO: 64 - DNA, 19 bp, synthetic construct
  source 1..19: mol_type = other DNA
aatttctactgttgtagat

SEQ ID NO: 65 - DNA, 20 bp, synthetic construct
  source 1..20: mol_type = other DNA
ggccaggcacagtggctcac

SEQ ID NO: 66 - DNA, 20 bp, synthetic construct
  source 1..20: mol_type = other DNA
ggccaggtgcggtggctcac

SEQ ID NO: 67 - DNA, 54 bp, synthetic construct
  source 1..54: mol_type = other DNA
  modified_base 1: mod_base = OTHER; note = phosphorylation modification
  modified_base 51..54: mod_base = OTHER; note = phosphorothioate linkage
acttcggtacgcgtcgacgtacggtcgacgagtcgcgcgtacgacgatcgagac

SEQ ID NO: 68 - DNA, 54 bp, synthetic construct
  source 1..54: mol_type = other DNA
  modified_base 1: mod_base = OTHER; note = phosphorylation modification
  modified_base 51..54: mod_base = OTHER; note = phosphorothioate linkage
gtctcgatcgtcgtacgcgcgactcgtcgaccgtacgtcgacgcgtaccgaagt

SEQ ID NO: 69 - DNA, 42 bp, synthetic construct
  source 1..42: mol_type = other DNA
  modified_base 1..4: mod_base = OTHER; note = phosphorothioate linkage
aactacacgacgctcttccgatctnnnwnnnwnnagtgacac

SEQ ID NO: 70 - DNA, 48 bp, synthetic construct
  source 1..48: mol_type = other DNA
  modified_base 48: mod_base = OTHER; note = phosphorylation modification
nnnnnngtgtcactnnwnnnwnnnagatcggaagagcgtcgtgtagtt

SEQ ID NO: 71 - DNA, 18 bp, synthetic construct
  source 1..18: mol_type = other DNA
gtcgacgagtcgcgcgta

SEQ ID NO: 72 - DNA, 18 bp, synthetic construct
  source 1..18: mol_type = other DNA
tcgtcgaccgtacgtcga

SEQ ID NO: 73 - DNA, 52 bp, synthetic construct
  source 1..52: mol_type = other DNA
  modified_base 49..52: mod_base = OTHER; note = phosphorothioate linkage
gtgactggagttcagacgtgtgctcttccgatctcgcgcgtacgacgatcga

SEQ ID NO: 74 - DNA, 54 bp, synthetic construct
  source 1..54: mol_type = other DNA
  modified_base 51..54: mod_base = OTHER; note = phosphorothioate linkage
gtgactggagttcagacgtgtgctcttccgatctatcgcgcgtacgacgatcga

SEQ ID NO: 75 - DNA, 55 bp, synthetic construct
  source 1..55: mol_type = other DNA
  modified_base 52..55: mod_base = OTHER; note = phosphorothioate linkage
gtgactggagttcagacgtgtgctcttccgatcttaacgcgcgtacgacgatcga

SEQ ID NO: 76 - DNA, 52 bp, synthetic construct
  source 1..52: mol_type = other DNA
  modified_base 49..52: mod_base = OTHER; note = phosphorothioate linkage
gtgactggagttcagacgtgtgctcttccgatcttacgtcgacgcgtaccga

SEQ ID NO: 77 - DNA, 54 bp, synthetic construct
  source 1..54: mol_type = other DNA
  modified_base 51..54: mod_base = OTHER; note = phosphorothioate linkage
gtgactggagttcagacgtgtgctcttccgatctgctacgtcgacgcgtaccga

SEQ ID NO: 78 - DNA, 55 bp, synthetic construct
  source 1..55: mol_type = other DNA
  modified_base 52..55: mod_base = OTHER; note = phosphorothioate linkage
gtgactggagttcagacgtgtgctcttccgatctatgtacgtcgacgcgtaccga

SEQ ID NO: 79 - DNA, 70 bp, synthetic construct
  source 1..70: mol_type = other DNA
aatgatacggcgaccaccgagatctacacnnnnnnnnacactctttccctacacgacgctcttccgatct

SEQ ID NO: 80 - DNA, 66 bp, synthetic construct
  source 1..66: mol_type = other DNA
caagcagaagacggcatacgagatnnnnnnnngtgactggagttcagacgtgtgctcttccgatct

SEQ ID NO: 81 - DNA, 17 bp, synthetic construct
  source 1..17: mol_type = other DNA
ccacaacggacgacttc

SEQ ID NO: 82 - DNA, 18 bp, synthetic construct
  source 1..18: mol_type = other DNA
ccacaacggacgacttct

SEQ ID NO: 83 - DNA, 19 bp, synthetic construct
  source 1..19: mol_type = other DNA
ccacaacggacgacttcta

SEQ ID NO: 84 - DNA, 20 bp, synthetic construct
  source 1..20: mol_type = other DNA
ccacaacggacgacttctat

SEQ ID NO: 85 - DNA, 21 bp, synthetic construct
  source 1..21: mol_type = other DNA
ccacaacggacgacttctatg

SEQ ID NO: 86 - DNA, 22 bp, synthetic construct
  source 1..22: mol_type = other DNA
ccacaacggacgacttctatga

SEQ ID NO: 87 - DNA, 23 bp, synthetic construct
  source 1..23: mol_type = other DNA
ccacaacggacgacttctatgac

SEQ ID NO: 88 - DNA, 24 bp, synthetic construct
  source 1..24: mol_type = other DNA
ccacaacggacgacttctatgacg

SEQ ID NO: 89 - DNA, 25 bp, synthetic construct
  source 1..25: mol_type = other DNA
ccacaacggacgacttctatgacga

SEQ ID NO: 90 - DNA, 3786 bp, synthetic construct
  source 1..3786: mol_type = other DNA
atggctcctaagaagaagcgcaaagtgatgagcaagctggagaagtttacaaactgctactccctgtctaagaccctgaggttcaaggccatccctgtgggcaagacccaggagaacatcgacaataagcggctgctggtggaggacgagaagagagccgaggattataagggcgtgaagaagctgctggatcgctactatctgtcttttatcaacgacgtgctgcacagcatcaagctgaagaatctgaacaattacatcagcctgttccggaagaaaaccagaaccgagaaggagaataaggagctggagaacctggagatcaatctgcggaaggagatcgccaaggccttcaagggcaacgagggctacaagtccctgtttaagaaggatatcatcgagacaatcctgccagagttcctggacgataaggacgagatcgccctggtgaacagcttcaatggctttaccacagccttcaccggcttctttgataacagagagaatatgttttccgaggaggccaagagcacatccatcgccttcaggtgtatcaacgagaatctgacccgctacatctctaatatggacatcttcgagaaggtggacgccatctttgataagcacgaggtgcaggagatcaaggagaagatcctgaacagcgactatgatgtggaggatttctttgagggcgagttctttaactttgtgctgacacaggagggcatcgacgtgtataacgccatcatcggcggcttcgtgaccgagagcggcgagaagatcaagggcctgaacgagtacatcaacctgtataatcagaaaaccaagcagaagctgcctaagtttaagccactgtataagcaggtgctgagcgatcgggagtctctgagcttctacggcgagggctatacatccgatgaggaggtgctggaggtgtttagaaacaccctgaacaagaacagcgagatcttcagctccatcaagaagctggagaagctgttcaagaattttgacgagtactctagcgccggcatctttgtgaagaacggccccgccatcagcacaatctccaaggatatcttcggcgagtggaacgtgatccgggacaagtggaatgccgagtatgacgatatccacctgaagaagaaggccgtggtgaccgagaagtacgaggacgatcggagaaagtccttcaagaagatcggctccttttctctggagcagctgcaggagtacgccgacgccgatctgtctgtggtggagaagctgaaggagatcatcatccagaaggtggatgagatctacaaggtgtatggctcctctgagaagctgttcgacgccgattttgtgctggagaagagcctgaagaagaacgacgccgtggtggccatcatgaaggacctgctggattctgtgaagagcttcgagaattacatcaaggccttctttggcgagggcaaggagacaaacagggacgagtccttctatggcgattttgtgctggcctacgacatcctgctgaaggtggaccacatctacgatgccatccgcaattatgtgacccagaagccctactctaaggataagttcaagctgtattttcagaaccctcagttcatgggcggctgggacaaggataaggagacagactatcgggccaccatcctgagatacggctccaagtactatctggccatcatggataagaagtacgccaagtgcctgcagaagatcgacaaggacgatgtgaacggcaattacgagaagatcaactataagctgctgcccggccctaataagatgctgccaaaggtgttcttttctaagaagtggatggcctactataaccccagcgaggacatccagaagatctacaagaatggcacattcaagaagggcgatatgtttaacctgaatgactgtcacaagctgatcgacttctttaaggatagcatctcccggtatccaaagtggtccaatgcctacgatttcaacttttctgagacagagaagtataaggacatcgccggcttttacagagaggtggaggagcagggctataaggtgagcttcgagtctgccagcaagaaggaggtggataagctggtggaggagggcaagctgtatatgttccagatctataacaaggacttttccgataagtctcacggcacacccaatctgcacaccatgtacttcaagctgctgtttgacgagaacaatcacggacagatcaggctgagcggaggagcagagctgttcatgaggcgcgcctccctgaagaaggaggagctggtggtgcacccagccaactcccctatcgccaacaagaatccagataatcccaagaaaaccacaaccctgtcctacgacgtgtataaggataagaggttttctgaggaccagtacgagctgcacatcccaatcgccatcaataagtgccccaagaacatcttcaagatcaatacagaggtgcgcgtgctgctgaagcacgacgataacccctatgtgatcggcatcgataggggcgagcgcaatctgctgtatatcgtggtggtggacggcaagggcaacatcgtggagcagtattccctgaacgagatcatcaacaacttcaacggcatcaggatcaagacagattaccactctctgctggacaagaaggagaaggagaggttcgaggcccgccagaactggacctccatcgagaatatcaaggagctgaaggccggctatatctctcaggtggtgcacaagatctgcgagctggtggagaagtacgatgccgtgatcgccctggaggacctgaactctggctttaagaatagccgcgtgaaggtggagaagcaggtgtatcagaagttcgagaagatgctgatcgataagctgaactacatggtggacaagaagtctaatccttgtgcaacaggcggcgccctgaagggctatcagatcaccaataagttcgagagctttaagtccatgtctacccagaacggcttcatcttttacatccctgcctggctgacatccaagatcgatccatctaccggctttgtgaacctgctgaaaaccaagtataccagcatcgccgattccaagaagttcatcagctcctttgacaggatcatgtacgtgcccgaggaggatctgttcgagtttgccctggactataagaacttctctcgcacagacgccgattacatcaagaagtggaagctgtactcctacggcaaccggatcagaatcttccggaatcctaagaagaacaacgtgttcgactgggaggaggtgtgcctgaccagcgcctataaggagctgttcaacaagtacggcatcaattatcagcagggcgatatcagagccctgctgtgcgagcagtccgacaaggccttctactctagctttatggccctgatgagcctgatgctgcagatgcggaacagcatcacaggccgcaccgacgtggattttctgatcagccctgtgaagaactccgacggcatcttctacgatagccggaactatgaggcccaggagaatgccatcctgccaaagaacgccgacgccaatggcgcctataacatcgccagaaaggtgctgtgggccatcggccagttcaagaaggccgaggacgagaagctggataaggtgaagatcgccatctctaacaaggagtggctggagtacgcccagaccagcgtgaagcacaaacgaccagctgcaacaaagaaggctggccaagcaaaaaagaagaaagattataaagatgatgacgataagtga

SEQ ID NO: 91 - DNA, 3714 bp, synthetic construct
  source 1..3714: mol_type = other DNA
gccttgttttccgagaactttgttgggtgttatccagtttctaagactttgaagtttgaactgcgcccagtgcccgaaacgaaagcatacctggatgatgataactgtccactgttggctggtgatctggagagagataaagcctacccggtggtgaaggaattgttggacaagtactataggtatttcattgacatttgtcttaaagataaaaagttgtcccccgaaaccattgacgaggcctaccaggcctacagtagccacgacgagaaatctcagaaggcactgaaggccgccagtcagaagctgaggaaggaggtcgccggatttttcgataaaaaaacccgcgaaacctactgtctcgacaagtacagtaatttgttggtgctcgacaagaaaaacgataataaagagaagggaacgcccaagacgatcccgtcagttctcaagaaatggcttgagaatgagtcaggattctccgagaaagaggtggaaaaatactcccaggctgtgcagacgtttaacggttttaccacatacttcatttcctacaaagaaaacagggagaacatgttcagcagtgaggagaaagcctctgccatctcttacagagttgttaatcagaacatgctgagatttttctccaatgtggaaaatttcgaaaacataaaaaagaagtaccctgacctttatcagacactgaaagaatttgaggaatcctttgtgcctgccgcctactcaagcctcctttctcaggccggtatagatgactataattacagagttatcggacgacctgcagaagacgaggatttcgccggagtcaatcgcctgataaatgagtataggcagaaaaatgccgtgcgaaaccggaacctgccagtgatggttactttgtacaaacagattctcagcgatcgggaaaatggtttcgggcgggacatcttggcttccgactcagaagcaaaagagtatattcgggggaagtactccaaggcttatgacaccgcctgccaactgactgtatttgtcaataattacatactgacagaagatctggacactgtgtacgttaagacccagcacctgaaggatatctctcaaagcattttcggagaatggtccgtccttgatgacgctatcaacaacggagggatcgacagcaagattatctcattgaaggctctggctgaggcattcgagaaatattgttcagcccttgatcccgatatggcccaaaaataccgggacgccttcagtttgaaggagtatctcaagaatccgcctgctgtggaacccagactgcctgaaacggccgagaaggttaccgcttataagcccgctatggatgagttgatctctctgataaggttttacaagcctttctatttgtacgacgggaagaagagactgcaggtgcctgccgagggaatggatttctctaatgagttcgacaaatatttcgaggacttgaaggaatttagtcacgcatacgataaagttcgaaattttgcaaccaagaaaccctacagcgtagagaagataaaactcaatttcaaccagccaacactcctggacgggtgggacgtgaacaaggagaaagagaatgcatcatttctctttgtcaaagagggcaagtactacctgggaattgccgacaagtccgataaaaatctgttcgatcggagtcgggatgccgtcaaagaggcactcactcttacttgcggcgaagattactatgagaaggtgcagtataagcagcttccgaacatacggatgaacctgcctcgcatagtttttaagcctaaatgggagaaggtctttgctcctataatcactgagagaatcagcgccattgagaaagaaaagctttacaccgcaggtgccaacgataggcaggcggtgatcgagtggatagacttcgtcaagtctgtccttgcgctgcactccgagtggaacaactatttccagtttaagtttaaggagacgtccgcatataaaaatacaaaagagttctacgacgatcttgataaccaggcttacatcttgagtaagatcaaaatcaacgcctcatatatccacaaactggttcgggagcgcaaattgtatctgtttcagatctattgtaaagacttttctgataagaaaaaaaagaccggcgccgacaatctccataccatgtattggagatgcctgttctccgatgaaaacttggcagctctcgcacagggacaggccccaatcataaagttgaacgggaaagctgaaatcttcctccgcgagccttctctcaaatatgaggcgacgcatccgaagaaccagccagtggccaacaagaatcccctgaactcaaaacgggagagcctgttcagctacgatctggcaaaaaatcgccgctttaccgagaggaagttgttcttccactgccccctgaccatcaactttaggacagctgcggaaggtgcgcggagcttcaatgagagagttaataggttcgtcgagaacaaccccgatatcagaataataggcattgataggggcgagagacacctgctgtactacactgtgatcaaccagagcggcaagattctggagcagggaagtctgaacaaaattagctcagattaccgaactcctgagggcgccgaggtggaaaaagtcactgactatcacgagctcttgaaccggaaagaaggagatagacgagctgcccgacagacatgggacaccatagaaaatatcaaggagcttaaagagggatacctcagccaggtggtgcataagctgactacactgatgattaaacacaatgctgtggtcgttttggagaaccttaatcaaaacttcaaacgctcccgcattaaggtcgagaagcaggtctatcagaagttcgagaaggctctgatcgataaactgaattatttggttttcaaggatagaccttacggagctcccggttcctatgccaatgggctgcagcttacggctcctttcgaatcctttgatagactcggcaagcagacaggggctctctattacgtggtgcccagctacacatcccacattgatcctaagactgggtttgtgaacctcctgggcgcaaagctccggtacgaaagcgtggtgaaagctcaggaaacaatgaagaagatcgggagtttcaagttcaaccccgagaggaattggtttgaaatcagcttcgactacggcgacttcgggatcgagatggcacatccccagtggaccgcatgtactcacggcgatgcttattggggatacaatcccaaggaaaaaagggccgttagctacgacgtgacggaagagattaaaaagctgctgacagaatacggaattgattatgggactggagcagatatcaaggaggccgctattcgacagaccaagcccgctttttggaagaccctgttttacatgctcaagctggcattgcagttgaggaataccgtggctggcactgataacgagaacgactacattctgtcaccagtcgagtatgaggccgggaagttctttgattctagaaaggctggagaggacgaaccccagaatgcagacgccaatggagcgtaccacatcgcgatgaaaggattgcaggcattgcggcgcatcgaggacggcaaagtgaacaaattcccgaagaacggtgagcgaaaagcttggctggcctttatgcaaaatagggaatatttgaaa

SEQ ID NO: 92 - DNA, 3816 bp, synthetic construct
  source 1..3816: mol_type = other DNA
atggccccaaagaagaagcggaaggtcgccttgttttccgagaactttgttgggtgttatccagtttctaagactttgaagtttgaactgcgcccagtgcccgaaacgaaagcatacctggatgatgataactgtccactgttggctggtgatctggagagagataaagcctacccggtggtgaaggaattgttggacaagtactataggtatttcattgacatttgtcttaaagataaaaagttgtcccccgaaaccattgacgaggcctaccaggcctacagtagccacgacgagaaatctcagaaggcactgaaggccgccagtcagaagctgaggaaggaggtcgccggatttttcgataaaaaaacccgcgaaacctactgtctcgacaagtacagtaatttgttggtgctcgacaagaaaaacgataataaagagaagggaacgcccaagacgatcccgtcagttctcaagaaatggcttgagaatgagtcaggattctccgagaaagaggtggaaaaatactcccaggctgtgcagacgtttaacggttttaccacatacttcatttcctacaaagaaaacagggagaacatgttcagcagtgaggagaaagcctctgccatctcttacagagttgttaatcagaacatgctgagatttttctccaatgtggaaaatttcgaaaacataaaaaagaagtaccctgacctttatcagacactgaaagaatttgaggaatcctttgtgcctgccgcctactcaagcctcctttctcaggccggtatagatgactataattacagagttatcggacgacctgcagaagacgaggatttcgccggagtcaatcgcctgataaatgagtataggcagaaaaatgccgtgcgaaaccggaacctgccagtgatggttactttgtacaaacagattctcagcgatcgggaaaatggtttcgggcgggacatcttggcttccgactcagaagcaaaagagtatattcgggggaagtactccaaggcttatgacaccgcctgccaactgactgtatttgtcaataattacatactgacagaagatctggacactgtgtacgttaagacccagcacctgaaggatatctctcaaagcattttcggagaatggtccgtccttgatgacgctatcaacaacggagggatcgacagcaagattatctcattgaaggctctggctgaggcattcgagaaatattgttcagcccttgatcccgatatggcccaaaaataccgggacgccttcagtttgaaggagtatctcaagaatccgcctgctgtggaacccagactgcctgaaacggccgagaaggttaccgcttataagcccgctatggatgagttgatctctctgataaggttttacaagcctttctatttgtacgacgggaagaagagactgcaggtgcctgccgagggaatggatttctctaatgagttcgacaaatatttcgaggacttgaaggaatttagtcacgcatacgataaagttcgaaattttgcaaccaagaaaccctacagcgtagagaagataaaactcaatttcaaccagccaacactcctggacgggtgggacgtgaacaaggagaaagagaatgcatcatttctctttgtcaaagagggcaagtactacctgggaattgccgacaagtccgataaaaatctgttcgatcggagtcgggatgccgtcaaagaggcactcactcttacttgcggcgaagattactatgagaaggtgcagtataagcagcttccgaacatacggatgaacctgcctcgcatagtttttaagcctaaatgggagaaggtctttgctcctataatcactgagagaatcagcgccattgagaaagaaaagctttacaccgcaggtgccaacgataggcaggcggtgatcgagtggatagacttcgtcaagtctgtccttgcgctgcactccgagtggaacaactatttccagtttaagtttaaggagacgtccgcatataaaaatacaaaagagttctacgacgatcttgataaccaggcttacatcttgagtaagatcaaaatcaacgcctcatatatccacaaactggttcgggagcgcaaattgtatctgtttcagatctattgtaaagacttttctgataagaaaaaaaagaccggcgccgacaatctccataccatgtattggagatgcctgttctccgatgaaaacttggcagctctcgcacagggacaggccccaatcataaagttgaacgggaaagctgaaatcttcctccgcgagccttctctcaaatatgaggcgacgcatccgaagaaccagccagtggccaacaagaatcccctgaactcaaaacgggagagcctgttcagctacgatctggcaaaaaatcgccgctttaccgagaggaagttgttcttccactgccccctgaccatcaactttaggacagctgcggaaggtgcgcggagcttcaatgagagagttaataggttcgtcgagaacaaccccgatatcagaataataggcattgataggggcgagagacacctgctgtactacactgtgatcaaccagagcggcaagattctggagcagggaagtctgaacaaaattagctcagattaccgaactcctgagggcgccgaggtggaaaaagtcactgactatcacgagctcttgaaccggaaagaaggagatagacgagctgcccgacagacatgggacaccatagaaaatatcaaggagcttaaagagggatacctcagccaggtggtgcataagctgactacactgatgattaaacacaatgctgtggtcgttttggagaaccttaatcaaaacttcaaacgctcccgcattaaggtcgagaagcaggtctatcagaagttcgagaaggctctgatcgataaactgaattatttggttttcaaggatagaccttacggagctcccggttcctatgccaatgggctgcagcttacggctcctttcgaatcctttgatagactcggcaagcagacaggggctctctattacgtggtgcccagctacacatcccacattgatcctaagactgggtttgtgaacctcctgggcgcaaagctccggtacgaaagcgtggtgaaagctcaggaaacaatgaagaagatcgggagtttcaagttcaaccccgagaggaattggtttgaaatcagcttcgactacggcgacttcgggatcgagatggcacatccccagtggaccgcatgtactcacggcgatgcttattggggatacaatcccaaggaaaaaagggccgttagctacgacgtgacggaagagattaaaaagctgctgacagaatacggaattgattatgggactggagcagatatcaaggaggccgctattcgacagaccaagcccgctttttggaagaccctgttttacatgctcaagctggcattgcagttgaggaataccgtggctggcactgataacgagaacgactacattctgtcaccagtcgagtatgaggccgggaagttctttgattctagaaaggctggagaggacgaaccccagaatgcagacgccaatggagcgtaccacatcgcgatgaaaggattgcaggcattgcggcgcatcgaggacggcaaagtgaacaaattcccgaagaacggtgagcgaaaagcttggctggcctttatgcaaaatagggaatatttgaaaaaacgacccgccgccacaaaaaaggccgggcaggcaaagaaaaaaaaggattacaaagacgatgacgacaagtaa

SEQ ID NO: 93 - DNA, 3714 bp, synthetic construct
  source 1..3714: mol_type = other DNA
gccttgttttccgagaactttgttgggtgttatccagtttctaagactttgaagtttgaactgcgcccagtgcccgaaacgaaagcatacctggatgatgataactgtccactgttggctggtgatctggagagagataaagcctacccggtggtgaaggaattgttggacaagtactataggtatttcattgacatttgtcttaaagataaaaagttgtcccccgaaaccattgacgaggcctaccaggcctacagtagccacgacgagaaatctcagaaggcactgaaggccgccagtcagaagctgaggaaggaggtcgccggatttttcgataaaaaaacccgcgaaacctactgtctcgacaagtacagtaatttgttggtgctcgacaagaaaaacgataataaagagaagggaacgcccaagacgatcccgtcagttctcaagaaatggcttgagaatgagtcaggattctccgagaaagaggtggaaaaatactcccaggctgtgcagacgtttaacggttttaccacatacttctcctcctacaaacgaaacagggagaacatgttcagcagtgaggagaaagcctctgccatctcttacagagttgttaatcagaacatgctgagatttttctccaatgtggaaaatttcgaaaacataaaaaagaagtaccctgacctttatcagacactgaaagaatttgaggaatcctttgtgcctgccgcctactcaagcctcctttctcaggccggtatagatgactataattacagagttatcggacgacctgcagaagacgaggatttcgccggagtcaatcgcctgataaatgagtataggcagaaaaatgccgtgcgaaaccggaacctgccagtgatggttactttgtacaaacagattctcagcgatcgggaaaatggtttcgggcgggacatcttggcttccgactcagaagcaaaagagtatattcgggggaagtactccaaggcttatgacaccgcctgccaactgactgtatttgtcaataattacatactgacagaagatctggacactgtgtacgttaagacccagcacctgaaggatatctctcaaagcattttcggagaatggtccgtccttgatgacgctatcaacaacggagggatcgacagcaagattatctcattgaaggctctggctgaggcattcgagaaatattgttcagcccttgatcccgatatggcccaaaaataccgggacgccttcagtttgaaggagtatctcaagaatccgcctgctgtggaacccagactgcctgaaacggccgagaaggttaccgcttataagcccgctatggatgagttgatctctctgataaggttttacaagcctttctatttgtacgacgggaagaagagactgcaggtgcctgccgagggaatggatttctctaatgagttcgacaaatatttcgaggacttgaaggaatttagtcacgcatacgataaagttcgaaattttgcaaccaagaaaccctacagcgtagagaagataaaactcaatttcaaccagccaacactcctgcgcgggtgggacgtgaacgtggagaaaaatcgggcatcatttctctttgtcaaagagggcaagtactacctgggaattgccgacaagtccgataaaaatctgttcgatcggagtcgggatgccgtcaaagaggcactcactcttacttgcggcgaagattactatgagaaggtgcagtatcggcagcttccgaacatacggatgaacctgcctcgcatagtttttaagcctaaatgggagaaggtctttgctcctataatcactgagagaatcagcgccattgagaaagaaaagctttacaccgcaggtgccaacgataggcaggcggtgatcgagtggatagacttcgtcaagtctgtccttgcgctgcactccgagtggaacaactatttccagtttaagtttaaggagacgtccgcatataaaaatacaaaagagttctacgacgatcttgataaccaggcttacatcttgagtaagatcaaaatcaacgcctcatatatccacaaactggttcgggagcgcaaattgtatctgtttcagatctattgtaaagacttttctgataagaaaaaaaagaccggcgccgacaatctccataccatgtattggagatgcctgttctccgatgaaaacttggcagctctcgcacagggacaggccccaatcataaagttgaacgggaaagctgaaatcttcctccgcgagccttctctcaaatatgaggcgacgcatccgaagaaccagccagtggccaacaagaatcccctgaactcaaaacgggagagcctgttcagctacgatctggcaaaaaatcgccgctttaccgagaggaagttgttcttccactgccccctgaccatcaactttaggacagctgcggaaggtgcgcggagcttcaatgagagagttaataggttcgtcgagaacaaccccgatatcagaataataggcattgataggggcgagagacacctgctgtactacactgtgatcaaccagagcggcaagattctggagcagggaagtctgaacaaaattagctcagattaccgaactcctgagggcgccgaggtggaaaaagtcactgactatcacgagctcttgaaccggaaagaaggagatagacgagctgcccgacagacatgggacaccatagaaaatatcaaggagcttaaagagggatacctcagccaggtggtgcataagctgactacactgatgattaaacacaatgctgtggtcgttttggagaaccttaatcaaaacttcaaacgctcccgcattaaggtcgagaagcaggtctatcagaagttcgagaaggctctgatcgataaactgaattatttggttttcaaggatagaccttacggagctcccggttcctatgccaatgggctgcagcttacggctcctttcgaatcctttgatagactcggcaagcagacaggggctctctattacgtggtgcccagctacacatcccacattgatcctaagactgggtttgtgaacctcctgggcgcaaagctccggtacgaaagcgtggtgaaagctcaggaaacaatgaagaagatcgggagtttcaagttcaaccccgagaggaattggtttgaaatcagcttcgactacggcgacttcgggatcgagatggcacatccccagtggaccgcatgtactcacggcgatgcttattggggatacaatcccaaggaaaaaagggccgttagctacgacgtgacggaagagattaaaaagctgctgacagaatacggaattgattatgggactggagcagatatcaaggaggccgctattcgacagaccaagcccgctttttggaagaccctgttttacatgctcaagctggcattgcagttgaggaataccgtggctggcactgataacgagaacgactacattctgtcaccagtcgagtatgaggccgggaagttctttgattctagaaaggctggagaggacgaaccccagaatgcagacgccaatggagcgtaccacatcgcgatgaaaggattgcaggcattgcggcgcatcgaggacggcaaagtgaacaaattcccgaagaacggtgagcgaaaagcttggctggcctttatgcaaaatagggaatatttgaaa

SEQ ID NO: 94 - DNA, 3816 bp, synthetic construct
  source 1..3816: mol_type = other DNA
atggccccaaagaagaagcggaaggtcgccttgttttccgagaactttgttgggtgttatccagtttctaagactttgaagtttgaactgcgcccagtgcccgaaacgaaagcatacctggatgatgataactgtccactgttggctggtgatctggagagagataaagcctacccggtggtgaaggaattgttggacaagtactataggtatttcattgacatttgtcttaaagataaaaagttgtcccccgaaaccattgacgaggcctaccaggcctacagtagccacgacgagaaatctcagaaggcactgaaggccgccagtcagaagctgaggaaggaggtcgccggatttttcgataaaaaaacccgcgaaacctactgtctcgacaagtacagtaatttgttggtgctcgacaagaaaaacgataataaagagaagggaacgcccaagacgatcccgtcagttctcaagaaatggcttgagaatgagtcaggattctccgagaaagaggtggaaaaatactcccaggctgtgcagacgtttaacggttttaccacatacttctcctcctacaaacgaaacagggagaacatgttcagcagtgaggagaaagcctctgccatctcttacagagttgttaatcagaacatgctgagatttttctccaatgtggaaaatttcgaaaacataaaaaagaagtaccctgacctttatcagacactgaaagaatttgaggaatcctttgtgcctgccgcctactcaagcctcctttctcaggccggtatagatgactataattacagagttatcggacgacctgcagaagacgaggatttcgccggagtcaatcgcctgataaatgagtataggcagaaaaatgccgtgcgaaaccggaacctgccagtgatggttactttgtacaaacagattctcagcgatcgggaaaatggtttcgggcgggacatcttggcttccgactcagaagcaaaagagtatattcgggggaagtactccaaggcttatgacaccgcctgccaactgactgtatttgtcaataattacatactgacagaagatctggacactgtgtacgttaagacccagcacctgaaggatatctctcaaagcattttcggagaatggtccgtccttgatgacgctatcaacaacggagggatcgacagcaagattatctcattgaaggctctggctgaggcattcgagaaatattgttcagcccttgatcccgatatggcccaaaaataccgggacgccttcagtttgaaggagtatctcaagaatccgcctgctgtggaacccagactgcctgaaacggccgagaaggttaccgcttataagcccgctatggatgagttgatctctctgataaggttttacaagcctttctatttgtacgacgggaagaagagactgcaggtgcctgccgagggaatggatttctctaatgagttcgacaaatatttcgaggacttgaaggaatttagtcacgcatacgataaagttcgaaattttgcaaccaagaaaccctacagcgtagagaagataaaactcaatttcaaccagccaacactcctgcgcgggtgggacgtgaacgtggagaaaaatcgggcatcatttctctttgtcaaagagggcaagtactacctgggaattgccgacaagtccgataaaaatctgttcgatcggagtcgggatgccgtcaaagaggcactcactcttacttgcggcgaagattactatgagaaggtgcagtatcggcagcttccgaacatacggatgaacctgcctcgcatagtttttaagcctaaatgggagaaggtctttgctcctataatcactgagagaatcagcgccattgagaaagaaaagctttacaccgcaggtgccaacgataggcaggcggtgatcgagtggatagacttcgtcaagtctgtccttgcgctgcactccgagtggaacaactatttccagtttaagtttaaggagacgtccgcatataaaaatacaaaagagttctacgacgatcttgataaccaggcttacatcttgagtaagatcaaaatcaacgcctcatatatccacaaactggttcgggagcgcaaattgtatctgtttcagatctattgtaaagacttttctgataagaaaaaaaagaccggcgccgacaatctccataccatgtattggagatgcctgttctccgatgaaaacttggcagctctcgcacagggacaggccccaatcataaagttgaacgggaaagctgaaatcttcctccgcgagccttctctcaaatatgaggcgacgcatccgaagaaccagccagtggccaacaagaatcccctgaactcaaaacgggagagcctgttcagctacgatctggcaaaaaatcgccgctttaccgagaggaagttgttcttccactgccccctgaccatcaactttaggacagctgcggaaggtgcgcggagcttcaatgagagagttaataggttcgtcgagaacaaccccgatatcagaataataggcattgataggggcgagagacacctgctgtactacactgtgatcaaccagagcggcaagattctggagcagggaagtctgaacaaaattagctcagattaccgaactcctgagggcgccgaggtggaaaaagtcactgactatcacgagctcttgaaccggaaagaaggagatagacgagctgcccgacagacatgggacaccatagaaaatatcaaggagcttaaagagggatacctcagccaggtggtgcataagctgactacactgatgattaaacacaatgctgtggtcgttttggagaaccttaatcaaaacttcaaacgctcccgcattaaggtcgagaagcaggtctatcagaagttcgagaaggctctgatcgataaactgaattatttggttttcaaggatagaccttacggagctcccggttcctatgccaatgggctgcagcttacggctcctttcgaatcctttgatagactcggcaagcagacaggggctctctattacgtggtgcccagctacacatcccacattgatcctaagactgggtttgtgaacctcctgggcgcaaagctccggtacgaaagcgtggtgaaagctcaggaaacaatgaagaagatcgggagtttcaagttcaaccccgagaggaattggtttgaaatcagcttcgactacggcgacttcgggatcgagatggcacatccccagtggaccgcatgtactcacggcgatgcttattggggatacaatcccaaggaaaaaagggccgttagctacgacgtgacggaagagattaaaaagctgctgacagaatacggaattgattatgggactggagcagatatcaaggaggccgctattcgacagaccaagcccgctttttggaagaccctgttttacatgctcaagctggcattgcagttgaggaataccgtggctggcactgataacgagaacgactacattctgtcaccagtcgagtatgaggccgggaagttctttgattctagaaaggctggagaggacgaaccccagaatgcagacgccaatggagcgtaccacatcgcgatgaaaggattgcaggcattgcggcgcatcgaggacggcaaagtgaacaaattcccgaagaacggtgagcgaaaagcttggctggcctttatgcaaaatagggaatatttgaaaaaacgacccgccgccacaaaaaaggccgggcaggcaaagaaaaaaaaggattacaaagacgatgacgacaagtaa

SEQ ID NO: 95 - DNA, 20 bp, synthetic construct
  source 1..20: mol_type = other DNA
actgtcgtttctggaagaga

SEQ ID NO: 96 - DNA, 20 bp, synthetic construct
  source 1..20: mol_type = other DNA
tgtatctatgagcacatgca

SEQ ID NO: 97 - DNA, 20 bp, synthetic construct
  source 1..20: mol_type = other DNA
gggagttaacagttacttcg

SEQ ID NO: 98 - DNA, 20 bp, synthetic construct
  source 1..20: mol_type = other DNA
gcattaggagcttgagccca

SEQ ID NO: 99 - DNA, 20 bp, synthetic construct
  source 1..20: mol_type = other DNA
gagacaacgctggccttttc

SEQ ID NO: 100 - DNA, 20 bp, synthetic construct
  source 1..20: mol_type = other DNA
aggagtgttcagtctccgtg

SEQ ID NO: 101 - DNA, 20 bp, synthetic construct
  source 1..20: mol_type = other DNA
gctcagcaggcacctgcctc

SEQ ID NO: 102 - DNA, 20 bp, synthetic construct
  source 1..20: mol_type = other DNA
tggttgcccaccctagtcat

SEQ ID NO: 103 - DNA, 20 bp, synthetic construct
  source 1..20: mol_type = other DNA
gggaggcctggagtcatggc

SEQ ID NO: 104 - DNA, 20 bp, synthetic construct
  source 1..20: mol_type = other DNA
cttctgacacaactgtgttc

SEQ ID NO: 105 - DNA, 20 bp, synthetic construct
  source 1..20: mol_type = other DNA
aggttgctagtgaacacagt

SEQ ID NO: 106 - DNA, 20 bp, synthetic construct
  source 1..20: mol_type = other DNA
ttctttaccataagcgaagg

SEQ ID NO: 107 - DNA, 20 bp, synthetic construct
  source 1..20: mol_type = other DNA
ggtagttttctggggtccga

SEQ ID NO: 108 - DNA, 20 bp, synthetic construct
  source 1..20: mol_type = other DNA
ttgcccaggctggagtgcag

SEQ ID NO: 109 - DNA, 20 bp, synthetic construct
  source 1..20: mol_type = other DNA
cttggacaagtcctggaagg

SEQ ID NO: 110 - DNA, 20 bp, synthetic construct
  source 1..20: mol_type = other DNA
aatggcaggcagagtgcctt

SEQ ID NO: 111 - DNA, 20 bp, synthetic construct
  source 1..20: mol_type = other DNA
gatacctgaataaactaaat

SEQ ID NO: 112 - DNA, 20 bp, synthetic construct
  source 1..20: mol_type = other DNA
ggtctggccttagtgatctt

SEQ ID NO: 113 - DNA, 20 bp, synthetic construct
  source 1..20: mol_type = other DNA
accatcagaggacacttgga

SEQ ID NO: 114 - DNA, 20 bp, synthetic construct
  source 1..20: mol_type = other DNA
cctctgggtaagttgccaaa

SEQ ID NO: 115 - DNA, 20 bp, synthetic construct
  source 1..20: mol_type = other DNA
gcaacttacccagaggcaaa

SEQ ID NO: 116 - DNA, 20 bp, synthetic construct
  source 1..20: mol_type = other DNA
cttgccattccccacttgaa

SEQ ID NO: 117 - DNA, 20 bp, synthetic construct
  source 1..20: mol_type = other DNA
ttgcaagccaccagagctat

SEQ ID NO: 118 - DNA, 20 bp, synthetic construct
  source 1..20: mol_type = other DNA
tcacttaggataggagaaga

SEQ ID NO: 119 - DNA, 20 bp, synthetic construct
  source 1..20: mol_type = other DNA
ttgcaaggctagtgctagtg

SEQ ID NO: 120 - DNA, 20 bp, synthetic construct
  source 1..20: mol_type = other DNA
tccaatcttgattccactga

SEQ ID NO: 121 - DNA, 20 bp, synthetic construct
  source 1..20: mol_type = other DNA
gatgaagtacaggtagcaac

SEQ ID NO: 122 - DNA, 20 bp, synthetic construct
  source 1..20: mol_type = other DNA
gaattggtagggcgtcacgt

SEQ ID NO: 123 - DNA, 20 bp, synthetic construct
  source 1..20: mol_type = other DNA
aaaacctatacaacacggaa

SEQ ID NO: 124 - DNA, 20 bp, synthetic construct
  source 1..20: mol_type = other DNA
tttctcggcaaccttggccc

SEQ ID NO: 125 - DNA, 20 bp, synthetic construct
  source 1..20: mol_type = other DNA
cctgtacaaatcaaggccac

SEQ ID NO: 126 - DNA, 20 bp, synthetic construct
  source 1..20: mol_type = other DNA
gcctggctcacctggacaac

SEQ ID NO: 127 - DNA, 20 bp, synthetic construct
  source 1..20: mol_type = other DNA
ctgttatgggcaaccctaag

SEQ ID NO: 128 - DNA, 20 bp, synthetic construct
  source 1..20: mol_type = other DNA
cagaagcctttagtgctaca

SEQ ID NO: 129 - DNA, 20 bp, synthetic construct
  source 1..20: mol_type = other DNA
gcagacaagctggcattact

SEQ ID NO: 130 - DNA, 20 bp, synthetic construct
  source 1..20: mol_type = other DNA
ccaatgtggtgtcatagacg

SEQ ID NO: 131 - DNA, 20 bp, synthetic construct
  source 1..20: mol_type = other DNA
aagctaactaggaagttgtt

SEQ ID NO: 132 - DNA, 20 bp, synthetic construct
  source 1..20: mol_type = other DNA
aaggtcaagatccattggtg

SEQ ID NO: 133 - DNA, 20 bp, synthetic construct
  source 1..20: mol_type = other DNA
agtgcatcaaagtttgggtc

SEQ ID NO: 134 - DNA, 20 bp, synthetic construct
  source 1..20: mol_type = other DNA
tcacagaaacactcaccgta

SEQ ID NO: 135 - DNA, 20 bp, synthetic construct
  source 1..20: mol_type = other DNA
tctatcagcgcagctactgc

SEQ ID NO: 136 - DNA, 20 bp, synthetic construct
  source 1..20: mol_type = other DNA
agatcgagtacatcttcaag

SEQ ID NO: 137 - DNA, 20 bp, synthetic construct
  source 1..20: mol_type = other DNA
ttctttggcaacttacccag

SEQ ID NO: 138 - DNA, 20 bp, synthetic construct
  source 1..20: mol_type = other DNA
gcaacttacccagaggcaaa

SEQ ID NO: 139 - DNA, 20 bp, synthetic construct
  source 1..20: mol_type = other DNA
tggttgcccaccctagtcat

SEQ ID NO: 140 - DNA, 20 bp, synthetic construct
  source 1..20: mol_type = other DNA
attaggagcttgagcccaga

SEQ ID NO: 141 - DNA, 20 bp, synthetic construct
  source 1..20: mol_type = other DNA
ttgcttggacaagtcctgga

SEQ ID NO: 142 - DNA, 19 bp, synthetic construct
  source 1..19: mol_type = other DNA
aatttctactcttgtagat

SEQ ID NO: 143 - DNA, 76 bp, synthetic construct
  source 1..76: mol_type = other DNA
gttttagagctagaaatagcaagttaaaataaggctagtccgttatcaacttgaaaaagtggcaccgagtcggtgc

SEQ ID NO: 144 - RNA, 39 nt, synthetic construct
  source 1..39: mol_type = other RNA
aatttctactgttgtagatnnnnnnnnnnnnnnnnnnnn

SEQ ID NO: 145 - RNA, 39 nt, synthetic construct
  source 1..39: mol_type = other RNA
aatttctactcttgtagatnnnnnnnnnnnnnnnnnnnn

SEQ ID NO: 146 - RNA, 96 nt, synthetic construct
  source 1..96: mol_type = other RNA
nnnnnnnnnnnnnnnnnnnngttttagagctagaaatagcaagttaaaataaggctagtccgttatcaacttgaaaaagtggcaccgagtcggtgc

SEQ ID NO: 147 - RNA, 39 nt, synthetic construct
  source 1..39: mol_type = other RNA
  modified_base 35..39: mod_base = OTHER; note = 2'-F nucleotide
aatttctactgttgtagatccacaacggacgacttctat

SEQ ID NO: 148 - RNA, 4151 nt, synthetic construct
  source 1..4151: mol_type = other RNA
aggagtctagagttaagaagctgggccgccaccatggccccaaagaagaagcggaaggtcgccttgttttccgagaactttgttgggtgttatccagtttctaagactttgaagtttgaactgcgcccagtgcccgaaacgaaagcatacctggatgatgataactgtccactgttggctggtgatctggagagagataaagcctacccggtggtgaaggaattgttggacaagtactataggtatttcattgacatttgtcttaaagataaaaagttgtcccccgaaaccattgacgaggcctaccaggcctacagtagccacgacgagaaatctcagaaggcactgaaggccgccagtcagaagctgaggaaggaggtcgccggatttttcgataaaaaaacccgcgaaacctactgtctcgacaagtacagtaatttgttggtgctcgacaagaaaaacgataataaagagaagggaacgcccaagacgatcccgtcagttctcaagaaatggcttgagaatgagtcaggattctccgagaaagaggtggaaaaatactcccaggctgtgcagacgtttaacggttttaccacatacttcatttcctacaaagaaaacagggagaacatgttcagcagtgaggagaaagcctctgccatctcttacagagttgttaatcagaacatgctgagatttttctccaatgtggaaaatttcgaaaacataaaaaagaagtaccctgacctttatcagacactgaaagaatttgaggaatcctttgtgcctgccgcctactcaagcctcctttctcaggccggtatagatgactataattacagagttatcggacgacctgcagaagacgaggatttcgccggagtcaatcgcctgataaatgagtataggcagaaaaatgccgtgcgaaaccggaacctgccagtgatggttactttgtacaaacagattctcagcgatcgggaaaatggtttcgggcgggacatcttggcttccgactcagaagcaaaagagtatattcgggggaagtactccaaggcttatgacaccgcctgccaactgactgtatttgtcaataattacatactgacagaagatctggacactgtgtacgttaagacccagcacctgaaggatatctctcaaagcattttcggagaatggtccgtccttgatgacgctatcaacaacggagggatcgacagcaagattatctcattgaaggctctggctgaggcattcgagaaatattgttcagcccttgatcccgatatggcccaaaaataccgggacgccttcagtttgaaggagtatctcaagaatccgcctgctgtggaacccagactgcctgaaacggccgagaaggttaccgcttataagcccgctatggatgagttgatctctctgataaggttttacaagcctttctatttgtacgacgggaagaagagactgcaggtgcctgccgagggaatggatttctctaatgagttcgacaaatatttcgaggacttgaaggaatttagtcacgcatacgataaagttcgaaattttgcaaccaagaaaccctacagcgtagagaagataaaactcaatttcaaccagccaacactcctggacgggtgggacgtgaacaaggagaaagagaatgcatcatttctctttgtcaaagagggcaagtactacctgggaattgccgacaagtccgataaaaatctgttcgatcggagtcgggatgccgtcaaagaggcactcactcttacttgcggcgaagattactatgagaaggtgcagtataagcagcttccgaacatacggatgaacctgcctcgcatagtttttaagcctaaatgggagaaggtctttgctcctataatcactgagagaatcagcgccattgagaaagaaaagctttacaccgcaggtgccaacgataggcaggcggtgatcgagtggatagacttcgtcaagtctgtccttgcgctgcactccgagtggaacaactatttccagtttaagtttaaggagacgtccgcatataaaaatacaaaagagttctacgacgatcttgataaccaggcttacatcttgagtaagatcaaaatcaacgcctcatatatccacaaactggttcgggagcgcaaattgtatctgtttcagatctattgtaaagacttttctgataagaaaaaaaagaccggcgccgacaatctccataccatgtattggagatgcctgttctccgatgaaaacttggcagctctcgcacagggacaggccccaatcataaagttgaacgggaaagctgaaatcttcctccgcgagccttctctcaaatatgaggcgacgcatccgaagaaccagccagtggccaacaagaatcccctgaactcaaaacgggagagcctgttcagctacgatctggcaaaaaatcgccgctttaccgagaggaagttgttcttccactgccccctgaccatcaactttaggacagctgcggaaggtgcgcggagcttcaatgagagagttaataggttcgtcgagaacaaccccgatatcagaataataggcattgataggggcgagagacacctgctgtactacactgtgatcaaccagagcggcaagattctggagcagggaagtctgaacaaaattagctcagattaccgaactcctgagggcgccgaggtggaaaaagtcactgactatcacgagctcttgaaccggaaagaaggagatagacgagctgcccgacagacatgggacaccatagaaaatatcaaggagcttaaagagggatacctcagccaggtggtgcataagctgactacactgatgattaaacacaatgctgtggtcgttttggagaaccttaatcaaaacttcaaacgctcccgcattaaggtcgagaagcaggtctatcagaagttcgagaaggctctgatcgataaactgaattatttggttttcaaggatagaccttacggagctcccggttcctatgccaatgggctgcagcttacggctcctttcgaatcctttgatagactcggcaagcagacaggggctctctattacgtggtgcccagctacacatcccacattgatcctaagactgggtttgtgaacctcctgggcgcaaagctccggtacgaaagcgtggtgaaagctcaggaaacaatgaagaagatcgggagtttcaagttcaaccccgagaggaattggtttgaaatcagcttcgactacggcgacttcgggatcgagatggcacatccccagtggaccgcatgtactcacggcgatgcttattggggatacaatcccaaggaaaaaagggccgttagctacgacgtgacggaagagattaaaaagctgctgacagaatacggaattgattatgggactggagcagatatcaaggaggccgctattcgacagaccaagcccgctttttggaagaccctgttttacatgctcaagctggcattgcagttgaggaataccgtggctggcactgataacgagaacgactacattctgtcaccagtcgagtatgaggccgggaagttctttgattctagaaaggctggagaggacgaaccccagaatgcagacgccaatggagcgtaccacatcgcgatgaaaggattgcaggcattgcggcgcatcgaggacggcaaagtgaacaaattcccgaagaacggtgagcgaaaagcttggctggcctttatgcaaaatagggaatatttgaaaaaacgacccgccgccacaaaaaaggccgggcaggcaaagaaaaaaaaggattacaaagacgatgacgacaagtaatagcggccgcggcccgatccagtgggcaggccaaggcctgctgggcccccgcggacccaggtgctctgggtcacggtccctgtccccgcgcccccgcttctgtctgccccattgtggctcctcaggctctctcccctgctctcccacctctacctccacccccacggatcctcaggaaaaaaaaaaaaaaaaaaaaaaaaaaaaaaaaaaaaaaaaaaaaaaaaaaaaaaaaaaaaatgcataaaaaaaaaaaaaaaaaaaaaaaaaaaaaaaaaaaaaaaaaaaaaaaaaaaaaaaaaaaa

SEQ ID NO: 149 - DNA, 39 bp, synthetic construct
  source 1..39: mol_type = other DNA
aatttctactgttgtagatggccaggcacagtggctcac

SEQ ID NO: 150 - protein, 1239 aa, synthetic construct
MALFSENFVGCYPVSKTLKFELRPVPETKAYLDDDNCPLLAGDLERDKAYPVVKELLDKYYRYFIDICLKDKKLSPETIDEAYQAYSSHDEKSQKALKAASQKLRKEVAGFFDKKTRETYCLDKYSNLLVLDKKNDNKEKGTPKTIPSVLKKWLENESGFSEKEVEKYSQAVQTFNGFTTYFSSYKRNRENMFSSEEKASAISYRVVNQNMLRFFSNVENFENIKKKYPDLYQTLKEFEESFVPAAYSSLLSQAGIDDYNYRVIGRPAEDEDFAGVNRLINEYRQKNAVRNRNLPVMVTLYKQILSDRENGFGRDILASDSEAKEYIRGKYSKAYDTACQLTVFVNNYILTEDLDTVYVKTQHLKDISQSIFGEWSVLDDAINNGGIDSKIISLKALAEAFEKYCSALDPDMAQKYRDAFSLKEYLKNPPAVEPRLPETAEKVTAYKPAMDELISLIRFYKPFYLYDGKKRLQVPAEGMDFSNEFDKYFEDLKEFSHAYDKVRNFATKKPYSVEKIKLNFNQPTLLRGWDVNVEKNRASFLFVKEGKYYLGIADKSDKNLFDRSRDAVKEALTLTCGEDYYEKVQYRQLPNIRMNLPRIVFKPKWEKVFAPIITERISAIEKEKLYTAGANDRQAVIEWIDFVKSVLALHSEWNNYFQFKFKETSAYKNTKEFYDDLDNQAYILSKIKINASYIHKLVRERKLYLFQIYCKDFSDKKKKTGADNLHTMYWRCLFSDENLAALAQGQAPIIKLNGKAEIFLREPSLKYEATHPKNQPVANKNPLNSKRESLFSYDLAKNRRFTERKLFFHCPLTINFRTAAEGARSFNERVNRFVENNPDIRIIGIDRGERHLLYYTVINQSGKILEQGSLNKISSDYRTPEGAEVEKVTDYHELLNRKEGDRRAARQTWDTIENIKELKEGYLSQVVHKLTTLMIKHNAVVVLENLNQNFKRSRIKVEKQVYQKFEKALIDKLNYLVFKDRPYGAPGSYANGLQLTAPFESFDRLGKQTGALYYVVPSYTSHIDPKTGFVNLLGAKLRYESVVKAQETMKKIGSFKFNPERNWFEISFDYGDFGIEMAHPQWTACTHGDAYWGYNPKEKRAVSYDVTEEIKKLLTEYGIDYGTGADIKEAAIRQTKPAFWKTLFYMLKLALQLRNTVAGTDNENDYILSPVEYEAGKFFDSRKAGEDEPQNADANGAYHIAMKGLQALRRIEDGKVNKFPKNGERKAWLAFMQNREYLK

SEQ ID NO: 151 - protein, 1239 aa, synthetic construct
MALFSENFVGCYPVSKTLKFELRPVPETKAYLDDDNCPLLAGDLERDKAYPVVKELLDKYYRYFIDICLKDKKLSPETIDEAYQAYSSHDEKSQKALKAASQKLRKEVAGFFDKKTRETYCLDKYSNLLVLDKKNDNKEKGTPKTIPSVLKKWLENESGFSEKEVEKYSQAVQTFNGFTTYFISYKRNRENMFSSEEKASAISYRVVNQNMLRFFSNVENFENIKKKYPDLYQTLKEFEESFVPAAYSSLLSQAGIDDYNYRVIGRPAEDEDFAGVNRLINEYRQKNAVRNRNLPVMVTLYKQILSDRENGFGRDILASDSEAKEYIRGKYSKAYDTACQLTVFVNNYILTEDLDTVYVKTQHLKDISQSIFGEWSVLDDAINNGGIDSKIISLKALAEAFEKYCSALDPDMAQKYRDAFSLKEYLKNPPAVEPRLPETAEKVTAYKPAMDELISLIRFYKPFYLYDGKKRLQVPAEGMDFSNEFDKYFEDLKEFSHAYDKVRNFATKKPYSVEKIKLNFNQPTLLRGWDVNREKENASFLFVKEGKYYLGIADKSDKNLFDRSRDAVKEALTLTCGEDYYEKVQYKQLPNIRMNLPRIVFKPKWEKVFAPIITERISAIEKEKLYTAGANDRQAVIEWIDFVKSVLALHSEWNNYFQFKFKETSAYKNTKEFYDDLDNQAYILSKIKINASYIHKLVRERKLYLFQIYCKDFSDKKKKTGADNLHTMYWRCLFSDENLAALAQGQAPIIKLNGKAEIFLREPSLKYEATHPKNQPVANKNPLNSKRESLFSYDLAKNRRFTERKLFFHCPLTINFRTAAEGARSFNERVNRFVENNPDIRIIGIDRGERHLLYYTVINQSGKILEQGSLNKISSDYRTPEGAEVEKVTDYHELLNRKEGDRRAARQTWDTIENIKELKEGYLSQVVHKLTTLMIKHNAVVVLENLNQNFKRSRIKVEKQVYQKFEKALIDKLNYLVFKDRPYGAPGSYANGLQLTAPFESFDRLGKQTGALYYVVPSYTSHIDPKTGFVNLLGAKLRYESVVKAQETMKKIGSFKFNPERNWFEISFDYGDFGIEMAHPQWTACTHGDAYWGYNPKEKRAVSYDVTEEIKKLLTEYGIDYGTGADIKEAAIRQTKPAFWKTLFYMLKLALQLRNTVAGTDNENDYILSPVEYEAGKFFDSRKAGEDEPQNADANGAYHIAMKGLQALRRIEDGKVNKFPKNGERKAWLAFMQNREYLK

SEQ ID NO: 152 - protein, 1239 aa, synthetic construct; identical to SEQ ID NO: 151

SEQ ID NO: 153 - protein, 1271 aa, synthetic construct
MAPKKKRKVALFSENFVGCYPVSKTLKFELRPVPETKAYLDDDNCPLLAGDLERDKAYPVVKELLDKYYRYFIDICLKDKKLSPETIDEAYQAYSSHDEKSQKALKAASQKLRKEVAGFFDKKTRETYCLDKYSNLLVLDKKNDNKEKGTPKTIPSVLKKWLENESGFSEKEVEKYSQAVQTFNGFTTYFSSYKRNRENMFSSEEKASAISYRVVNQNMLRFFSNVENFENIKKKYPDLYQTLKEFEESFVPAAYSSLLSQAGIDDYNYRVIGRPAEDEDFAGVNRLINEYRQKNAVRNRNLPVMVTLYKQILSDRENGFGRDILASDSEAKEYIRGKYSKAYDTACQLTVFVNNYILTEDLDTVYVKTQHLKDISQSIFGEWSVLDDAINNGGIDSKIISLKALAEAFEKYCSALDPDMAQKYRDAFSLKEYLKNPPAVEPRLPETAEKVTAYKPAMDELISLIRFYKPFYLYDGKKRLQVPAEGMDFSNEFDKYFEDLKEFSHAYDKVRNFATKKPYSVEKIKLNFNQPTLLRGWDVNVEKNRASFLFVKEGKYYLGIADKSDKNLFDRSRDAVKEALTLTCGEDYYEKVQYRQLPNIRMNLPRIVFKPKWEKVFAPIITERISAIEKEKLYTAGANDRQAVIEWIDFVKSVLALHSEWNNYFQFKFKETSAYKNTKEFYDDLDNQAYILSKIKINASYIHKLVRERKLYLFQIYCKDFSDKKKKTGADNLHTMYWRCLFSDENLAALAQGQAPIIKLNGKAEIFLREPSLKYEATHPKNQPVANKNPLNSKRESLFSYDLAKNRRFTERKLFFHCPLTINFRTAAEGARSFNERVNRFVENNPDIRIIGIDRGERHLLYYTVINQSGKILEQGSLNKISSDYRTPEGAEVEKVTDYHELLNRKEGDRRAARQTWDTIENIKELKEGYLSQVVHKLTTLMIKHNAVVVLENLNQNFKRSRIKVEKQVYQKFEKALIDKLNYLVFKDRPYGAPGSYANGLQLTAPFESFDRLGKQTGALYYVVPSYTSHIDPKTGFVNLLGAKLRYESVVKAQETMKKIGSFKFNPERNWFEISFDYGDFGIEMAHPQWTACTHGDAYWGYNPKEKRAVSYDVTEEIKKLLTEYGIDYGTGADIKEAAIRQTKPAFWKTLFYMLKLALQLRNTVAGTDNENDYILSPVEYEAGKFFDSRKAGEDEPQNADANGAYHIAMKGLQALRRIEDGKVNKFPKNGERKAWLAFMQNREYLKKRPAATKKAGQAKKKKDYKDDDDK

SEQ ID NO: 154 - protein, 1271 aa, synthetic construct
MAPKKKRKVALFSENFVGCYPVSKTLKFELRPVPETKAYLDDDNCPLLAGDLERDKAYPVVKELLDKYYRYFIDICLKDKKLSPETIDEAYQAYSSHDEKSQKALKAASQKLRKEVAGFFDKKTRETYCLDKYSNLLVLDKKNDNKEKGTPKTIPSVLKKWLENESGFSEKEVEKYSQAVQTFNGFTTYFISYKRNRENMFSSEEKASAISYRVVNQNMLRFFSNVENFENIKKKYPDLYQTLKEFEESFVPAAYSSLLSQAGIDDYNYRVIGRPAEDEDFAGVNRLINEYRQKNAVRNRNLPVMVTLYKQILSDRENGFGRDILASDSEAKEYIRGKYSKAYDTACQLTVFVNNYILTEDLDTVYVKTQHLKDISQSIFGEWSVLDDAINNGGIDSKIISLKALAEAFEKYCSALDPDMAQKYRDAFSLKEYLKNPPAVEPRLPETAEKVTAYKPAMDELISLIRFYKPFYLYDGKKRLQVPAEGMDFSNEFDKYFEDLKEFSHAYDKVRNFATKKPYSVEKIKLNFNQPTLLRGWDVNREKENASFLFVKEGKYYLGIADKSDKNLFDRSRDAVKEALTLTCGEDYYEKVQYKQLPNIRMNLPRIVFKPKWEKVFAPIITERISAIEKEKLYTAGANDRQAVIEWIDFVKSVLALHSEWNNYFQFKFKETSAYKNTKEFYDDLDNQAYILSKIKINASYIHKLVRERKLYLFQIYCKDFSDKKKKTGADNLHTMYWRCLFSDENLAALAQGQAPIIKLNGKAEIFLREPSLKYEATHPKNQPVANKNPLNSKRESLFSYDLAKNRRFTERKLFFHCPLTINFRTAAEGARSFNERVNRFVENNPDIRIIGIDRGERHLLYYTVINQSGKILEQGSLNKISSDYRTPEGAEVEKVTDYHELLNRKEGDRRAARQTWDTIENIKELKEGYLSQVVHKLTTLMIKHNAVVVLENLNQNFKRSRIKVEKQVYQKFEKALIDKLNYLVFKDRPYGAPGSYANGLQLTAPFESFDRLGKQTGALYYVVPSYTSHIDPKTGFVNLLGAKLRYESVVKAQETMKKIGSFKFNPERNWFEISFDYGDFGIEMAHPQWTACTHGDAYWGYNPKEKRAVSYDVTEEIKKLLTEYGIDYGTGADIKEAAIRQTKPAFWKTLFYMLKLALQLRNTVAGTDNENDYILSPVEYEAGKFFDSRKAGEDEPQNADANGAYHIAMKGLQALRRIEDGKVNKFPKNGERKAWLAFMQNREYLKKRPAATKKAGQAKKKKDYKDDDDK

SEQ ID NO: 155 - protein, 1271 aa, synthetic construct; identical to SEQ ID NO: 154

SEQ ID NO: 156 - DNA, 3816 bp, synthetic construct
  source 1..3816: mol_type = other DNA
atggccccaaagaagaagcggaaggtcgccttgttttccgagaactttgttgggtgttatccagtttctaagactttgaagtttgaactgcgcccagtgcccgaaacgaaagcatacctggatgatgataactgtccactgttggctggtgatctggagagagataaagcctacccggtggtgaaggaattgttggacaagtactataggtatttcattgacatttgtcttaaagataaaaagttgtcccccgaaaccattgacgaggcctaccaggcctacagtagccacgacgagaaatctcagaaggcactgaaggccgccagtcagaagctgaggaaggaggtcgccggatttttcgataaaaaaacccgcgaaacctactgtctcgacaagtacagtaatttgttggtgctcgacaagaaaaacgataataaagagaagggaacgcccaagacgatcccgtcagttctcaagaaatggcttgagaatgagtcaggattctccgagaaagaggtggaaaaatactcccaggctgtgcagacgtttaacggttttaccacatacttcatttcctacaaacgaaacagggagaacatgttcagcagtgaggagaaagcctctgccatctcttacagagttgttaatcagaacatgctgagatttttctccaatgtggaaaatttcgaaaacataaaaaagaagtaccctgacctttatcagacactgaaagaatttgaggaatcctttgtgcctgccgcctactcaagcctcctttctcaggccggtatagatgactataattacagagttatcggacgacctgcagaagacgaggatttcgccggagtcaatcgcctgataaatgagtataggcagaaaaatgccgtgcgaaaccggaacctgccagtgatggttactttgtacaaacagattctcagcgatcgggaaaatggtttcgggcgggacatcttggcttccgactcagaagcaaaagagtatattcgggggaagtactccaaggcttatgacaccgcctgccaactgactgtatttgtcaataattacatactgacagaagatctggacactgtgtacgttaagacccagcacctgaaggatatctctcaaagcattttcggagaatggtccgtccttgatgacgctatcaacaacggagggatcgacagcaagattatctcattgaaggctctggctgaggcattcgagaaatattgttcagcccttgatcccgatatggcccaaaaataccgggacgccttcagtttgaaggagtatctcaagaatccgcctgctgtggaacccagactgcctgaaacggccgagaaggttaccgcttataagcccgctatggatgagttgatctctctgataaggttttacaagcctttctatttgtacgacgggaagaagagactgcaggtgcctgccgagggaatggatttctctaatgagttcgacaaatatttcgaggacttgaaggaatttagtcacgcatacgataaagttcgaaattttgcaaccaagaaaccctacagcgtagagaagataaaactcaatttcaaccagccaacactcctgcgcgggtgggacgtgaaccgggagaaagagaatgcatcatttctctttgtcaaagagggcaagtactacctgggaattgccgacaagtccgataaaaatctgttcgatcggagtcgggatgccgtcaaagaggcactcactcttacttgcggcgaagattactatgagaaggtgcagtataagcagcttccgaacatacggatgaacctgcctcgcatagtttttaagcctaaatgggagaaggtctttgctcctataatcactgagagaatcagcgccattgagaaagaaaagctttacaccgcaggtgccaacgataggcaggcggtgatcgagtggatagacttcgtcaagtctgtccttgcgctgcactccgagtggaacaactatttccagtttaagtttaaggagacgtccgcatataaaaatacaaaagagttctacgacgatcttgataaccaggcttacatcttgagtaagatcaaaatcaacgcctcatatatccacaaactggttcgggagcgcaaattgtatctgtttcagatctattgtaaagacttttctgataagaaaaaaaagaccggcgccgacaatctccataccatgtattggagatgcctgttctccgatgaaaacttggcagctctcgcacagggacaggccccaatcataaagttgaacgggaaagctgaaatcttcctccgcgagccttctctcaaatatgaggcgacgcatccgaagaaccagccagtggccaacaagaatcccctgaactcaaaacgggagagcctgttcagctacgatctggcaaaaaatcgccgctttaccgagaggaagttgttcttccactgccccctgaccatcaactttaggacagctgcggaaggtgcgcggagcttcaatgagagagttaataggttcgtcgagaacaaccccgatatcagaataataggcattgataggggcgagagacacctgctgtactacactgtgatcaaccagagcggcaagattctggagcagggaagtctgaacaaaattagctcagattaccgaactcctgagggcgccgaggtggaaaaagtcactgactatcacgagctcttgaaccggaaagaaggagatagacgagctgcccgacagacatgggacaccatagaaaatatcaaggagcttaaagagggatacctcagccaggtggtgcataagctgactacactgatgattaaacacaatgctgtggtcgttttggagaaccttaatcaaaacttcaaacgctcccgcattaaggtcgagaagcaggtctatcagaagttcgagaaggctctgatcgataaactgaattatttggttttcaaggatagaccttacggagctcccggttcctatgccaatgggctgcagcttacggctcctttcgaatcctttgatagactcggcaagcagacaggggctctctattacgtggtgcccagctacacatcccacattgatcctaagactgggtttgtgaacctcctgggcgcaaagctccggtacgaaagcgtggtgaaagctcaggaaacaatgaagaagatcgggagtttcaagttcaaccccgagaggaattggtttgaaatcagcttcgactacggcgacttcgggatcgagatggcacatccccagtggaccgcatgtactcacggcgatgcttattggggatacaatcccaaggaaaaaagggccgttagctacgacgtgacggaagagattaaaaagctgctgacagaatacggaattgattatgggactggagcagatatcaaggaggccgctattcgacagaccaagcccgctttttggaagaccctgttttacatgctcaagctggcattgcagttgaggaataccgtggctggcactgataacgagaacgactacattctgtcaccagtcgagtatgaggccgggaagttctttgattctagaaaggctggagaggacgaaccccagaatgcagacgccaatggagcgtaccacatcgcgatgaaaggattgcaggcattgcggcgcatcgaggacggcaaagtgaacaaattcccgaagaacggtgagcgaaaagcttggctggcctttatgcaaaatagggaatatttgaaaaaacgacccgccgccacaaaaaaggccgggcaggcaaagaaaaaaaaggattacaaagacgatgacgacaagtaa

SEQ ID NO: 157 - DNA, 3816 bp, synthetic construct; identical to SEQ ID NO: 156
  source 1..3816: mol_type = other DNA

SEQ ID NO: 158 - RNA, 42 nt, synthetic construct
  source 1..42: mol_type = other RNA
aatttctactattgtagatnnnnnnnnnnnnnnnnnnnnnnn

SEQ ID NO: 159 - RNA, 42 nt, synthetic construct
  source 1..42: mol_type = other RNA
aatttctactatcgtagatnnnnnnnnnnnnnnnnnnnnnnn

SEQ ID NO: 160 - RNA, 42 nt, synthetic construct
  source 1..42: mol_type = other RNA
aatttctactgttgtagatnnnnnnnnnnnnnnnnnnnnnnn

SEQ ID NO: 161 - RNA, 42 nt, synthetic construct
  source 1..42: mol_type = other RNA
catttctacttttgtagatnnnnnnnnnnnnnnnnnnnnnnn

SEQ ID NO: 162 - RNA, 42 nt, synthetic construct
  source 1..42: mol_type = other RNA
aatttctacttttgtagatnnnnnnnnnnnnnnnnnnnnnnn

SEQ ID NO: 163 - RNA, 42 nt, synthetic construct
  source 1..42: mol_type = other RNA
gatttctacttttgtagatnnnnnnnnnnnnnnnnnnnnnnn

SEQ ID NO: 164 - RNA, 42 nt, synthetic construct
  source 1..42: mol_type = other RNA
aatttctactttcgtagatnnnnnnnnnnnnnnnnnnnnnnn

SEQ ID NO: 165 - protein, 237 aa, synthetic construct
KLGKQSGCLFYIPAWNTSKIDPVTGFTNQLDTRYQNVEKALQFFGRFDSIRYNKEKDWFEFMLDYDKFTTKAEGTQTRWTLCTYGTRIRSFRNDINKWVPKDVVLTEEFKQAFAAANIDVNGNLKDAICNLNEKAHLEELMHLVGLLVQLRNSKTNTEIDYMLSPVADKNGVFYDSRTCPDTLPKDADANGAYNIARKGIWVIRKIQEDQTNDKVDLKITNKEWLAFAQQKPYLKHL

SEQ ID NO: 166 - protein, 234 aa, synthetic construct
KTQKGAIRQCGFIFYIPAWRTSKIDPVTGFVPMLRCQYESIVESKKFFGKFDSIYYDATGKYFVFQTDFTKFNTESKGGIQKWDICTYGDRIYAPRTKDRNNNPVSERVNLTEEMKSLFVSHNINIQGDIKAGIMQQTDKEFFESLHRLLRLTLQIRNSKKSTGKDYEDYIISPVMGKDGRFFDSRNADATQPKDADANGAYNIARKGLMLLRQIQAQEKQDLSNGKWLEFAQR

SEQ ID NO: 167 - protein, 238 aa, synthetic construct
KLGKQSGFLFYIPAWNTSKIDPVTGFVNLLDTRCQNKNKAKAFFAKFDSIRYNKDKDWFEFALDYDKFGSKAEGTRTKWILCTQGKRIKTFRNAEKNFQWYNQVIDLTKDLKQLFAQYDIDINGNLKEAISKQTKKTFFDDLMGLLKLTLQMRNSITRTETDYLISPVADEIGNFYDSRTCGPSLPENADANGAFNIARKGLMIIEQIKASEDLSKLKFDISNKSWLNFAQQKPYKHE

SEQ ID NO: 168 - protein, 244 aa, synthetic construct
KLGKQSGILFYLPAAYTSKIDPLTGFINQVPWKYTNLEAAQKWIGNFKRISYHAELDLFEWVLDQRDFLSNPAGCKHQWVLRMDNQERWHWNSKAEGTNGKGAYSPIRLKEQFEDLLARQNISYNGNSKLLEDIQAVKDASFFKTLFFFLRTLTQLRYNNGKSGAEEEDFILSAVANPGMEPFDTRKAKQGLPLDADANGAYHIAMKGLMTLGQLKEKPNGKLEIPKLDKSAWLCFMQQRSNQG

SEQ ID NO: 169 - protein, 275 aa, synthetic construct
KIGKQTGIIFYTQASYTSVTDPLTGFRKNIYVSNSAPIEKIKVAINNFEEIGWDDVVQSYFFNYDTKNFSNGNDIQSKKWKIYAKVPRIKREKVNGYWQYNDVDPNDMLEKLFRLYEFKNLRSNMLEQIHAKETNGELRGERDFDNKPRNFYQSLVYILNLILQLRNSTSKQIKLNEKEDVYETGEDVDFIASPAYPFFTTLSSKSNSNLAGFENRFTGLNHDQEQKKFNGDANGAYNIARKGIIILERITKNSDKPNLYISNEDWDKFVSQSYP

SEQ ID NO: 170 - protein, 246 aa, synthetic construct
KYKQKQCGFLFYVPAWNTSKIDPLTGFVDFLKPKYESIEKAQNFLAKFKSIRYNPAKQWFEFTFDYKDFTDKAEGSRTRWTLCSTNQPRYRWNPKAANGKGAQEAVNVTQALEDLFSAHNIAYGSADDLVTAITAQSSADFYKALLRAMVTLLALRHNNGKAGTDEQDYILSPVEPFFDSRLADDSRPKDADANGAYHIAKKGLWVLQQINELQGDDWKNLKLAISNKQWLQFAQQLNADISPKVS

SEQ ID NO: 171 - protein, 251 aa, synthetic construct
DIEGKKQFGIMFYTKADYTSQTDPVTGWRKTIYLKKGSEESIKKQILDNFDDIKFDGKDYYFKYTDRNTNKPWELYSGKDGESLDRYYRELVHENSDKKWVPKEQNLKKILDGLFEGFDKEKSLLEQLRQGKNPNKINDRTSWESLRFVIDLIQQIRNSGKTEEDADFILSPVRDKNGNHFDSRKINLNLPTSGDANGAYNIARKGIIMCEHIKRDLSLFISDEEWSAWLAGREIWENWIKKNEKNLKRKQ

SEQ ID NO: 172 - protein, 234 aa, synthetic construct
THDVQCGCIFYVSAWNTSKIDPVTGFVNMLDTRFENVSSSRSFFSHFDSIRYNVAKDWFEFTFDYNNFHKKLEGTKTRWTLCTYGTRILTQRNDRNQWESREVDITDEFKKAFVAAGIDIHGNLKDAICALDKRVHLEQLMRLMKLLLQLRNSIINQDTDYILSPVAENGVFYDSRRHADNLPIDADANGAYNIARKGLYLLRKIKADRSAKPELCISNKEWLRFVQDKSNPGD

SEQ ID NO: 173 - protein, 253 aa, synthetic construct
DVGKQCGWLFYVPAAYTSKIDPKTGFANLLNMKDLTNVAKKRMFFSKFKDIHFDSGSNDFAFTFDYKDFDGKGAAEMTQTEWTVWSNGKRIFYNSKHHSTEEINVSEKLKELFDASKIDYKTENNIFESIMEVGADLKEGEKPSKEIANFWDSLLWYFKLILQMRNSIPNSETDYLISPVKAEDGTFFDSRVQKELGKDSKLPIDADANGAYHIALKGLYLLKNDFNLNDKGYIENISNAEWFRFVQERDYLN

SEQ ID NO: 174 - protein, 233 aa, synthetic construct
DIENRKQFGVVLYTRANYTSQTDPVTGWRKTIYLKTGSEDIIKKQIFESFSNIYFDGQDYIFTYTDKNIDRKWELYSGKNGKSLDRYRGRKVNNKGWVIDKINIQEILDKIFINFEICYGEKNNIFEKLKAGVELEKYNEYTAWESLRFAIDIIQQIRNTGIESSDSDFLLSPIRVNNLHFDSRLASENQPNSGDANGAYNIARKGIIMANYIKQGVSPFVSDIDWDNFVTKN

SEQ ID NO: 175 - protein, 271 aa, synthetic construct
KLGKQSGILFYTTAAYTSTTDPVTGFLKNVYKPYANVVDSSDFWKSFDEIHYDVKKERFEFTYDLRKIKNKQVDRDKDEAKVSRSVWTVCSCVERSRYVKRKANEEQKQGAASKSIGKMGEYEVFVVTDKLKALFEKESISYQSGEDIRSQLSNRIDKKDAKLHRELIYYFGTLLNMRVTDKQSESGTDENDYILSPIEPFFDSRKGYEKLPVNGDANGAYNIARKGIAILNRIHESDSGDKVDLMIRKKDWQDFVQNEEVVQKQMGKLDL

SEQ ID NO: 176 - protein, 236 aa, synthetic construct
RLGKQTGALYYVVPSYTSHIDPKTGFVNLLGAKLRYESVVKAQETMKKIGSFKFNPERNWFEISFDYGDFGIEMAHPQWTACTHGDAYWGYNPKEKRAVSYDVTEEIKKLLTEYGIDYGTGADIKEAAIRQTKPAFWKTLFYMLKLALQLRNTVAGTDNENDYILSPVEYEAGKFFDSRKAGEDEPQNADANGAYHIAMKGLQALRRIEDGKVNKFPKNGERKAWLAFMQNREYLK

SEQ ID NO: 177 - protein, 246 aa, synthetic construct
DIGNQCGFLFYVPAAYTSKIDPSTGFVNLINFNKYKNSNDRRKLICSFDKICFVQNENLFKFSIDYEKLCPNSKMPVKKWDIFSYGNRIVREDLKHIPMKENPKYDPTEELKTLFSSKEIEYQKGQNLLEIISTKDMTEDKEFWNSLFNIFKAILQMRNSLTNSPVDRLLSPVKGKDGSFFDTDKVEGTKFEKLKDADANGAYNIALKGLLVLKKNDSVKTDKDMKNVKNVSLEDWLKFVQVTLRV

SEQ ID NO: 178 - protein, 58 aa, synthetic construct
IRKVAPNNTSKTCSKCGHLNNYKFPHFKCEKCNFKENADYNAALNISNPKLKSTKEEP

SEQ ID NO: 179 - protein, 63 aa, synthetic construct
VIKIDPQYTSQRCSECGNIDSGNRIGQAIFKCRACGYEANADYNAARNIAIPNIDKIIAESIK

SEQ ID NO: 180 - protein, 80 aa, synthetic construct
LVFVDPRNTSRQCPACGHAERANRPTQALFRCVACGYSGAADYVAAVNIAVRGWAAVNRPYLGEASRVSLHGSVPGSPRL

SEQ ID NO: 181 - protein, 67 aa, synthetic construct
VVHVDPRHTSQGCSACGHISRKNRPDQTTFCCTSCGFAEHADVNAARNIAQRGATGRAVSHAADDVA

SEQ ID NO: 182 - protein, 70 aa, synthetic construct
LVRVNPAYTSRTCADCGHRERTNRVSQARFVCRSCGVVAHADLNASRNIARLGEAAWNAGRESRVPAASP

SEQ ID NO: 183 - protein, 62 aa, synthetic construct
VVFVDPAHTSRTCADCGHRERADRVDQALFICRGCGVVAHADRNASRNIARKGEVAWDAGRR

SEQ ID NO: 184 - DNA, 24 bp, synthetic construct
  source 1..24: mol_type = other DNA
tntntggttgcccaccctagtcat

SEQ ID NO: 185 - DNA, 24 bp, synthetic construct
  source 1..24: mol_type = other DNA
tttgtggttgcccaccctagtcat

SEQ ID NO: 186 - DNA, 24 bp, synthetic construct
  source 1..24: mol_type = other DNA
tntngcaacttacccagaggcaaa

SEQ ID NO: 187 - DNA, 24 bp, synthetic construct
  source 1..24: mol_type = other DNA
tttggcaacttacccagaggcaaa